SEQUENCE LISTING (28 sequences)

SEQ ID NO: 1 - protein, 182 aa, Homo sapiens
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 2 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPTQGASPSYAS

SEQ ID NO: 3 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPTQGAMPSYAS

SEQ ID NO: 4 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPTPPDVGSSDPLSMVGPSQGRSPSYAS

SEQ ID NO: 5 - protein, 182 aa, synthetic construct
  REGION 1..182: note = mutant
MHPIPTSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 6 - protein, 182 aa, synthetic construct
  REGION 1..182: note = mutant
MHPTPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 7 - protein, 182 aa, synthetic construct
  REGION 1..182: note = mutant
MHPIPDSSPTLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 8 - protein, 182 aa, synthetic construct
  REGION 1..182: note = mutant
MHPIPDSSPLLQFGGQVRQRYLYTDDAQQTEAHLEIREDGTVGGAADQSPTSLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 9 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPTVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 10 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPTGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 11 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPTACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 12 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPTHLPGNKSPHRDPAPRGPARFLPLPGLPPALPEPPGILAPQPPDVGSSDPLSMVGPSQGRSPSYAS

SEQ ID NO: 13 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPTNKSPHRDPAPRGPARFLPLPGLPPALPEPPGILAPQPPDVGSSDPLSMVGPSQGRSPSYAS

SEQ ID NO: 14 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPTRDPAPRGPARFLPLPGLPPALPEPPGILAPQPPDVGSSDPLSMVGPSQGRSPSYAS

SEQ ID NO: 15 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPTPRGPARFLPLPGLPPALPEPPGILAPQPPDVGSSDPLSMVGPSQGRSPSYAS

SEQ ID NO: 16 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPTGPARFLPLPGLPPALPEPPGILAPQPPDVGSSDPLSMVGPSQGRSPSYAS

SEQ ID NO: 17 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPTRFLPLPGLPPALPEPPGILAPQPPDVGSSDPLSMVGPSQGRSPSYAS

SEQ ID NO: 18 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPTPGLPPALPEPPGILAPQPPDVGSSDPLSMVGPSQGRSPSYAS

SEQ ID NO: 19 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPTLPPALPEPPGILAPQPPDVGSSDPLSMVGPSQGRSPSYAS

SEQ ID NO: 20 - protein, 183 aa, synthetic construct
  REGION 1..183: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPTPALPEPPGILAPQPPDVGSSDPLSMVGPSQGRSPSYAS

SEQ ID NO: 21 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPTLPEPPGILAPQPPDVGSSDPLSMVGPSQGRSPSYAS

SEQ ID NO: 22 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPTPPGILAPQPPDVGSSDPLSMVGPSQGRSPSYAS

SEQ ID NO: 23 - protein, 183 aa, synthetic construct
  REGION 1..183: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPTPGILAPQPPDVGSSDPLSMVGPSQGRSPSYAS

SEQ ID NO: 24 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPTILAPQPPDVGSSDPLSMVGPSQGRSPSYAS

SEQ ID NO: 25 - protein, 183 aa, synthetic construct
  REGION 1..183: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPTPDVGSSDPLSMVGPSQGRSPSYAS

SEQ ID NO: 26 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPTVGSSDPLSMVGPSQGRSPSYAS

SEQ ID NO: 27 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPTSMVGPSQGRSPSYAS

SEQ ID NO: 28 - protein, 182 aa, synthetic construct
  REGION 1..182: note = mutant
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPTYAS

(19) United States
(12) Patent Application Publication
(10) Pub. No.: US 2026/0201005 A1
(43) Pub. Date: Jul. 16, 2026

(54) METHODS OF TREATMENT OF NASH USING MUTANT FGF-21 PEPTIDE CONJUGATES

(71) Applicant: 89Bio, Inc., San Francisco, CA (US)

(72) Inventors: Harry H. Mansbach, Kentfield, CA (US); Maya Margalit, Rehovot (IL)

(73) Assignee: 89Bio, Inc., San Francisco, CA (US)

(21) Appl. No.: 19/102,634

(22) PCT Filed: Aug. 24, 2023
(86) PCT No.: PCT/US2023/072811

§ 371 Date: Feb. 10, 2025

Related U.S. Application Data
(60) Provisional application No. 63/373,352, filed on Aug. 24, 2022.
(60) Provisional application No. 63/373,694, filed on Aug. 26, 2022.
(60) Provisional application No. 63/382,058, filed on Nov. 2, 2022.
(60) Provisional application No. 63/482,078, filed on Jan. 30, 2023.
(60) Provisional application No. 63/494,011, filed on Apr. 4, 2023.
(60) Provisional application No. 63/510,041, filed on Jun. 23, 2023.

(51) Int. Cl. C07K 14/50; A61K 9/00; A61K 38/00; A61K 47/54; A61K 47/60; A61P 1/16

(52) U.S. Cl. CPC C07K 14/50; A61K 9/0019; A61K 47/549; A61K 47/60; A61P 1/16; A61K 38/00

(57) ABSTRACT

Therapeutic regimens and uses of mutant Fibroblast Growth Factor-21 (FGF-21) peptide conjugates in the treatment of nonalcoholic steatohepatitis are provided.

### RELATED APPLICATIONS

This application claims the benefit of and priority to U.S. Provisional Patent Application Ser. No. 63/373,352, filed Aug. 24, 2022, U.S. Provisional Patent Application Ser. No. 63/373,694, filed Aug. 26, 2022, U.S. Provisional Patent Application Ser. No. 63/382,058, filed Nov. 2, 2022, U.S. Provisional Patent Application Ser. No. 63/482,078, filed Jan. 30, 2023, U.S. Provisional Patent Application Ser. No. 63/494,011, filed Apr. 4, 2023, and U.S. Provisional Patent Application Ser. No. 63/510,041, filed Jun. 23, 2023, the disclosure of each of which is incorporated herein by reference in their entireties.

### SEQUENCE LISTING

The instant application contains a Sequence Listing which includes the file entitled 180234-011706.xml, 40,011 bytes in size, which was created Aug. 20, 2023, the contents of which are incorporated herein by reference.

### FIELD OF THE INVENTION

Therapeutic regimens and uses of mutant Fibroblast Growth Factor-21 (FGF-21) peptide conjugates comprising a polyethylene glycol (PEG) moiety attached to a mutant FGF-21 peptide via a glycosyl moiety thereof are provided.

### BACKGROUND OF THE INVENTION

FGF-21 is an endocrine hormone that is naturally found as a monomeric non-glycosylated protein. Together with FGF-19 and FGF-23, FGF-21 belongs to the endocrine-acting sub-family while the remaining of the 18 mammalian FGF ligands are grouped into five paracrine-acting sub-families.

### SUMMARY OF THE INVENTION

Provided herein are methods of treating nonalcoholic steatohepatitis (NASH) in a subject in need thereof. In some embodiments, the methods comprise administering once a week to the subject in need thereof a pharmaceutical composition comprising from about 15 mg to about 30 mg of a mutant Fibroblast Growth Factor-21 (FGF-21) peptide conjugate and a pharmaceutically acceptable carrier, wherein the mutant FGF-21 peptide conjugate comprises i) a mutant FGF-21 peptide comprising the amino acid sequence of SEQ ID NO: 2, ii) a glycosyl moiety, and iii) a 20 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG, wherein administration of the pharmaceutical composition results in at least one of: a reduction of liver fat, an improvement of liver fibrosis score, resolution of NASH, ≥2 point improvement of NAFLD Activity score, improvement of VCTE score, improvement of FAST score, improvement of FIB-4 score, a reduction of the liver size as assessed by Magnetic resonance imaging-Proton density fat fraction, a reduction of the levels of one or more biomarkers comprising Pro-C3, alanine transaminase (ALT), Enhanced LiverFibrosis (ELF) panel, CK-18, inflammation marker high-sensitivity C-reactive protein (hs-CRP), Hemoglobin Alc (HbA1c), non-HDL-c, LDL-c, and Triglycerides, and an increase of the levels of HDL-c and/or adiponectin.

In some embodiments, the subject is a human subject.

In some embodiments, the pharmaceutical composition is administered sub-subcutaneously.

In some embodiments, the glycosyl moiety comprises at least one of an N-acetylgalactosamine (GalNAc) residue, a galactose (Gal) residue, a sialic acid (Sia) residue, a 5-amine analogue of a Sia residue, a mannose (Man) residue, mannosamine, a glucose (Glc) residue, an N-acetylglucosamine (GlcNAc) residue, a fucose residue, a xylose residue, or a combination thereof. In some embodiments, the glycosyl moiety comprises at least one N-acetylgalactosamine (GalNAc) residue, at least one galactose (Gal) residue, at least one sialic acid (Sia) residue, or a combination thereof. In some embodiments, the at least one Sia residue is a nine-carbon carboxylated sugar. In some embodiments, the at least one Sia residue is N-acetyl-neuraminic acid (2-keto-5-acetamido-3,5-dideoxy-D-glycero-D-galactononulopyranos-1-onic acid (Neu5Ac), N-glycolylneuraminic acid (Neu5Gc), 2-keto-3-deoxy-nonulosonic acid (KDN), or a 9-substituted sialic acid. In some embodiments, the 9-substituted sialic acid is 9-O-lactyl-Neu5Ac, 9-O-acetyl-Neu5Ac, 9-deoxy-9-fluoro-Neu5Ac, or 9-azido-9-deoxy-Neu5Ac. In some embodiments, the glycosyl moiety comprises the structure-GalNAc-Sia-.

In some embodiments, the 20 kDa PEG moiety is attached to the glycosyl moiety by a covalent bond to a linker, wherein the linker comprises at least one amino acid residue. In some embodiments, the at least one amino acid residue is a glycine (Gly). In some embodiments, the mutant FGF-21 peptide conjugate comprises the structure-GalNAc-Sia-Gly-PEG (20 kDa). In some embodiments, the mutant FGF-21 peptide conjugate comprises the structure:

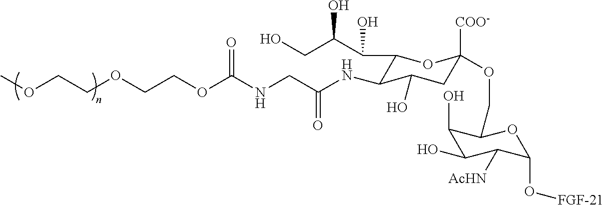

wherein n is an integer selected from 450 to 460.

In some embodiments, the 20 kDa PEG is a linear PEG. In other embodiments, the 20 kDa PEG is a branched PEG. In some embodiments, the 20 kDa PEG is a 20 kDa methoxy-PEG.

In some embodiments, the mutant FGF-21 peptide conjugate displays an equal or higher potency than wild type FGF-21 when tested in vitro in KLB-FGFR1, KLB-FGFR2 and KLB-FGFR3 expressing cells.

In some embodiments, the method comprises administering once a week to the subject in need thereof the pharmaceutical composition comprising from about 25 to about 30 mg of the mutant FGF-21 peptide conjugate.

In some embodiments, the method comprises administering once a week to the subject in need thereof the pharmaceutical composition comprising about 30 mg of the mutant FGF-21 peptide conjugate.

Provided herein are methods of treating nonalcoholic steatohepatitis (NASH) in a subject in need thereof, comprising administering once every two weeks to the subject in need thereof a pharmaceutical composition comprising from about 18 mg to about 44 mg of a mutant Fibroblast Growth Factor-21 (FGF-21) peptide conjugate and a pharmaceutically acceptable carrier, wherein the mutant FGF-21 peptide conjugate comprises i) a mutant FGF-21 peptide comprising the amino acid sequence of SEQ ID NO: 2, ii) a glycosyl moiety, and iii) a 20 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG, wherein administration of the pharmaceutical composition results in at least one of a reduction of liver fat, an improvement in liver fibrosis score, NASH resolution, ≥2 point improvement of NAFLD Activity score, improvement of VCTE score, improvement of FAST score, improvement of FIB-4 score, a reduction of the liver size as assessed by Magnetic resonance imaging-Proton density fat fraction, a reduction of the levels of one or more biomarkers comprising Pro-C3, alanine transaminase (ALT), Enhanced Liver Fibrosis (ELF) panel, CK-18, inflammation marker high-sensitivity C-reactive protein (hs-CRP), Hemoglobin A1c (HbA1c), Triglycerides, LDL-c, non-HDL-c, and an increase of the levels of HDL-c and/or adiponectin.

In some embodiments, the subject is a human subject.

In some embodiments, the pharmaceutical composition is administered sub-subcutaneously.

In some embodiments, the glycosyl moiety comprises at least one of an N-acetylgalactosamine (GalNAc) residue, a galactose (Gal) residue, a sialic acid (Sia) residue, a 5-amine analogue of a Sia residue, a mannose (Man) residue, mannosamine, a glucose (Glc) residue, an N-acetylglucosamine (GlcNAc) residue, a fucose residue, a xylose residue, or a combination thereof. In some embodiments, the glycosyl moiety comprises at least one N-acetylgalactosamine (GalNAc) residue, at least one galactose (Gal) residue, at least one sialic acid (Sia) residue, or a combination thereof. In some embodiments, the at least one Sia residue is a nine-carbon carboxylated sugar. In some embodiments, the at least one Sia residue is N-acetyl-neuraminic acid (2-keto-5-acetamido-3,5-dideoxy-D-glycero-D-galactononulopyranos-1-onic acid (Neu5 Ac), N-glycolylneuraminic acid (Neu5Gc), 2-keto-3-deoxy-nonulosonic acid (KDN), or a 9-substituted sialic acid. In some embodiments, the 9-substituted sialic acid is 9-O-lactyl-Neu5Ac, 9-O-acetyl-Neu5Ac, 9-deoxy-9-fluoro-Neu5Ac, or 9-azido-9-deoxy-Neu5Ac. In some embodiments, the glycosyl moiety comprises the structure-GalNAc-Sia-.

In some embodiments, the 20 kDa PEG moiety is attached to the glycosyl moiety by a covalent bond to a linker, wherein the linker comprises at least one amino acid residue. In some embodiments, the at least one amino acid residue is a glycine (Gly). In some embodiments, the mutant FGF-21 peptide conjugate comprises the structure -GalNAc-Sia-Gly-PEG (20 kDa). In some embodiments, the mutant FGF-21 peptide conjugate comprises the structure:

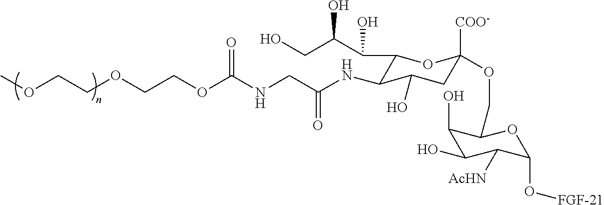

wherein n is an integer selected from 450 to 460.

In some embodiments, the 20 kDa PEG is a linear or branched PEG. In some embodiments, the 20 kDa PEG is a 20 kDa methoxy-PEG.

In some embodiments, the mutant FGF-21 peptide conjugate displays an equal or higher potency than wild type FGF-21 when tested in vitro in KLB-FGFR1, KLB-FGFR2 and KLB-FGFR3 expressing cells.

In some embodiments, the method comprises administering once a week to the subject in need thereof the pharmaceutical composition comprising from about 40 to about 50 mg of the mutant FGF-21 peptide conjugate.

In some embodiments, the method comprises administering once every two weeks to the subject in need thereof the pharmaceutical composition comprising about 44 mg of the mutant FGF-21 peptide conjugate.

### BRIEF DESCRIPTION OF THE DRAWINGS

FIG. 1 represents the single ascending dose (SAD) study design according to some embodiments.

FIG. 2 is a table showing the baseline demographics design according to some embodiments.

FIG. 3 is a table showing the baseline laboratory parameters according to some embodiments.

FIG. 4 is a table showing the safety summary according to some embodiments.

FIGS. 5A and 5B show the pharmacokinetics of the compounds described herein as single dose according to some embodiments. PK profiles are generally dose proportional with T_{1/2} range from about 53 hours to 100 hours.

FIGS. 6A and 6B are graphs showing the mean percentage change at day 8 and day 15 of rom baseline in triglycerides (%) according to some embodiments.

FIG. 7 is a graph showing the mean percentage change over time of from baseline in triglycerides (%) according to some embodiments.

FIGS. 8A and 8B are graphs showing the mean percentage change at day 8 and day 15 of rom baseline in total cholesterol (%) according to some embodiments.

FIGS. 9A and 9B are graphs showing the mean percentage change at day 8 and day 15 of rom baseline in LDL cholesterol (%) according to some embodiments.

FIGS. 10A and 10B are graphs showing the mean percentage change at day 8 and day 15 of rom baseline in HDL cholesterol (%) according to some embodiments.

FIG. 11 is a table showing comparison of study population to other FGF-21 SAD studies.

FIG. 12 is a table showing the phase 1 study comparison vs other FGF-21 products.

FIG. 13 is a table showing the phase 1 study comparison vs other FGF-21 products.

FIG. 14 is a table showing the phase 1 study comparison vs thyroid hormone receptor beta agonists and FXR.

FIG. 15 shows the pERK Functional Assay standard procedure according to some embodiments.

FIGS. 16A and 16C show the results of the pERK functional assay in KLB only expressing cells using FGF-21. FIGS. 16B and 16D show the results of the pERK functional assay in KLB only expressing cells using BIO89-100 according to some embodiments. In KLB expressing cells, FGF-21 and BIO89-100 were not active in ERK phosphorylation (pERK).

FIGS. 17A and 17C show the results of the pERK functional assay in KLB/FGFR1 expressing cells using FGF-21. FIGS. 17B and 17D show the results of the pERK functional assay in KLB/FGFR1 expressing cells using BIO89-100 according to some embodiments. Both FGF-21 and BIO89-100 activate KLB-FGFR1 cells. BIO89-100 showed 15 to 20 fold increase in potency sensitivity over FGF-21 in pERK assay.

FIGS. 18A and 18C show the results of the pERK functional assay in KLB/FGFR2 expressing cells using FGF-21. FIGS. 18B and 18D show the results of the pERK functional assay in KLB/FGFR2 expressing cells using BIO89-100 according to some embodiments. Both FGF-21 and BIO89-100 activate KLB-FGFR2 cells. BIO89-100 showed 3 to 5 fold increase in potency sensitivity over FGF-21 in pERK assay.

FIGS. 19A and 19C show the results of the pERK functional assay in KLB/FGFR3 expressing cells using FGF-21. FIGS. 19B and 19D show the results of the pERK functional assay in KLB/FGFR3 expressing cells using BIO89-100 according to some embodiments. In KLB/FGFR3 expressing cells, BIO89-100 showed a comparable potency to FGF-21.

FIGS. 20A and 20C show the results of the pERK functional assay in KLB/FGFR4 expressing cells using FGF-21. FIGS. 20B and 20D show the results of the pERK functional assay in KLB/FGFR4 expressing cells using BIO89-100 according to some embodiments. In KLB/FGFR4 expressing cells, FGF-21 and BIO89-100 were almost not active, with less than 2-fold increase in pERK at the highest concentration of 3000 nM.

FIG. 21 shows a summary of FGF-21 vs BIO89-100 according to some embodiments showing that BIO89-100 was more potent than FGF-21 but with similar activity.

FIGS. 22A and 22C show the results of the pERK functional assay in KLB only expressing cells using FGF-19. FIGS. 22B and 22D show the results of the pERK functional assay in KLB only expressing cells using FGF-21 according to some embodiments. In KLB expressing cells, FGF-19 and FGF-21 were not active (about 2-fold increase of pERK at 3000 nM).

FIGS. 23A and 23C show the results of the pERK functional assay in KLB/FGFR1 expressing cells using FGF-19. FIGS. 23B and 23D show the results of the pERK functional assay in KLB/FGFR1 expressing cells using FGF-21 according to some embodiments.

FIGS. 24A and 24C show the results of the pERK functional assay in KLB/FGFR2 expressing cells using FGF-19. FIGS. 24B and 24D show the results of the pERK functional assay in KLB/FGFR2 expressing cells using FGF-21 according to some embodiments.

FIGS. 25A and 25C show the results of the pERK functional assay in KLB/FGFR3 expressing cells using FGF-19. FIGS. 25B and 25D show the results of the pERK functional assay in KLB/FGFR3 expressing cells using FGF-21 according to some embodiments.

FIGS. 26A and 26C show the results of the pERK functional assay in KLB/FGFR4 expressing cells using FGF-19. FIGS. 26B and 26D show the results of the pERK functional assay in KLB/FGFR4 expressing cells using FGF-21 according to some embodiments. There was no activity of FGF-21 while activity and potency for FGF-19.

FIGS. 27A and 27D show the results of the pERK functional assay in KLB only expressing cells using EGF. FIGS. 27B and 27E show the results of the pERK functional assay in KLB only expressing cells using FGF-21. FIGS. 27C and 27F show the results of the pERK functional assay in KLB only expressing cells using FGF-23.

FIGS. 28A and 28D show the results of the pERK functional assay in KLB/FGFR1 expressing cells using EGF. FIGS. 28B and 28E show the results of the pERK functional assay in KLB/FGFR1 expressing cells using FGF-21. FIGS. 28C and 28F show the results of the pERK functional assay in in KLB/FGFR1 expressing cells using FGF-23.

FIGS. 29A and 29D show the results of the pERK functional assay in KLB/FGFR2 expressing cells using EGF. FIGS. 29B and 29E show the results of the pERK functional assay in KLB/FGFR2 expressing cells using FGF-21. FIGS. 29C and 29F show the results of the pERK functional assay in in KLB/FGFR2 expressing cells using FGF-23.

FIGS. 30A and 30D show the results of the pERK functional assay in KLB/FGFR3 expressing cells using EGF. FIGS. 30B and 30E show the results of the pERK functional assay in KLB/FGFR3 expressing cells using FGF-21. FIGS. 30C and 30F show the results of the pERK functional assay in in KLB/FGFR3 expressing cells using FGF-23.

FIGS. 31A and 31D show the results of the pERK functional assay in KLB/FGFR4 expressing cells using EGF. FIGS. 31CB and 31E show the results of the pERK functional assay in KLB/FGFR4 expressing cells using FGF-21. FIGS. 31C and 31F show the results of the pERK functional assay in in KLB/FGFR4 expressing cells using FGF-23.

FIGS. 32A-32C provide a summary of the pERK functional assay in KLB only, KLB/FGFR1, KLB/FGFR2, KLB/FGFR3, and KLB/FGFR4 expressing cells.

FIGS. 33A-33F show the comparison of FGF-21 and BIO89-100 potency.

FIGS. 34A-34F show the potency of FGF-21 in FGFR expressing cells (dose response curve of 3 independent experiments).

FIGS. 35A-35G show the potency of FGF-21 in FGFR expressing cells (mean of 3 independent experiments).

FIGS. 36A-36F show the potency of BIO89-100 in FGFR expressing cell (dose response curve of 2 independent experiments) s.

FIGS. 37A-37G show the potency of BIO89-100 in FGFR expressing cells (mean of 2 independent experiments).

FIG. 38A shows Phase 1b/2a NASH Trial Design

FIG. 38B is a table showing the baseline characteristics according to some embodiments.

FIG. 39A is a graph showing the mean reduction in liver fat vs. baseline. FIG. 39B is a table showing the relative reduction in liver fat in different cohorts. FIG. 39A and FIG. 39B show that BIO89-100 demonstrated robust liver fat reduction in high responder rates.

FIG. 40A is a graph showing the mean percent change vs. baseline. FIG. 40B is a graph showing ALT-absolute change value vs. baseline.

FIG. 41A is a graph showing ≥2 pt improvement in NAS. *with ≥2 pt improvement in ballooning or inflammation. FIG. 41B is a graph showing responder rates by NAS component.

FIG. 42 is a graph changes on key histological efficacy endpoints.

FIG. 43A is a graph showing VCTE, FAST score, FIB-4 score and Pro-C3 mean relative change from baseline. Values in bars are absolute change versus baseline. **p<0.01; ***p<0.001.

FIG. 43B is a graph showing responders by clinically relevant thresholds.

FIG. 44A is a graph showing the absolute change in HbA1c. FIG. 44B is a graph showing the change in adiponectin. FIG. 44C is a graph showing the weight change. P value for change from baseline based on MMRM analysis; all data are from cohort 7. **p<0.01; ***p<0.001.

FIG. 45 is a graph showing the TG, LDL-C, non-HDL-C and HDL-C percent change from baseline at week 20. *p<0.05; ***p<0.001.

FIG. 46A are graphs showing improvement in liver health. FIG. 46B, and FIG. 46C are graphs showing improvement in co-morbidities associated with NASH.

FIG. 47 is a table showing the phase 2b NASH trial design.

FIGS. 48A-48B: Arithmetic mean pegozafermin serum concentration-time profiles across dose regimens. Shown as steady-state value on day 29 for QW (FIG. 48A) and Q2W (FIG. 48B) regimen. Day 57 concentrations (i.e. 336 hours after the day 43 dose rather than the day 29 dose) were used as an equivalent trough values for the Q2W regimen (FIG. 48B)

FIG. 49: Correlation of percent change from baseline in alanine aminotransferase (ALT) levels at week 13 with percent change in MRI-PDFF at week 13.

FIG. 50: Responder analysis of hepatic steatosis as assessed by MRI-PDFF (pharmacodynamics-MRI population).

FIG. 51: Summary of protocol amendments.

FIG. 52: Additional supporting text for methods

FIG. 53: Dosing and assessment schedule (cohorts 1-4).

FIG. 54: Dosing and assessment schedule (cohorts 5 and 6).

FIG. 55: Population analysis sets.

FIG. 56: Change in hepatic fat fraction (Pharmacodynamics population-MRI population).

FIG. 57: Change in hepatic volume (Pharmacodynamics population).

FIG. 58: Change in alanine aminotransferase (ALT) levels (Pharmacodynamics population).

FIG. 59: Change in alanine aminotransferase (ALT) levels in patients with baseline ALT levels >45 U/L (Pharmacodynamics population).

FIG. 60: Change in aspartate aminotransferase (AST) levels (Pharmacodynamics population).

FIG. 61: Change in N-terminal propeptide of type III collagen (PRO-C3) levels (Pharmacodynamics population)

FIG. 62: Change in triglyceride levels (Pharmacodynamics population).

FIG. 63: Change in low-density lipoprotein cholesterol (LDL-C) levels (Pharmacodynamics population)

FIG. 64: Change in high-density lipoprotein cholesterol (HDL-C) levels (Pharmacodynamics population).

FIG. 65: Change in non-high-density lipoprotein cholesterol (non-HDL-C) levels (Pharmacodynamics population).

FIG. 66: Change in Homeostatic Model Assessment of Insulin Resistance (HOMA-IR) levels (Pharmacodynamics population).

FIG. 67: Change in glucose levels (Pharmacodynamics population).

FIG. 68: Change in glycated haemoglobin (HbA1C) levels (Pharmacodynamics population).

FIG. 69: Change in weight (Pharmacodynamics population).

FIG. 70: Change in adiponectin levels (Pharmacodynamics population)

FIG. 71: Change in free fatty acid (FFA) levels (Pharmacodynamics population).

FIG. 72: Change in adipose tissue insulin resistance index (Adipo-IR) levels (Pharmacodynamics population).

FIG. 73: Participant randomisation by site (Randomised population).

FIG. 74: Demographics and baseline characteristics of the BC-NASH and PNASH subpopulations (Randomised population).

FIG. 75: Demographics and baseline characteristics of participants who had a ≥30% relative reduction versus placebo in hepatic fat fraction (MRI-PDFF responders) versus those who did not (MRI-PDFF non-responders) (Pharmacodynamics-MRI population).

FIG. 76: Study design

FIG. 77: Primary analysis (Central Reader): Pegozafermin robustly improved NAFLD activity score (NAS) and all components of NAS.

FIG. 78: Primary Analysis (Central Reader): PGZ Demonstrated Clinically Meaningful Changes on Key Histological Efficacy Endpoints.

FIG. 79: Number of visible tumor nodules in STAM™ male mice treated with vehicle, pegozafermin or sorafenib.

FIG. 80: Phase 2b trial design. ¹Improvement in liver fibrosis by ≥1 stage and no worsening of steatohepatitis defined as no increase in NAS for ballooning, inflammation and steatosis. ²Resolution of steatohepatitis is defined as absent fatty liver disease and isolated or simple steatosis without steatohepatitis and a NAS score of 0-1 for inflammation, 0 for ballooning and any value for steatosis. *Some placebo patients were randomized in the extension phase to receive pegozafermin.

FIG. 81: Biopsy reading method design.

FIG. 82: Patient disposition and analysis sets.

FIG. 83: Baseline characteristics across does groups.

FIG. 84: Pegozafermin Demonstrated Statistical Significance on Fibrosis Improvement with Weekly and Every two-week Dosing. *Relative risk presented is calculated by dividing the drug response by placebo response. Relative risk calculated using statistical methods show similar results.

FIG. 85: Pre-Specified Additional Analyses Confirm Robustness of Primary Efficacy Results (Fibrosis Improvement Without Worsening of NASH at Week 24).

FIG. 86: Descriptive Analysis Data of Cirrhotic (F4) Patients from Clinical Trial. ¹Improvement in liver fibrosis by ≥1 stage and no worsening of steatohepatitis defined as no increase in NAS for ballooning, inflammation and steatosis.

FIG. 87: NASH Resolution Data. * Relative risk presented is calculated by dividing the drug response by placebo response. Relative risk calculated using statistical methods show similar results.

FIG. 88: Pre-Specified Additional Analyses Confirm Robustness of Primary Efficacy Results (NASH Resolution Without Worsening of Fibrosis at Week 24).

FIG. 89: Graph showing ≥2-point NAS Improvement and no Worsening of Fibrosis at Week 24. ¹Full Analysis set. Analysis by Cochran-Mantel-Haenzel (CMH) test stratified by T2DM status (yes vs no) and fibrosis stage (F2 vs F3). ***p<0.001 versus placebo.

FIG. 90A: Mean Relative Reduction in Liver Fat vs Baseline. at Week 24. ¹Analysis via mixed model repeated measure (MMRM). ***p<0.001 versus placebo. FIG. 90B: Proportion of Patients Achieving ≥50% Reductions in Liver Fat at Week 24. ²Analysis by Cochran-Mantel-Haenzel (CMH) test stratified by T2DM status (yes vs no) and fibrosis stage (F2 vs F3). ***p<0.001 versus placebo.

FIG. 91A: Mean Relative Reduction in ALT vs Baseline at Week 24. FIG. 91B: Mean Relative Reduction in AST vs Baseline at Week 24. Analysis via mixed model repeated measure (MMRM). ***p<0.001 versus placebo.

FIG. 92A: cT1 Responders at Week 24. FIG. 92B. VCTE Absolute Change from Baseline at Week 24. FIG. 92C: PRO-C3 Percent Change from Baseline at Week 24. Full Analysis Set for Fibroscan and PRO-C3 and MRI-PDFF analysis set for cT1, Analysis via MMRM for cT1 and PRO-C3, ANCOVA for VCTE. A patient is designated as a cT1 responder with ≥80 msec reduction as compared with baseline. cT1 was performed at sites where available). *p<0.05, **p<0.01, ***p<0.001 versus placebo.

FIGS. 93A-93B: Change in HbA1c from Baseline at Week 24. Full Analysis Set for either overall population or FAS with baseline HbA1c≥6.5%. Analysis via MMRM. *p<0.05, ***p<0.001 versus placebo

FIGS. 94A-96C: Percent changes in Serum Lipids from Baseline at Week 24 (FIG. 94A: triglycerides; FIG. 94B: Non-HDL Cholesterol; FIG. 94C: HDL Cholesterol). Full Analysis Set via Eltren test fro triglycerides (reported as a median) and MMRM, Subjects with missing week 24 triglycerides are excluded from the non-parametric analysis. Non-HDL-cholesterol and HDL-cholesterol (reported as LS means) with changes in baseline (absolute or %) as dependent variables. p<0.05, **p<0.01, ***p<0.001 versus placebo.

FIG. 95: Treatment Related TEAEs.

FIG. 96A is a graph showing Fibrosis Improvement ≥1 Stage Without nWorsening of NASH.

FIG. 96B is a graph showing NASH Resolution Without Worsening of Fibrosis.

FIG. 97 are graphs showing key markers (ELF score, VCTE, ALT, HbA1c, MRI-PDFF) change on patient on background GLP1.

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure are intended to be illustrative, and not restrictive.

### DETAILED DESCRIPTION

Nonalcoholic fatty liver disease (NAFLD) and Non-alcoholic steatohepatitis (NASH) are chronic conditions where fat is deposited in the liver with subsequent liver damage and inflammation. To date there are no specific therapies for these disorders.

### Definitions

For the sake of clarity and readability, the following definitions are provided. Any technical feature mentioned for these definitions may be read on each and every embodiment of the disclosure. Additional definitions and explanations may be specifically provided in the context of these embodiments. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry, and nucleic acid chemistry and hybridization are those well-known and commonly employed in the art. Standard techniques are used for nucleic acid and peptide synthesis. The techniques and procedures are generally performed according to conventional methods in the art and various general references (e.g., Sambrook et al., 1989, Molecular Cloning: A Laboratory Manual, 2d ed. Cold Spring Harbor Laboratory Press, Cold Spring Harbor, NY), which are provided throughout this document.

Enzyme: Enzymes are catalytically active biomolecules that perform biochemical reactions such as the transfer of glycosyl moieties or modified glycosyl moieties from the respective glycosyl donors to an amino acid of FGF-21 or to another glycosyl moiety attached to the peptide.

Protein: A protein typically comprises one or more peptides or polypeptides. A protein is typically folded into a 3-dimensional form, which may be required for the protein to exert its biological function. The sequence of a protein or peptide is typically understood to be in the order, i.e. the succession of its amino acids.

Recombinant protein: The term “recombinant protein” refers to proteins produced in a heterologous system, that is, in an organism that naturally does not produce such a protein, or a variant of such a protein, i.e. the protein or peptide is “recombinantly produced”. Typically, the heterologous systems used in the art to produce recombinant proteins are bacteria (e.g., Escherichia (E.) coli), yeast (e.g., Saccharomyces (S.) cerevisiae) or certain mammalian cell culture lines.

Expression host: An expression host denotes an organism which is used for recombinant protein production. General expression hosts are bacteria, such as E. coli, yeasts, such as Saccharomyces cerevisiae or Pichia pastoris, or also mammal cells, such as human cells.

RNA, mRNA: RNA is the usual abbreviation for ribonucleic acid. It is a nucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the RNA sequence.

DNA: DNA is the usual abbreviation for deoxyribonucleic acid. It is a nucleic acid molecule, i.e. a polymer consisting of nucleotide monomers. These nucleotides are usually deoxy-adenosine-monophosphate, deoxy-thymidine-monophosphate, deoxy-guanosine-monophosphate and deoxy-cytidine-monophosphate monomers which are—by themselves—composed of a sugar moiety (deoxyribose), a base moiety and a phosphate moiety, and polymerized by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA-sequence. DNA may be single-stranded or double-stranded. In the double stranded form, the nucleotides of the first strand typically hybridize with the nucleotides of the second strand, e.g. by A/T-base-pairing and G/C-base-pairing.

Sequence of a nucleic acid molecule/nucleic acid sequence: The sequence of a nucleic acid molecule is typically understood to be in the particular and individual order, i.e. the succession of its nucleotides.

Sequence of amino acid molecules/amino acid sequence: The sequence of a protein or peptide is typically understood to be in the order, i.e. the succession of its amino acids.

Sequence identity: Two or more sequences are identical if they exhibit the same length and order of nucleotides or amino acids. The percentage of identity typically describes the extent, to which two sequences are identical, i.e. it typically describes the percentage of nucleotides that correspond in their sequence position to identical nucleotides of a reference sequence, such as a native or wild type sequence. For the determination of the degree of identity, the sequences to be compared are considered to exhibit the same length, i.e. the length of the longest sequence of the sequences to be compared. This means that a first sequence consisting of 8 nucleotides/amino acids is 80% identical to a second sequence consisting of 10 nucleotides/amino acids comprising the first sequence. In other words, in the context of the present disclosure, identity of sequences particularly relates to the percentage of nucleotides/amino acids of a sequence, which have the same position in two or more sequences having the same length. Gaps are usually regarded as non-identical positions, irrespective of their actual position in an alignment.

Newly introduced amino acids: “Newly introduced amino acids” denote amino acids which are newly introduced into an amino acid sequence in comparison to a native/wild type amino acid sequence. Usually by mutagenesis, the native amino acid sequence is changed in order to have a certain amino acid side chain at a desired position within the amino acid sequence. In the present disclosure, in particular the amino acid threonine is newly introduced into the amino acid sequence on the C-terminal side adjacent to a proline residue.

Functional group: The term is to be understood according to the skilled person's general understanding in the art and denotes a chemical moiety which is present on a molecule, in particular on the peptide or amino acid of the peptide or glycosyl residue attached to the peptide, and which may participate in a covalent or non-covalent bond to another chemical molecule, i.e. which allows e.g. the attachment of a glycosyl residue or PEG.

Native amino acid sequence: The term is to be understood according to the skilled person's general understanding in the art and denotes the amino acid sequence in the form of its occurrence in nature without any mutation or amino acid amendment by man. It is also called “wild-type sequence”. “Native FGF-21” or “wild-type FGF-21” denotes FGF-21 having the amino acid sequence as it occurs in nature, such as the (not mutated) amino acid sequence of human FGF-21 as depicted in SEQ ID NO: 1. The presence or absence of an N-terminal methionine, which depends on the used expression host, usually does not change the status of a protein being considered as having its natural or native/wild-type sequence.

Mutated: The term is to be understood according to the skilled person's general understanding in the art. An amino acid sequence is called “mutated” if it contains at least one additional, deleted or exchanged amino acid in its amino acid sequence in comparison to its natural or native amino acid sequence, i.e. if it contains an amino acid mutation. Mutated proteins are also called mutants. In the present disclosure, a mutated FGF-21 peptide is particularly a peptide having an amino acid exchange adjacent to a proline residue on the C-terminal side of the proline residue. Thereby a consensus sequence for O-linked glycosylation is introduced into FGF-21 such that the mutant FGF-21 peptide comprises a newly introduced O-linked glycosylation side. Amino acid exchanges are typically denoted as follows: S¹⁷²T which means that the amino acid serine at position 172, such as in the amino acid sequence of SEQ ID NO: 1, is exchanged by the amino acid threonine.

Pharmaceutically effective amount: A pharmaceutically effective amount in the context of the disclosure is typically understood to be an amount that is sufficient to induce a pharmaceutical effect.

Therapy/treatment: The term “therapy” refers to “treating” or “treatment” of a disease or condition, inhibiting the disease (slowing or arresting its development), providing relief from the symptoms or side-effects of the disease (including palliative treatment), and relieving the disease (causing regression of the disease).

Therapeutically effective amount or effective amount: is an amount of a compound that is sufficient to treat a disease or condition, inhibit the disease or condition, provide relief from symptoms or side-effects of the disease, and/or cause regression of the disease or condition.

Half-life: The term “half-life”, as used herein in the context of administering a mutant FGF-21 peptide and/or conjugate thereof, is defined as the time required for the plasma concentration of a drug, i.e. of the mutant FGF-21 peptide and/or conjugate, in a subject to be reduced by one half.

O-linked glycosylation: “O-linked glycosylation” takes place at serine or threonine residues (Tanner et al., Biochim. Biophys. Acta. 906:81-91 (1987); and Hounsell et al, Glycoconj. J. 13:19-26 (1996)). In some embodiments, O-linked glycosylation sites, which are amino acid motifs in the amino acid sequence of a peptide which are recognized by glycosyl transferases as attachment points for glycosyl residues, include the amino acid motif proline-threonine (PT) not present in the native/wild-type amino acid sequence. In particular, the threonine residue is newly introduced adjacent to a proline and on the C-terminal side of a proline residue. The glycosyl moiety is then attached to the —OH group of the threonine residue by the glycosyl transferase.

Newly introduced O-linked glycosylation side: “Newly introduced O-linked glycosylation side” denotes an O-linked glycosylation side which did not exist in the native or wild-type FGF-21 before introducing a threonine adjacent to and on the C-terminal side of a proline residue as described herein.

Adjacent: Adjacent denotes the amino acid immediately next to another amino acid in the amino acid sequence, either on the N-terminal or on the C-terminal side of the respective amino acid. In the present disclosure, e.g. the newly introduced threonine residue is adjacent to a proline residue on the C-terminal side of a proline residue.

Glycosyl moiety: A glycosyl moiety is a moiety consisting of one or more, identical or different glycosyl residues which links the mutant FGF-21 peptide to a polyethylene glycol (PEG), thereby forming a conjugate comprising a peptide, glycosyl moiety and PEG. The glycosyl moiety can be a mono-, di-, tri-, or oligoglycosyl moiety. The glycosyl moiety may comprise one or more sialic acid residues, one or more N-acetylgalactosamine (GalNAc) residues, one or more galactose (Gal) residues and others. The glycosyl moiety may be modified, such as with a PEG or methoxy-PEG (m-PEG), an alkyl derivative of PEG.

Glycoconjugation: “Glycoconjugation”, as used herein, refers to the enzymatically mediated conjugation of a PEG-modified glycosyl moiety to an amino acid or glycosyl residue of a (poly)peptide, e.g. a mutant FGF-21 of the present disclosure. A subgenus of “glycoconjugation” is “glyco-PEGylation” in which the modifying group of the modified glycosyl moiety is PEG or m-PEG. The PEG may be linear or branched. Typically, a branched PEG has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched PEG can be represented in general form as R(-PEG-OX) m in which R represents the core moiety, such as glycerol or pentaerythritol, X represents a capping group or an end group, and m represents the number of arms. The terms “glyco-PEG” and “glycosyl-PEG” are used interchangeably and denote a chemical moiety consisting of PEG or methoxy-PEG (mPEG or m-PEG), one or more glycosyl residues (or glycosyl moieties), and optionally a linker between PEG/methoxy-PEG and the glycosyl moieties, such as an amino acid, e.g. glycine. An example of a glycosyl-PEG/glyco-PEG moiety is PEG-sialic acid (PEG-Sia). It should be noted that the terms “glyco-PEG” and “glycosyl-PEG” as well as “PEG-sialic acid” and “PEG-Sia” as well as similar terms for glyco-PEG moieties may or may not include a linker between PEG and the glycosyl moiety or moieties, i.e. “PEG-sialic acid” encompasses e.g. PEG-sialic acid as well as PEG-Gly-sialic acid as well as mPEG-Gly-sialic acid.

Sequence motif: A sequence motif denotes a short amino acid sequence, such as that comprising only two amino acids, which is present at any possible position in a longer amino acid sequence, such as in the amino acid sequence of human FGF-21. Sequence motifs are e.g. denoted as P¹⁷²T which means that the proline at position 172 is followed C-terminally immediately by a threonine residue.

Sialic acid: The term “sialic acid” or “Sia” refers to any member of a family of nine-carbon carboxylated sugars. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). A second member of the family is N-glycolylneuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261:11550-11557). Also included are 9-substituted sialic acids such as a 9-0-C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see e.g. Varki, Glycobiology 2:25-40 (1992)).

Pharmaceutically acceptable excipient: “Pharmaceutically acceptable” excipient includes any material, which when combined with the mutant FGF-21 peptide conjugate of the disclosure retains the conjugates' activity and is non-reactive with a subject's immune systems. Examples include, but are not limited to, any of the standard pharmaceutical excipients such as a phosphate buffered saline solution, water, salts, emulsions such as oil/water emulsion, and various types of wetting agents.

Pharmaceutical container: A “pharmaceutical container” is a container which is suitable for carrying a pharmaceutical composition and typically made of an inert material and sterile.

Administering: The term “administering” means oral administration, inhalation, administration as a suppository, topical contact, intravenous, intraperitoneal, intramuscular, intralesional, intranasal or subcutaneous administration, or the implantation of a slow-release device e.g., a mini-osmotic pump, to the subject. Administration is by any route including parenteral, and transmucosal (e.g. oral, nasal, vaginal, rectal, or transdermal). Parenteral administration includes e.g. intravenous, intramuscular, intraarteriole, intradermal, subcutaneous, intraperitoneal, intraventricular and intracranial. Other modes of delivery include, but are not limited to, the use of liposomal formulations, intravenous infusion, transdermal patches, etc.

Diabetes and diabetes related diseases: “Diabetes” is a well-known and well-characterized disease often referred to as diabetes mellitus. The term describes a group of metabolic diseases in which the person has high blood glucose levels (blood sugar), either because insulin production is inadequate, or because the body's cells do not respond properly to insulin, or both. Patients with high blood sugar will typically experience polyuria (frequent urination), they will become increasingly thirsty (polydipsia) and hungry (polyphagia). “Diabetes related diseases” are diseases characterized by the same symptoms such as obesity, polyuria, polydipsia and polyphagia.

Diabetes type 2: “Diabetes type 2” is the most common form of diabetes/diabetes mellitus. Diabetes type 2 most commonly develops in adulthood and is more likely to occur in people who are overweight and physically inactive. Unlike type 1 diabetes, which currently cannot be prevented, many of the risk factors for type 2 diabetes can be modified. The International Diabetes Foundation lists four symptoms that signal the need for diabetes testing: a) frequent urination, b) weight loss, c) lack of energy and d) excessive thirst. Insulin resistance is usually the precursor to diabetes type 2 a condition in which more insulin than usual is needed for glucose to enter the cells. Insulin resistance in the liver results in more glucose production while resistance in peripheral tissues means glucose uptake is impaired.

Nonalcoholic fatty liver disease (NAFLD) and Non-alcoholic steatohepatitis (NASH): are a condition where fat is deposited in the liver with subsequent liver damage and inflammation.

Metabolic syndrome: a defined cluster of risk factors (biochemical and physiological changes) that are associated with the development of type 2 diabetes and cardiovascular disease.

Abbreviations used herein include: PEG, poly(ethyleneglycol); PPG, poly(propyleneglycol); Ara, arabinosyl; Fru, fructosyl; Fuc, fucosyl; Gal, galactosyl; GalNAc, N-acetylgalactosaminyl; Glc, glucosyl; GlcNAc, N-acetylglucosaminyl; Man, mannosyl; ManAc, mannosaminyl acetate; Xyl, xylosyl; NeuAc, sialyl or N-acetylneuraminyl; Sia, sialyl or N-acetylneuraminyl; and derivatives and analogues thereof.

Natural FGF-21 has a comparatively short half-life in vivo, with a reported circulating half-life ranging from 0.5 to 4 hours in rodents and non-human primates, which limits its clinical applicability. The half-life of recombinant human FGF-21 is 1-2 hours. To improve pharmacokinetic properties of FGF-21, various half-life extension strategies have been developed.

See also WO2019/043457, the entire content of which is incorporated herein in its entirety.

### PEGylation

In glycoPEGylation, a PEG moiety may be transferred to an amino acid or glycosyl residue attached to an amino acid of the protein or peptide using a glycosyltransferase. The general final structure is protein-glycosyl moiety-optional further linker-PEG. In some embodiments, final structure is protein-(N-, C- or internal) amino acid of the protein-one or more glycosyl residues-optional linker (e.g., amino acid linker)-linear or branched PEG moiety of various lengths, wherein the glycosyl moiety may comprise one or more glycosyl residues. The one or more glycosyl residues comprising at least part of the structure linking the protein to the PEG moiety may be any possible glycosyl residue. A diverse array of methods for glycoPEGylating proteins are known in the art and are described in detail herein below.

In some embodiments, Fibroblast Growth Factor-21 (FGF-21) peptide conjugates comprise:
i) a mutant FGF-21 peptide comprising at least one threonine (T) residue adjacent to at least one proline (P) residue on the C-terminal side of said at least one proline residue, thereby forming at least one O-linked glycosylation site which does not exist in the corresponding native FGF-21, wherein the corresponding native FGF-21 has an amino acid sequence that is at least 95% identical to SEQ ID NO: 1, and
ii) a 20 kDa polyethylene glycol (PEG), wherein said 20 kDa PEG is covalently attached to said mutant FGF-21 peptide at said at least one threonine residue via at least one glycosyl moiety.

In some embodiments, the mutant FGF-21 peptide conjugate comprises a mutant FGF-21 peptide comprising the amino acid sequence PT. In some embodiments, the mutant FGF-21 peptide comprises at least one amino acid sequence selected from the group consisting of P172T, P156T, P5T, P3T, P9T, P50T, P61T, P79T, P91T, P116T, P129T, P131T, P134T, P139T, P141T, P144T, P145T, P148T, P150T, P151T, P158T, P159T, P166T, P178T and combinations thereof, wherein the positions of proline and threonine are based on the amino acid sequence as depicted in SEQ ID NO: 1. In some embodiments, the mutant FGF-21 peptide comprises at least one amino acid sequence selected from the group consisting of P172T, P156T, P5T and combinations thereof, for example consisting of P172T, P156T and combinations thereof, wherein the positions of proline and threonine are based on the amino acid sequence as depicted in SEQ ID NO: 1. In some embodiments, the proline residue is located between amino acid 145 and the C-terminus of the mutant FGF-21 peptide, wherein the position of amino acid 145 is based on the amino acid sequence as depicted in SEQ ID NO: 1.

In some embodiments, the mutant FGF-21 peptide comprises the amino acid sequence P172T, wherein the positions of proline and threonine are based on the amino acid sequence as depicted in SEQ ID NO: 1.

In some embodiments, the mutant FGF-21 peptide comprises the mutations S173T and R176A, wherein the positions of the amino acids S and R are based on the amino acid sequence as depicted in SEQ ID NO: 1, particularly the mutant FGF-21 peptide comprises the amino acid sequence as depicted in SEQ ID NO: 2.

In some embodiments, the mutant FGF-21 peptide comprises the mutation Q157T, wherein the position of the amino acid Q is based on the amino acid sequence as depicted in SEQ ID NO: 1, particularly the mutant FGF-21 peptide comprises the amino acid sequence as depicted in SEQ ID NO: 4.

In some embodiments, the mutant FGF-21 peptide comprises the mutation D6T, wherein the position of the amino acid D is based on the amino acid sequence as depicted in SEQ ID NO: 1, particularly the mutant FGF-21 peptide comprises the amino acid sequence as depicted in SEQ ID NO: 5.

In some embodiments, the mutant FGF-21 peptide comprises an amino acid sequence selected from the group consisting of SEQ ID NOs: 2 to 28. In some embodiments, the mutant FGF-21 peptide comprises an amino acid sequence selected from the group consisting of SEQ ID NOs: 2 to 5. In some embodiments, the mutant FGF-21 peptide comprises an amino acid sequence selected from the group consisting of SEQ ID NOs: 2 to 4. In some embodiments, the mutant FGF-21 peptide comprises an amino acid sequence selected from the group consisting of SEQ ID NO: 2.

In some embodiments, the mutant FGF-21 peptide conjugate comprises at least one glycosyl moiety comprising N-acetylgalactosamine (GalNAc), galactose (Gal) and/or sialic acid (Sia). In some embodiments, the at least one glycosyl moiety comprises the structure-GalNAc-Sia-.

In some embodiments, the mutant FGF-21 peptide conjugate comprises a 20 kDa PEG moiety which is attached to the at least one glycosyl moiety via an amino acid residue, particularly glycine (Gly). In some embodiments, the mutant FGF-21 peptide conjugate comprises the structure -GalNAc-Sia-Gly-PEG (20 kDa). In some embodiments, the mutant FGF-21 peptide conjugate comprises the structure:

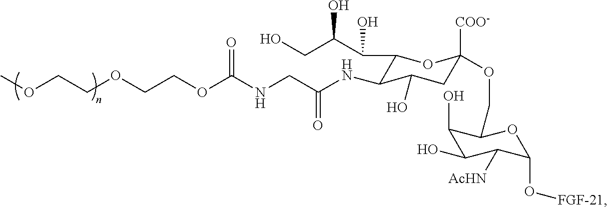

wherein n is an integer selected from 450 to 460.

In some embodiments, the mutant FGF-21 peptide conjugate comprises a 20 kDa PEG which is a linear or branched PEG, particularly a linear PEG. some embodiments, the 20 kDa PEG is a 20 kDa methoxy-PEG.

In some embodiments, encompassed herein is a pharmaceutical composition comprising at least one mutant FGF-21 peptide conjugate described herein and a pharmaceutically acceptable carrier. In some embodiments, the mutant FGF-21 peptide conjugate is present in a concentration in the range from 0.1 mg/mL to 50 mg/mL. In some embodiments, the mutant FGF-21 peptide conjugate is present in a concentration in the range from 1 mg/mL to 45 mg/mL, from 10 mg/mL to 40 mg/mL, for example 26±4 mg/mL. The buffering agent may be a Tris buffer. The buffering agent may be present in a concentration from 1 mM to 100 mM, from 2 mM to 75 mM, 5 mM to 50 mM, from 10 mM to 25 mM, for example 16±2 mM. The pH may be in the range from 6.0 to 8.5, from 6.5 to 8.0, from 6.75 to 8.0, for example 7.5±0.3. The pharmaceutical composition may further comprise a salt, including but not limited to an inorganic salt, for example NaCl. The pharmaceutical composition may comprise a salt which is present in a concentration from 30 mM to 200 mM, from 40 mM to 150 mM, from 50 mM to 100 mM, for example 56±2 mM. The pharmaceutical composition may further comprise a tonicity modifying agent. Suitable tonicity modifying agents include, but not limited to, glycerol, amino acids, sodium chloride, proteins, or sugars and sugar alcohols. In some embodiments, the tonicity modifying agent is a sugar, for example sucrose. The tonicity modifying agent is present in a concentration of 50 mM to 200 mM, of 100 mM to 175 mM, of 135 mM to 160 mM, for example 150±2 mM. The pharmaceutical composition may further comprise a surfactant, for example non-ionic surfactant. The surfactant or non-ionic surfactant may be a polysorbate-based non-ionic surfactant, for example polysorbate 20 or polysorbate 80. The surfactant or non-ionic surfactant may be present in a concentration of 0.01 mg/mL to 1 mg/mL of 0.05 to 0.5 mg/mL and for example 0.2±0.02 mg/mL.

In some embodiments, the pharmaceutical composition comprises 0.1 mg/mL to 50 mg/mL of mutant FGF-21 peptide conjugate, 1 mM to 100 mM buffering agent, for example Tris buffer, 30 mM to 200 mM salt, for example NaCl, 50 mM to 200 mM tonicity modifying agent, for example sucrose, and 0.01 mg/mL to 1 mg/mL surfactant or non-ionic surfactant, for example polysorbate 20, and has a pH of 6.0 to 8.5.

A pharmaceutical container comprising at least one of the mutant FGF-21 peptide conjugates described herein and/or a pharmaceutical composition comprising same are also encompassed herein. Suitable pharmaceutical containers include, without limitation, a syringe, vial, infusion bottle, ampoule, carpoule, a syringe equipped with a needle protection system, and a carpoule within an injection pen.

### FGF-21 Conjugates

In some embodiments, exemplary conjugates of a modified sugar and a mutant FGF-21 peptide are presented. In some embodiments, mutant FGF-21 peptide conjugates were made comprising a mutant FGF peptide and at least one modified sugar, wherein a first of the at least one modified sugar is linked to an amino acid of the peptide through a glycosyl linking group. As described herein, the amino acid to which the glycosyl linking group is attached is mutated to create a site recognized by the glycosyltransferase.

In another exemplary embodiment, a mutant FGF-21 peptide conjugate can comprise a mutant FGF-21 peptide and a glycosyl group attached to the mutated amino acid residue of the mutant FGF-21 peptide.

In an exemplary embodiment, the glycosyl group is an intact glycosyl linking group. In another exemplary embodiment, the glycosyl group further comprises a modifying group. In another exemplary embodiment, the modifying group is a non-glycosidic modifying group. In another exemplary embodiment, the modifying group does not include a naturally occurring saccharide moiety.

### Modified Sugars

In an exemplary embodiment, mutant FGF-21 peptides are reacted with a modified sugar, thus forming a peptide conjugate. A modified sugar comprises a “sugar donor moiety” as well as a “sugar transfer moiety”. The sugar donor moiety is any portion of the modified sugar that will be attached to the peptide, either through a glycosyl moiety or amino acid moiety, as a conjugate described herein. The sugar donor moiety includes those atoms that are chemically altered during their conversion from the modified sugar to the glycosyl linking group of the mutant FGF-21 peptide conjugate.

For modified sugars described herein, the saccharyl moiety may be a saccharide, a deoxy-saccharide, an amino-saccharide, or an N-acyl saccharide. The term “saccharide” and its equivalents, “saccharyl,” “sugar,” and “glycosyl” refer to monomers, dimers, oligomers and polymers. The sugar moiety is also functionalized with a modifying group. The modifying group is conjugated to the saccharyl moiety, typically, through conjugation with an amine, sulfhydryl or hydroxyl, e.g., primary hydroxyl, moiety on the sugar. In an exemplary embodiment, the modifying group is attached through an amine moiety on the sugar, e.g., through an amide, a urethane or a urea that is formed through the reaction of the amine with a reactive derivative of the modifying group.

Any saccharyl moiety can be utilized as the sugar donor moiety of the modified sugar. The saccharyl moiety can be a known sugar, such as mannose, galactose or glucose, or a species having the stereochemistry of a known sugar. The general formulae of these modified sugars are:

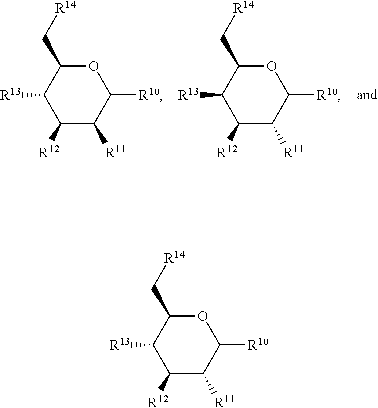

Other saccharyl moieties that are useful in methods described herein include, but are not limited to fucose and sialic acid, as well as amino sugars such as glucosamine, galactosamine, mannosamine, the 5-amine analogue of sialic acid and the like. The saccharyl moiety can be a structure found in nature or it can be modified to provide a site for conjugating the modifying group. For example, in one embodiment, the modified sugar provides a sialic acid derivative in which the 9-hydroxy moiety is replaced with an amine. The amine is readily derivatized with an activated analogue of a selected modifying group. Examples of modified sugars useful in methods described herein are presented in PCT Patent Application No. PCT/US05/002522, which is incorporated herein by reference in its entirety.

In a further exemplary embodiment, the disclosure utilizes modified sugars in which the 6-hydroxyl position is converted to the corresponding amine moiety, which bears a linker-modifying group cassette such as those set forth above. Exemplary glycosyl groups that can be used as the core of these modified sugars include Gal, GalNAc, Glc, GlcNAc, Fuc, Xyl, Man, and the like. A representative modified sugar according to this embodiment is set forth below:

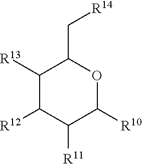

in which R¹¹-R¹⁴ are members independently selected from H, OH, C(O)CH₃, NH, and NH C(O)CH₃. R¹⁰ is a link to, e.g., another glycosyl residue (—O-glycosyl). R¹⁴ is OR¹, NHR¹ or NH-L-R¹. R¹ and NH-L-R¹ are as described herein.

In a still further exemplary embodiment, the glycosyl groups used as the core of modified sugars in which the 6-hydroxyl position is converted to the corresponding amine moiety include Gal and/or GalNAc.

### Glycosyl Linking Groups

In an exemplary embodiment, mutant FGF-21 peptide conjugates comprising a modified sugar described herein and a mutant FGF peptide are presented. In this embodiment, the sugar donor moiety (such as the saccharyl moiety and the modifying group) of the modified sugar becomes a “glycosyl linking group”. The “glycosyl linking group” can alternatively refer to the glycosyl moiety which is interposed between the peptide and the modifying group.

In the exemplary embodiments that follow, the disclosure is illustrated by reference to the use of selected derivatives of furanose and pyranose. Those of skill in the art will appreciate that the structures and compositions set forth are generally applicable across the genus of glycosyl linking groups and modified sugars. The glycosyl linking group can, therefore, comprise virtually any mono- or oligo-saccharide.

In an exemplary embodiment, methods described herein utilize a glycosyl linking group that has the formula:

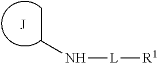

in which J is a glycosyl moiety, L is a bond or a linker and R¹ is a modifying group, e.g., a polymeric modifying group. Exemplary bonds are those that are formed between an NH₂ moiety on the glycosyl moiety and a group of complementary reactivity on the modifying group. For example, when R¹ includes a carboxylic acid moiety, this moiety may be activated and coupled with the NH₂ moiety on the glycosyl residue affording a bond having the structure NHC(O)R¹. J in some embodiments is a glycosyl moiety that is “intact”, not having been degraded by exposure to conditions that cleave the pyranose or furanose structure, e.g. oxidative conditions, e.g., sodium periodate.

Exemplary linkers include alkyl and heteroalkyl moieties. The linkers include linking groups, for example acyl-based linking groups, e.g., —C(O)NH—, —OC(O)NH—, and the like. The linking groups are bonds formed between components of the conjugates, e.g., between the glycosyl moiety and the linker (L), or between the linker and the modifying group (R¹). Other exemplary linking groups are ethers, thioethers and amines. For example, in one embodiment, the linker is an amino acid residue, such as a glycine residue. The carboxylic acid moiety of the glycine is converted to the corresponding amide by reaction with an amine on the glycosyl residue, and the amine of the glycine is converted to the corresponding amide or urethane by reaction with an activated carboxylic acid or carbonate of the modifying group.

An exemplary species of NH-L-R¹ has the formula: —NH{C(O)(CH₂)ₐNH}ₛ{C(O)(CH₂)_{b}(OCH₂CH₂)_{c}O(CH₂)_{d}NH}ₜR¹, in which the indices s and t are independently 0 or 1. The indices a, b and d are independently integers from 0 to 20, and c is an integer from 1 to 2500. Other similar linkers are based on species in which an —NH moiety is replaced by another group, for example, —S, —O or —CH₂. As is understood in the art, one or more of the bracketed moieties corresponding to indices s and t can be replaced with a substituted or unsubstituted alkyl or heteroalkyl moiety.

In some embodiments, compounds described herein may comprise NH-L-R′, wherein NH-L-R′ is: NHC(O)(CH₂)ₐNHC(O)(CH₂)_{b}(OCH₂CH₂)_{c}O(CH₂)_{d}NHR¹, NHC(O)(CH₂)_{b}(OCH₂CH₂)_{c}O(CH₂)_{d}NHR¹, NHC(O)O(CH₂)_{b}(OCH₂CH₂)_{c}O(CH₂)_{d}NHR¹, NH(CH₂)ₐNHC(O)(CH₂)_{b}(OCH₂CH₂)_{c}CO(CH₂)_{d}NHR¹, NHC(O)(CH₂)ₐNHR¹, NH(CH₂)ₐNHR¹, and NHR¹. In these formulae, the indices a, b and d are independently selected from the integers from 0 to 20, for example from 1 to 5. The index c is an integer from 1 to about 2500.

In an exemplary embodiment, c is selected such that the PEG moiety is approximately 1 kD, 5 kD, 10, kD, 15 kD, 20 kD, 25 kD, 30 kD, 35 kD, 40 kD, 45 kD, 50 kD, 55 kD, 60 kD or 65 kD.

In some embodiments, the c is selected such that the PEG moiety ranges from 15-25 kD, 16-25 kD, 17-25 kD, 18-25 kD, 19-25 kD, 20-25 kD, 21-25 kD, 22-25 kD, 23-25 kD, 24-25 kD, 15-20 kD, 16-20 kD, 17-20 kD, 18-20 kD, 19-20 kD, 20-30 kD, 21-30 kD, 22-30 kD, 23-30 kD, 24-30 kD, 25-30 kD, 26-30 kD, 27-30 kD, 28-30 kD, 29-30 kD. In some embodiments, the c is selected such that the PEG moiety is 20 kD, 22 kD, 23 kD, 24 kD, 25 kD, 26 kD, 27 kD, 28 kD, 29 kD, or 30 kD.

For the purposes of clarity, the glycosyl linking groups in the remainder of this section are based on a sialyl moiety. However, one of skill in the art will recognize that another glycosyl moiety, such as mannosyl, galactosyl, glucosyl, or fucosyl, could be used in place of the sialyl moiety.

In an exemplary embodiment, the glycosyl linking group is an intact glycosyl linking group, in which the glycosyl moiety or moieties forming the linking group are not degraded by chemical (e.g., sodium metaperiodate) or enzymatic (e.g., oxidase) processes. Selected conjugates of the disclosure include a modifying group that is attached to the amine moiety of an amino-saccharide, e.g., mannosamine, glucosamine, galactosamine, sialic acid etc. In an exemplary embodiment, the disclosure provides a peptide conjugate comprising an intact glycosyl linking group having a formula that is selected from:

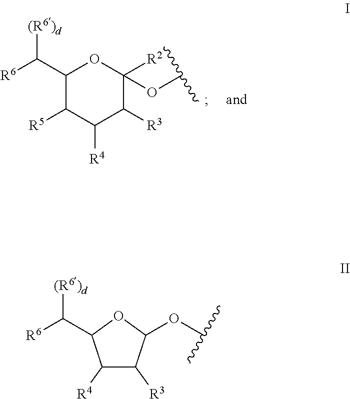

In Formulae I R² is H, CH₂OR⁷, COOR⁷ or OR⁷, in which R⁷ represents H, substituted or unsubstituted alkyl or substituted or unsubstituted heteroalkyl. When COOR⁷ is a carboxylic acid or carboxylate, both forms are represented by the designation of the single structure COO⁻ or COOH. In Formulae I and II, the symbols R³, R⁴, R⁵, R⁶ and R^{6′} independently represent H, substituted or unsubstituted alkyl, OR⁸, NHC(O)R⁹. The index d is 0 or 1. R⁸ and R⁹ are independently selected from H, substituted or unsubstituted alkyl, substituted or unsubstituted heteroalkyl, sialic acid or polysialic acid. At least one of R³, R⁴, R⁵, R⁶ or R^{6′} includes a modifying group. This modifying group can be a polymeric modifying moiety e.g., PEG, linked through a bond or a linking group. In an exemplary embodiment, R⁶ and R^{6′}, together with the carbon to which they are attached are components of the pyruvyl side chain of sialic acid. In a further exemplary embodiment, the pyruvyl side chain is functionalized with the polymeric modifying group. In another exemplary embodiment, R⁶ and R^{6′}, together with the carbon to which they are attached are components of the side chain of sialic acid and the polymeric modifying group is a component of R⁵.

Exemplary modifying group-intact glycosyl linking group cassettes according to this motif are based on a sialic acid structure, such as those having the formulae:

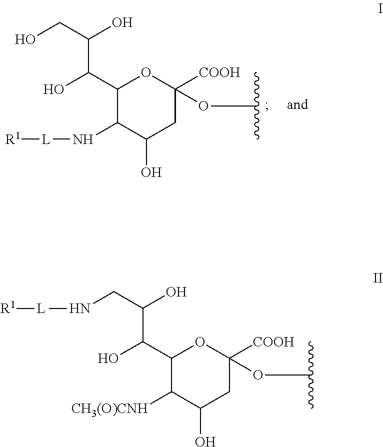

In the formulae above, R¹ and L are as described above. Further detail about the structure of exemplary R¹ groups is provided below.

In still a further exemplary embodiment, the conjugate is formed between a peptide and a modified sugar in which the modifying group is attached through a linker at the 6-carbon position of the modified sugar. Thus, illustrative glycosyl linking groups according to this embodiment have the formula:

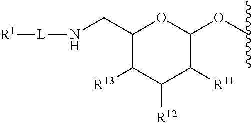

in which the radicals are as discussed above. Glycosyl linking groups include, without limitation, glucose, glucosamine, N-acetyl-glucosamine, galactose, galactosamine, N-acetylgalactosamine, mannose, mannosamine, N-acetyl-mannosamine, and the like.

In some embodiments, the present disclosure provides a mutant FGF-21 peptide conjugate comprising the following glycosyl linking group:

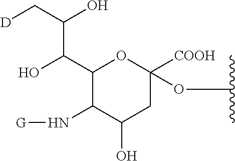

wherein D is a member selected from —OH and R¹-L-HN—; G is a member selected from H and R¹-L- and —C(O)(C₁-C₆)alkyl; R¹ is a moiety comprising a straight-chain or branched poly(ethylene glycol) residue; and L is a linker, e.g., a bond (“zero order”), substituted or unsubstituted alkyl and substituted or unsubstituted heteroalkyl. In exemplary embodiments, when D is OH, G is and when G is —C(O)(C₁-C₆)alkyl, D is R¹-L-NH—.

In some embodiments, the peptide conjugate includes a glycosyl linking group having the formula:

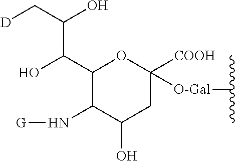

In some embodiments, the glycosyl linking group has the formula:

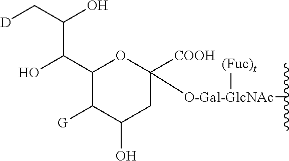

in which the index t is 0 or 1.

In some embodiments, the glycosyl linking group has the formula:

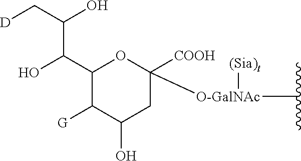

in which the index t is 0 or 1.

In some embodiments, the glycosyl linking group has the formula:

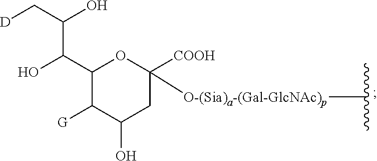

in which the index p represents and integer from 1 to 10; and a is either 0 or 1.

In some embodiments, a glycoPEGylated peptide conjugate is selected from the formulae set forth below:

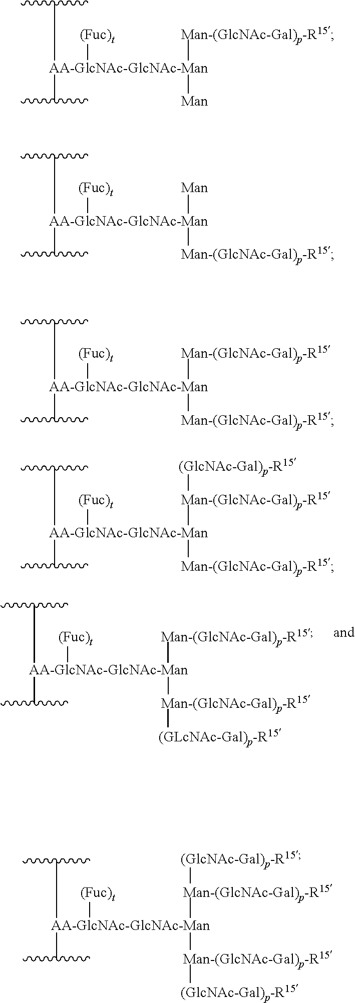

In the formulae above, the index t is an integer from 0 to 1 and the index p is an integer from 1 to 10. The symbol R^{15′} represents H, OH (e.g., Gal-OH), a sialyl moiety, a sialyl linking group (i.e., sialyl linking group-polymeric modifying group (Sia-L-R¹), or a sialyl moiety to which is bound a polymer modified sialyl moiety (e.g., Sia-Sia-L-R¹) (“Sia-Sia^{P}”)). Exemplary polymer modified saccharyl moieties have a structure according to Formulae I and II. An exemplary peptide conjugate of the disclosure will include at least one glycan having a R^{15′} that includes a structure according to Formulae I or II. The oxygen, with the open valence, of Formulae I and II is, in some embodiments, attached through a glycosidic linkage to a carbon of a Gal or GalNAc moiety. In a further exemplary embodiment, the oxygen is attached to the carbon at position 3 of a galactose residue. In an exemplary embodiment, the modified sialic acid is linked α2,3-to the galactose residue. In another exemplary embodiment, the sialic acid is linked α2,6-to the galactose residue.

In an exemplary embodiment, the sialyl linking group is a sialyl moiety to which is bound a polymer modified sialyl moiety (e.g., Sia-Sia-L-R¹) (“Sia-Sia^{P}”). Here, the glycosyl linking group is linked to a galactosyl moiety through a sialyl moiety:

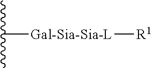

An exemplary species according to this motif is prepared by conjugating Sia-L-R¹ to a terminal sialic acid of a glycan using an enzyme that forms Sia-Sia bonds, e.g., CST-11, ST8Sia-II, ST8Sia-III and ST8Sia-IV.

In another exemplary embodiment, the glycans on the peptide conjugates have a formula that is selected from the group:

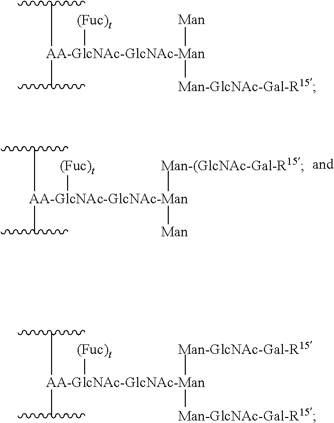

and combinations thereof.

In each of the formulae above, R^{15′} is as discussed above. Moreover, an exemplary mutant FGF-21 peptide conjugate described herein will include at least one glycan with an R¹⁵ moiety having a structure according to Formulae I or II.

In another exemplary embodiment, the glycosyl linking group comprises at least one glycosyl linking group having the formula:

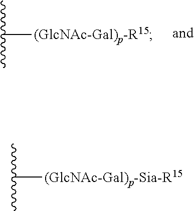

wherein R¹⁵ is said sialyl linking group; and the index p is an integer selected from 1 to 10.

In an exemplary embodiment, the glycosyl linking moiety has the formula:

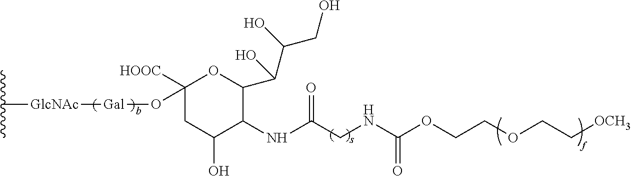

in which b is an integer from 0 to 1. The index s represents an integer from 1 to 10; and the index f represents an integer from 1 to 2500.

In an exemplary embodiment, the polymeric modifying group is PEG. In another exemplary embodiment, the PEG moiety has a molecular weight of 20-30 kDa. In exemplary embodiments, the PEG moiety has a molecular weight of 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, 30 kDa, 31 kDa, 32 kDa, or 33 kDa. In another exemplary embodiment, the PEG moiety has a molecular weight of 20 kDa. In another exemplary embodiment, the PEG moiety has a molecular weight of 30 kDa. In another exemplary embodiment, the PEG moiety has a molecular weight of about 5 kDa. In another exemplary embodiment, the PEG moiety has a molecular weight of about 10 kDa. In another exemplary embodiment, the PEG moiety has a molecular weight of about 40 kDa.

In an exemplary embodiment, the glycosyl linking group is a linear 10 kDa-PEG-sialyl, and one or two of these glycosyl linking groups are covalently attached to the peptide.

In an exemplary embodiment, the glycosyl linking group is a linear 20 kDa-PEG-sialyl, and one or two of these glycosyl linking groups are covalently attached to the peptide. In an exemplary embodiment, the glycosyl linking group is a linear 30 kDa-PEG-sialyl, and one or two of these glycosyl linking groups are covalently attached to the peptide. In an exemplary embodiment, the glycosyl linking group is a linear 5 kDa-PEG-sialyl, and one, two or three of these glycosyl linking groups are covalently attached to the peptide. In an exemplary embodiment, the glycosyl linking group is a linear 40 kDa-PEG-sialyl, and one or two of these glycosyl linking groups are covalently attached to the peptide.

In some embodiments, a mutant FGF-21 peptide is pegylated in accordance with methods described herein. In some embodiments, the mutant FGF-21 peptide comprises the mutations S¹⁷²T and R¹⁷⁶A, wherein the positions of the amino acids S and R are based on the amino acid sequence as depicted in SEQ ID NO: 1. In some embodiments, the mutant FGF-21 peptide comprises the amino acid sequence as depicted in SEQ ID NO: 2. As detailed herein above, the at least one glycosyl moiety attached to the threonine residue and linking the newly introduced threonine residue to the PEG moiety may virtually be any possible glycosyl moiety. The only limitation is that it should be able to attach to threonine and that it should be able to be attached to PEG or m-PEG, for example via a linker, e.g. an amino acid residue, e.g. glycine. In some embodiments, the at least one glycosyl moiety comprises N-acetylgalactosamine (GalNAc), galactose (Gal) and/or sialic acid (Sia). In some embodiments, the at least one glycosyl moiety comprises the structure-GalNAc-Sia-, i.e. two glycosyl moieties, namely GalNAc and Sia, wherein the PEG residue may be attached to GalNAc or Sia. The glycosyl moiety which is not attached to the PEG moiety may be attached to the newly introduced threonine residue.

In some embodiments, the 20 kDa PEG moiety is attached to the at least one glycosyl linker via a linker, e.g. an amino acid residue, particularly a small amino acid, such as alanine or glycine (Gly). Hence, the PEG or m-PEG moiety is attached to the amino acid and the amino acid is attached to a glycosyl moiety, such as Sia. The glycosyl moiety is attached to the amino acid linker, if present, and to the newly introduced threonine residue in the mutant FGF-21 amino acid sequence. The amino acid residue is attached to PEG and the glycosyl residue via a method described in WO 03/031464 which is incorporated herein by reference.

In some embodiments, the mutant FGF-21 peptide (e.g., SEQ ID NO: 2) conjugate comprises the structure-GalNAc-Sia-Gly-PEG (20 kDa), wherein GalNAc is attached, e.g. to a newly introduced threonine residue and to Sia. Sia is further attached via a glycine residue to a PEG of 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, 30 kDa, 31 kDa, 32 kDa, or 33 kDa.

In some embodiments, the mutant FGF-21 peptide (e.g., SEQ ID NO: 2) conjugate comprises the structure-GalNAc-Sia-Gly-PEG (20 kDa), wherein GalNAc is attached, e.g. to a newly introduced threonine residue and to Sia. Sia is further attached via a glycine residue to a PEG of 20 kDa, 21 kDa, 22 kDa, 23 kDa, 24 kDa, 25 kDa, 26 kDa, 27 kDa, 28 kDa, 29 kDa, or 30 kDa.

In some embodiments, the mutant FGF-21 peptide (e.g., SEQ ID NO: 2) conjugate comprises the structure-GalNAc-Sia-Gly-PEG (20 kDa), wherein GalNAc is attached, e.g. to a newly introduced threonine residue and to Sia. Sia is further attached via a glycine residue to a PEG of 20 kDa, 25 kDa, or 30 kDa.

In some embodiments, the mutant FGF-21 peptide (e.g., SEQ ID NO: 2) conjugate comprises the structure-GalNAc-Sia-Gly-PEG (20 kDa), wherein GalNAc is attached, e.g. to a newly introduced threonine residue and to Sia. Sia is further attached via a glycine residue to a PEG of 20 kDa or 30 kDa.

In some embodiments, the mutant FGF-21 peptide (e.g., SEQ ID NO: 2) conjugate comprises the structure-GalNAc-Sia-Gly-PEG (20 kDa), wherein GalNAc is attached, e.g. to a newly introduced threonine residue and to Sia. Sia is further attached via a glycine residue to a PEG of 20 kDa.

In some embodiments, the mutant FGF-21 peptide conjugate comprises the structure:

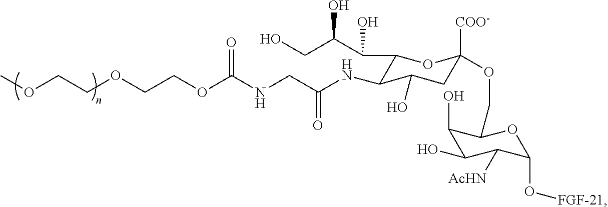

wherein n is an integer selected from 450 to 460.

In some embodiments, the 20 kDa PEG may be linear or branched. In some embodiments, the 20 kDa PEG is a linear 20 kDa PEG. In some embodiments, the 20 kDa PEG is a 20 kDa methoxy-PEG (mPEG, m-PEG). PEG and mPEG of different molecular weight can be obtained from various suppliers, such as from JenKem Technology USA, Plano, TX, USA, or Merckle Biotec, Ulm, Germany. It is understood in the art that PEG 20 kDa means that the size of the PEG residues is 20 kDa in average and that the majority of the PEG residues are 20 kDa in size.

### Mutant FGF-21 Peptides and Conjugates Thereof

As described herein, variants of Fibroblast Growth Factor-21 (FGF-21) having surprising properties, including variants having exceptionally long half-lives are produced, which variants are peptide conjugates comprising
i) a mutant FGF-21 peptide comprising at least one threonine (T) residue adjacent to at least one proline (P) residue on the C-terminal side of the at least one proline residue, thereby forming at least one O-linked glycosylation site which does not exist in the corresponding native FGF-21, wherein the corresponding native FGF-21 has an amino acid sequence that is at least 95% identical to SEQ ID NO: 1, and
ii) a 20-30 kDa polyethylene glycol (PEG), wherein said 20-30 kDa PEG is covalently attached to said mutant FGF-21 peptide at the at least one threonine residue via at least one glycosyl moiety.

For the attachment of the 20-30 kDa PEG residue, a threonine residue is introduced into the amino acid sequence of native FGF-21 adjacent to and on the C-terminal side of a proline residue which is already present in the amino acid sequence of native FGF-21, i.e. is a native proline residue. For this purpose, either (i) an additional threonine may be introduced immediately next to the native proline residue or (ii) the native amino acid which is present in the native amino acid sequence of FGF-21 adjacent to and located on the C-terminal side of a native proline residue is exchanged for a threonine residue. In the present disclosure, option (ii) is an exemplary embodiment. As described herein, more than one threonine residue may be introduced adjacent and C-terminal to a proline residue which is already present. A mutant FGF-21 of the present disclosure may thus comprise both threonine residues which have been additionally introduced and threonine residues which have been introduced instead of a native amino acid.

By the introduction of a new threonine residue on the C-terminal side and adjacent to a proline residue, a consensus sequence for O-glycosylation enzyme is formed. Because proline residues are typically found on the surface of proteins (in, e.g., turns, kinks, and/or loops), a design that calls for O-glycosylation and PEGylation thereto using a PEG-glycosyl moiety in close proximity to a proline residue benefits from the relative accessibility of the target attachment site for the glycosyl transferase that transfers the glycosyl or glycol-PEG moiety and the potential to accommodate the conjugated glycosyl and/or PEG structure without disruption of protein structure.

For introduction of the threonine residues into the native amino acid sequence of FGF-21, the general methods include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

In some embodiments, the native FGF-21 amino acid sequence corresponds to the native amino acid sequence of human FGF-21 depicted in SEQ ID NO: 1.

In some embodiments, the mutant FGF-21 peptide comprises the amino acid sequence PT, i.e. a threonine residue C-terminally adjacent to a proline residue. The sequence PT is not present in the native FGF-21 amino acid sequence.

Optionally, the mutant FGF-21 peptide comprises at least one amino acid sequence selected from the group consisting of P¹⁷²T (e.g. SEQ ID NO: 2 or 3), P¹⁵⁶T (e.g. SEQ ID NO: 4), PST (e.g. SEQ ID NO: 5), P³T (e.g. SEQ ID NO: 6), POT (e.g. SEQ ID NO: 7), P⁵⁰T (e.g. SEQ ID NO: 8), P⁶¹T (e.g. SEQ ID NO: 9), P⁷⁹T (e.g. SEQ ID NO: 10), P⁹¹T (e.g. SEQ ID NO: 11), P¹¹⁶T (e.g. SEQ ID NO: 12), P¹²⁰T (e.g. SEQ ID NO: 13), P¹²⁵T (e.g. SEQ ID NO: 14), P¹²⁹T (e.g. SEQ ID NO: 15), P¹³¹T (e.g. SEQ ID NO: 16), P¹³⁴T (e.g. SEQ ID NO: 17), P¹³⁹T (e.g. SEQ ID NO: 18), P¹⁴¹T (e.g. SEQ ID NO: 19), P¹⁴⁴T (e.g. SEQ ID NO: 20, P¹⁴⁵T (e.g. SEQ ID NO: 21), P¹⁴⁸T (e.g. SEQ ID NO: 22), P¹⁵⁰T (e.g. SEQ ID NO: 23), P¹⁵¹T (e.g. SEQ ID NO: 24), P¹⁵⁸T (e.g. SEQ ID NO: 25), P¹⁵⁹T (e.g. SEQ ID NO: 26), P¹⁶⁶T (e.g. SEQ ID NO: 27), P¹⁷⁸T (e.g. SEQ ID NO: 28), and combinations thereof, wherein the positions of proline and threonine are based on the native amino acid sequence of FGF-21 as depicted in SEQ ID NO: 1. In some embodiments, the mutant FGF-21 peptide comprises at least one amino acid sequence selected from the group consisting of P¹⁷²T, P¹⁵⁶T, P⁵T and combinations thereof, for example consisting of P¹⁷²T, P¹⁵⁶T and combinations thereof, and particularly the mutant FGF-21 peptide comprises the sequence motif P¹⁷²T, based on the amino acid sequence as depicted in SEQ ID NO: 1, wherein the positions of proline and threonine are based on the amino acid sequence as depicted in SEQ ID NO: 1.

In some embodiments, the proline residue is located between amino acid 145 and the C-terminus of the mutant FGF-21 peptide, wherein the position of amino acid 145 is based on the amino acid sequence as depicted in SEQ ID NO: 1. As demonstrated by results presented herein, the C-terminus of FGF-21 surprisingly tolerates attachment of PEG and in particular of glycosyl-PEG moieties. This was unexpected since the literature reports that the intact C-terminus is necessary for β-Klotho binding of FGF-21.

In some embodiments, the mutant FGF-21 peptide comprises the mutations S172T and R¹⁷⁶A, wherein the positions of the amino acids S and R are based on the amino acid sequence as depicted in SEQ ID NO: 1, particularly the mutant FGF-21 peptide comprises the amino acid sequence as depicted in SEQ ID NO: 2. The mutation R¹⁷⁶A has been found beneficial to the protein's overall stability after introducing the O-linked glycosylation site at threonine 173. By this mutation, the relatively large arginine side chain was removed and replaced by the small side chain of alanine. It is assumed that the smaller side chain of alanine interferes less with the voluminous glycosyl-PEG moiety to be attached to thindicae mutated FGF-21 peptide.

In an alternative embodiment, the mutant FGF-21 peptide comprises the mutation Q¹⁵⁷T, wherein the position of the amino acid Q is based on the amino acid sequence as depicted in SEQ ID NO: 1, particularly the mutant FGF-21 peptide comprises the amino acid sequence as depicted in SEQ ID NO: 4, or the mutation D6T, wherein the position of the amino acid D is based on the amino acid sequence as depicted in SEQ ID NO: 1, particularly the mutant FGF-21 peptide comprises the amino acid sequence as depicted in SEQ ID NO: 5.

In some embodiments, the mutant FGF-21 peptide conjugate comprises an amino acid sequence selected from the group consisting of SEQ ID NOs: 2 to 28, more particularly an amino acid sequence selected from the group consisting of SEQ ID NOs: 2 to 5, even more particularly an amino acid sequence selected from the group consisting of SEQ ID NOs: 2 to 4, and most particularly the mutant FGF-21 peptide comprises the amino acid sequence as depicted in SEQ ID NO: 2.

### Pharmaceutical Compositions and Methods of Treating

In some embodiments, the pharmaceutical compositions comprise the mutant FGF-21 peptide conjugate and a pharmaceutically acceptable carrier, such as water or a physiologically compatible buffer. The pharmaceutical compositions typically comprise a therapeutically effective or pharmaceutically active amount of the mutant FGF-21 peptide conjugate as active agent.

In some embodiments, the pharmaceutical compositions are suitable for use in a variety of drug delivery systems. Suitable formulations for use in the present disclosure are found in Remington's Pharmaceutical Sciences, Mack Publishing Company, Philadelphia, PA, 17^{th} ed. (1985). The pharmaceutical compositions are intended for parenteral, intranasal, topical, oral, or local administration, such as by subcutaneous injection, aerosol inhalation, or transdermal adsorption, for prophylactic and/or therapeutic treatment. Commonly, the pharmaceutical compositions are administered parenterally, e.g., subcutaneously or intravenously.

In some embodiments, the disclosure provides compositions for parenteral administration which comprise the mutant FGF-21 peptide conjugate dissolved or suspended in an acceptable carrier, such as an aqueous carrier, e.g., water, buffered water, saline, phosphate buffered saline (PBS) and the like. The compositions may also contain detergents such as Tween 20 and Tween 80; stabilizers such as mannitol, sorbitol, sucrose, and trehalose; and preservatives such as EDTA and m-cresol. The compositions may contain pharmaceutically acceptable auxiliary substances as required to approximate physiological conditions, such as pH adjusting and buffering agents, tonicity adjusting agents, wetting agents, detergents and the like.

In some embodiments, the pharmaceutical compositions may be sterilized by conventional sterilization techniques or may be sterile filtered. The resulting aqueous solutions may be packaged for use as is, or lyophilized, the lyophilized preparation being combined with a sterile aqueous carrier prior to administration. The compositions containing the FGF peptide conjugates can be administered for prophylactic and/or therapeutic treatments of diabetes or diabetes related diseases, for example for the treatment of diabetes type 2, NASH and metabolic syndrome. In therapeutic applications, compositions are administered to a subject already suffering from a disease or condition related to diabetes, in an amount sufficient to cure or at least partially arrest the symptoms of the disease and its complications. An amount adequate to accomplish this is defined as a “therapeutically effective amount” and usually depends on the patient's state of health and weight. Efficacious doses range from 0.1 mg/kg to 6 mg/kg when tested in various animal models of NASH and type 2 diabetes.

In some embodiments, the present disclosure provides methods for treating a disease and/or a disorder or symptoms thereof which comprise administering a therapeutically effective amount of a compound (a mutant FGF-21 peptide conjugate described herein) or a pharmaceutical composition comprising same to a subject (e.g., a mammal such as a human). Thus, one embodiment is a method for treating a subject suffering from diabetes or a diabetes related disease (e.g., diabetes type 2, NAFLD, NASH or metabolic syndrome) or a symptom thereof. The method includes the step of administering to the mammal an amount of a compound described herein in an amount sufficient to treat the disease or disorder or symptom thereof or a composition comprising same, under conditions such that the disease or disorder is treated. In some embodiments, the method comprises administering subcutaneously to the mammal an amount of a compound described herein in an amount sufficient to treat the disease or disorder or symptom thereof or a composition comprising same, under conditions such that the disease or disorder is treated.

In some embodiments, the mutant FGF-21 peptide conjugate (e.g., 89Bio-100, also referred to as Pegozafermin) is administered to a human subject at a therapeutic dosing regimen of from about 3 mg to about 30 mg once every week. In some embodiments, the mutant FGF-21 peptide conjugate (e.g., 89Bio-100) is administered to a human subject at a therapeutic dosing regimen of from about 18 mg to about 44 mg once every two weeks. In some embodiments, the mutant FGF-21 peptide conjugate (e.g., 89Bio-100) is administered to a human subject at a therapeutic dosing regimen of a single dose at 0.45 mg, 1.2 mg, 3 mg, 9.1 mg, 15 mg, 18.2 mg, 39 mg or 78 mg, or placebo at a 6:2 ratio (7:3 ratio for the 9.1 mg dose). See also Example 1 and Example 4 below.

In some embodiments, the therapeutic dosing regimen comprises a range of about 3 mg to about 44 mg, a range of about 3 mg to about 36 mg; a range of about 3 mg to about 30 mg; a range of about 3 mg to about 27 mg; a range of about 3 mg to about 18 mg; a range of about 3 mg to about 15 mg; a range of about 9 mg to about 44 mg, a range of about 9 mg to about 36 mg, a range of about 9 mg to about 30 mg, a range of about 9 mg to about 27 mg; a range of about 9 mg to about 18 mg; a range of about 9 mg to about 15 mg; a range of about 15 mg to about 44 mg, a range of about 15 mg to about 36 mg, a range of about 15 mg to about 30 mg, a range of about 15 mg to about 27 mg; a range of about 15 mg to about 18 mg; a range of about 18 mg to about 44 mg, a range of about 18 mg to about 36 mg, a range of about 18 mg to about 30 mg, a range of 18 mg to 27 mg; a range of about 18 mg to about 30 mg, a range of about 18 mg to about 27 mg; a range about 3 mg to about 9 mg; a range of about 9 mg to about 15 mg; a range of about 9 mg to about 18 mg; a range of about 18 mg to about 27 mg; a range of about 27 mg to about 30 mg; a range of about 18 mg to about 27 mg; a range of about 3 mg to about 18 mg; a range of about 18 mg to about 36 mg. In some embodiments, the therapeutic dosing regimen comprises a range of about 3 mg to about 50 mg; a range of about 5 mg to about 50 mg; a range of about 10 mg to about 50 mg; a range of about 20 mg to about 50 mg; a range of about 30 mg to about 50 mg; or a range of about 40 mg to about 50 mg; and any whole integer within any of the indicated ranges. In some embodiments, the therapeutic dosing regimen comprises a range of about 5 mg to about 40 mg; a range of about 10 mg to about 40 mg; a range of about 20 mg to about 40 mg; a range of about 30 mg to about 40 mg; or a range of about 35 mg to about 40 mg; and any whole integer within any of the indicated ranges. In some embodiments, the therapeutic dosing regimen comprises a range of about 5 mg to about 30 mg; a range of about 10 mg to about 30 mg; a range of about 20 mg to about 30 mg; or a range of about 25 mg to about 30 mg; and any whole integer within any of the indicated ranges. In some embodiments, the therapeutic dosing regimen comprises a range of about 10 mg to about 20 mg; or a range of about 15 mg to about 20 mg; and any whole integer within any of the indicated ranges. In some embodiments, the therapeutic dosing regimen comprises a dose of about 3 mg; about 9 mg; about 15 mg, about 18 mg; about 27 mg, about 30 mg, about 36 mg or about 44 mg. In some embodiments, the therapeutic dosing regimen comprises a dose of about 3 mg; about 4 mg; about 5 mg; about 6 mg; about 7 mg; about 8 mg; about 9 mg; about 10 mg; about 11 mg; about 12 mg; about 13 mg; about 14 mg; about 15 mg, about 16 mg; about 17 mg; about 18 mg; about 19 mg; about 20 mg; about 21 mg; about 22 mg; about 23 mg; about 24 mg; about 25 mg; about 26 mg; about 27 mg, about 28 mg; about 29 mg; about 30 mg, about 31 mg; about 32 mg; about 33 mg; about 34 mg; about 35 mg; about 36 mg; about 37 mg; about 38 mg; about 39 mg; about 40 mg; about 41 mg; about 42 mg; about 43 mg; or about 44 mg. The term “about” as used herein refers to an amount equal to 10% more or 10% less of the particularly indicated amount. For example, about 10 mg refers to a range of 9.0-11 mg. In some embodiments, the therapeutic dosing regimen comprises a dose of 9.1 mg; about 18.2 mg; or about 39 mg.

The aforementioned therapeutic dosing regimens may be administered to a human in need thereof to treat at least one of diabetes (e.g., diabetes type 2), NAFLD, NASH, or metabolic syndrome. In some embodiments, the aforementioned therapeutic dosing regimens are administered to a human in need thereof to reduce triglyceride levels. In some embodiments, the aforementioned therapeutic dosing regimens are administered to a human to reduce triglyceride levels. (see for example, Example 1)

The aforementioned therapeutic dosing regimens may be administered to a human in need thereof to treat NASH. In some embodiments, the aforementioned therapeutic dosing regimens are administered to a human in need thereof to reduce liver fat. In some embodiments, the aforementioned therapeutic dosing regimens are administered to a human in need thereof to reduce ALT. In some embodiments, the aforementioned therapeutic dosing regimens are administered to a human in need thereof to reduce NAFLD Activity scores. In some embodiments, the aforementioned therapeutic dosing regimens are administered to a human in need thereof to reduce improve fibrosis. In some embodiments, the aforementioned therapeutic dosing regimens are administered to a human in need thereof to improve HbA1c levels. In some embodiments, the aforementioned therapeutic dosing regimens result in an increase of adiponectin levels. In some embodiments, the aforementioned therapeutic dosing regimens are administered to a human in need thereof to reduce weight. In some embodiments, the aforementioned therapeutic dosing regimens are administered to a human in need thereof to improve lipid parameters. In some embodiments, the aforementioned therapeutic dosing regimens are administered to a human in need thereof to reduce triglyceride levels. In some embodiments, the aforementioned therapeutic dosing regimens are administered to a human to reduce triglyceride levels. (see for example, Example 5).

The aforementioned therapeutic dosing regimens may also be administered to a human in need thereof to reduce cravings for sugary foods and/or beverages. (see for example, Example 2)

In some embodiments, a pharmaceutical composition comprising any one of or at least one of the mutant FGF-21 peptide conjugates described herein and a pharmaceutically acceptable carrier is presented. The mutant FGF-21 peptide conjugate may be present in the pharmaceutical composition in a concentration in a range from 0.1 mg/mL to 50 mg/mL, from 1 mg/mL to 45 mg/mL, from 10 mg/mL to 40 mg/mL, for example of 26±4 mg/mL. In some embodiments, the pharmaceutical composition further comprises a buffering agent, for example a Tris buffer. In some embodiments, the buffering agent is present in a concentration from 1 mM to 100 mM, from 2 mM to 75 mM, from 5 mM to 50 mM, from 10 mM to 25 mM, for example of 16±2 mM. In some embodiments, the pH is in the range from 6.0 to 8.5, from 6.5 to 8.0, from 6.75 to 8.0, for example 7.5±0.3. In some embodiments, the pharmaceutical composition further comprises a salt, for example an inorganic salt, e.g. NaCl. In some embodiments, the salt is present in a concentration from 30 mM to 200 mM, from 40 mM to 150 mM, from 50 mM to 100 mM, for example 56±2 mM. The pharmaceutical composition may further comprise a tonicity modifying agent. Such tonicity modifying agents include, without limitation, glycerol, amino acids, sodium chloride, proteins, sugars and sugar alcohols. In some embodiments, the tonicity modifying agent is a sugar, for example, the tonicity modifying agent is sucrose. In some embodiments, the tonicity modifying agent is present in a concentration of 50 mM to 200 mM, more of 100 mM to 175 mM, of 135 mM to 160 mM, for example, 150±2 mM. In some embodiments, the pharmaceutical composition further comprises a surfactant. In some embodiments, the surfactant is a non-ionic surfactant. In some embodiments, the surfactant or non-ionic surfactant is a polysorbate-based non-ionic surfactant, for example polysorbate 20 or polysorbate 80. In some embodiments, the surfactant or non-ionic surfactant is present in a concentration of 0.01 mg/mL to 1 mg/mL, of 0.05 to 0.5 mg/mL, for example 0.2±0.02 mg/mL.

In some embodiments, the pharmaceutical composition comprises 0.1 mg/mL to 50 mg/mL of mutant FGF-21 peptide conjugate, 1 mM to 100 mM buffering agent, for example Tris buffer, 30 mM to 200 mM salt, for example NaCl, 50 mM to 200 mM tonicity modifying agent, for example sucrose, and 0.01 mg/mL to 1 mg/mL surfactant or non-ionic surfactant, for example polysorbate 20, and has a pH of 6.0 to 8.5.

In some embodiments, the pharmaceutical composition is a liquid pharmaceutical composition comprising at least one mutant FGF-21 peptide conjugate and a pharmaceutically acceptable carrier. In some embodiments, the mutant FGF-21 peptide conjugate is present in a concentration in the range from 0.1 mg/mL to 50 mg/mL. In some embodiments, the mutant FGF-21 peptide conjugate is present in a concentration in the range from 10 mg/mL to 48 mg/mL. In some embodiments the mutant FGF-21 peptide conjugate is present in a concentration of 26-4 mg/mL. For example, the FGF-21 peptide conjugate is present at a concentration of about 22, 23, 24, 25, 26, 27, 28, 29, 30, 31, 32, 33, 34, 35, 36 mg/mL. In some embodiments, the mutant FGF-21 peptide conjugate is present in a 36±6 mg/mL. For example, the FGF-21 peptide conjugate is present at a concentration of about 30, 32, 34, 36, 38, 40, 42 mg/mL.

In some embodiments, the liquid pharmaceutical composition comprises 10-48 mg/mL FGF-21 peptide conjugate, for example about 10 mg/mL, 12 mg/mL, 14, mg/mL, 15 mg/mL, 16 mg/mL 18 mg/mL, 20 mg/mL, 22 mg/mL, 24 mg/mL, 26 mg/mL, 28 mg/mL, 30 mg/mL, 32 mg/mL, 34 mg/mL, 36 mg/mL, 38 mg/mL, 40 mg/mL, 42 mg/mL, 44 mg/mL, 66 mg/mL, 48 mg/mL.

In some embodiments, liquid pharmaceutical composition comprises or consists of from about 10 mg/mL to about 48 mg/mL of a mutant Fibroblast Growth Factor-21 (FGF-21) peptide conjugate, from about 50 mM to about 500 mM arginine; from about 0.01 to about 0.1% (w/v) Polysorbate 80 (PS-80) or Polysorbate 20 (PS-20); about 20 mM buffer, pH 7-8; and a pharmaceutically acceptable carrier. In some embodiments, liquid pharmaceutical composition comprises or consists of from about 10 mg/mL to about 48 mg/mL of a mutant Fibroblast Growth Factor-21 (FGF-21) peptide conjugate, from about 150 mM to about 500 mM arginine; from about 0.01 to about 0.1% (w/v) Polysorbate 80 (PS-80) or Polysorbate 20 (PS-20); about 20 mM buffer, pH 7-8; and a pharmaceutically acceptable carrier. In some embodiments, the formulation has an osmolality between about 250 mOsmol/kg to about 510 mOsmol/kg. In some embodiments, the liquid formulation comprises or consists of from 10 mg/mL to 48 mg/ml of a mutant Fibroblast Growth Factor-21 (FGF-21) peptide conjugate comprising a mutant FGF-21 peptide comprising the amino acid sequence of SEQ ID NO: 2, a glycosyl moiety, and a 20 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG; from 50 mM to 500 mM Arginine; from 0.01 to 0.1% (w/v) Polysorbate 80 (PS-80) or Polysorbate 20 (PS-20); 20 mM buffer, pH 7-8; and a pharmaceutically acceptable carrier. In some embodiments, the liquid formulation comprises or consists of from 10 mg/mL to 48 mg/mL of a mutant Fibroblast Growth Factor-21 (FGF-21) peptide conjugate comprising a mutant FGF-21 peptide comprising the amino acid sequence of SEQ ID NO: 2, a glycosyl moiety, and a 20 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG; from 150 mM to 500 mM Arginine; from 0.01 to 0.1% (w/v) Polysorbate 80 (PS-80) or Polysorbate 20 (PS-20); 20 mM buffer, pH 7-8; and a pharmaceutically acceptable carrier. In some embodiments, the formulation has an osmolality between about 250 mOsmol/kg to about 550 mOsmol/kg. In some embodiments, the liquid pharmaceutical composition comprising or consisting of from about 10 mg/ml to about 48 mg/ml of a mutant Fibroblast Growth Factor-21 (FGF-21) peptide conjugate comprising a mutant FGF-21 peptide comprising the amino acid sequence of SEQ ID NO: 2, a glycosyl moiety, and a 20 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG; from about 50 mM to about 500 mM arginine, from about 50 mM to about 250 mM alanine, about 50 mM to about 250 mM proline, about 50 mM to about 250 mM glycine, about 50 mM to about 250 mM MgCl₂, about 1% to about 5% (v/v) glycerol, about 1% to 5% (v/v) PEG 400, or combination thereof; from about 0.01 to about 0.1% (w/v) Polysorbate 80 (PS-80) or Polysorbate 20 (PS-20); about 20 mM buffer at pH 7-8; and a pharmaceutically acceptable carrier. In some embodiments, the liquid pharmaceutical composition comprising or consisting of from about 10 mg/mL to about 48 mg/mL of a mutant Fibroblast Growth Factor-21 (FGF-21) peptide conjugate comprising a mutant FGF-21 peptide comprising the amino acid sequence of SEQ ID NO: 2, a glycosyl moiety, and a 20 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG; from about 150 mM to about 500 mM arginine, from about 50 mM to about 250 mM alanine, about 50 mM to about 250 mM proline, about 50 mM to about 250 mM glycine, about 50 mM to about 250 mM MgCl₂, about 1% to about 5% (v/v) glycerol, about 1% to 5% (v/v) PEG 400, or combination thereof, from about 0.01 to about 0.1% (w/v) Polysorbate 80 (PS-80) or Polysorbate 20 (PS-20); about 20 mM buffer at pH 7-8; and a pharmaceutically acceptable carrier. In some embodiments, the weight ratio of mutant FGF-21 to arginine is from about 0.6 to about 0.7, from about 0.6 to about 0.8, about 0.6 to about 0.9, from about 0.6 to about 1, e.g. about 0.6, 0.7, 0.8, 0.9, 0.1. In some embodiments, the molar ratio of mutant FGF-21 to arginine is from about from about 0.006 to about 0.008, 0.006 to about 0.009, 0.006 to about 0.010, from about 0.007 to about 0.008, from about 0.007 to about 0.009, from about 0.007 to about 0.010, e.g about 0.006, 0.007, 0.008, 0.009.

In some embodiments, the liquid formulation has an osmolality of about 250 mOsmol/kg to about 550 mOsmol/kg.

Liquid pharmaceutical compositions in some embodiments comprise 20 mg/mL PEG-FGF21 in 20 mM Tris, 150 mM Arginine, 0.02% (w/v) PS-80, pH 7.5. Liquid pharmaceutical formulations in some embodiments comprise 20 mg/mL PEG-FGF21 in 20 mM Phosphate, 150 mM Arginine, 0.02% (w/v) PS-80, pH 7.5. In some embodiments, the composition has an osmolality between about 250 mOsm/kg to about 380 mOsm/kg. In some embodiments, the composition has an osmolality of about 300 mOsm/kg. Liquid pharmaceutical compositions in some embodiments comprise 28 mg/mL PEG-FGF21 in 20 mM Tris, 275 mM Arginine, 0.02% (w/v) PS-80, pH 7-8. In some embodiments, the composition has an osmolality of about 505 mOsm/kg. Liquid pharmaceutical formulations in some embodiments comprise 18-44 mg/mL PEG-FGF21 in 20 mM Tris, 200-350 mM Arginine, 0.02% (w/v) PS-80, pH 7.0-pH 7.5. In some embodiments, the liquid pharmaceutical composition comprises about 20 mg/mL PEG-FGF21, about 150 mM arginine HCl, about 20 mM Tris, 0.02% (w/v) PS-80, wherein pH is about 7.5 and has an osmolality is about 300 mOsm/kg. In some embodiments, the liquid pharmaceutical composition comprises about 28 mg/mL PEG-FGF21, about 260 mM Arginine HCl, about 20 mM Tris, about 0.02% (w/v) PS80, wherein pH is about 7.1. In some embodiments, the liquid pharmaceutical composition comprises about 28 mg/mL PEG-FGF21, about 260 mM Arginine HCl, about 20 mM Tris, about 0.02% (w/v) PS80, wherein pH is about 7.1 and has an osmolality of about 505 mOsm/kg. In some embodiments, the liquid pharmaceutical composition comprises about 36 mg/mL PEG-FGF21, about 270 mM Arginine HCl, about 20 mM Tris, about 0.02% (w/v) PS80, wherein pH is about 7.1. In some embodiments, the liquid pharmaceutical composition comprises about 36 mg/mL PEG-FGF21, about 270 mM Arginine HCl, about 20 mM Tris, about 0.02% (w/v) PS80, wherein pH is about 7.1 and has a osmolality is about 530 mOsm/kg. In some embodiments, the liquid pharmaceutical composition comprises 36 mg/mL PEG-FGF21, 200 mM Arginine HCl, 20 mM Tris, 0.02% (w/v) PS80, wherein pHis about 7.1. In some embodiments, the liquid pharmaceutical composition comprises 36 mg/mL PEG-FGF21, 200 mM Arginine HCl, 20 mM Tris, 0.02% (w/v) PS80, wherein pH is about 7.1 and has an osmolality is about 421 mOsm/kg. In some embodiments, the liquid pharmaceutical composition comprises about 42 mg/mL PEG-FGF21, about 270 mM Arginine HCl, about 20 mM Tris, about 0.02% (w/v) PS80, wherein pH is about 7.1. In some embodiments, the liquid pharmaceutical composition comprises about 42 mg/mL PEG-FGF21, about 270 mM Arginine HCl, about 20 mM Tris, about 0.02% (w/v) PS80, wherein pH is about 7.1 and has an osmolality is about 528 mOsm/kg. In some embodiments, the liquid pharmaceutical composition comprises 44 mg/mL mutant FGF21, 200 mM Arginine HCl, 20 mM Tris, 0.02% (w/v) PS80, wherein pH is 7.1. In some embodiments, the liquid pharmaceutical composition comprises 44 mg/mL PEG-FGF21, 200 mM Arginine HCl, 20 mM Tris, 0.02% (w/v) PS80, wherein pH is 7.1 and has an osmolality is about 455 mOsm/kg. In some embodiments, the liquid pharmaceutical composition comprises 44 mg/mL PEG-FGF21, 230 mM Arginine HCl, 20 mM Tris, 0.02% (w/v) PS80, wherein pHis 7.1. In some embodiments, the liquid pharmaceutical composition comprises 44 mg/mL PEG-FGF21, 230 mM Arginine HCl, 20 mM Tris, 0.02% (w/v) PS80, wherein pHis 7.1 and has an osmolality is about 485 mOsm/kg.

In some embodiments, the liquid composition further comprises a surfactant. In some embodiments, the surfactant comprises cetrimonium bromide, sodium gluconate or combination thereof. In some embodiments, the liquid formulation comprises from about 0.05% to about 0.1% (w/v) cetrimonium bromide, from about 0.05% to about 0.1% (w/v) sodium gluconate or combination thereof.

In some embodiments, the liquid pharmaceutical composition further comprising one or more active agent. In some embodiments, the PEG-FGF21 is co-formulated with one or more active agent. In some embodiments, the one or more active agent can comprise a peptide, a small molecule or combinations thereof. In some embodiments, the one or more active agent can comprise an hormone. For example, the one or more agents can comprise oxyntomodulin, insulin, leptin, glucagon. eroxisome proliferator-activated receptor (PPAR) agonists, FXR (Farnesoid X receptor) agonists, Thyroid Hormone Receptor-Beta (TRβ) Agonists, Sodium glucose co-transporter 2 (SGLT2) Inhibitors, analogs thereof, or combinations thereof. As used herein an “analog” is a molecule having a modification including one or more amino acid substitutions, deletions, inversions or additions when compared with wild type peptide sequence.

The buffering agent may be present in a concentration from 1 mM to 100 mM. In some embodiments, the buffering agent is present at a concentration ranging from 2 mM to 75 mM, 5 mM to 50 mM, 10 mM to 25 mM, 14 to 22 mM. In some embodiments, the buffering agent is present at a concentration of about 14, 16, 18, 20, 22, 24, 26, 30, 32, 34, 36, 38, 40 mM or more. For example, the buffering agent is present at a concentration of about 20 mM. The pH may be in the range from 6.0 to 8.5, from 6.5 to 8.0, from 6.75 to 8.0, from 7.1 to 8. The buffering agent may be a Tris phosphate buffer. For example, the buffering agent can have a pH of 7, 7.1, 7.2, 7.3, 7.4, 7.5, 7.6, 7.7, 7.8, 7.9 or 8.

The liquid pharmaceutical composition may further comprise a tonicity modifying agent. Suitable tonicity modifying agents include glycerol, amino acids, sodium chloride, proteins, or sugars and sugar alcohols. For example, the modifying agent comprise arginine, such as arginine HCl or arginine sulfate. The tonicity modifying agent is present in a concentration of 50 mM to 500 mM. For example, the modifying agent (e.g. arginine HCL) comprises from 150 mM to 500 mM arginine, 150 to 275 mM or 245 to 275 mM. In some embodiments, modifying agent comprise arginine, such as arginine HCl or arginine sulfate is present at a concentration between 31.6 mg/ml (150 mM) and 54.8 mg/ml (260 mM).

The liquid pharmaceutical composition may further comprise a non-ionic surfactant. The non-ionic surfactant may be a polysorbate-based non-ionic surfactant such as polysorbate 20 or polysorbate 80. In some embodiments, the surfactant is polysorbate 80. The non-ionic surfactant may be present in a concentration of 0.01% (w/v) to 1% (w/v). For example, the non-ionic surfactant may be present in a concentration of 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% (w/v).

In some embodiments, the liquid pharmaceutical composition may further comprise cetrimonium bromide, sodium gluconate or combination thereof. For example, the composition may comprise from 0.05% to 0.1% (w/v) cetrimonium bromide, from 0.05% to 0.1% (w/v) sodium gluconate or combination thereof.

In an embodiment, the liquid pharmaceutical composition comprises 10 mg/mL to 50 mg/mL of mutant FGF-21 peptide conjugate, 1 mM to 100 mM buffering agent, for example Tris buffer, 150 mM to 500 mM tonicity arginine, and 0.02% to 1% (w/v) polysorbate-based non-ionic surfactant, for example, polysorbate 80, and has a pH of 7.0 to 8.0.

In some embodiments, the liquid formulation comprises 0.02% (w/v) PS80 (0.2 mg/ml). In some embodiments, the buffer is Tris or phosphate buffer. In some embodiments, the liquid formulation comprises 20 mM Tris buffer. In some embodiments, the liquid formulation comprises 15 mg/ml of mutant FGF-21. In some embodiments, the liquid formulation comprises 28 mg/ml of mutant FGF-21. In some embodiments, the liquid formulation comprises 30 mg/ml of mutant FGF-21. In some embodiments, the liquid formulation comprises 36 mg/ml of mutant FGF-21. In some embodiments, the liquid formulation comprises 44 mg/ml of mutant FGF-21. In some embodiments, the liquid formulation comprises from 150 mM to 275 mM arginine. In some embodiments, arginine is arginine HCl or arginine sulfate. In some embodiments, the pH is 7.1.

In some embodiments, the liquid pharmaceutical composition comprises about 20 mg/mL mutant FGF21, about 150 mM arginine HCl, about 20 mM Tris, 0.02% (w/v) PS-80 and wherein pH is about 7.5. In some embodiments, the liquid pharmaceutical composition comprises about 28 mg/mL mutant FGF21, about 260 mM arginine HCl, about 20 mM Tris, about 0.02% (w/v) PS80 and wherein pH is about 7.1. In some embodiments, the liquid pharmaceutical composition comprises about 36 mg/mL mutant FGF21, about 270 mM arginine HCl, about 20 mM Tris, about 0.02% (w/v) PS80 and wherein pH is about 7.1. In some embodiments, the liquid pharmaceutical composition comprises 36 mg/mL mutant FGF21, 200 mM arginine HCl, 20 mM Tris, 0.02% (w/v) PS80 and wherein pH is about 7.1 In some embodiments, the liquid pharmaceutical composition comprises about 42 mg/mL mutant FGF21, about 270 mM arginine HCl, about 20 mM Tris, about 0.02% (w/v) PS80 and wherein pH is about 7.1. In some embodiments, the liquid pharmaceutical composition comprises 44 mg/mL mutant FGF21, 200 mM arginine HCl, 20 mM Tris, 0.02% (w/v) PS80 and wherein pH is 7.1. In some embodiments, the liquid pharmaceutical composition comprises 44 mg/mL mutant FGF21, 230 mM arginine HCl, 20 mM Tris, 0.02% (w/v) PS80 and wherein pH is 7.1.

In some embodiments, the pharmaceutical composition is a liquid pharmaceutical composition comprising: (a) from 10 mg/ml to 48 mg/ml of a mutant Fibroblast Growth Factor-21 (FGF-21) peptide conjugate comprising a mutant FGF-21 peptide comprising the amino acid sequence of SEQ ID NO: 2, a glycosyl moiety, and a 20 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG; (b) from 50 mM to 500 mM arginine; (c) from 0.01% to 0.1% (w/v) Polysorbate 80 (PS-80) or Polysorbate 20 (PS-20); (d) from 5 to 25 mM buffer, pH 7-8; and (e) a pharmaceutically acceptable carrier.

In some embodiments, the pharmaceutical composition is a liquid pharmaceutical composition comprising: (a) from 10 mg/ml to 48 mg/ml of a mutant Fibroblast Growth Factor-21 (FGF-21) peptide conjugate comprising a mutant FGF-21 peptide comprising the amino acid sequence of SEQ ID NO: 2, a glycosyl moiety, and a 20 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG; (b) from 150 mM to 500 mM arginine; (c) from 0.01% to 0.1% (w/v) Polysorbate 80 (PS-80) or Polysorbate 20 (PS-20); (d) from 5 to 25 mM buffer, pH 7-8; and (e) a pharmaceutically acceptable carrier.

In some embodiments, the liquid pharmaceutical composition comprises: (a) from 10 mg/ml to 48 mg/ml of a mutant Fibroblast Growth Factor-21 (FGF-21) peptide conjugate comprising a mutant FGF-21 peptide comprising the amino acid sequence of SEQ ID NO: 2, a glycosyl moiety, and a 20 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG; (b) from 150 mM to 500 mM arginine, from 50 mM to 250 mM alanine, 50 mM to 250 mM proline, 50 mM to 250 mM glycine, 50 mM to 250 mM MgCl₂, 1 to 5% (v/v) glycerol, 1 to 5% (v/v) PEG 400, or combination thereof; (c) from 0.01% to 0.1% (w/v) Polysorbate 80 (PS-80) or Polysorbate 20 (PS-20); (d) a buffer having a pH of 7-8; and (e) a pharmaceutically acceptable carrier.

In some embodiments, the liquid formulation, further comprises a surfactant. In some embodiments, the surfactant comprises cetrimonium bromide, sodium gluconate or combination thereof. In some embodiments, the liquid formulation comprises from 0.05% to 0.1% (w/v) cetrimonium bromide, from 0.05% to 0.1% (w/v) sodium gluconate or combination thereof.

In some embodiments, the buffer is Tris or phosphate buffer. In some embodiments, the liquid formulation comprises 20 mM Tris buffer. In some embodiments, the pH of the liquid formulation is from 7.0 to 7.5.

In some embodiments, the liquid pharmaceutical composition comprises from 20 to 44 mg/ml of the mutant FGF-21 peptide conjugate.

In some embodiments, the liquid pharmaceutical composition comprises from 150 mM to 275 mM arginine. In some embodiments, the arginine in the liquid pharmaceutical composition comprises arginine HCl, arginine sulfate or combination thereof. In some embodiments, the weight ratio of mutant FGF-21 peptide conjugate to arginine is from 0.6 to 0.9. In some embodiments, the molar ratio of mutant FGF-21 peptide conjugate to arginine is from about 0.006 to about 0.009.

In some embodiments, the liquid pharmaceutical composition comprises about 28 mg/mL mutant FGF-21 peptide conjugate, about 260 mM arginine HCl, about 20 mM Tris, 0.02% (w/v) PS-80 and wherein pH is about 7.1.

In some embodiments, the liquid pharmaceutical composition comprises about 20 mg/mL mutant FGF-21 peptide conjugate, about 150 mM arginine HCl, about 20 mM Tris, 0.02% (w/v) PS-80 and wherein pH is about 7.5.

In some embodiments, the liquid pharmaceutical composition comprises about 36 mg/mL mutant FGF-21 peptide conjugate, about 200 mM arginine HCl, about 20 mM Tris, 0.02% (w/v) PS-80 and wherein pH is about 7.1.

In some embodiments, the liquid pharmaceutical composition comprises about 44 mg/mL mutant FGF-21 peptide conjugate, about 200 mM arginine HCl, about 20 mM Tris, 0.02% (w/v) PS-80 and wherein pH is about 7.1.

In some embodiments, the liquid pharmaceutical composition comprises about 44 mg/mL mutant FGF-21 peptide conjugate, about 230 mM arginine HCl, about 20 mM Tris, 0.02% (w/v) PS-80 and wherein pH is about 7.1.

In some embodiments, the liquid formulation has an osmolality of about 250 mOsmol/kg to about 550 mOsmol/kg.

In some embodiments, the liquid formulation is a liquid formulation as described in U.S. Pat. No. 11,596,669 which is incorporated by reference in its entirety.

In some embodiments, also encompassed herein is a pharmaceutical container comprising any one of or at least one of a mutant FGF-21 peptide conjugate described herein or a pharmaceutical composition comprising same. Exemplary such pharmaceutical containers include, without limitation, a syringe, vial, infusion bottle, ampoule, carpoule, a syringe equipped with a needle protection system, or a carpoule within an injection pen.

### Method of Treatment

In some embodiments, methods for treating (e.g., controlling, relieving, ameliorating, alleviating, or slowing the progression of) or methods for preventing (e.g., delaying the onset of or reducing the risk of developing) diabetes and related diseases, particularly diabetes type 2, NAFLD, non-alcoholic steatohepatitis (NASH) and/or metabolic syndrome in a subject in need thereof are disclosed.

In some embodiments, a method for treating diabetes and related diseases, particularly diabetes type 2, NAFLD, non-alcoholic steatohepatitis (NASH) and/or metabolic syndrome is presented, the method comprising administering to a subject in need thereof a therapeutically effective amount of a mutant FGF-21 peptide conjugate described herein or a pharmaceutical composition comprising at least one of the mutant FGF-21 peptide conjugates described herein. In some embodiments, the subject in need thereof is a human subject.

In some embodiments, a method for treating diabetes or a diabetes related disease is disclosed, comprising administering to a subject in need thereof an amount of a mutant FGF-21 peptide conjugate described herein or obtainable by a method described herein or a pharmaceutical composition comprising same. The diabetes or the diabetes related disease may comprise at least one of diabetes type 2, NAFLD, NASH, or metabolic syndrome. In some embodiments, a method for treating NASH is disclosed, comprising administering to a subject in need thereof an amount of a mutant FGF-21 peptide conjugate described herein or obtainable by a method described herein or a pharmaceutical composition comprising same. In some embodiments, a method for treating NAFLD is disclosed, comprising administering to a subject in need thereof an amount of a mutant FGF-21 peptide conjugate described herein or obtainable by a method described herein or a pharmaceutical composition comprising same. In some embodiments, a method for preventing the progression of NASH is disclosed, comprising administering to a subject in need thereof an amount of a mutant FGF-21 peptide conjugate described herein or obtainable by a method described herein or a pharmaceutical composition comprising same. In some embodiments, a method for preventing the progression of NAFLD is disclosed, comprising administering to a subject in need thereof an amount of a mutant FGF-21 peptide conjugate described herein or obtainable by a method described herein or a pharmaceutical composition comprising same. In some embodiments, the subject is a human subject. In some embodiments, the administering reduces HbA1C levels, wherein reducing HbA1C levels is indicative of a durable reduction in blood glucose levels over time. A variety of exemplary indicators are known in the art and described herein including, without limitation, a reduction in glucose, insulin, body weight, serum lipids (total cholesterol, LDL, Triglycerides), liver enzymes (ALT, AST), liver weight, relative liver weight (% body weight), NAFLD Activity Score (NAS), fibrosis score (e.g., liver fibrosis), pro-inflammatory cytokines (e.g., IL1β, MCP-1), fibrosis biomarkers (αSMA, Collagen 1 alpha), hepatic cholesterol, hepatic triglycerides, and hepatic fatty acids. Increases in at least one of adiponectin (e.g. high molecular weight (HMW)) or HDL are also indicators of clinical efficacy of compounds and compositions described herein. In some embodiments, the method of treatment of the method for preventing the progression of the disease is assessed by Magnetic resonance imaging-Proton density fat fraction to determine the liver size (e.g. reduction of liver size).

The NAFLD fibrosis score is used to distinguish between patients with nonalcoholic fatty liver disease who have (F3-F4) and do not have (F0-F2) advanced fibrosis. A fibrosis score of F0 to F1 (2 to 7 kPa) means there is little or no scarring on the liver. A fibrosis score of F2 (7.5 to 10 kPa) indicates moderate scarring that has spread outside the liver. A fibrosis score of F3 (10 to 14 kPa) indicates severe scarring which has spread and disrupts normal blood flow. A fibrosis score of F4 (14 kPa or higher) means late-stage scarring or cirrhosis, where the scarring is permanent and the damage is irreversible.

Methods of treatment of NASH

In some embodiments, methods for treating (e.g., controlling, relieving, ameliorating, alleviating, or slowing the progression of) or methods for preventing (e.g., delaying the onset of or reducing the risk of developing) non-alcoholic steatohepatitis (NASH) in a subject in need thereof are disclosed.

In some embodiments, a method for treating non-alcoholic steatohepatitis (NASH) is presented, the method comprising administering to a subject in need thereof a therapeutically effective amount of a mutant FGF-21 peptide conjugate described herein or a pharmaceutical composition comprising at least one of the mutant FGF-21 peptide conjugates described herein. In some embodiments, the subject in need thereof is a human subject.

In some embodiments, the step of administering comprises administering an effective dose of the composition that results in NASH resolution (NAS score of 0-1 for inflammation, 0 for ballooning).

In some embodiments, the step of administering comprises administering an effective dose of the composition that results in at least one of the following: reduction of at the level of markers of NASH (e.g. serum markers), reduction of symptoms associated with NASH, reduction of liver fibrosis, reduction in NAS score.

In some embodiments, a method for treating NASH is disclosed, comprising administering to a subject in need thereof an amount of a mutant FGF-21 peptide conjugate described herein or obtainable by a method described herein or a pharmaceutical composition comprising same. In some embodiments, a method for preventing the progression of NASH is disclosed, comprising administering to a subject in need thereof an amount of a mutant FGF-21 peptide conjugate described herein or obtainable by a method described herein or a pharmaceutical composition comprising same. In some embodiments, the subject is a human subject. In some embodiments, the administering reduces HbA1C levels, wherein reducing HbA1C levels is indicative of a durable reduction in blood glucose levels over time. A variety of exemplary indicators are known in the art and described herein including, without limitation, a reduction in glucose, insulin, body weight, serum lipids (total cholesterol, LDL, Triglycerides), liver enzymes (ALT, AST), liver weight, relative liver weight (% body weight), NAFLD Activity Score (NAS), fibrosis score (e.g., liver fibrosis), FIB-4 score, FAST score, pro-inflammatory cytokines (e.g., IL1β, MCP-1), fibrosis biomarkers (αSMA, Collagen 1 alpha), hepatic cholesterol, hepatic triglycerides, and hepatic fatty acids. Increases in at least one of adiponectin (e.g. high molecular weight (HMW)) or HDL are also indicators of clinical efficacy of compounds and compositions described herein. In some embodiments, the method of treatment of the method for preventing the progression of the disease is assessed by Magnetic resonance imaging-Proton density fat fraction to determine the liver size (e.g. reduction of liver size).

In some embodiments, the indicators or endpoints used to determine the efficacy of the treatment include one or more of the following:
NAS score
NASH resolution
Fibrosis improvement.

In some embodiments, the biomarkers used to determine the efficacy of the treatment include one or more of the following:
Fibrosis-4 (FIB-4) score
FibroScan-AST (FAST) score
Vibration-Controlled Transient Elastography (VCTE) to assess fibrosis and steatosis
Triglycerides
Non-high density lipoprotein (Non-HDL) cholesterol
High density lipoprotein (HDL-c)
Low density lipoprotein (LDL-c)
Hemoglobin A1c (HbA1c)
Homeostatic Model Assessment for Insulin Resistance (HOMA-IR)
Liver function tests: alanine transaminase (ALT), aspartate transaminase (AST)
Adiponectin
N-Terminal Propeptide of Type III Collagen (Pro-C3)
Free fatty acids and Adipo-IR (fasting free fatty acids x fasting insulin)
inflammation marker high-sensitivity C-reactive protein (hs-CRP)
Total cholesterol
OGTT including C-peptide, glucose, and insulin
IGF-1, total
CK-18
Enhanced LiverFibrosis (ELF) panel.

In some embodiments, encompassed herein is any one of the mutant FGF-21 peptide conjugates described herein or a pharmaceutical composition comprising same for use in a method for treating diabetes or a diabetes related disease. The diabetes or the diabetes related disease may comprise at least one of diabetes type 2, NAFLD, NASH, or metabolic syndrome. In some embodiments, the diabetes or the diabetes related disease afflicts a human subject. In some embodiments, the use reduces HbA1C levels, wherein reducing HbA1C levels is indicative of a durable reduction in blood glucose levels over time. A variety of exemplary indicators are known in the art and described herein including, without limitation, a reduction in glucose, insulin, body weight, serum lipids (total cholesterol, LDL, Triglycerides), liver enzymes (ALT, AST), liver weight, relative liver weight (% body weight), NAFLD Activity Score (NAS), fibrosis score (e.g., liver fibrosis), pro-inflammatory cytokines (e.g., IL1β, MCP-1), fibrosis biomarkers (αSMA, Collagen 1 alpha), hepatic cholesterol, hepatic triglycerides, and hepatic fatty acids. Increases in at least one of high molecular weight (HMW) adiponectin (e.g. high molecular weight (HMW)) or HDL are also indicators of clinical efficacy of compounds and compositions described herein.

In some embodiments, use of a mutant FGF-21 peptide conjugate described herein in the preparation of a medicament for use in a method for treating diabetes or a diabetes related disease is presented. The diabetes or the diabetes related disease may comprise at least one of diabetes type 2, NAFLD, NASH, or metabolic syndrome. In some embodiments, the diabetes or the diabetes related disease afflicts a human subject. In some embodiments, the use reduces HbA1C levels, wherein reducing HbA1C levels is indicative of a durable reduction in blood glucose levels over time. A variety of exemplary indicators are known in the art and described herein including, without limitation, a reduction in glucose, insulin, body weight, serum lipids (total cholesterol, LDL, Triglycerides), liver enzymes (ALT, AST), liver weight, relative liver weight (% body weight), NAFLD Activity Score (NAS), fibrosis score (e.g., liver fibrosis), pro-inflammatory cytokines (e.g., IL1β, MCP-1), fibrosis biomarkers (αSMA, Collagen 1 alpha), hepatic cholesterol, hepatic triglycerides, and hepatic fatty acids. Increases in at least one of adiponectin (e.g. high molecular weight (HMW)) or HDL are also indicators of clinical efficacy of compounds and compositions described herein.

In some embodiments, a mutant Fibroblast Growth Factor-21 (FGF-21) peptide conjugate is presented comprising
i) a mutant FGF-21 peptide comprising the amino acid sequence of SEQ ID NO: 2,
ii) a glycosyl moiety, wherein the glycosyl moiety comprises the structure -GalNAc-Sia-, and
iii) a 30 kDa polyethylene glycol (PEG),
   wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 30 kDa PEG by a covalent bond between a second site of the glycosyl moiety and the 30 kDa PEG. In some embodiments, the 30 kDa PEG moiety is attached to the glycosyl moiety by a covalent bond to a linker, wherein the linker comprises at least one amino acid residue. Exemplary amino acids, include: polar, but neutral amino acids (e.g., serine, threonine, cysteine, tyrosine, asparagine, and glutamine) and non-polar amino acids with relatively simple side chains (e.g. glycine, alanine, valine, leucine). In some embodiments, the at least one amino acid residue is at least one glycine (Gly). In some embodiments, the mutant FGF-21 peptide conjugate comprises the structure-GalNAc-Sia-Gly-PEG (30 kDa). A mutant FGF-21 peptide conjugate described herein may comprise a 30 kDa PEG which is a linear or branched PEG. In some embodiments, the 30 kDa PEG is a linear PEG. In some embodiments, the 30 kDa PEG is a 30 kDa methoxy-PEG. In some embodiments, single or multiple administrations of the compositions can be carried out with dose levels and pattern being selected by the treating physician. In any event, the pharmaceutical compositions should provide a quantity of the mutant FGF-21 peptide conjugate of this disclosure sufficient for an effective treatment of the subject in need of such treatment.

In the pharmaceutical composition, in some embodiments, the mutant FGF-21 peptide conjugate is present in a concentration in the range from 0.1 mg/mL to 50 mg/mL.

All components of the pharmaceutical composition as well as the specific concentrations of the components have carefully selected after testing very many different conditions, compounds and concentrations thereof. Hence, the pharmaceutical composition disclosed herein is not an arbitrary selection of compounds and compound concentrations but a specific and rational selection of conditions which have been found to be most optimal for an aqueous pharmaceutical composition containing the mutant FGF-21 peptide conjugate or mutant FGF-21 peptide according to the disclosure for use as a medicament.

In some embodiments, the pharmaceutical composition comprises a buffering agent, such as a phosphate or Tris buffer, particularly a Tris buffer, e.g. Tris(hydroxymethyl)aminomethane (THAM). Optionally, the buffering agent is present in a concentration from 1 mM to 100 mM, from 2 mM to 75 mM, from 5 mM to 50 mM, from 10 mM to 25 mM, for example 16±2 mM. Tris buffer was selected since solubility of the protein was found to be better than for other buffer systems and it is suitable to keep the pH at pH 7.5. This pH seems the most optimal one for prolonged storage of the PEGylated mutant FGF-21 peptide conjugate. Moreover, probability of Tris crystallization at lower temperatures is lower than that of phosphate based buffering agents.

In some embodiments, the mutant FGF-21 peptide conjugate may undergo precipitation of the pH is below 6.0. Optionally, the pH of the pharmaceutical composition is in the range from 6.0 to 8.5, from 6.5 to 8.0, from 6.75 to 8.0, from 7.0 to 8.0, for example 7.5±0.3 as lowest fragmentation in SDS-PAGE and least aggregation in SEC was observed if the pH is in the range of 7-8. This pH has also been identified to be optional with respect to viscosity. As the pH of a solution may depend on the temperature of the solution, the pH should be adapted and measured at 25±2° C. The pH is adjusted with HCl. The pharmaceutical composition may further comprise a salt, particularly an inorganic salt, for example NaCl. Optionally, the salt is present in a concentration from 30 mM to 200 mM, from 40 mM to 150 mM, from 50 mM to 100 mM, for example 56±2 mM. The presence of a salt (e.g. NaCl), is beneficial to reduce viscosity which is increased in PEG containing samples. For the same reason, it is also beneficial to include sorbitol and/or glycine.

In some embodiments, the pharmaceutical composition may further comprise a tonicity modifying agent. The tonicity modifying agent may be selected from the group consisting of glycerol, amino acids, sodium chloride, proteins, sugars and sugar alcohols. In some embodiments, the tonicity modifying agent is a sugar (e.g., sucrose). A tonicity modifying agent, in particular sucrose, was found to have an advantageous effect on the pharmaceutical composition as it reduces aggregation of the active agent, namely the mutant FGF-21 peptide (conjugate).

The tonicity modifying agent, for example sucrose, may be present in a concentration of 50 mM to 200 mM, of 100 mM to 175 mM, of 135 mM to 160 mM, for example 150±2 mM.

Further, the pharmaceutical composition, in some embodiments, may comprise a surfactant, e.g. a non-ionic surfactant. In some embodiments, the surfactant or non-ionic surfactant is a polysorbate-based non-ionic surfactant, e.g. polysorbate 20 or polysorbate 80. A surfactant, e.g. polysorbate 20 or polysorbate 80, was found to reduce sub-visible particles below 10 μm and thus seems to have a stabilizing effect on the pharmaceutical composition.

The surfactant or non-ionic surfactant, for example, polysorbate 20 or polysorbate 80, is optionally present in a concentration of 0.01 mg/mL to 1 mg/mL, 0.05 to 0.5 mg/mL, for example, in a concentration of 0.2±0.02 mg/mL. Polysorbate 20 or 80 were found to stabilize the formulation to aggregation.

In some embodiments, a pharmaceutical composition comprises 0.1 to 50 mg/mL, for example 33±7 mg/mL of mutant FGF-21 peptide conjugate; 1 mM to 100 mM, for example 20=2 mM, buffering agent, for example a Tris buffer; 30 mM to 200 mM, for example 70±2 mM, salt, particularly NaCl; and has a pH of 7.5±0.3 (measured at 25±2° C.).

In some embodiments, the pharmaceutical composition comprises 0.1 to 50 mg/mL, for example 26±4 mg/mL of mutant FGF-21 peptide conjugate; 1 mM to 100 mM, for example 16±2 mM, buffering agent, such as a Tris buffer; 30 mM to 200 mM, for example 56±2 mM, salt, for example NaCl; 50 mM-200 mM tonicity modifying agent, for example sucrose; and 0.01 to 1 mg/mL, for example 0.2±0.02 mg/mL, surfactant or non-ionic surfactant (e.g. polysorbate 20); and has a pH of 7.5±0.3 (measured at 25±2° C.).

In some embodiments, a pharmaceutical container comprising the mutant FGF-21 peptide conjugate of the disclosure and as described herein or the pharmaceutical composition of the disclosure and as described herein. In some embodiments, the pharmaceutical container is a syringe, vial, infusion bottle, ampoule, carpoule, a syringe equipped with a needle protection system, or a carpoule within an injection pen.

After protein expression, optional purification, the PEG residue is attached to the mutant FGF-21 peptide, specifically at the newly introduced threonine residue via at least one glycosyl moiety and optionally via at least one amino acid residue which is present between the PEG and the glycosyl residue.

To obtain high yield expression of a nucleic acid encoding a mutant FGF-21 of the present disclosure, one typically subclones a polynucleotide encoding the mutant Fibroblast Growth Factor into an expression vector that contains a strong promoter to direct transcription, a transcription/translation terminator and a ribosome binding site for translational initiation. Suitable bacterial promoters are described, e.g., in Sambrook and Russell, supra, and Ausubel et al, supra. Bacterial expression systems for expressing the native or mutant FGF-21 are available in, e.g., Escherichia coli (E. coli), Bacillus sp., Salmonella, and Caulobacter. Kits for such expression systems are commercially available. Eukaryotic expression systems for mammalian cells, yeast, and insect cells are commercially available. In one embodiment, the eukaryotic expression vector is an adenoviral vector, an adeno-associated vector, or a retroviral vector. In some embodiments, the mutant FGF-21 peptide is recombinantly produced in E. coli cells, i.e. the expression host is E. coli.

In some embodiments, the present disclosure also provides the mutant FGF-21 peptide conjugate and/or the pharmaceutical composition for use as a medicament and for use in the treatment of diabetes and related diseases, particularly diabetes type 2, nonalcoholic fatty liver disease (NAFLD), non-alcoholic steatohepatitis (NASH) and/or metabolic syndrome. For example, some embodiments also provide the use of the mutant FGF-21 peptide conjugate of the disclosure and/or the use of the pharmaceutical composition of the disclosure for the treatment of diabetes and related diseases, particularly diabetes type 2, NAFLD, NASH and/or metabolic syndrome.

In some embodiments is provided a method of treating diabetes and related diseases, particularly diabetes type 2, NASH, nonalcoholic fatty liver disease (NAFLD), and/or metabolic syndrome comprising administering to a subject in need thereof an amount of the mutant FGF-21 peptide conjugate according to the disclosure or the pharmaceutical composition according to the disclosure. In some embodiments, the subject is a human subject.

NASH is a chronic liver disease, characterized histologically by hepatic steatosis in ≥5% of hepatocytes, injury (ballooning). It is part of the spectrum of NAFLD that includes NASH, and cirrhosis resultant from fatty liver. NAFLD is a common chronic liver disease in Western countries, which can progress to cirrhosis and is associated with an increased mortality risk in general and an increased cardiovascular disease mortality risk in particular. In North America, the prevalence of NAFLD is estimated at ~24%. NAFLD patients tend to be obese, with insulin resistance and/or type 2 diabetes mellitus (T2DM), dyslipidemia, hypertriglyceridemia, and hypertension, and NAFLD is increasingly recognized as the liver disease component of the metabolic syndrome (MetS) (Chalasani, 2018).

NAFLD is usually asymptomatic, unless progression to cirrhosis has occurred. It is often diagnosed by demonstration of hepatic steatosis on liver imaging (e.g., ultrasound or magnetic resonance imaging (MRI)) in subjects, commonly with features of the metabolic syndrome, in whom no alternative etiology for liver fat accumulation can be identified (e.g., alcoholic liver disease, medications). Validated noninvasive tests for diagnosis of steatohepatitis are not currently available, and a liver biopsy is still needed to diagnose the inflammation and cellular ballooning features of NASH (Torres, 2012).

Current pharmacological treatment of NAFLD has limited efficacy and therefore, there is a pressing need to develop more effective and safe agents for this common and life-threatening disease. Obeticholic acid (OCA), a selective agonist of the farnesoid X receptors, appears to have promise as a therapeutic agent for the management of NAFLD. The Farnesoid X Receptor Ligand Obeticholic Acid in NASH Treatment (FLINT) trial in patients with NASH, revealed that OCA administration is associated with improvements in liver histology, as well as weight loss and reduction in blood pressure. Although its adverse effects on lipid profile and insulin sensitivity are noteworthy, OCA might be considered in selected patients with NAFLD/NASH, particularly those with adequately controlled glucose and lipid levels.

With respect to indicators demonstrating clinical efficacy of compounds and compositions described herein, a variety of exemplary indicators are known in the art and described herein including, without limitation, a reduction in HbA1c, glucose and Insulin, body weight, serum lipids (total cholesterol, LDL, Triglycerides), liver enzymes (ALT, AST), liver weight, relative liver weight (% body weight), NAFLD Activity Score (NAS), fibrosis score (e.g., liver fibrosis), pro-inflammatory cytokines (e.g., IL1β, MCP-1), fibrosis biomarkers (αSMA, Collagen 1 alpha), hepatic cholesterol, hepatic triglycerides, and hepatic fatty acids. Increases in at least one of adiponectin (e.g. high molecular weight (HMW)) or HDL are also indicators of clinical efficacy of compounds and compositions described herein. Accordingly, a change (as indicated above) in at least one of the indicators reflects clinical efficacy of a compound or composition described herein.

In some embodiments, the therapeutic efficacy of a compound or composition described herein is determined based on a reduction in at least one of serum triglyceride levels or serum insulin levels.

HOMA-IR is, for example, is an indicator of the presence and extent of insulin resistance in a subject. It is an accurate indicator of the dynamic between baseline (fasting) blood sugar and insulin levels responsive thereto. It is referred to as an insulin resistance calculator. For humans, a healthy range is 1.0 (0.5-1.4). Less than 1.0 indicates that a subject is insulin-sensitive, which is ideal; above 1.9 indicates that a subject is exhibiting early insulin resistance; above 2.9 indicates that a subject is exhibiting significant insulin resistance. HOMA-IR blood code calculation is determined as follows: insulin uIU/mL (mU/L) X glucose (mg/dL)=HOMA-IR. The calculation requires U.S. standard units. To convert from international SI units: for insulin: pmol/L to uIU/mL, divide (÷) by 6; for glucose: mmol/L to mg/dL, multiply (X) by 8.

Some embodiments relate to dosage regimen whereby an effective amount of a mutant FGF-21 peptide conjugate described herein or a pharmaceutical composition comprising a therapeutically effective amount of a mutant FGF-21 peptide conjugate is administered to the subject in need thereof. In some embodiments, from about 3 mg to about 30 mg of a mutant FGF-21 peptide conjugate described herein or a pharmaceutical composition comprising from about 3 mg to about 30 mg of a mutant FGF-21 peptide conjugate is administered to the subject in need thereof once a week. In some embodiments, the effective amount of mutant FGF-21 peptide conjugate can be in a range of about 3 mg to about 30 mg; a range of about 9 mg to about 30 mg; a range of about 15 mg to about 30 mg; a range of about 18 mg to about 30 mg; a range of about 3 mg to about 27 mg; a range of about 9 mg to about 27 mg; a range of about 15 mg to about 27 mg; a range of about 18 mg to about 27 mg; a range of about 3 mg to about 9 mg; a range of about 9 mg to about 15 mg; a range of about 9 mg to about 18 mg; a range of about 18 mg to about 27 mg; a range of about 18 mg to about 30 mg; a range of 3 mg to 18 mg and is administered once a week. For example, the effective amount of mutant FGF-21 peptide conjugate can be about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, or about 30 mg.

In some embodiments, from about 18 mg to about 44 mg of a mutant FGF-21 peptide conjugate described herein or a pharmaceutical composition comprising from about 18 mg to about 44 mg of a mutant FGF-21 peptide conjugate is administered to the subject in need thereof once every two weeks. For example, the effective amount of mutant FGF-21 peptide conjugate can be about 18 mg, about 19 mg, about 20 mg, about 21 mg, about 22 mg, about 23 mg, about 24 mg, about 25 mg, 2 about 6 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, or about 44 mg.

In some embodiments, a therapeutically effective amount of a mutant FGF-21 peptide conjugate described herein or a pharmaceutical composition comprising a therapeutically effective amount of a mutant FGF-21 peptide conjugate is administered twice per day, once per day, every two days, three times per week, once per week, once every two weeks, once every three weeks, or once per month.

Long duration efficacy of mutant FGF-21 peptide conjugates described herein is evidenced by the surprisingly long half-life (between about 55 to about 100 hours) determined for these conjugates in animal model systems. Native FGF21 has a short life (~2 hours), limiting the potential to use it as a therapeutic agent. In humans, the effects of pegbelfermin (Pegylated FGF-21 having a half-life of 19-24 hours) showed a lower efficacy in the lipid measurements (% change vs. baseline) when dosed weekly vs daily.

Long duration efficacy of mutant FGF-21 peptide conjugates described herein, in turn, makes it possible to administer the mutant FGF-21 peptide conjugates less frequently, for example one a week or once every two weeks. Accordingly, in some embodiments, a mutant FGF-21 peptide conjugate described herein or a composition comprising same is administered to a subject in need thereof at a frequency of equal to or lower than once per week or once per 2 weeks. For example, the mutant FGF-21 peptide conjugate described herein or a composition comprising same may be administered to a subject in need thereof once every 7 days, once every 8 days, once every 9 days, once every 10 days, once every 11 days, once every 12 days, once every 13 days, once every 14 days, once every 15 days, once every 16 days, once every 17 days, once every 18 days, once every 19 days, once every 20 days, once every 21 days, once every 22 days, once every 22 days, once every 23 days, once every 24 days, once every 25 days, once every 26 days, once every 27 days, once every 28 days, once every 29 days, once every 30 days, or once every 31 days.

In an exemplary embodiment, the compounds described herein and compositions comprising same is administered to a subject in need thereof at a frequency of once a week. In an exemplary embodiment, the compounds described herein and compositions comprising same is administered to a subject in need thereof at a frequency of once every two weeks.

In another exemplary therapeutic regimen, compounds described herein and compositions comprising same are following a course of “induction” therapy, which calls for more frequent administration such as twice a week or weekly at the onset of the treatment regimen followed by maintenance therapy, which may involve weekly, once every two weeks or once a month administration. Such regimen is effective in that the initial induction therapy improves the subject's condition to a manageable level that is acceptable with regard to achieving a clinical state that is acceptable for maintenance of the disease/condition. Thereafter, the maintenance therapy is used to preserve the level of wellness at the maintenance level.

Therapeutic efficacy of a compound and/or composition for treating diabetes and related diseases, particularly diabetes type 2, nonalcoholic fatty liver disease (NAFLD), non-alcoholic steatohepatitis (NASH) and/or metabolic syndrome may be evaluated using a variety of parameters and assays known by persons of skill in the art and described herein (see Example 1 and Example 4). Therapeutic efficacy of a compound and/or composition for treating non-alcoholic steatohepatitis (NASH) may be evaluated using a variety of parameters and assays known by persons of skill in the art and described herein (see Example 5 and Example 6).

Measuring HbA1C is considered a standard assay for measuring glycemic index of a subject over a long duration. It is, therefore, a stable indicator of glycemic index, reflecting glucose levels over the course of approximately the last 3-4 months. Accordingly, a subject who has diabetes (e.g., diabetes type 2) may be defined by the percent HbA1C determined in a suitable assay.

For a healthy person without diabetes, the normal range for the hemoglobin Alc level is between 4% and 5.6%. Hemoglobin Alc levels between 5.7% and 6.4% indicate that a person has a higher chance of developing diabetes. Levels of 6.5% or higher indicate that a person has diabetes.

In some embodiments, HbA1C is measured with HPLC by using the Glycated hemoglobin test system (BIO-RAD, Hercules, CA, USA). Blood samples (e.g., 1.0 mL/per time) may be collected from the cephalic or saphenous vein into BD Vacutainer® K2-EDTA tubes. Samples may be stored immediately at 4 degrees C. or maintained on wet ice and analyzed on the same day the blood was collected. HbA1c levels in the blood may be measured by persons skilled in the art with HPLC by using the Glycated hemoglobin test system (BIO-RAD, Hercules, CA, USA).

With regard to NASH, this condition is currently diagnosed by biopsy. There are some surrogate biomarkers however, that are considered predictive of NASH, such as liver fat (determined by MRI), liver enzymes (ALT and ALT/AST ratio), and fibrosis biomarkers, such as pro-C3, non-invasive tests (NITs), such as VCTE, FAST, FIB-4. (see Examples 4-6)

In some embodiments, the subject in need thereof is on background GLP1 treatment and are administered mutant FGF-21 peptide conjugates described herein one or twice a week as described herein. (See Example 12).

In some embodiments, the method described herein results in a decrease in the development of HCC tumor nodules and tumor burden.

### EXAMPLES

### Example 1: Mutant FGF21-GalNAc-SA-PEG-20 kDa (BIO89-100) Single Ascending Dose

### Study in Humans

Methods: In an embodiment, a total of 58 healthy subjects were treated with subcutaneous (SC) BIO89-100 (7 dose levels) or placebo, in a single-center study to assess safety, tolerability, PK, immunogenicity and exploratory pharmacodynamics. Subjects were randomized to receive a single dose of either BIO89-100 at 0.45 mg, 1.2 mg, 3 mg, 9.1 mg, 18.2 mg, 39 mg or 78 mg, or placebo at a 6:2 ratio (7:3 ratio for the 9.1 mg dose). Subjects were followed for 4 weeks with frequent assessments initially and then weekly from Day 8 through Day 29.

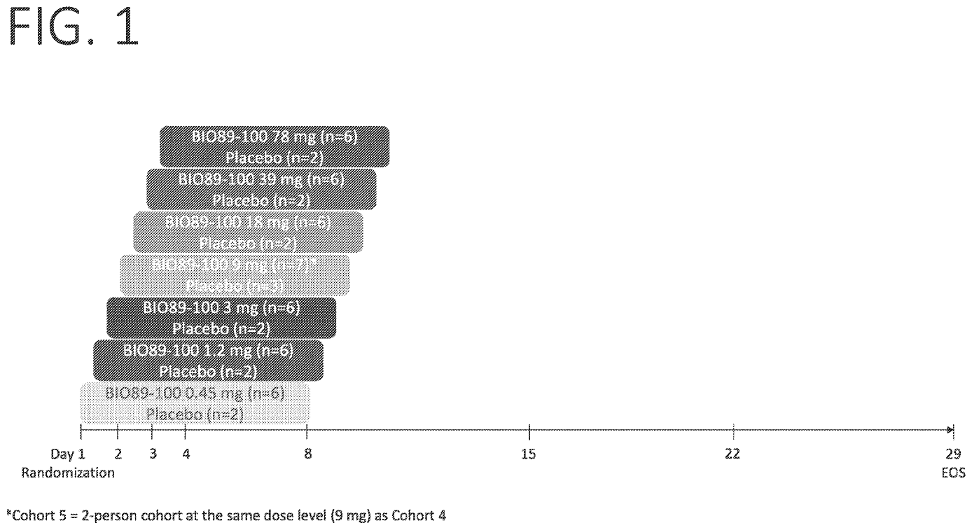

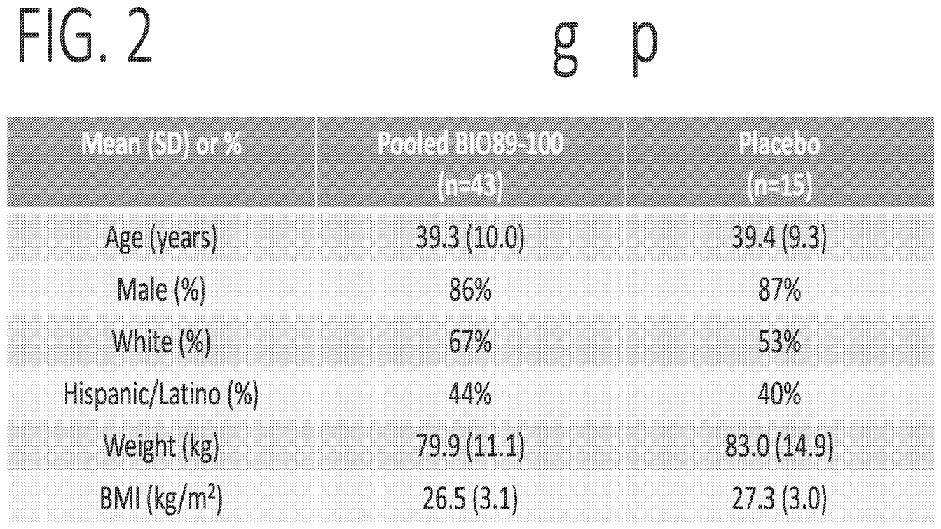

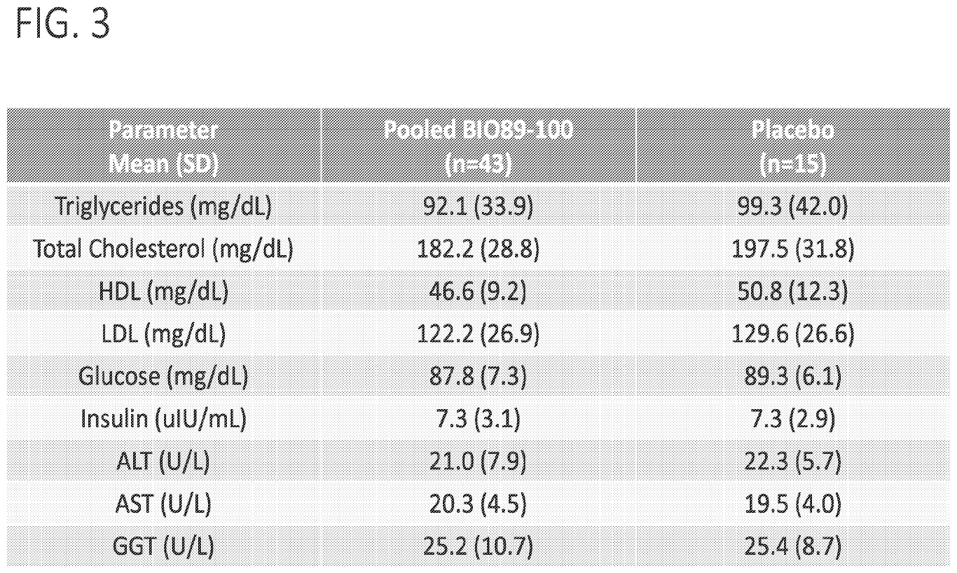

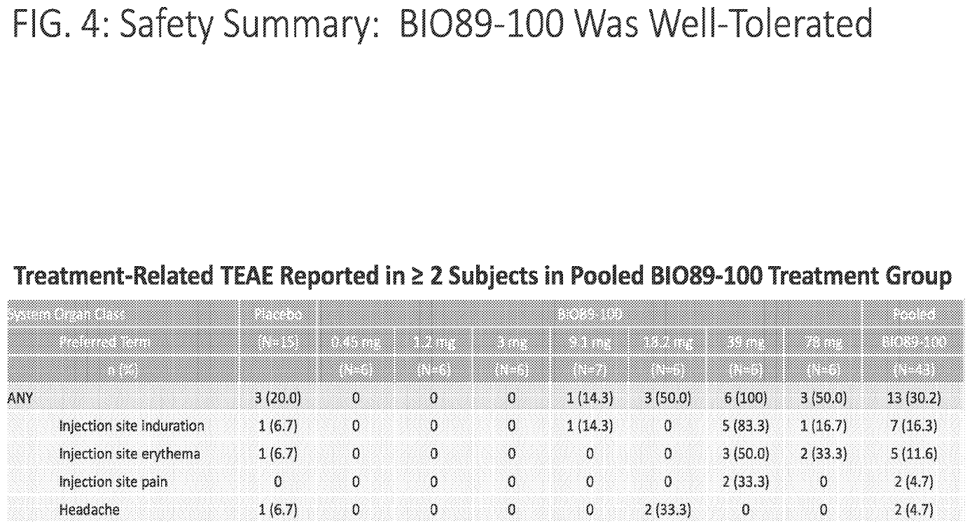

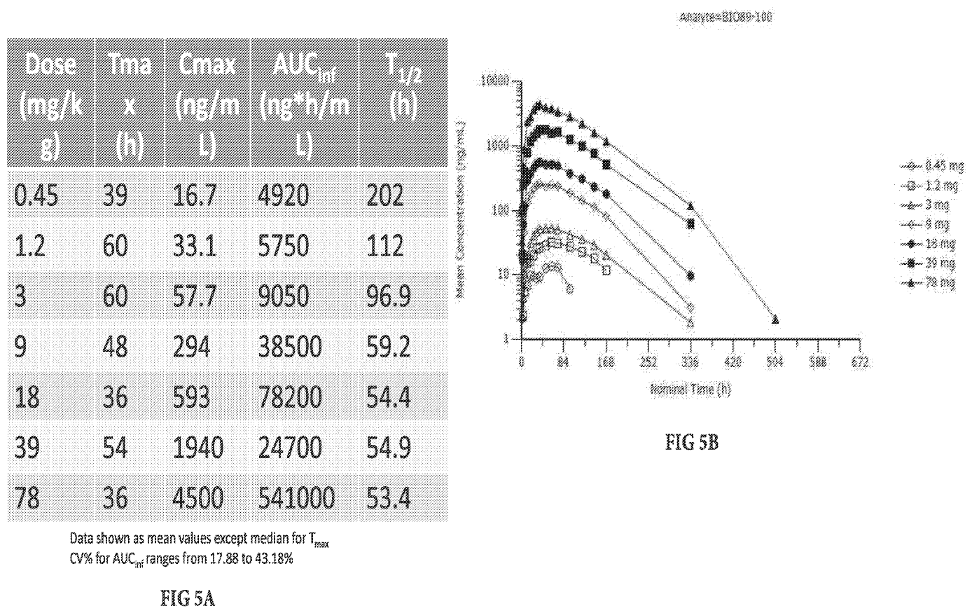

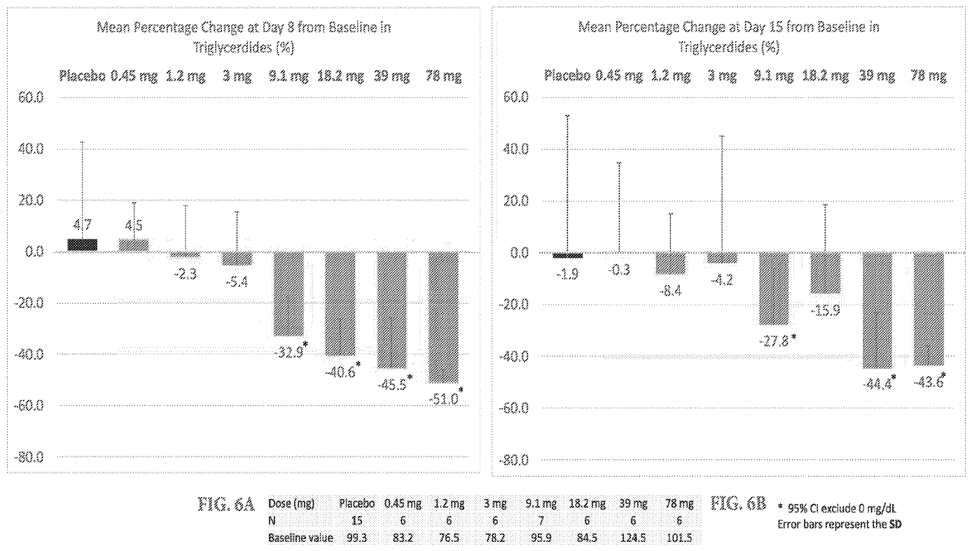

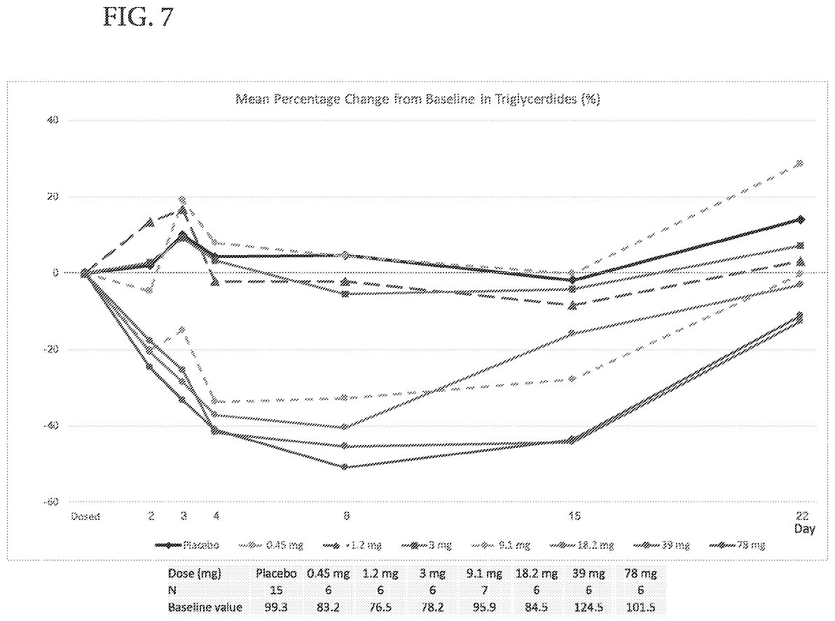

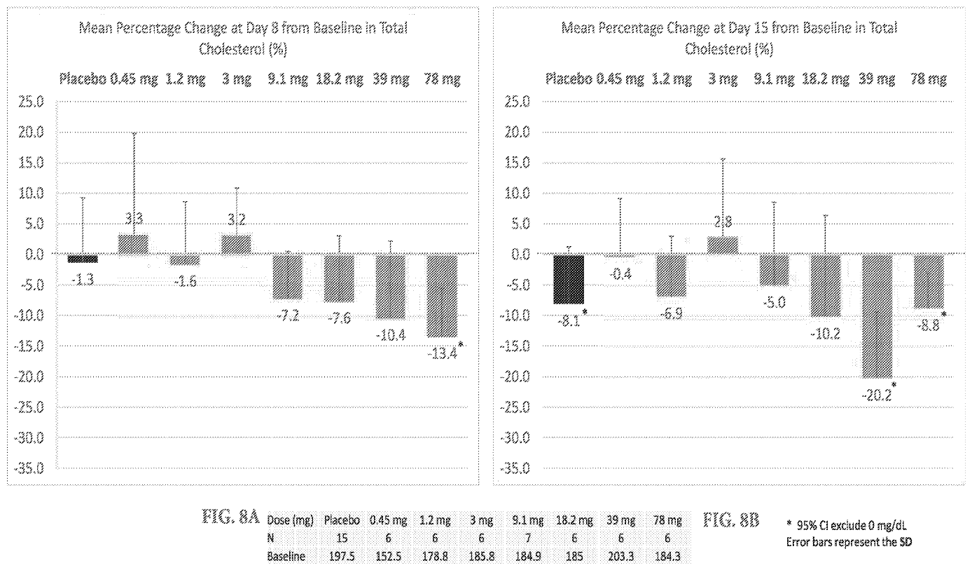

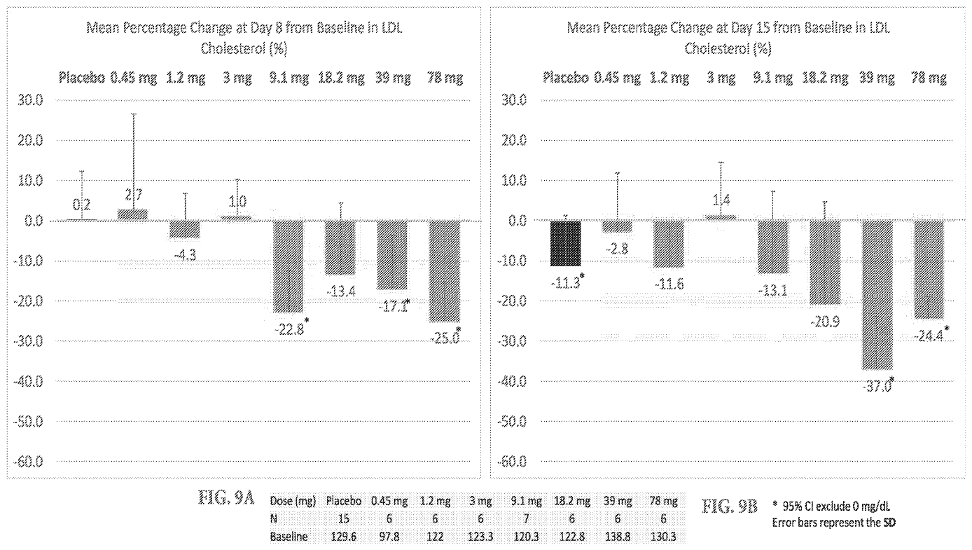

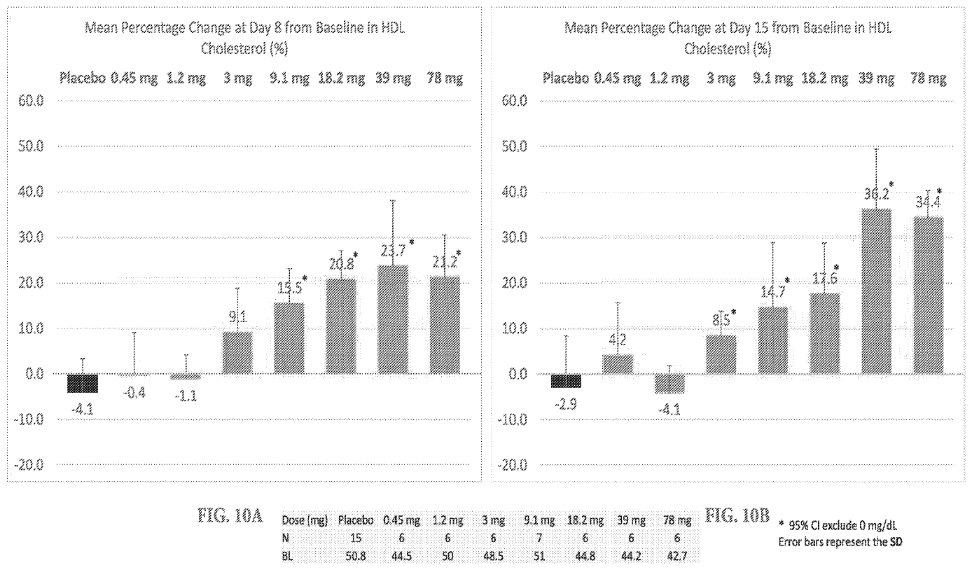

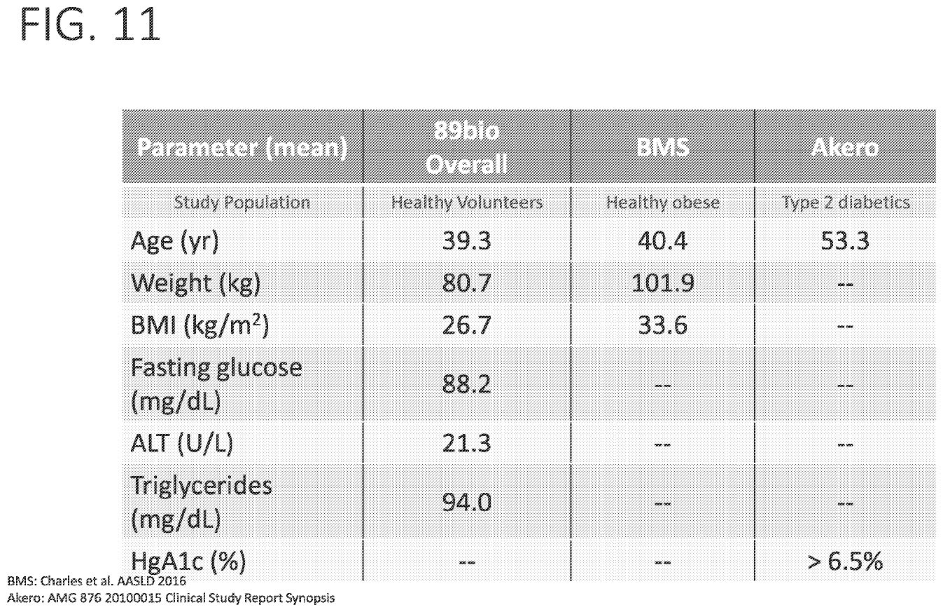

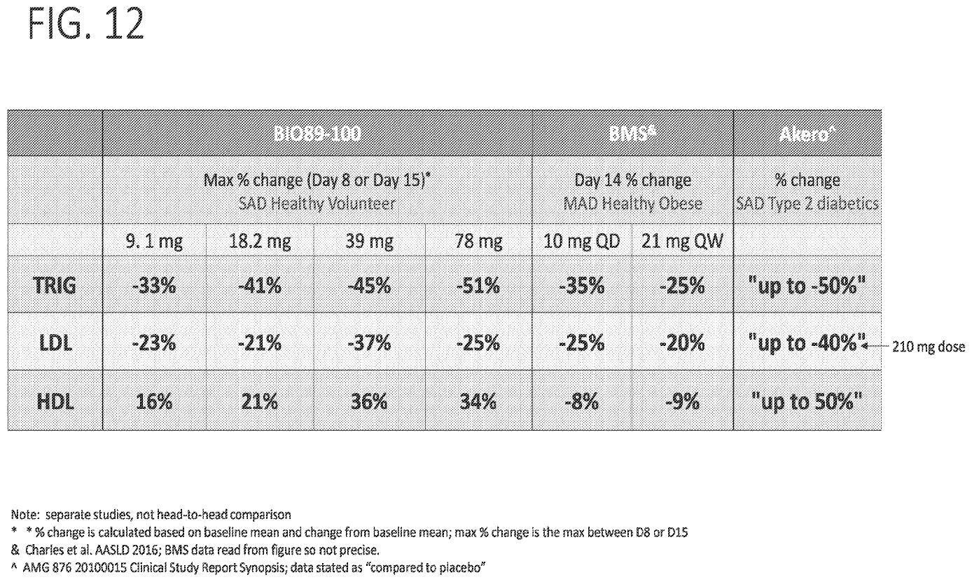

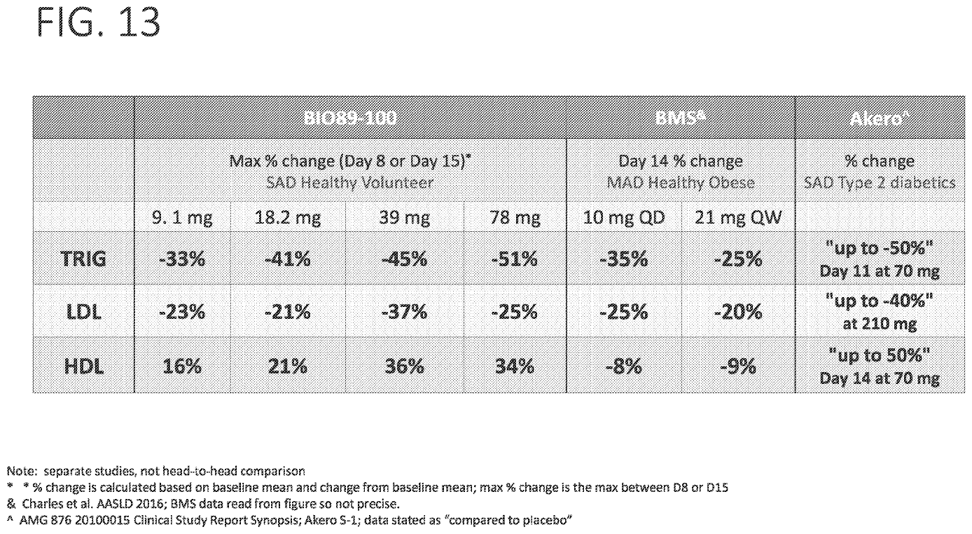

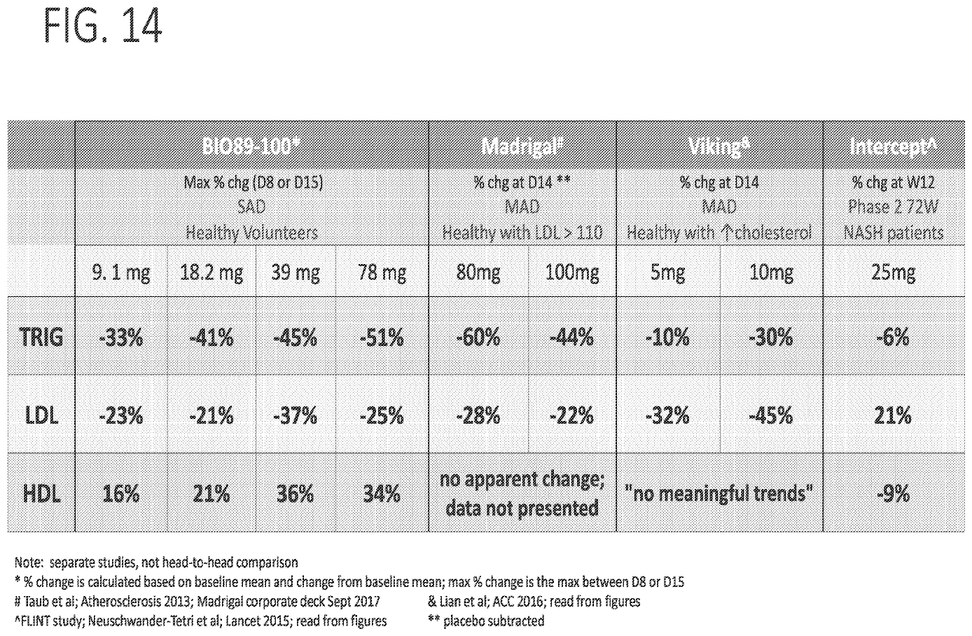

Results: FIGS. 1-14 present results from a single ascending dose (SAD) studies in humans of some embodiments of the present disclosure. Baseline demographics were similar between pooled BIO89-100 treated subjects (N=43) and pooled placebo (N=15), as were mean baseline laboratory parameters (within normal range). Mean age was 39.3 years, mean BMI was 26.7 kg/m², and 86% of subjects were male. There were no deaths, SAEs, discontinuations due to AEs or AE-related dose changes. Among subjects who received BIO89-100, the most common treatment-related adverse events, occurring in ≥2 subjects in the pooled BIO89-100 group, were injection site reactions and headache, all reported as mild. No clinically meaningful trends were observed in laboratory or other safety-related parameters. The PK of BIO89-100 was generally dose proportional, with an average elimination half-life ~53-100 hours. At single doses of 9.1 mg and higher, BIO89-100 demonstrated significant improvements versus baseline in key lipid parameters measured at 8 and 15 days following dosing. The mean changes versus baseline included reduction in triglycerides (up to 51%, Table 1), reduction in LDL-C (up to 37%), and increase in HDL-C (up to 36%).

TABLE 1
Serum triglycerides - Mean change from
baseline (%) on Day 8 and Day 15

| | Placebo | 9.1 mg | 18.2 mg | 39 mg | 78 mg |
|---|---|---|---|---|---|
| | N = 15 | N = 7 | N = 6 | N = 6 | N = 6 |
| Mean at Baseline | 99.3 | 95.9 | 84.5 | 124.5 | 101.5 |
| (BL [SD]; mg/dL) | (42.0) | (33.4) | (30.4) | (45.8) | (15.8) |
| % change from BL | 4.7 | −32.9 | −40.6 | −45.5 | −51.0 |
| to Day 8 (SD) | (38.0) | (15.2) | (14.2) | (19.7) | (4.9) |
| % change from BL | −1.9 | −27.8 | −15.9 | −44.4 | −43.6 |
| to Day 15 (SD) | (54.9) | (21.6) | (34.4) | (22.4) | (7.7) |

### Additional Assessments:

Body weight was only assessed at baseline and end of study
No significant changes noted
Not informative about potential for body weight reduction

ALT and AST: baseline values in normal range (mean=21.3 and 20.1 U/L)
No significant reductions observed

Fasting glucose and insulin: baseline values in normal range (mean=88.2 mg/dL and 7.3 uIU/mL)
No significant reductions observed
Likely related to FGF21 mechanism of action as insulin sensitizer

### SAD Study Summary

BIO89-100 is well tolerated at single doses up to 78 mg SC
Incidence of overall adverse events and treatment-related adverse events did not differ notably among treatment groups across dose range
Exception: Injection site AEs occurred more in high dose groups (39 mg and 78 mg)
No trends indicative of clinically important adverse effects of BIO89-100 on laboratory or other safety-related clinical parameters were apparent
PK profiles are generally dose-proportional with T½ ranges ~53 to 100 hours
BIO89-100-related effects on lipid PD parameters were noted at doses 9.1 mg and higher
Concentration dependent trends observed with key PD parameters up to 39 mg dose without additional benefit in the 78 mg group
PD effects consistent across lipid parameters and generally consistent within subjects
Data supports dosing both once a week and once every two weeks

Conclusions: BIO89-100 at single doses up to 78 mg was safe and well tolerated in healthy subjects, with a favorable PK profile, and was associated with significant improvements in triglycerides, LDL and HDL.

### Example 2: Weekly Subcutaneous Administration of BIO89-100, a Novel glycoPEGylated-Fibroblast Growth Factor 21 (FGF-21) Analogue, Inhibits Sweetness Preference in Obese Cynomolgus Monkeys

Background: BIO89-100, a novel glycoPEGylated analogue of FGF-21, is being developed for the treatment of nonalcoholic steatohepatitis (NASH). FGF-21 regulates carbohydrate and lipid metabolism; FGF-21 and a long acting analogue were shown to regulate sweetness and alcohol preference in mice, and sweetness preference in monkeys. The objective of this study was to assess the effect of BIO89-100 on sweetness preference in obese cynomolgus monkeys.

Methods: Obese cynomolgus monkeys (mean age—12.6 yrs; weight—10.8 kg) were trained on the 2-bottle test prior to dosing. After acclimation for 2 weeks to 2 bottles of drinking water, monkeys were acclimated for 2 additional weeks to having a bottle of drinking water and a bottle of sweet water (3% sucrose). Baseline data was collected after which BIO89-100 (N=3) at a dose of 1 mg/kg or vehicle (N=3) was administered sc weekly (qW) over 3 weeks, followed by 2 weeks wash-out. The preference for sweet vs. non-sweet water was monitored by measuring daily fluid intake. Clinical assessments and laboratory tests were also performed.

Results: Before introducing sweet water, mean water consumption was 235 mL/day. After introduction of sweet water, mean fluid consumption increased significantly to 650 mL/day, consisting almost exclusively of sweet water. After administration of BIO89-100, preference for sweet water markedly decreased within one day and continued to decrease to a point that a negligible amount of sweet water was consumed (mean 40 mL/day). After end of treatment (wash-out), preference for sweet water gradually re-emerged. Control animals preferred sweet water throughout the study period. In BIO89-100-treated monkeys, decreases in body weight (up to −13%), food intake (up to −60%), triglycerides (up to −78%), and alanine transaminase (up to −44%), and an increase in high-density lipoprotein (up to 39%) compared to baseline were observed. Control animals showed no decrease in body weight, food intake or any other change in blood lipids.

Conclusion: Weekly administration of BIO89-100 to obese monkeys resulted in a significant decrease in sweetness preference and in improvements in metabolic and liver-related lab parameters. These results demonstrate that FGF-21 analogues such as BIO89-100 may be used for treatment modality for NASH. Decreased preference for sugar in humans may, furthermore, confer an additional important benefit in NASH patients.

### Example 3—Mechanism of Action Via Potent FGF Receptor Agonism

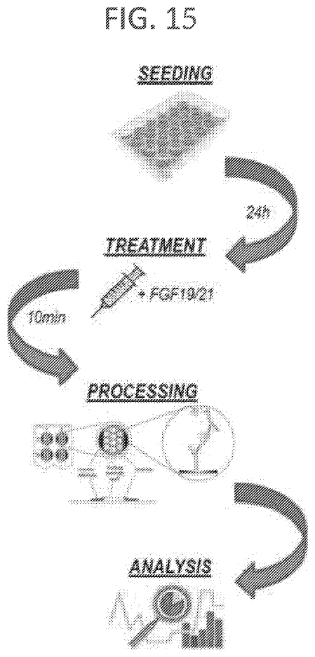

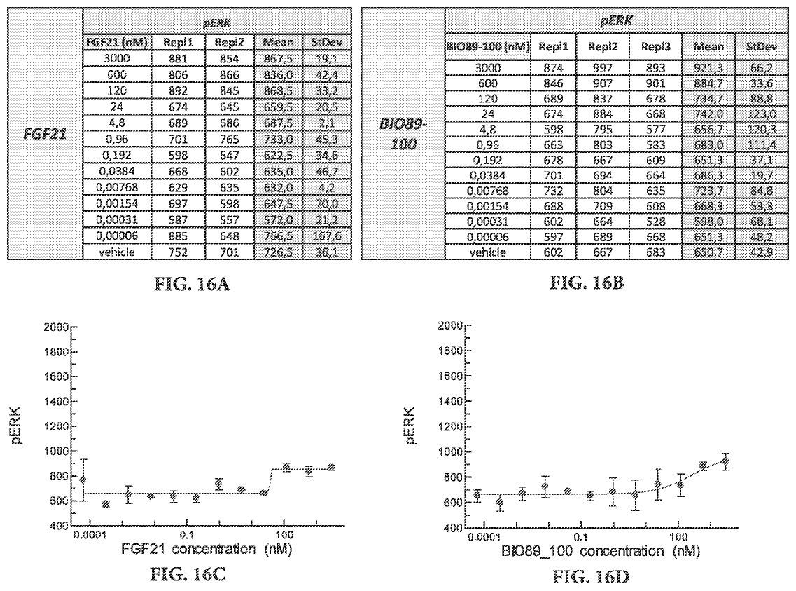

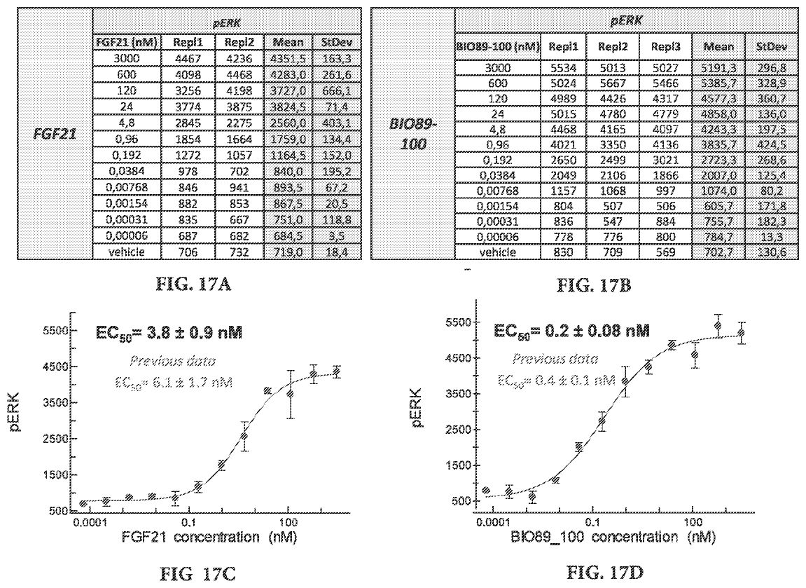

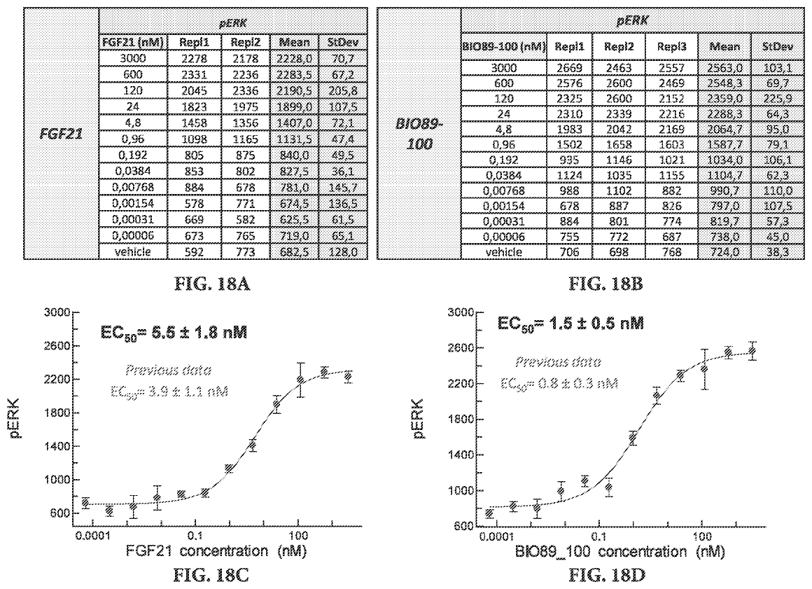

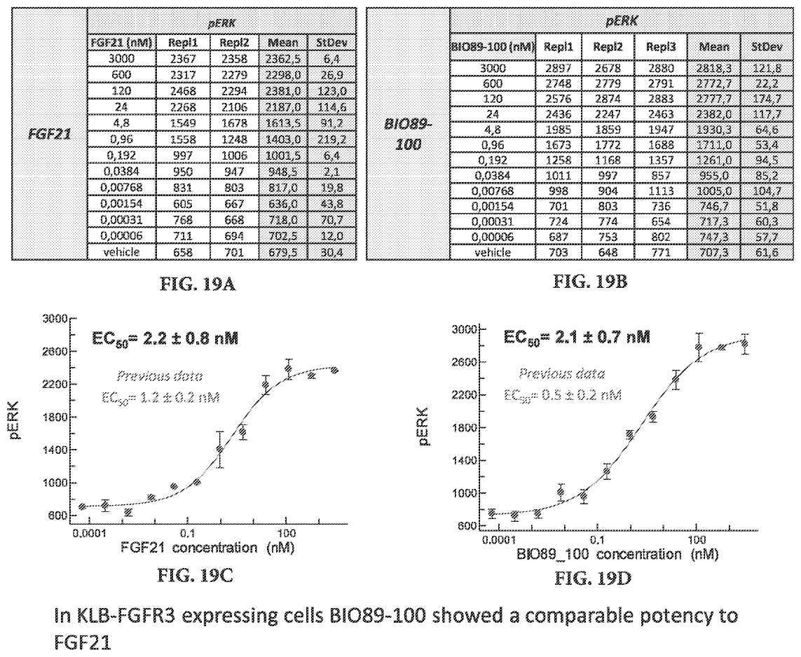

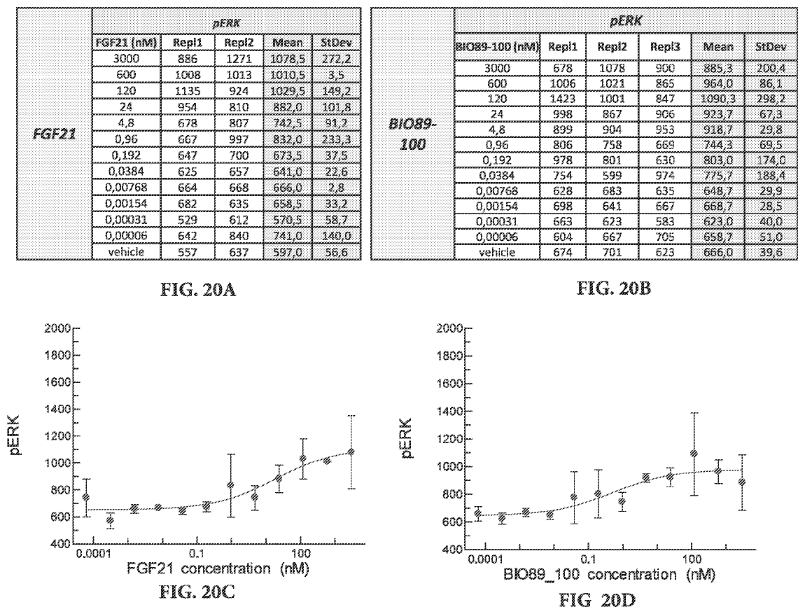

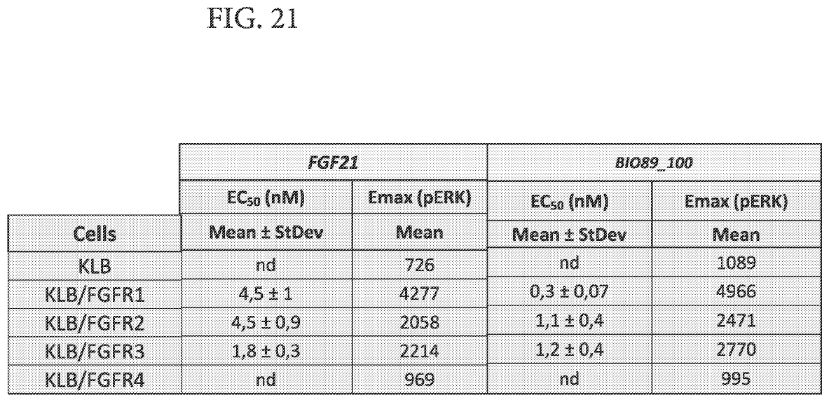

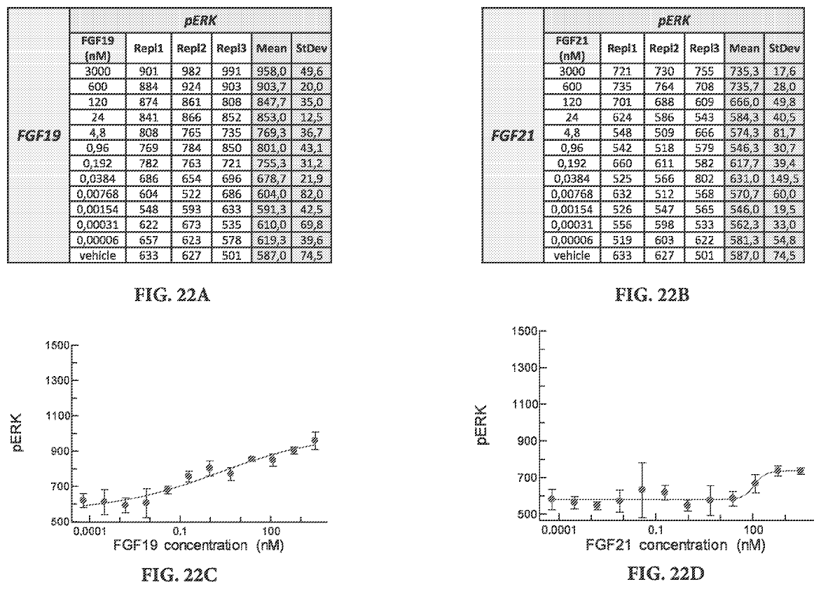

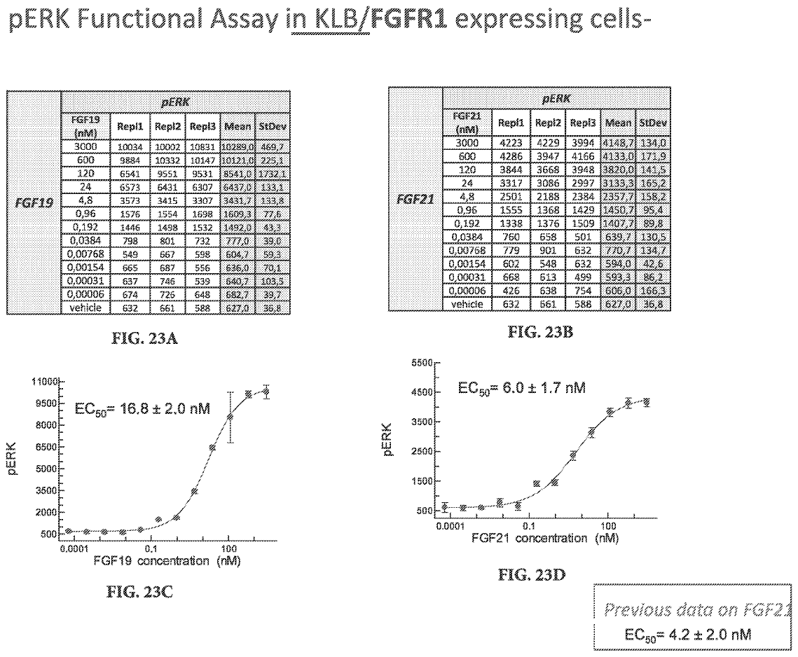

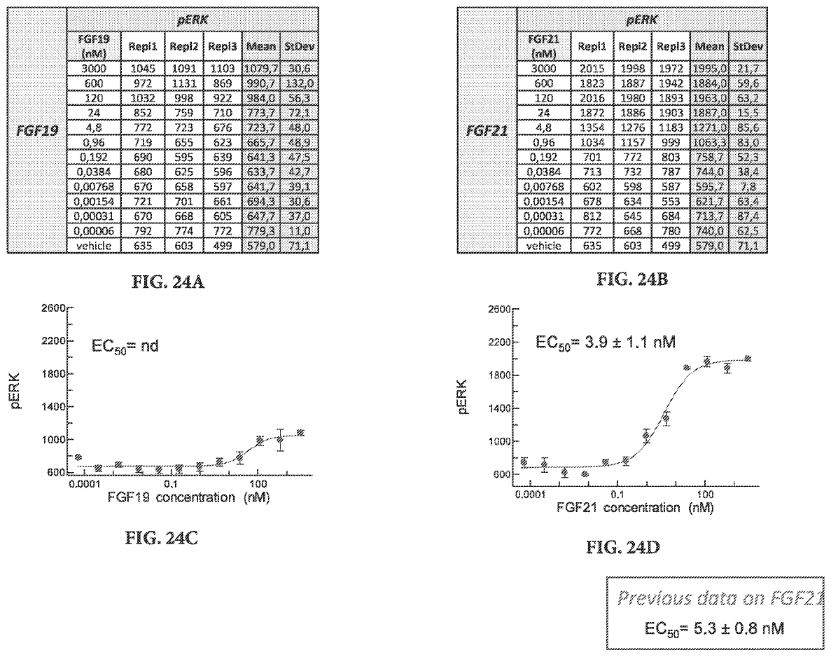

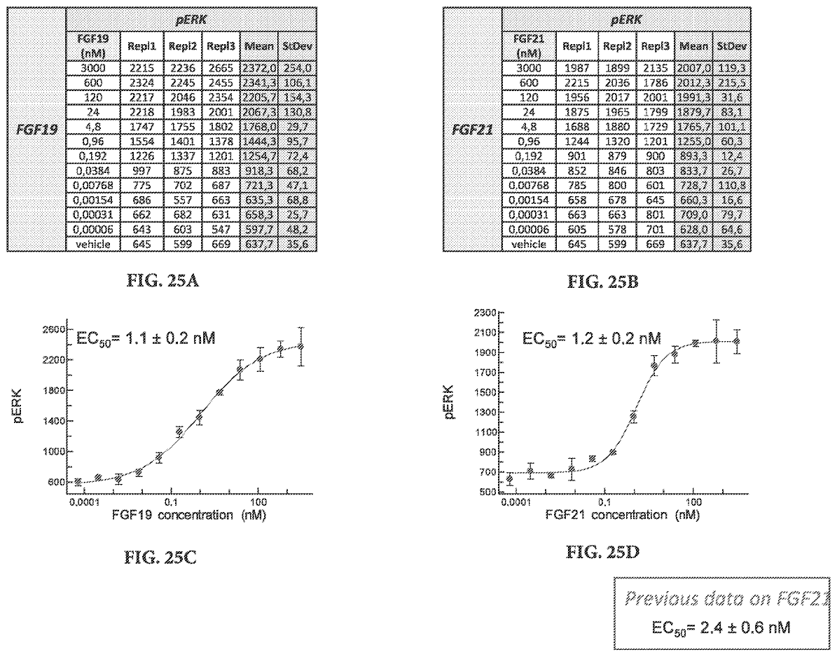

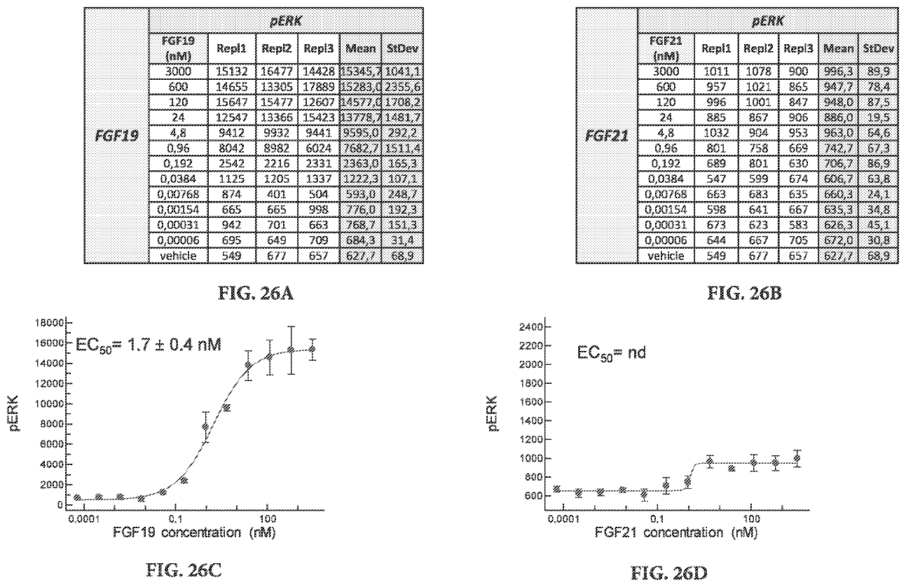

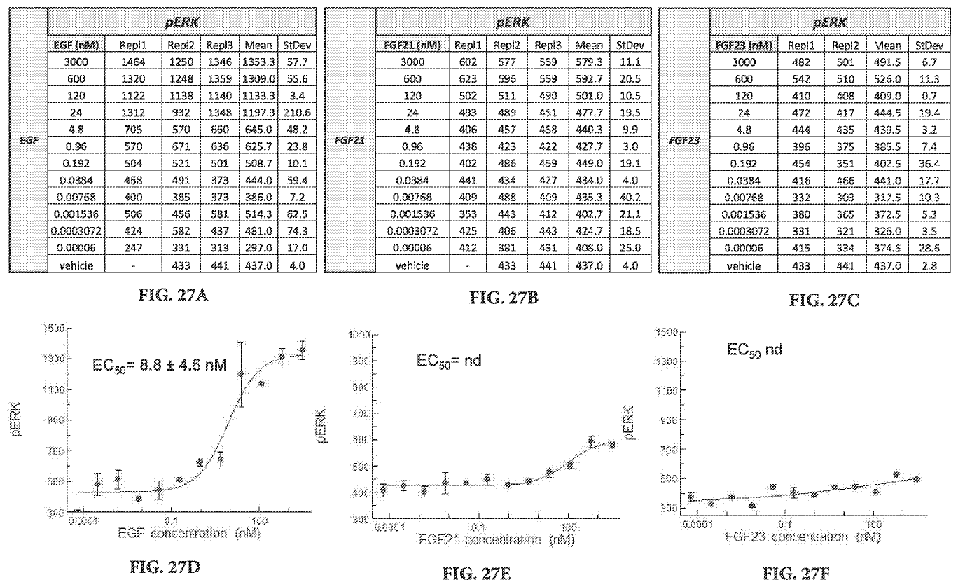

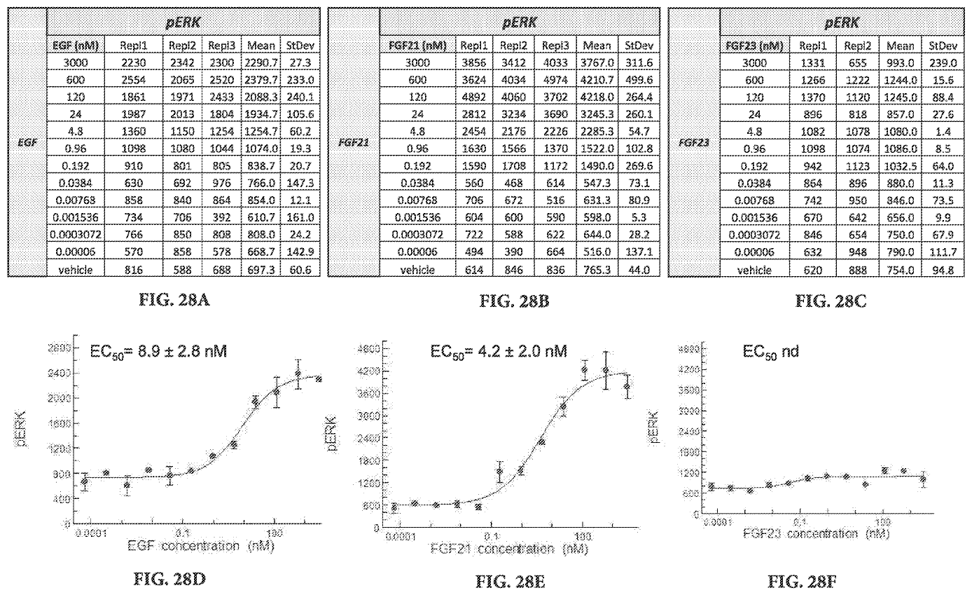

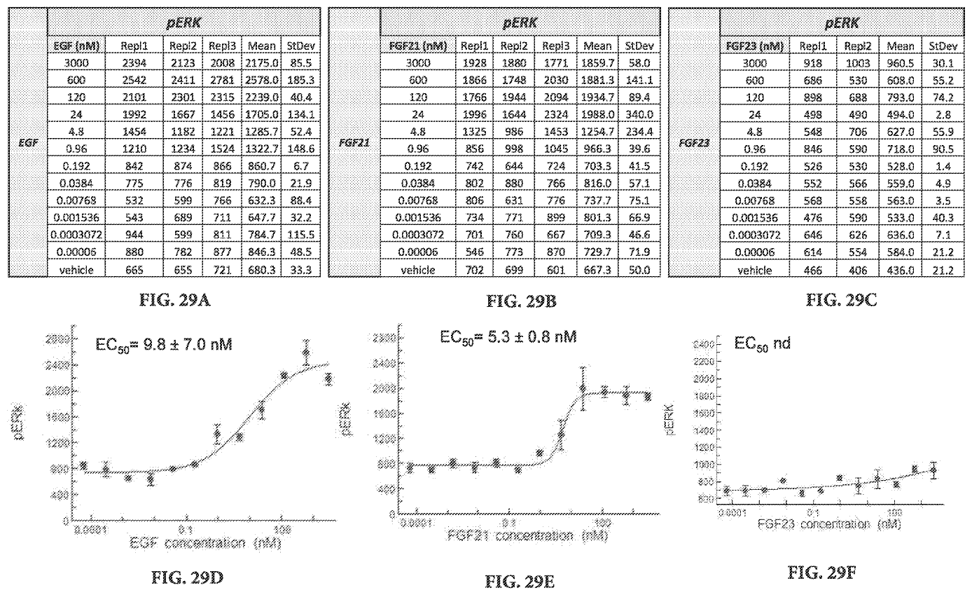

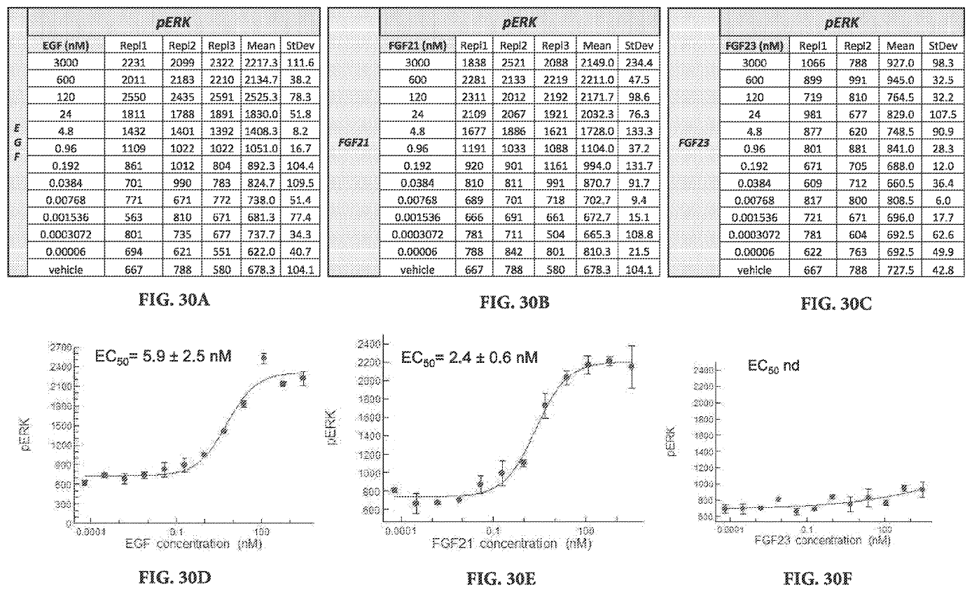

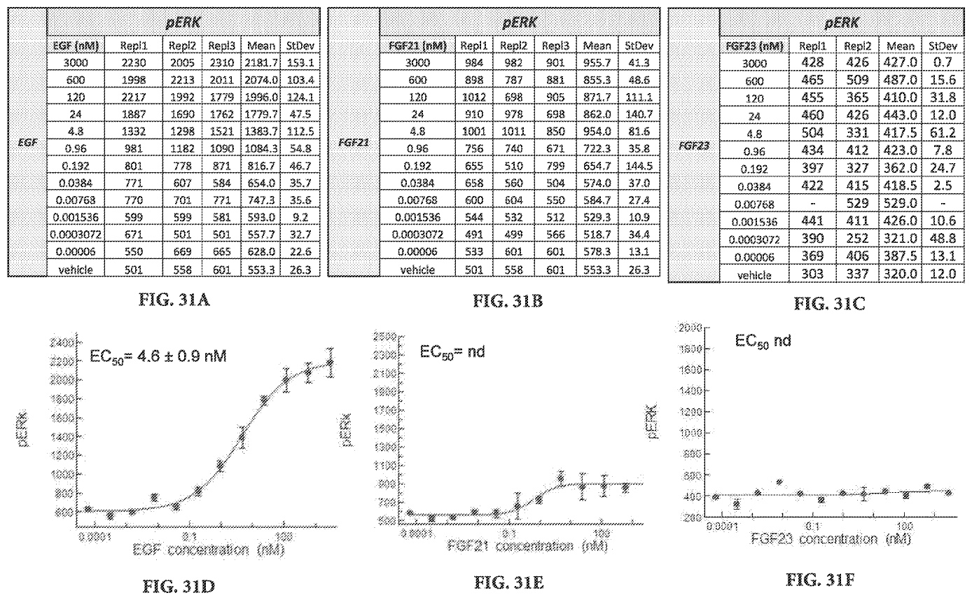

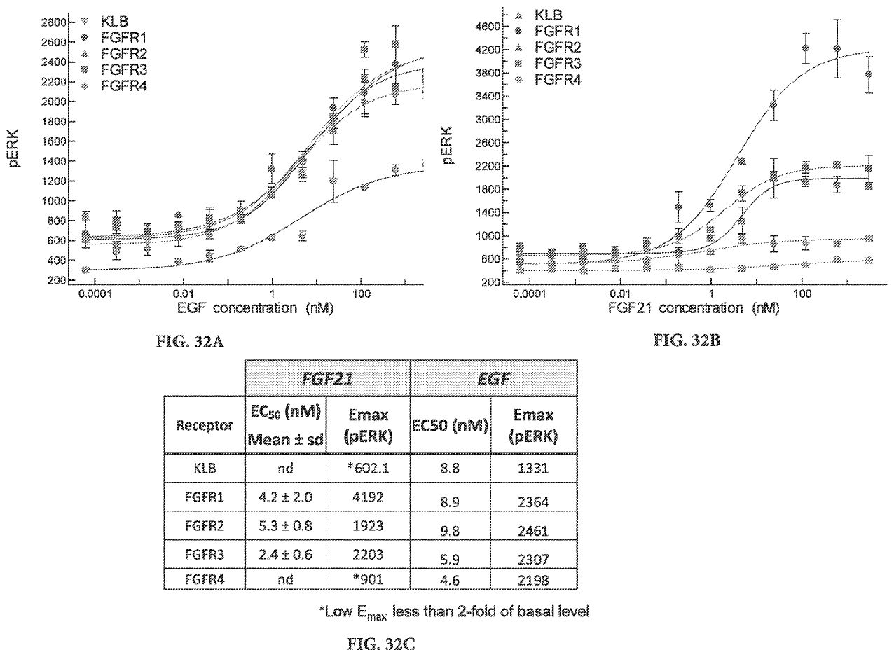

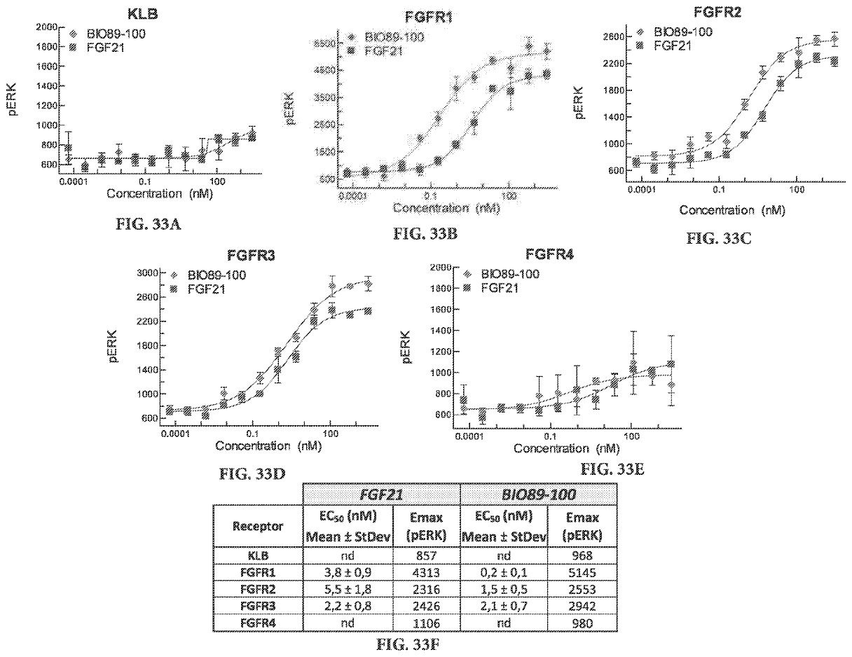

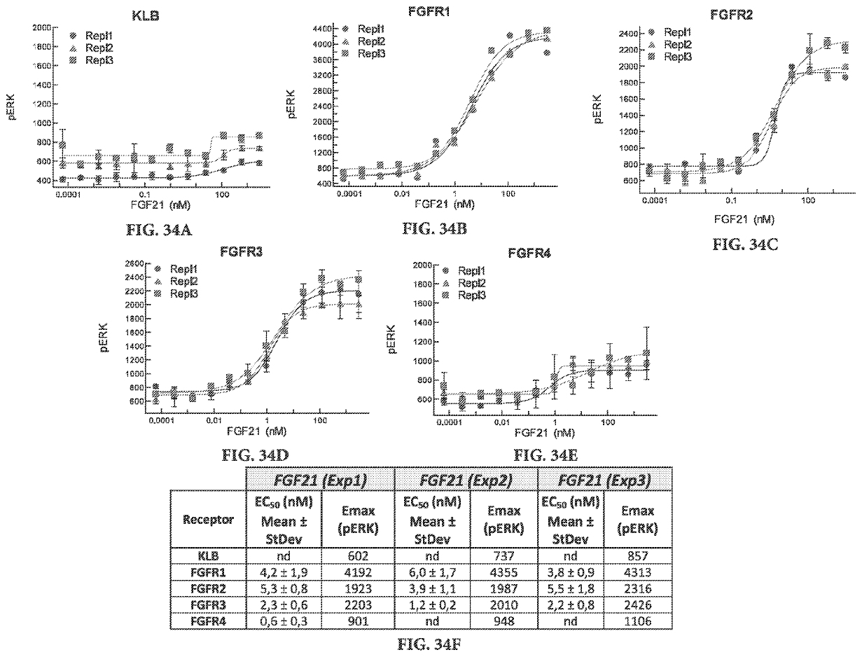

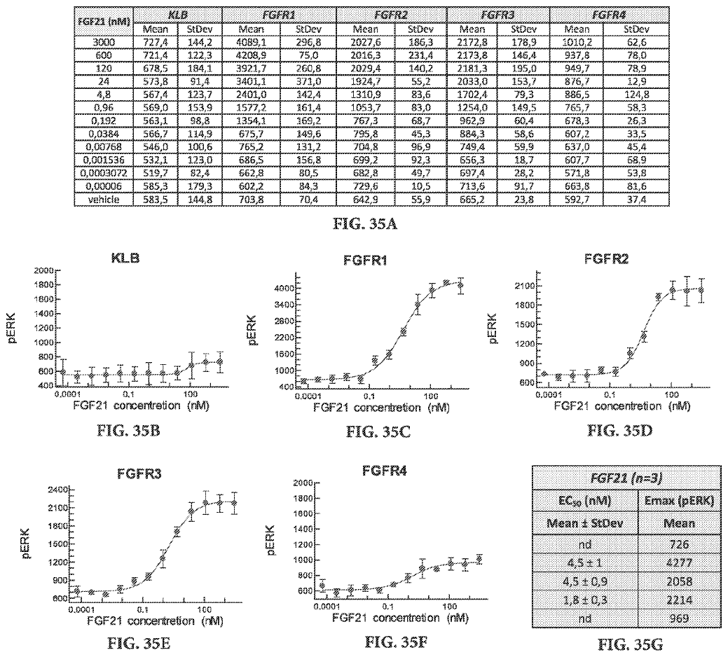

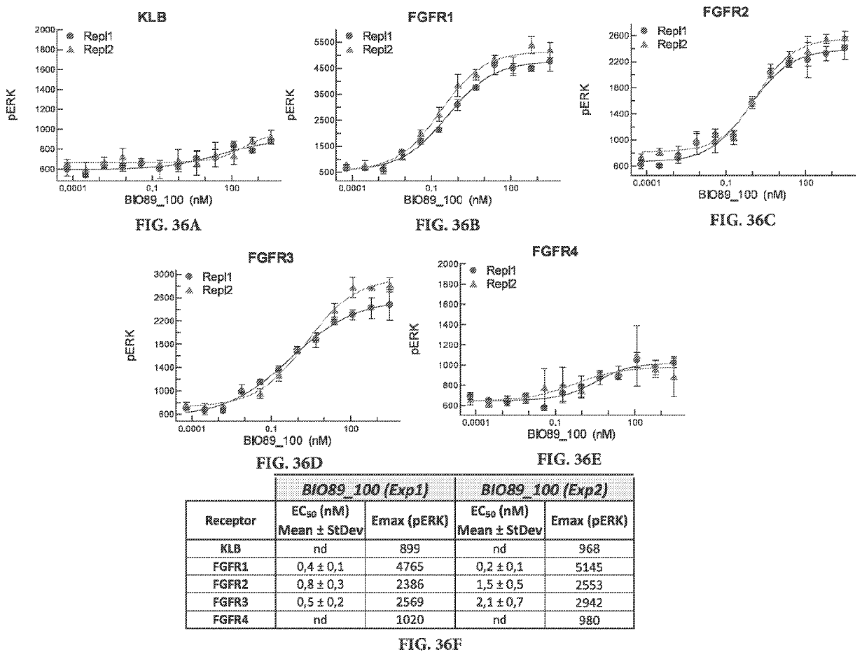

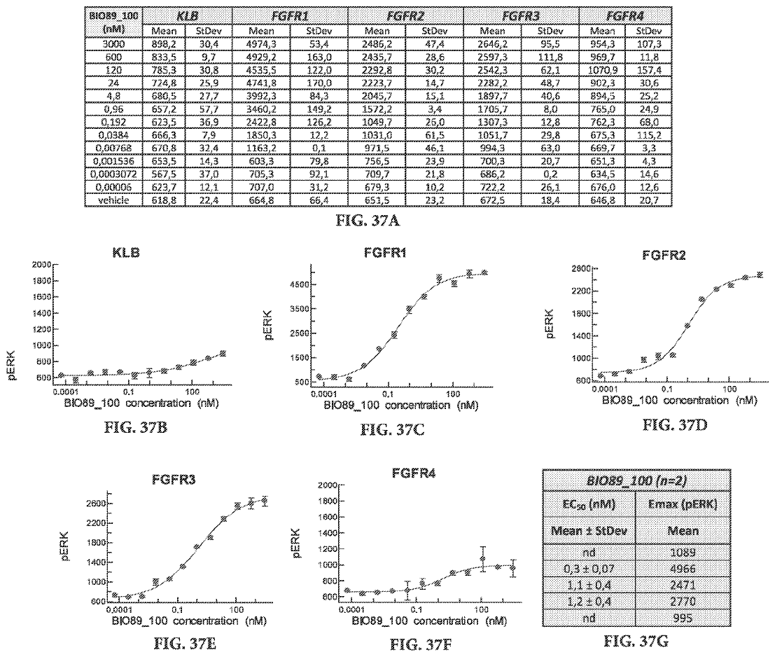

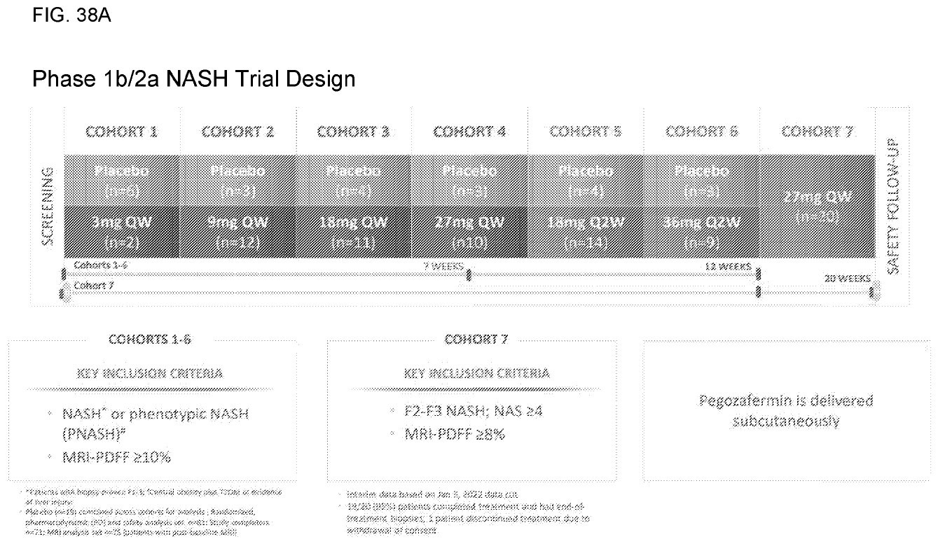

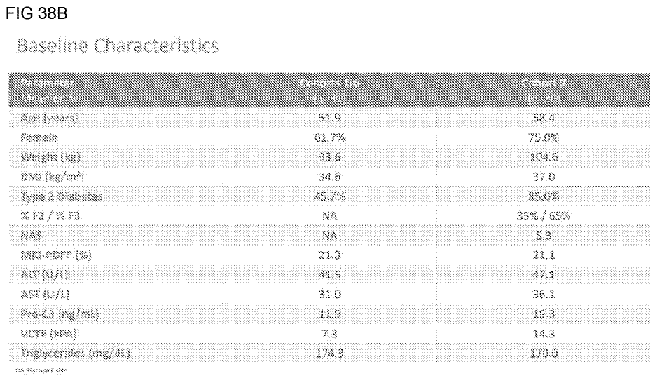

FIGS. 15-37G present results of the potency of the mutant Fibroblast Growth Factor-21 (FGF-21) peptide conjugate.

### PERK Functional Assay:

1. L6 cells expressing KLB/FGFR1 were seeded in a 96-well plate format (104 cells/well) 24 hours before treatment
2. On the day of the assay, cells were starved for 2 hr in serum-free medium, then treated with the ligand (or vehicle alone) at the indicated concentrations and the indicated time points (triplicates)
3. All tubes, plates and pipette tips used for the assay were pre-coated overnight with 1% BSA (in PBS)
4. FGF-21 and BIO89-100 dilutions were made in PBS1X pH7.4 containing 0.1% BSA
5. Following treatment cells were transferred on ice, washed twice in ice-cold PBS and lysed in MSD* lysis buffer, according to Manufacturer's protocol
6. Samples were processed for pERK/ERK levels by Mesoscale

### Results

The data showed that in KLB only and KLB-FGFR4 expressing cells, FGF21 and BIO89-100 were almost non active with only a slight increase of pERK at the highest concentration tested (3,000 nM).

FGF21 and BIO89-100 were highly active in cells expressing FGFR1, FGFR2 and FGFR3.

BIO89-100 was more potent than FGF21 in KLB-FGFR2 and KLB-FGFR1 expressing cells.

BIO89-100 had a comparable potency to FGF21 in KLB-FGFR3 expressing cells.

FGF-19 was highly active in cell expressing FGFR4 but also with FGFR1 and FGFR3.

The negative control, FGF-23, was not active in all cells with no difference between KLB only and the 4 receptors.

The positive control EGF was active with high potency in cells transfected with the 4 receptors

### Conclusion

Preclinical data demonstrates that BIO89-100 has similar activity to native FGF21 at FGF receptors 1c, 2c and 3c, suggesting that BIO89-100 could reproduce the beneficial metabolic benefits of the native hormone, which may translate into clinical benefits for patients with NASH.

Activation of the FGF receptors 1c, 2c and 3c, together with the co-receptor β-klotho, are critical to the signaling of FGF21 and are believed to be responsible for the beneficial metabolic effects observed. In an in vitro study of receptor agonism, BIO89-100 was shown to have activity at very low nanomolar concentrations in cells co-expressing β-klotho and any of FGF receptors 1c, 2c or 3c (FIGS. 15-37G). The EC50 (concentration at which one half of the maximal FGF receptor agonist effect is observed) for BIO89-100 was similar across FGF receptors 1c, 2c and 3c and comparable or superior to that of native FGF21 in this functional assay. An EC50 could not be determined for native FGF21 or BIO89-100 at FGF receptor R4.

### Example 4

Study BIO89-100-002 is a randomized, double-blind, placebo-controlled, multiple ascending dose (MAD) study to evaluate the safety, tolerability, PK and PD profiles and immunogenicity of BIO89-100 administered SC in approximately 83 subjects with NASH, or with NAFLD who are at a high risk of NASH. This multi-site study consists of 6 cohorts, and evaluates 2 dosing schedules, weekly (QW; Cohorts 1 to 4) and every 2 weeks (Q2W; Cohorts 5 and 6) (Table 2).

There are 2 dose escalation decisions. After Cohort 1 completes the Day 36 visit, subjects can be randomized into Cohorts 2 and 5 (both cohorts to start concurrently). After at least 8 subjects from both Cohort 2 and Cohort 5, including at least 1 subject on placebo in each cohort, complete the Day 36 visit, subjects can be randomized into Cohorts 3, 4 and 6 (all three cohorts to start concurrently).

Cohorts 1 to 4 (weekly regimen): On Day-1, eligible subjects are randomized (as described above) and treated with weekly (QW) SC injection of study intervention starting on Day 1 and continuing through Day 85.

Cohorts 5 and 6 (every 2 weeks regimen): On Day-1, eligible subjects are randomized (as described above) and treated with SC injection of study intervention every 2 weeks (Q2W) starting on Day 1 and continuing through Day 85.

Subjects in all cohorts are followed up on Day 92 (1 week post last dose of study intervention) and Day 113, 4 weeks post last dose of study intervention (End of Study visit).

TABLE 2
Dose Escalation Cohorts

| | Dose | | | Number of Subjects | |
|---|---|---|---|---|---|
| Cohort | Level^{a} | | Frequency and Route of Administration | BIO89-100 | Placebo |
| 1 | 3 | mg | Weekly (QW), SC to abdomen (1 | 6 | 2 |
| | | | injection) Days 1, 8, 15, 22, 29, 36, 43, | | |
| | | | 50, 57, 64, 71, | | |
| | | | 78, and 85 | | |
| | | | Total doses (initial 4 weeks + 8 | | |
| | | | week extension): 5 + 8 | | |
| 2 | 9 | mg | QW, SC to abdomen (1 injection) | 12 | 3 |
| | | | Days 1, 8, 15, 22, 29, 36, 43, 50, 57, 64, 71, | | |
| | | | 78, and 85 | | |
| | | | Total doses (initial 4 weeks + 8 | | |
| | | | week extension): 5 + 8 | | |
| 3 | 18 | mg | QW, SC to abdomen (1 injection) | 14 | 4 |
| | | | Days 1, 8, 15, 22, 29, 36, 43, 50, 57, 64, 71, | | |
| | | | 78, and 85 | | |
| | | | Total doses (initial 4 weeks + 8 | | |
| | | | week extension): 5 + 8 | | |
| 4 | 27 | mg | QW, SC to abdomen (2 injections) | 9 | 3 |
| | | | Days 1, 8, 15, 22, 29, 36, 43, 50, 57, 64, 71, | | |
| | | | 78, and 85 | | |
| | | | Total doses (initial 4 weeks + 8 | | |
| | | | week extension): 5 + 8 | | |
| 5 | 18 | mg | Every 2 weeks (Q2W), SC to | 14 | 4 |
| | | | abdomen (1 injection) | | |
| | | | Days 1, 15, 29, 43, 57, 71 and 85. | | |
| | | | Total doses (initial 4 weeks + 8 | | |
| | | | week extension): 3 + 4 | | |
| 6 | 36 | mg | Q2W, SC to abdomen (2 | 9 | 3 |
| | | | injections) Days 1, 15, 29, 43, | | |
| | | | 57, 71 and 85. | | |
| | | | Total doses (initial 4 weeks + 8 | | |
| | | | week extension): 3 + 4 | | |

^{a}The actual doses will be ±5% the mg dose due to technical considerations related to drug withdrawal from the vials into the syringes for injection. This difference is considered negligible for subject exposure.

Change and percentage change from baseline in the following biomarkers/PD parameters:

### Anthropomorphic Measurements:

Body weight
Waist circumference
Waist/hip ratio

### Laboratory Parameters

Triglycerides
Non-high density lipoprotein (Non-HDL) cholesterol
High density lipoprotein (HDL-c)
Low density lipoprotein (LDL-c)
Hemoglobin Alc (HbA1c)
Homeostatic Model Assessment for Insulin Resistance (HOMA-IR)
Liver function tests: alanine transaminase (ALT), aspartate transaminase (AST)
Adiponectin
N-Terminal Propeptide of Type III Collagen (Pro-C3)
Free fatty acids and Adipo-IR (fasting free fatty acids x fasting insulin)
inflammation marker high-sensitivity C-reactive protein (hs-CRP)
Total cholesterol
OGTT including C-peptide, glucose, and insulin
IGF-1, total
CK-18
Enhanced LiverFibrosis (ELF) panel

### Imaging Measures

Magnetic Resonance Imaging-Whole liver Proton Density Fat Fraction (MRI-PDFF)
Liver volume
Abdominal visceral fat
Abdominal subcutaneous fat
Fibroscan CAP score
Fibroscan VCTE score

### Enhanced Liver Fibrosis (ELF) Panel:

The enhanced liver fibrosis (ELF) blood test has recently been recommended by the National Institute for Health and Care Excellence to test for advanced fibrosis in NAFLD. The ELF test involves calculating a score from the concentrations of serum biomarkers: tissue inhibitor of matrix metalloproteinases-1 (TIMP-1), amino-terminal propeptide of procollagen type III (P3NP), and hyaluronic acid (HA).

### N-Terminal Propeptide of Type III Collagen (Pro-C3)

N-protease cleaved N-terminal propeptide of type 3 procollagen (P3NP) neo-epitope (Pro-C3) is derived from the synthesis of type 3 collagen. Pro-C3 appears to correlate with liver fibrosis stage, fibrosis regression and response to treatment both as a single test and as part of algorithms Magnetic Resonance Imaging-Whole liver Proton Density Fat Fraction (MRI-PDFF)

MRI-PDFF is as a noninvasive, quantitative, and accurate measure of liver fat content (imaging-based biomarker) to assess treatment response in NASH clinical studies. This technology enables post-processing of MRI data into parametric map of PDFF (Antaros Medical, Sweden) to provide accurate and quantitative measures of liver fat.

### Liver Volume

A dedicated Axial 3 dimensions (3D) T1-weighted scan with or without fat suppression is positioned to cover the entire liver. Analysis is done using a semiautomated software to delineate the outer borders of the liver and the liver volume will be calculated in liters.

### Visceral Abdominal Fat (VAT)/Subcutaneous Abdominal Fat (SAT)

A 2 or 3 point gradient echo Dixon imaging is performed in the axial plane centered at the L4-L5 interface covering approximately ±10 cm in the feet-head direction. Water and fat images are reconstructed and the visceral and subcutaneous adipose tissue volumes in the abdominal region are quantified using a semi-automated software giving adipose tissue volume as output in liters.

### PDFF

The PDFF is determined using a 6 echo gradient echo pulse sequence covering the liver in the axial plane. Analysis is performed by semi-automatic contouring of the liver in every slice avoiding major vessels and bile ducts. The method applies multi-peak lipid spectral models and simultaneous quantification and correction for T2*. The liver fat value (PDFF) is the mean value of all voxels in the identified volume of interest.

### Example 5: Phase 1b/2a Proof-of-Concept Study Evaluating Pegozafermin (Formerly BIO89-100) for the Treatment of NASH

NASH is a serious liver condition with significant co-morbidities. See Table 3. No treatments are currently available. There are 16.5 million cases projected to grow to 27 million cases by 2030. NASH is expected to become the leading cause of liver transplant.

TABLE 3

| Co-morbidity | Prevalence in NASH population |
|---|---|
| Hypertriglyceridemia | 83% |
| Obesity | 82% |
| Hyperlipidemia/Dyslipidemia | 72% |
| Metabolic syndrome | 71% |
| Type 2 diabetes | 44% |

Phase 1b/2a NASH trial design is shown in FIG. 38A. The key inclusion for cohorts 1-6 and cohort 7 are shown on FIG. 38B. Cohort 7 include fibrosis stage F2 and F3 NASH patients having a NAFLD Activity score (NAS) score ≥4 and MRI-PDFF ≥8%. The NAS can range from 0 to 8 and is calculated by the sum of scores of steatosis (0-3), lobular inflammation (0-3) and hepatocyte ballooning (0-2). FIG. 38B is a table showing the Baseline characteristics.

In a single-arm cohort, biopsy-confirmed, fibrosis stage F2 and F3 NASH patients were treated once weekly for 20 weeks with 27 mg of pegozafermin. At baseline, 65% of patients were fibrosis stage F3.

Of the 20 patients enrolled, 19 received an end-of-treatment biopsy and the results from these 19 patients were as follows:

TABLE 4
Histology Results

| | |
|---|---|
| 2-point or greater improvement in NAS without worsening of fibrosis¹ (primary | 63% |
| endpoint) | |
| 2-point or greater improvement in NAS¹ | 74% |
| NASH resolution without worsening of fibrosis | 32% |
| One-stage improvement of fibrosis without worsening of NASH | 26% |
| NASH resolution or fibrosis improvement | 47% |

NAS = NAFLD Activity Score
¹A 2-point improvement in NAS score required a 1-point improvement in either ballooning or inflammation

TABLE 5
Non-invasive tests (NITs) [marker of]

| | Mean change from | Responder rates by clinically |
|---|---|---|
| | baseline at Week 20 | relevant thresholds |
| MRI-PDFF [liver fat content]¹ | −64%*** | 100%/78% [≥30%/≥50%] |
| ALT (Alanine aminotransferase) | −46%*** | 71%³ [≥17 U/L] |
| [liver damage]² | | |
| FAST Score [risk for advanced | −76%*** | 88% [≤0.35] |
| fibrosis]⁴ | | |
| VCTE [liver stiffness]⁵ | −31%*** | 72% [>20% decrease] |
| Pro-C3 [collagen deposition]⁶ | −20%*** | 63% [>15% decrease] |

***p < 0.001
¹Changes from baseline ≥30% and ≥50% have been correlated with NASH improvement
²ALT changes ≥17 U/L have been correlated with histological improvement
³In patients with elevated ALT as defined by ≥30 U/L in women and ≥40 U/L in men (n = 14)
⁴FAST score is a composite of imaging and blood markers and measured on 0-1 scale, a score ≤0.35 predicts Fibrosis Stage F0/F1 and NAS <4
⁵VCTE is a Fibroscan assessment, >20% reduction has been correlated with fibrosis improvement
⁶Pro-C3 is a blood-based measurement, >15% reduction has been correlated with fibrosis improvement.

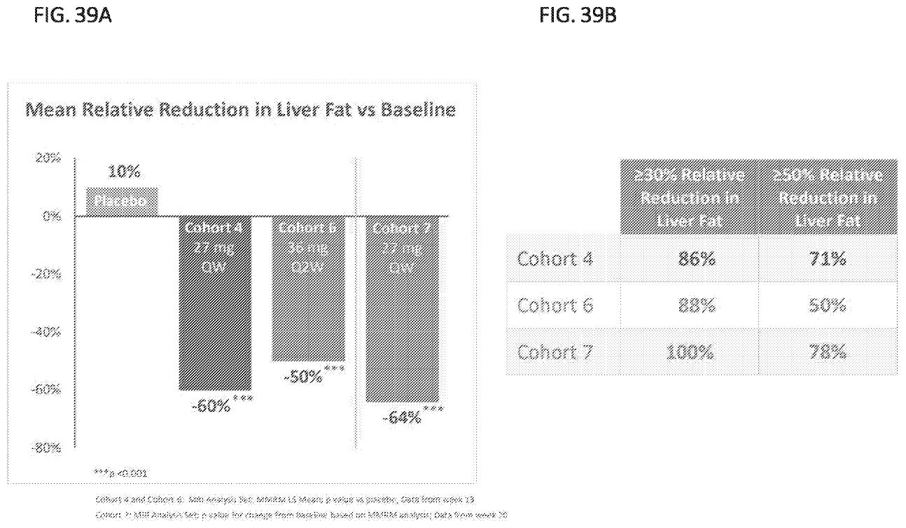

FIG. 39A and FIG. 39B show that BIO89-100 demonstrated robust liver fat reduction with high responder rates.

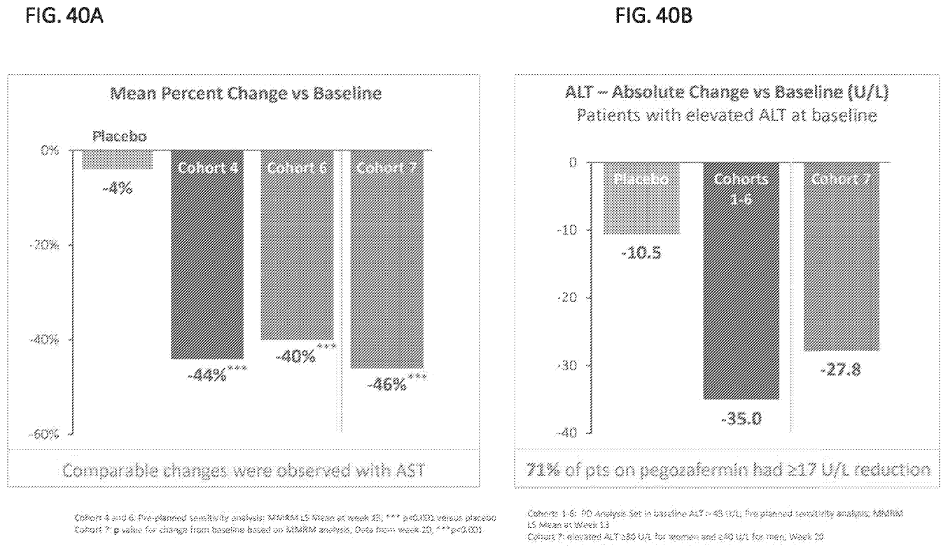

FIG. 40A and FIG. 40B show that BIO89-100 significantly reduced Alanine aminotransferase (ALT) with greater reduction in patients with elevated baseline ALT.

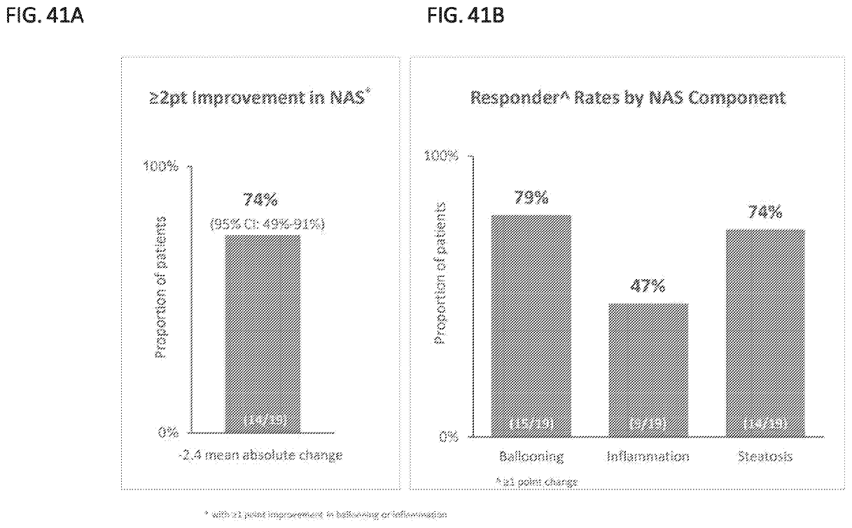

FIG. 41A and FIG. 41B show that BIO89-100 robustly improved NAFLD activity score (NAS) and all components of NAS. 63% of patients had ≥2 point improvement in NAS and no worsening of fibrosis (with ≥1 point improvement in ballooning or inflammation) (primary endpoint). 100% of patients had improvement or no change in ballooning and inflammation.

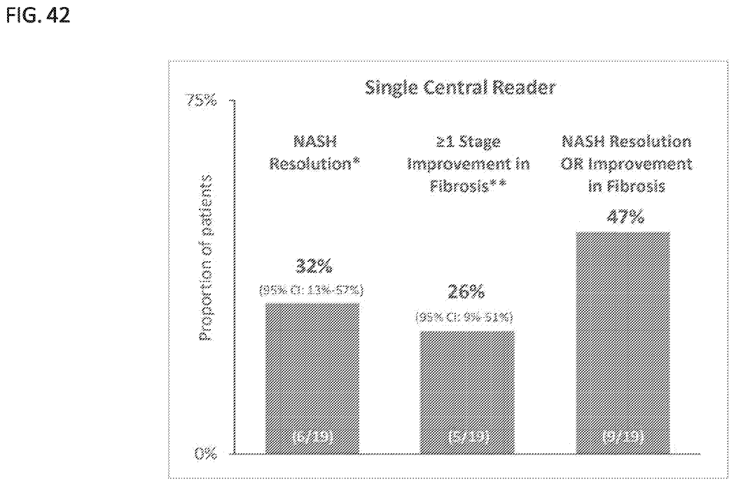

FIG. 42 shows that BIO89-100 demonstrated clinically meaningful changes on key histological efficacy endpoints. In the three-panel read, NASH resolution was up to 47% (range: 26-47%); Fibrosis improvement was up to 42% (range: 12-42%), and 2-point NAS improvement: up to 79% (range: 68-79%).

Results also showed clinically meaningful and significant changes across key non-invasive tests (NITs) associated with fibrosis, risk of fibrosis or NASH resolution.

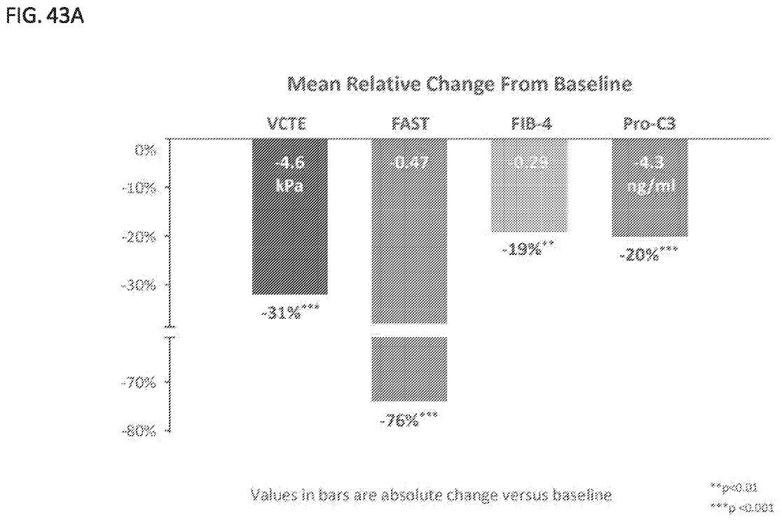

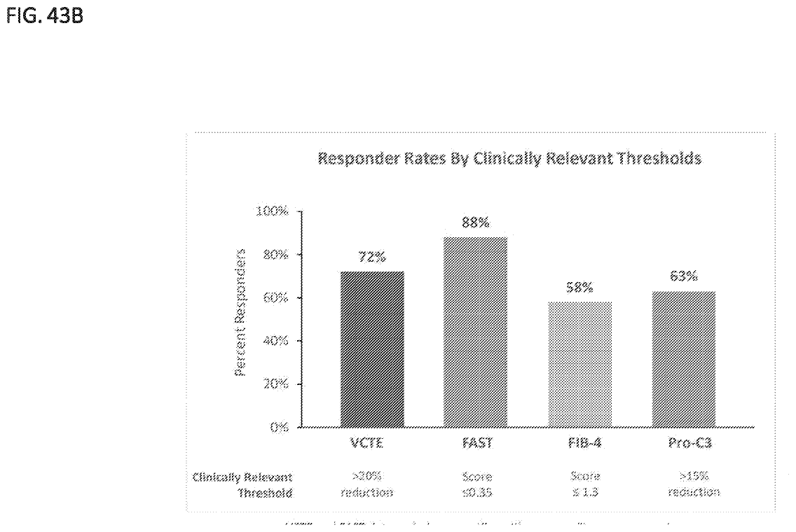

FIG. 43A shows that BIO89-100 substantially improved scores across Non-Invasive Tests (NITs) correlated with advanced fibrosis. NIT descriptions are as follow:
VCTE: Liver stiffness measure using FibroScan®
FAST score: Liver stiffness
(VCTE) and steatosis (CAP) using FibroScan® plus AST; 0-1 scale
FIB-4 score: Composite serum marker/age measure
Pro-C3: Collagen deposition serum biomarker

Clinically relevant thresholds are as followed:
VCTE: >20% reduction correlates with fibrosis improvement
FAST score: Score ≤0.35 predicts Fibrosis Stage F0/F1 and NAS <4
FIB-4 score: Score <1.3 predicts Fibrosis Stage F0/F1
Pro-C3: >15% reduction correlates with fibrosis improvement

FIG. 43B shows that Bio89-100 had high percentages of responders based on clinically relevant thresholds for NITS.

In addition to significant improvement in liver health, treatment with pegozafermin also had significant positive effects on glycemic control, lipids, and body weight.

TABLE 6
Cardio-metabolic endpoints

| | Mean change from baseline at Week 20 |
|---|---|
| HbA1c absolute change¹ | −0.9%** |
| Triglycerides² | −32%*** |
| LDL-C | −13%* |
| HDL-C | +23%*** |
| Body Weight | −4%*** |

*p < 0.05;
**p < 0.01;
***p < 0.001
¹In patients with HbA1c ≥6.5% at baseline (n = 10); patients were all on concomitant diabetes medications
²In patients with elevated triglycerides at baseline (n = 11); reduction was −26% across total population

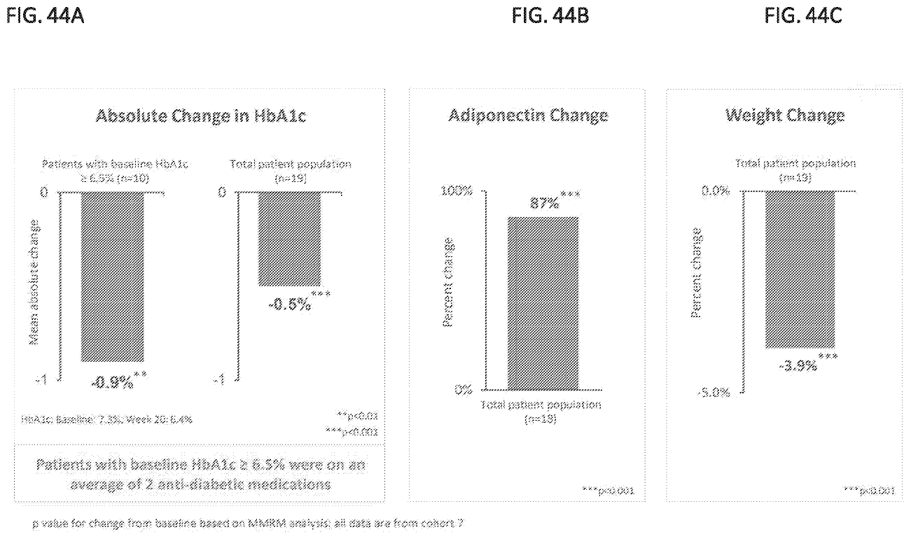

FIGS. 44A, 44B and 44C show that BIO89-100 demonstrated clinically meaningful improvement on HbA1c and adiponectin with notable body weight reduction.

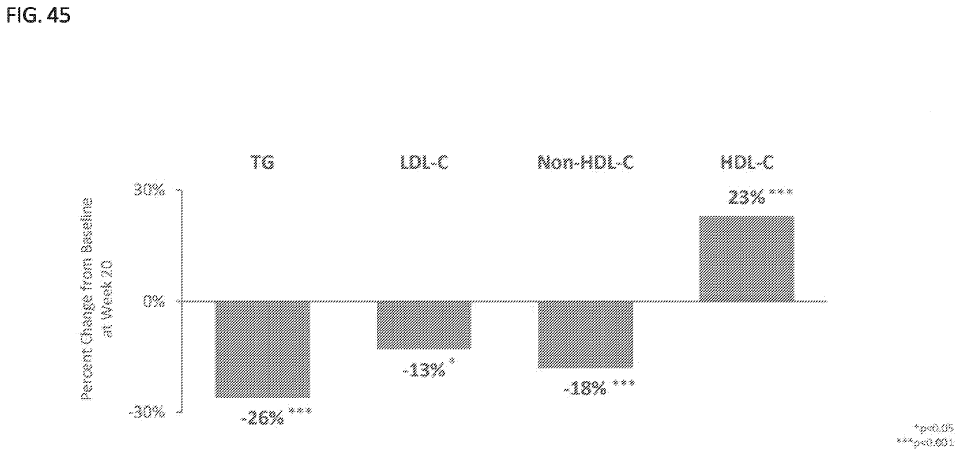

FIG. 45 shows that BIO89-100 demonstrated clinically meaningful reduction in lipid parameters such as TG, LDL-C, Non-HDL-C and meaning full increase is HDL-C.

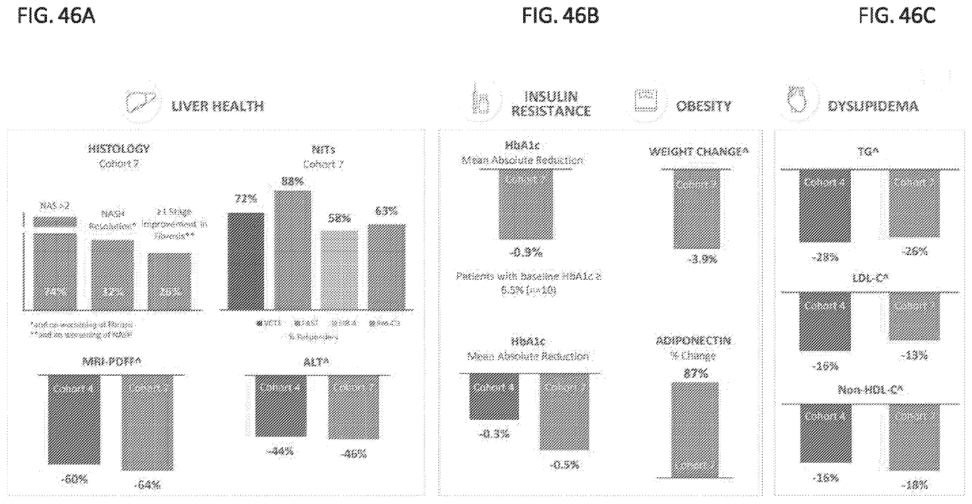

FIGS. 46A, 46B and 46C show that BIO89-100 improves many markers of liver health and co-morbidities associated with NASH.

In 83 patients treated with BIO89-100 across the full Phase 1b/2a study, BIO89-100 continues to be generally well tolerated with a favorable safety profile. There have been no drug-related serious adverse events, only one treatment-related discontinuation, no tremors and no hypersensitivity reactions have been observed. There was no adverse effect on blood pressure or heart rate. In the open-label histology cohort the most commonly reported treatment-related adverse events were nausea, diarrhea, vomiting and injection site reactions, most of which were graded as mild and/or of short duration.

### Example 6: Phase 2b NASH Trial Design

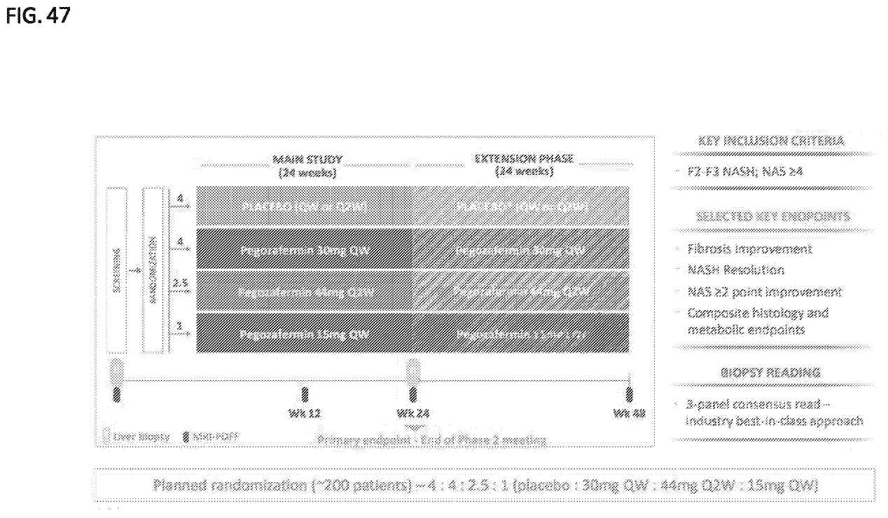

FIG. 47 show Phase 2b NASH trial design.

A Randomized, Double-Blind, Placebo-Controlled Study to Evaluate the Efficacy, Safety and Tolerability of pegozafermin in Subjects with Biopsy-Confirmed Nonalcoholic Steatohepatitis (NASH)

Doses tested: Placebo, 30 mg pegozafermin weekly, 15 mg pegozfermin weekly, 44 mg pegozafermin every two weeks

Duration: 24 weeks treatment to primary endpoint followed by blinded 24-week treatment in extension phase
Number of subjects: Approximately 200 patients
Key inclusion criteria: F2-F3 NASH; NAS ≥4
Selected key endpoints:
   One stage Fibrosis Improvement with no worsening of NASH
   NASH Resolution with no worsening of Fibrosis
   NAS ≥2 point improvement
   Composite histology and metabolic endpoints

Biopsy reading methodology: Baseline and end of treatment biopsy (24 weeks) read by 3-panel pathologists using consensus read.

### Example 7: Safety, Pharmacokinetics and Pharmacodynamics of Pegozafermin, a glycoPEGylated Analogue of Fibroblast Growth Factor 21, in Patients with Non-Alcoholic Steatohepatitis: A Multicentre, Randomised, Double-Blind, Placebo-Controlled, Phase 1b/2a Multiple Ascending Dose Study

### Abstract

### Background

The aim of this study was to evaluate the safety and efficacy of pegozafermin (BIO89-100), a glycoPEGylated fibroblast growth factor analogue, in participants with biopsy-confirmed non-alcoholic steatohepatitis (BC-NASH) or non-alcoholic fatty liver disease and high risk of NASH (referred to in this study as phenotypic NASH [PNASH]).

### Methods

This multicentre, randomised, double-blind, placebo-controlled, phase 1b/2a multiple ascending dose study enrolled adults (21-75 years) with BC-NASH/PNASH across 12 clinical sites in the USA. Patients were randomised to receive subcutaneously administered pegozafermin (3, 9, 18 or 27 mg once weekly [QW]; 18 or 36 mg once every two weeks [Q2W]) or placebo for 12 weeks. Primary endpoints were safety, tolerability and pharmacokinetics. Secondary endpoints comprised effects of immunogenicity (anti-drug antibodies [ADAs]), and week-13 changes from baseline in hepatic parameters (magnetic resonance imaging proton-density fat fraction [MRI-PDFF]alanine aminotransferase [ALT], aspartate transaminase [AST] and N-terminal propeptide of type III collagen [PRO-C3]), lipids (non-high-density lipoprotein cholesterol [non-HDL-D], high-density lipoprotein cholesterol [HDL-D] and low-density and lipoprotein cholesterol [LDL-C]), adiponectin, free fatty acids, insulin resistance, adipose tissue insulin resistance and weight (ClinicalTrials.gov number: NCT04048135).

### Findings

Between Jul. 29, 2019, and Mar. 18, 2020, 275 participants were screened and 81 (15 [18.5%] with BC-NASH) were randomised (62 received pegozafermin; 19 received placebo). The most common treatment-related adverse event (AE) was mild increased appetite (10/63 [15.9%] for pooled pegozafermin vs 0/18 [0.0%] for pooled placebo), which was not associated with weight gain. No treatment-related serious AEs or deaths occurred. Dose-proportional pharmacokinetics were observed. ADAs were detected in 41 (65.1%) of 63 participants treated with pegozafermin; emergence appeared dose-dependent and there was no evidence of effects on PK, PD, or safety profiles. Pegozafermin statistically significantly improved hepatic parameters versus pooled placebo, with the greatest reductions observed in the 27 mg QW cohort (n=7) for hepatic fat fraction (MRI-PDFF) (LS mean absolute reduction of −13.5% vs +1.4%; difference: −14.9% [95% CI: −20.1, −9.7]; p<0.0001), ALT levels (LS mean relative reduction of −43.7% vs −4.2%; difference: −39.5% [95% CI: −59.9, −19.2]; p=0.0002) and AST levels (LS mean relative reduction of −37.9% vs −4.2%; difference: −33.5% [95% CI: −51.4, −15.5]; p=0.0004), and 85.7% (6/7) achieving a ≥30% relative reduction in MRI-PDFF versus none for pooled placebo (p<0.0001). LS mean differences with pegozafermin 27 mg QW versus pooled placebo were −25.4% for triglycerides (95% CI: −48.4, −2.4; p=0.0308), −17.5% for non-HDL-C (95% CI: −30.6, −4.4; p=0.0095), −17.6% for LDL-C (95% CI: −32.7, −2.6; p=0.0224), +65.1% for adiponectin (95% CI: +36.6, +93.7; p<0.0001), −30.9% for PRO-C3 (95% CI: −57.4, −4.5; p=0.0227) and −2.2% (95% CI: −4.2, −0.12; p=0.0380) for weight. Statistically significant effects were not observed for other secondary endpoints assessed in the 27 mg QW cohort.

### Interpretation

Pegozafermin was generally well-tolerated and associated with clinically meaningful reductions in liver fat, measures of liver function and lipids. Further evaluation of pegozafermin in NASH is warranted.

### Research in Context

Evidence Before this Study

An estimated 27 million individuals in the USA (~8% of the general population) will have non-alcoholic steatohepatitis (NASH) by 2030. Management strategies for NASH, a key risk factor for cirrhosis, hepatocellular carcinoma and cardiovascular events, and a leading cause of liver transplantation, are based predominantly on lifestyle modification, with no approved disease-modifying drugs yet available. Fibroblast growth factor (FGF21), an endogenous metabolic hormone, is a key regulator of glucose and lipid metabolism. Administration of FGF21 analogues has been highlighted as a potential treatment strategy for NASH. On 22 Nov. 2021, we searched PubMed for randomised controlled trials of FGF21 analogue-based therapies for the treatment of NASH, using the following search string: (non-alcoholic fatty liver disease [MeSH] OR non-alcoholic fatty liver disease OR NAFLD OR steatohepatitis OR NASH OR fatty liver) AND (Receptors, Fibroblast Growth Factor [MeSH] OR fibroblast growth factor 21 OR FGF21 OR FGF-21). Two randomised, double-blind, placebo-controlled, phase 2a trials were identified that assessed the effects of 12-16 weeks of treatment with pegbelfermin (polyethylene glycol-conjugated [PEGylated] recombinant human FGF21) and efruxifermin (fusion of human IgG1 Fc domain with modified human FGF21), respectively, given at least weekly via subcutaneous injection to patients with biopsy-confirmed NASH (BC-NASH). Each study reported statistically significant reductions with study drug versus placebo in hepatic fat fraction, liver transaminases (alanine aminotransferase and aspartate aminotransferase), and lipid metabolism (triglycerides, low- and high-density lipoprotein cholesterol and adiponectin). Statistically significant reductions in PRO-C3 (a marker of fibrosis) levels were also reported in both studies. The most common adverse events (AEs) occurring more frequently with pegbelfermin or efruxifermin than with placebo were gastrointestinal (GI) in nature. Absolute reductions in hepatic fat fraction reported in the efruxifermin study (−12.3 to −14.1% [efruxifermin] vs +0.3% [placebo]) were higher than those reported in the pegbelfermin study (−6.8% to −5.2% pegbelfermin vs −1.3% [placebo]), but GI-related AEs appeared to be substantially more frequent with efruxifermin than with pegbelfermin.

Added Value of this Study

In this randomised, double-blind, placebo-controlled, phase 1b/2a proof of concept study, multiple ascending doses of the glycoPEGylated FGF21 analogue pegozafermin (BIO89-100) were administered once-daily (QW) or once every two weeks (Q2W) to participants with non-alcoholic fatty liver disease (NAFLD) at high risk of NASH (referred to in this study as phenotypic NASH) or BC-NASH for 12 weeks. The most common treatment-related AE was mild increased appetite, which was not associated with weight gain. No other GI-related AEs were reported at a higher frequency in the pegozafermin treatment groups compared with placebo. Statistically significant, absolute reductions in hepatic fat fraction of up to −13.5% and −9.7% were observed at week 13 with pegozafermin QW and Q2W dosing, respectively, compared with an increase of +1.4% with placebo. A high proportion (up to 88%) of participants had at least a 30% reduction in hepatic fat fraction, a threshold shown to correlate with histological improvements and reduced fibrosis progression in previous studies. Consistent with previous FGF21 analogue trials, improvements in liver transaminases, the fibrosis marker PRO-C3 and lipid metabolism, were observed with pegozafermin compared with placebo. These data indicate that pegozafermin may combine the promising efficacy of an FGF21 analogue for the treatment of NASH, with potential added benefits of a milder AE profile and the possibility of Q2W dosing.

### Implications of all the Available Evidence

The results of the current trial further support the therapeutic potential of FGF21 analogues in patients with NASH, a disease for which the unmet medical need is high. The beneficial effect of these molecules on liver-related parameters, combined with attenuation of metabolic perturbations that underlie NASH pathology and risk factors for cardiovascular disease (a leading cause of death in these patients), are promising. The efficacy and safety of pegozafermin 15 mg QW, 30 mg QW and 44 mg Q2W are currently being assessed in patients with NASH (NAFLD Activity Score [NAS]≥4) and fibrosis (stage 2 or 3) in the ongoing phase 2 ENLIVEN study (ClinicalTrials.gov number: NCT04929483).

### Introduction

Non-alcoholic steatohepatitis (NASH), the progressive form of non-alcoholic fatty liver disease (NAFLD), is a chronic disease characterised by steatosis in at least 5% of hepatocytes, lobular inflammation, and hepatocyte ballooning, with or without fibrosis.¹ The current global prevalence is 1.5-6.5% in the general population; in the United States, the prevalence of NASH is expected to increase from 16.5 million cases in 2015 (~5% of the general population) to 27 million (~8%) by 2030.^{2, 3} NASH, which is expected to become the leading cause of liver transplantation in the near future (and is currently the leading cause in women),⁴ progresses to fibrosis and cirrhosis in approximately 20% of patients, and 45% of patients with cirrhosis will progress to decompensated cirrhosis within 10 years.⁵⁻⁷

Despite advances in understanding the pathological mechanisms in NASH, there are currently no approved disease-modifying pharmacological interventions; lifestyle modification continues to be the recommended strategy for disease management.⁸ Fibrosis progression is a strong predictor of mortality and liver-related morbidity in patients with NASH and fibrosis improvement is an important goal of treatment.⁹ Notably, relative reductions in hepatic fat of at least 30%, as assessed by magnetic resonance imaging proton-density fat fraction (MRI-PDFF) in NASH clinical trials, correlate with histological improvements and reduced fibrosis progression.¹⁰⁻¹²

Fibroblast growth factor 21 (FGF21) is an endogenous metabolic hormone secreted by the liver, and is a key regulator of energy expenditure and glucose and lipid metabolism via activation of various FGF receptors (FGFRs) in metabolically-active organs.¹³⁻¹⁵ In patients with insulin resistance and NASH (and possibly in some genetic variants, such as rs499765), (Jiang, 2014) circulating and tissue levels of FGF21 are increased and correlate with disease severity, suggesting FGF21 resistance, which may be overcome by administration of pharmacological doses of FGF21. On this basis, administration of exogenous FGF21 has been explored as a method of treating obesity-associated insulin-resistance disorders, including NASH.¹⁶⁻¹⁸

Pharmacological administration of FGF21 has been shown to have beneficial effects in patients with NASH. These include: increased hepatic insulin sensitivity; stimulation of fatty acid oxidation; inhibition of de novo lipogenesis; and decreased delivery of triglyceride-enriched very low-density lipoprotein (VLDL) via downregulation of VLDL receptor expression in hepatocytes.¹⁹ However, native FGF21 has a short half-life (~2 hours), limiting its therapeutic potential. In animals, glycoPEGylation of FGF21 greatly increases its in vivo half-life while maintaining similar effects to endogenous FGF21 with a lower dosing frequency and total cumulative exposure.^{20, 21}

Pegozafermin (BIO89-100) is a glycoPEGylated FGF21 analogue that has an N-terminal methionine residue, two point-mutations, and a single 20 kDa linear polyethylene glycol (PEG) covalently attached via a glycosyl moiety. In two NASH animal models (mice and spontaneously diabetic monkeys), pegozafermin conferred liver-related and metabolic benefits, including improved transaminase levels and liver histology, decreased body weight, improved glycemic parameters and lipid profiles, and increased adiponectin levels.^{21, 22} A first-in-human, phase 1, single ascending dose study in healthy volunteers demonstrated that subcutaneous administration of pegozafermin was associated with a half-life of 55-100 hours, supporting investigation of dosing both once per week (QW) and once every 2 weeks (Q2W). 23 Statistically significant beneficial changes in triglycerides, low-density lipoprotein cholesterol (LDL-C), high-density lipoprotein cholesterol (HDL-C), and adiponectin levels were also observed in this study.²³ These early studies suggested that patients with NASH may derive benefit from treatment with pegozafermin.

Here we present data from a phase 1b/2a study that aimed to evaluate the safety, tolerability, and pharmacokinetic (PK) and pharmacodynamic (PD) effects of multiple ascending doses of pegozafermin in participants with biopsy-confirmed NASH (BC-NASH) or with NAFLD and at high risk of NASH (referred to henceforward as phenotypic NASH [PNASH]).

### Methods

### Study Design and Participants

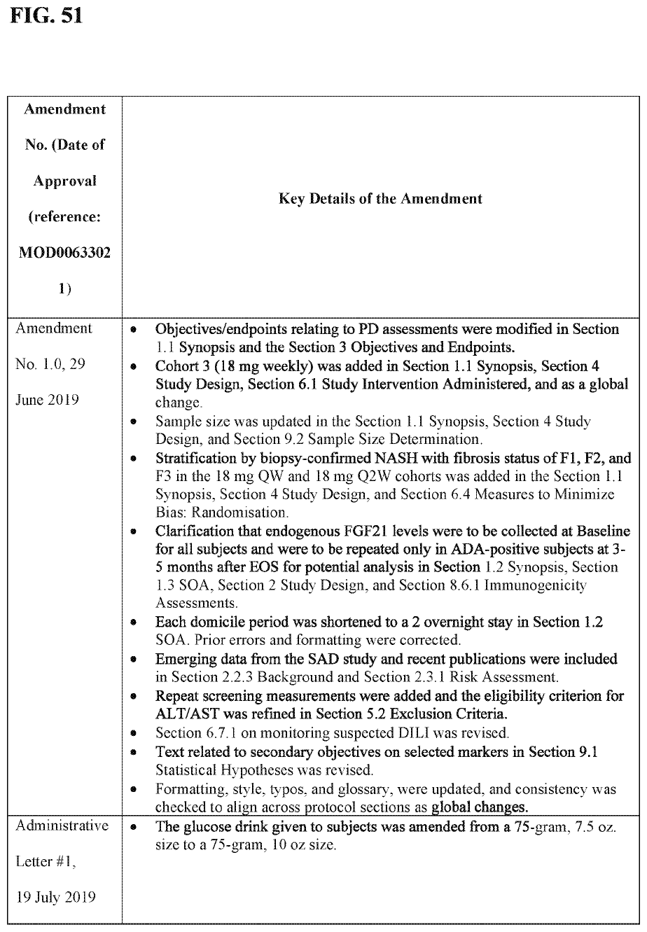

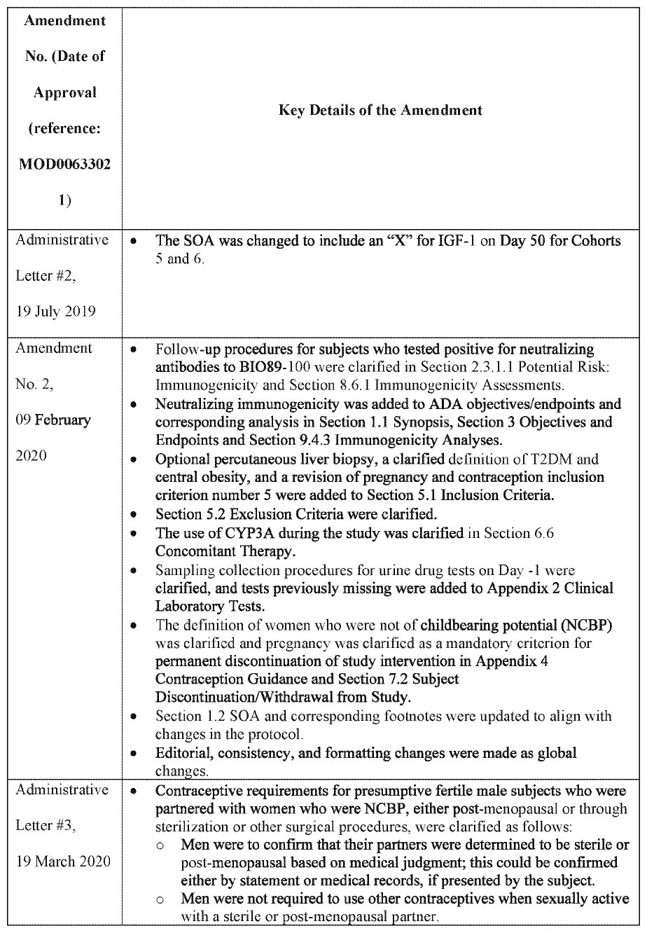

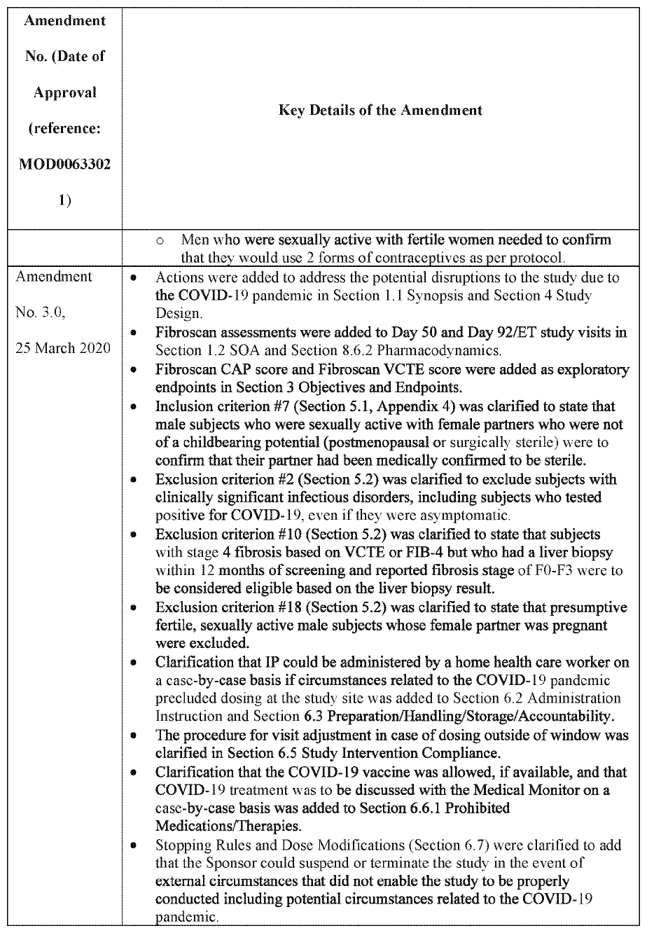

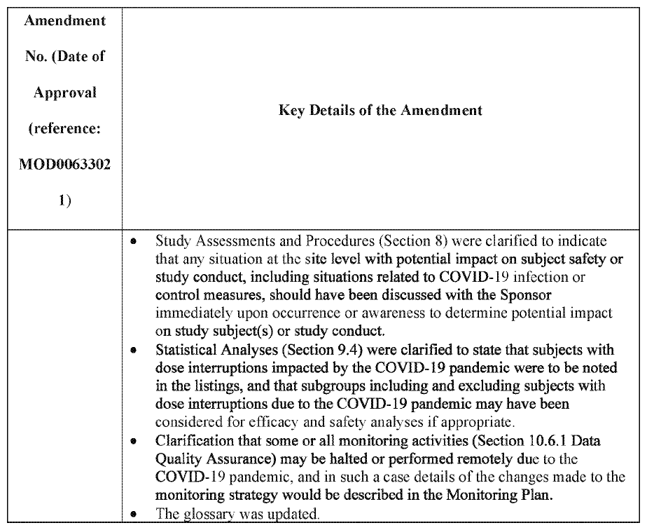

This was a randomised, double-blind, placebo-controlled, multiple ascending dose, proof-of-concept, phase 1b/2a study conducted at 12 clinical sites in the United States from 29 Jul. 2019 to 3 Aug. 2020. The study protocol, and amendments (FIG. 51), were approved by the Institutional Review Board or Independent Ethics Committee for each site. All participants provided written informed consent. The trial was registered with ClinicalTrials.gov (NCT04048135).

Adults 21 to 75 years of age with an MRI-PDFF of ≥10% and a body mass index (BMI) of at least 25 kg/m² were enrolled. Participants were required to have either BC-NASH with NASH Clinical Research Network (CRN) fibrosis stage 1, 2, or 3 based on biopsy performed in the 24 months before screening, or PNASH if biopsy was not available. PNASH was defined as: obesity (BMI >30 kg/m²) with either type 2 diabetes mellitus (T2DM; fasting plasma glucose ≥126 mg/dL, 2-hour plasma glucose in a 75 g oral glucose tolerance test ≥200 mg/dL, or glycated haemoglobin [HbA1c] ≥6.5% [48 mmol/mol]) or evidence of liver injury (increased alanine aminotransferase [ALT; ≥40 U/L in men or ≥30 U/L in women]; and/or FibroScan [Echosens, Waltham, MA, USA] vibration-controlled transient elastography score ≥7 kPa). Individuals were excluded if they had liver disease other than NASH, evidence of cirrhosis, cardiovascular or cerebrovascular disease, or any illness that, in the opinion of the investigator, might confound the results of the study or pose an additional risk to the patient. Individuals with any clinically significant abnormality at screening in laboratory parameters, electrocardiogram (ECG), or vital signs were also excluded.

### Randomisation and Masking

The principal investigators at each study site enrolled participants. Eligible individuals were centrally randomised within cohorts (with allocation concealment) using an Interactive Web Response System (IWRS; developed, deployed and supported by ProSciento, Inc.), to one of six cohorts in the order they were enrolled. The ‘dummy’ subject randomisation and inventory schedules were tested in the IWRS to ensure the system performed according to protocol requirements. The final, actual subject randomisation and inventory schedules were then imported into the IWRS by an unblinded statistician. Periodic review of the randomisation was performed by the unblinded statistician throughout the study.

Participants, principal investigators, other study personnel and the study sponsor were blinded to the treatment assignments throughout the study. Pegozafermin and placebo were prepared in syringes at each study site by an unblinded pharmacist. Unblinded pharmacists were not involved in any other study-related procedures. Syringes containing pegozafermin or placebo appeared identical, and study drug was administered by blinded site staff.

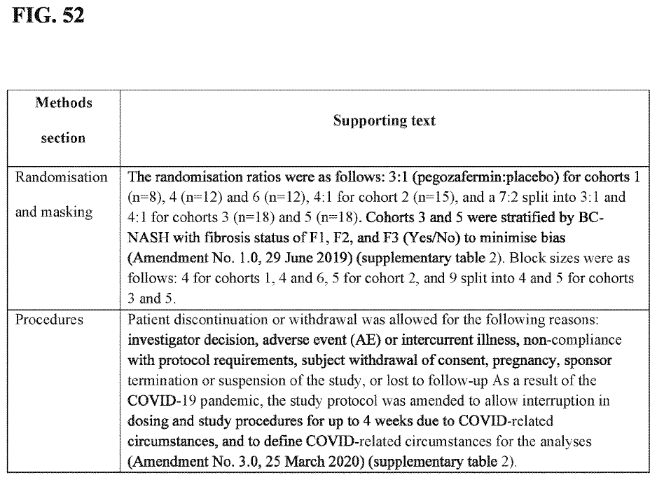

Two dosing regimens were evaluated: once weekly (QW; 3 mg [cohort 1], 9 mg [cohort 2], 18 mg [cohort 3], 27 mg [cohort 4]) and once every 2 weeks (Q2W; 18 mg [cohort 5], 36 mg [cohort 6]) (randomisation ratios and block sizes are reported in FIG. 52). Within each cohort, participants were randomised to pegozafermin or placebo and treated for 12 weeks, with the first dose administered on day 1 and the last dose administered on day 85 (13 doses for the QW regimens and seven doses for the Q2W regimens). A Safety Monitoring Committee (SMC) was set up to review participants' safety and for dose escalation decisions. The SMC was composed of the sponsor's medical monitor (MM), the clinical research organization medical monitor (LM), and at least one principal investigator (BBF). There were two planned SMC meetings for dose escalation decisions, and additional post-hoc meetings could be held as needed. A blinded safety review was conducted by the SMC after participants in cohort 1 completed the day 36 visit. If no safety concerns were identified, randomisation of additional participants into cohorts 2 and 5 was initiated. If no safety concerns were identified after at least eight participants from both cohort 2 and cohort 5 had completed the day 36 visit, including at least one participant receiving placebo in each cohort, randomisation of additional participants into cohorts 3, 4, and 6 was initiated.

### Procedures

Patients were treated QW (cohorts 1, 2, 3, and 4) or Q2W (cohorts 5 and 6) with one or two subcutaneous injections of pegozafermin or placebo in the abdomen starting on day 1 and continuing through day 85. Administration of pegozafermin or placebo was performed by qualified study personnel (allowed reasons for study discontinuation, withdrawal, or interruption are provided in FIG. 52).

AEs were continuously monitored throughout the study (FIG. 51 and FIG. 52) and were coded using the Medical Dictionary for Regulatory Activities version 23.0. The SMC reviewed blinded safety data (AEs, clinical laboratory parameters, vital signs, and electrocardiograms [ECG])

Blood sampling for PK analyses were initiated on day 1 (first dosing day), as well as on day 29 (fifth dosing day for QW cohorts; third dosing day for Q2W cohorts) when steady-state serum pegozafermin concentrations were achieved. Samples were taken pre-dose (days 1 and 29), and post-dose at 6, 12, 24 (days 2 and 30), 48 (days 3 and 31), 72 (days 4 and 32), 96 (days 5 and 33) and 168 hours (days 8 and 36). For the Q2W cohorts, an additional PK blood sample was taken 336-hours after the day 43 dose (rather than the day 29 dose), but was incorporated into the day 29, steady-state PK analysis as the ‘trough’ value for pegozafermin exposure.

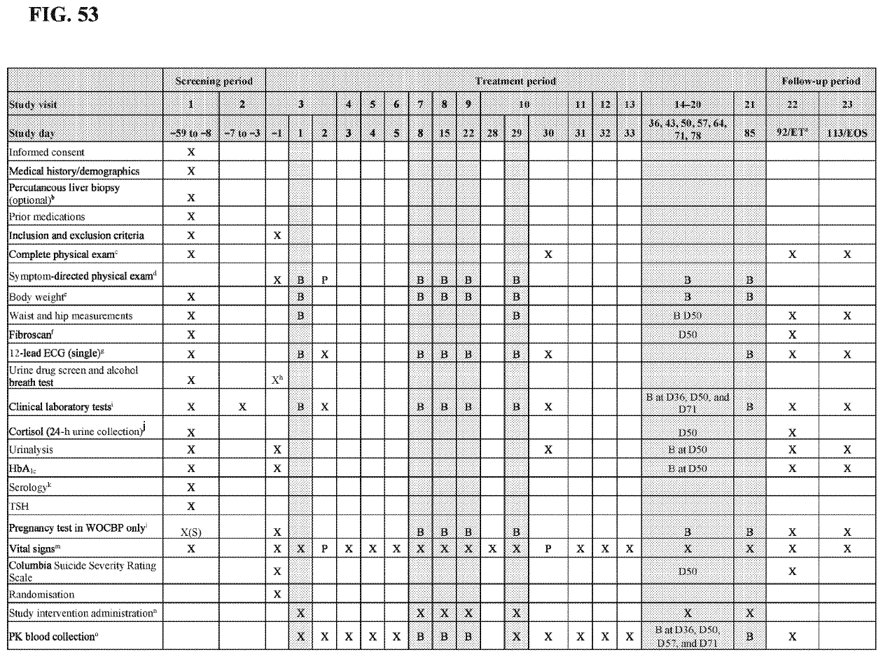

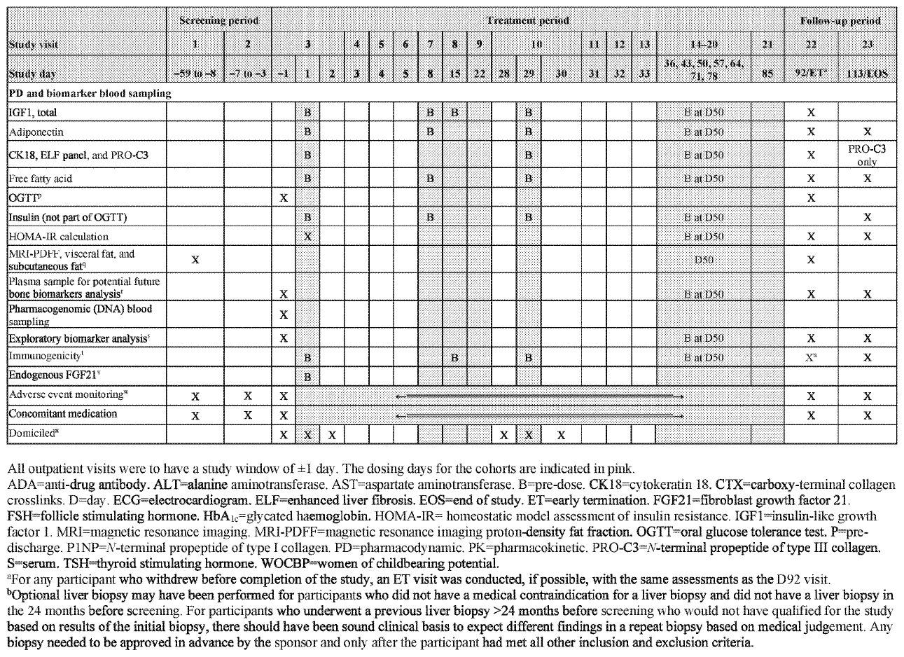

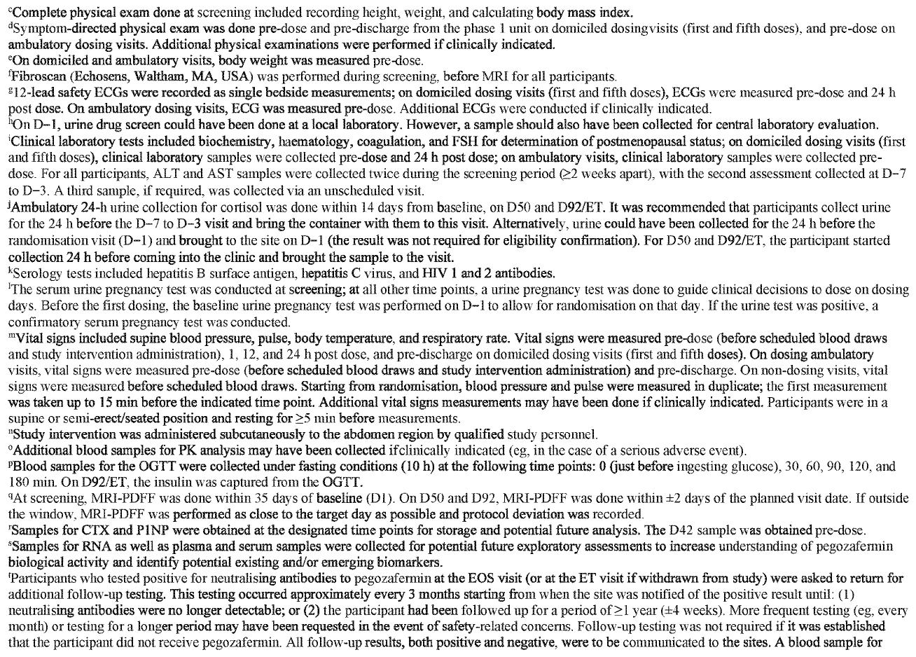

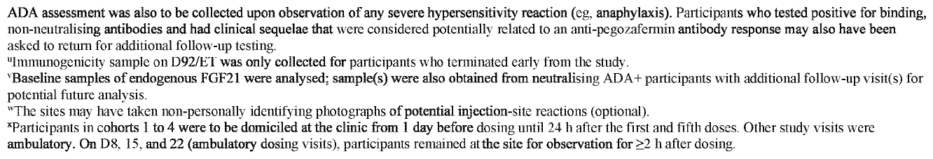

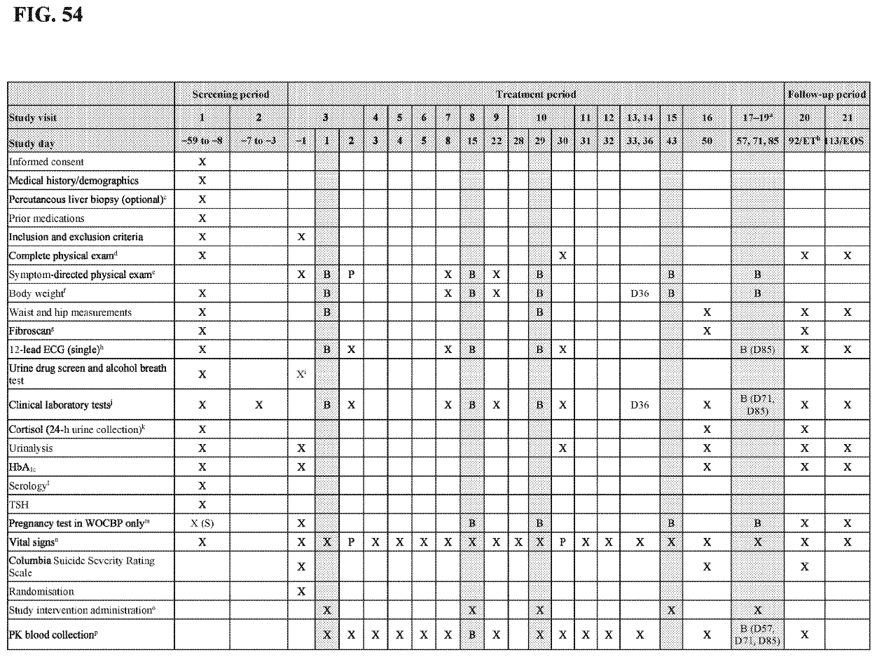

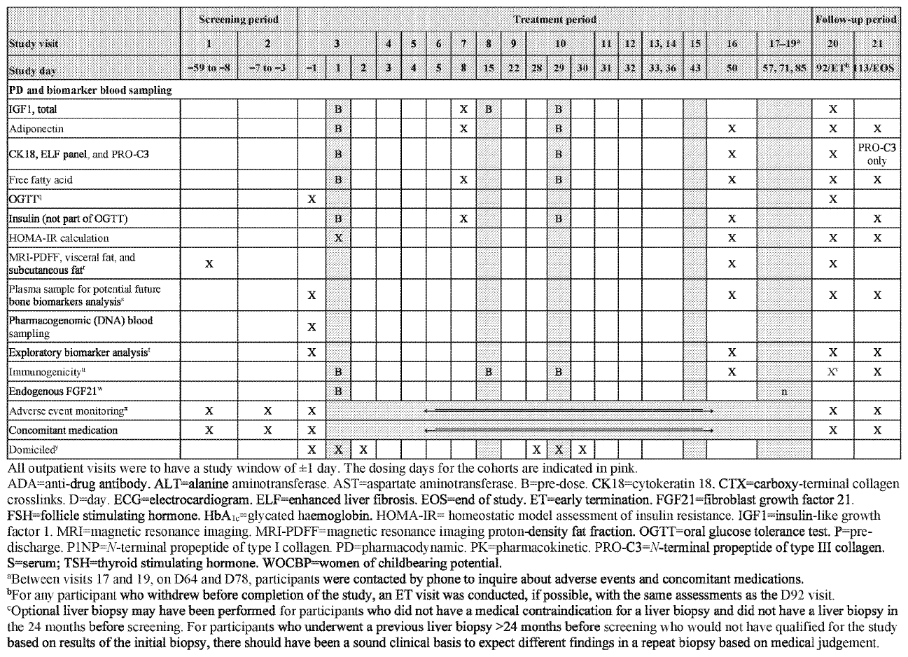

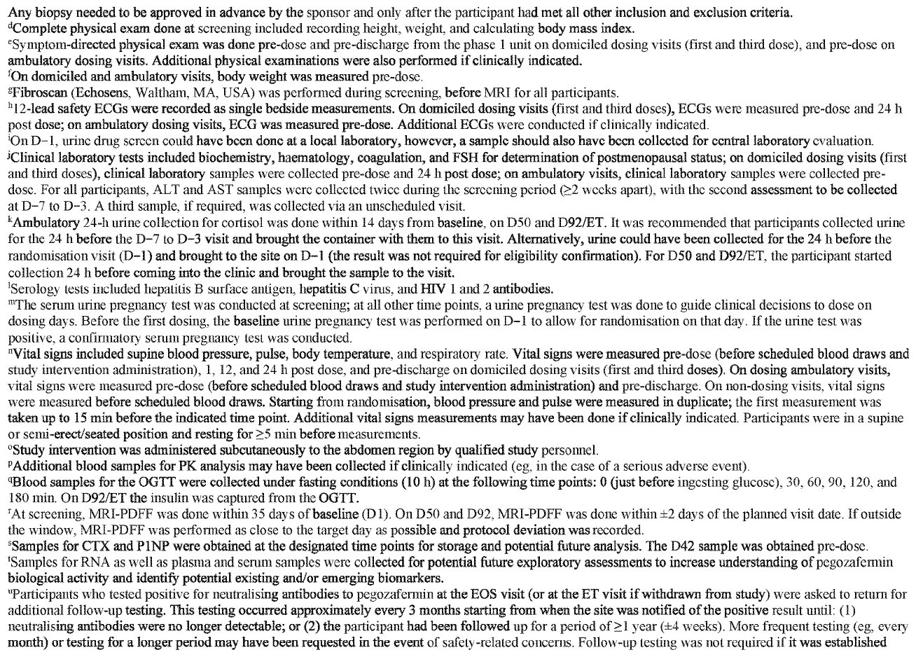

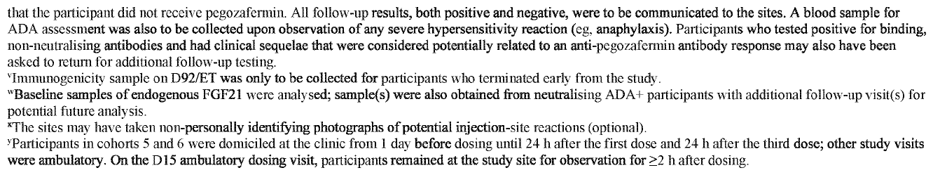

PD assessments (MRI-PDFF, hepatic and metabolic biomarkers etc.), laboratory tests, 12-lead ECG and vital signs were assessed as per schedule (FIG. 53 and FIG. 54).

### Outcomes

The primary endpoints of the study were safety and tolerability of pegozafermin, assessed by the frequency and severity of adverse events (AEs) and serious adverse events (SAEs) and the number of patients who discontinued owing to AEs and treatment-related AEs, and the PK of pegozafermin, determined by the maximal observed serum concentrations (Cₘₐₓ) within a dosing interval, area under the serum drug concentration-time curve from time zero to time of last measurable concentration within a dosing interval (AUCₗₐₛₜ), time to achieve Cₘₐₓ (tₘₐₓ), terminal elimination half-life (tv), and accumulation ratios (AUCₗₐₛₜ on day 29 [steady-state]/AUCₗₐₛₜ on day 1).

Absolute and percentage changes from baseline in hepatic fat fraction (MRI-PDFF) were key secondary endpoints. Additional secondary endpoints included absolute and percentage changes from baseline in body weight, triglycerides, HDL-C, non-HDL-C, LDL-C, HbA1c, homeostatic model assessment of insulin resistance (HOMA-IR), adipose insulin resistance (Adipo-IR), liver function tests (ALT and aspartate transaminase [AST]), adiponectin and N-terminal propeptide of type III collagen (PRO-C3). The immunogenicity of pegozafermin, measured by the incidence and characteristics of anti-drug antibodies (ADA) after dosing (eg, titre and/or binding specificity to the PEG component of pegozafermin, and neutralising immunogenicity), as well as potential effects of ADAs on serum pegozafermin concentrations and safety, were also assessed as secondary endpoints. In addition, absolute and percentage changes from baseline in hepatic volume (by MRI) were key exploratory endpoints.

Other safety endpoints were incidences of, and clinically significant shifts in, vital signs, physical examination findings, ECG data, and clinical laboratory parameters including complete blood count, biochemistry, cortisol, and urinalysis.

### Statistical Analysis

No formal sample size calculation was performed for the primary endpoints as the number of participants (n=81) was considered adequate for this phase 1b/2a study to achieve the safety, tolerability, and PK objectives. In relation to changes in hepatic fat fraction (key secondary endpoint), a power calculation showed that either 9, 12, or 14 participants in a dose cohort, compared with a pooled placebo group of 19 participants, would provide ~89%, ~93%, and ~95% power, respectively, to detect differences between treatment groups of 30% in terms of the mean percentage change in MRI-PDFF from baseline, assuming a standard deviation of 25% for this endpoint in each group. These calculations were based on a two-sample t-test with one-sided 5% (two-sided 10%) type I error probability.

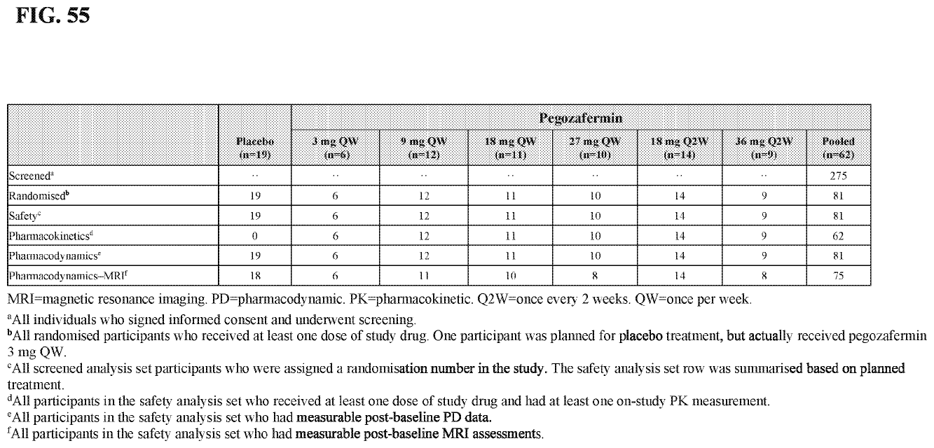

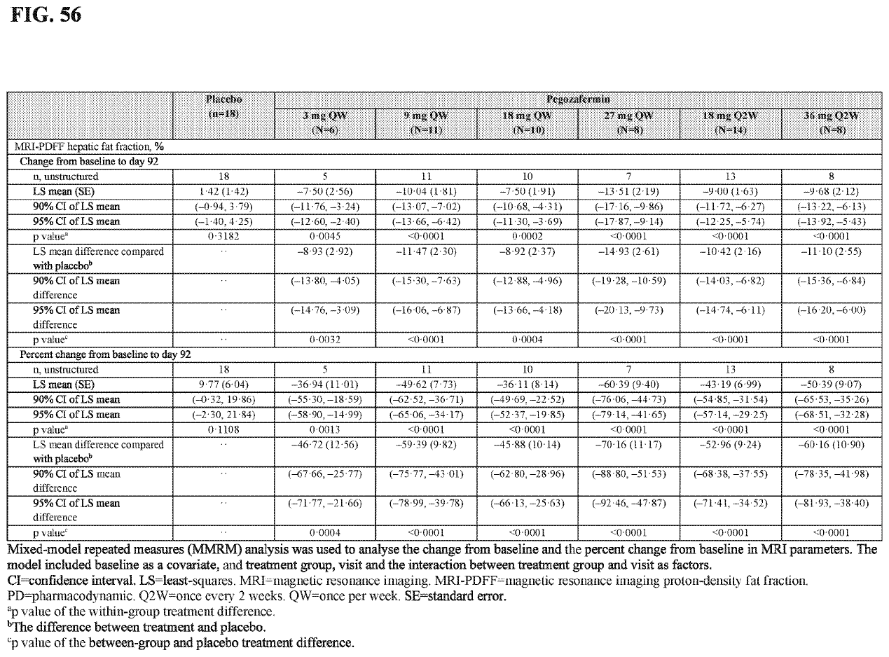

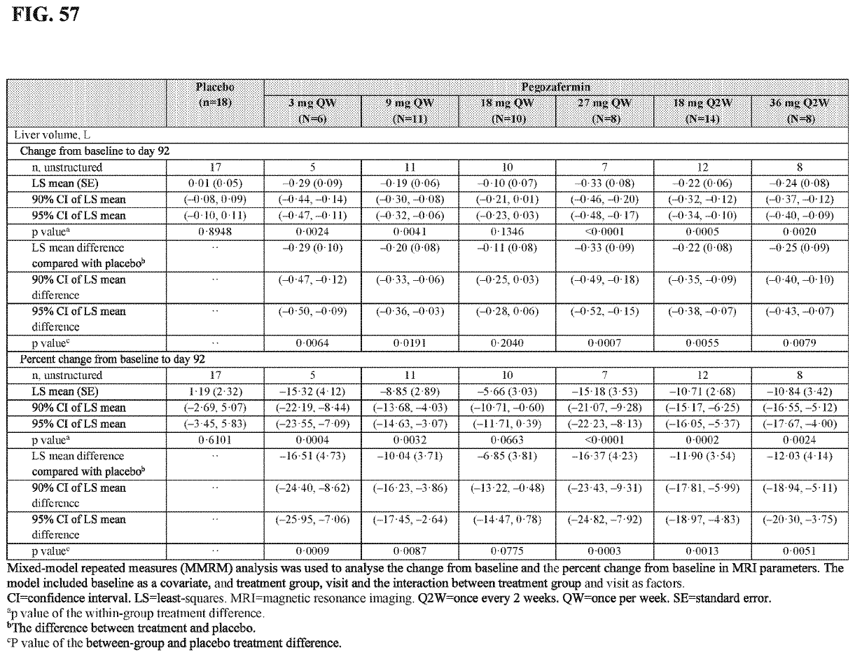

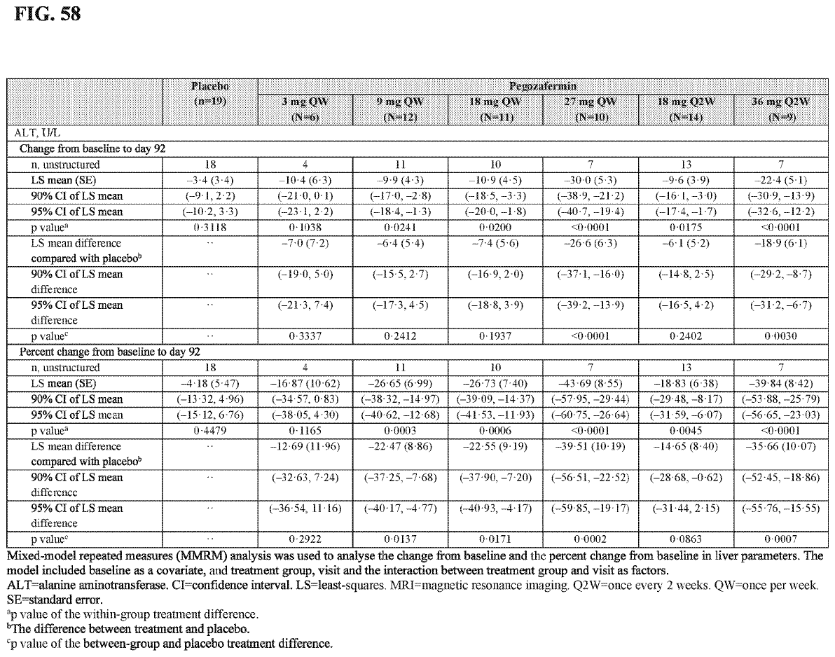

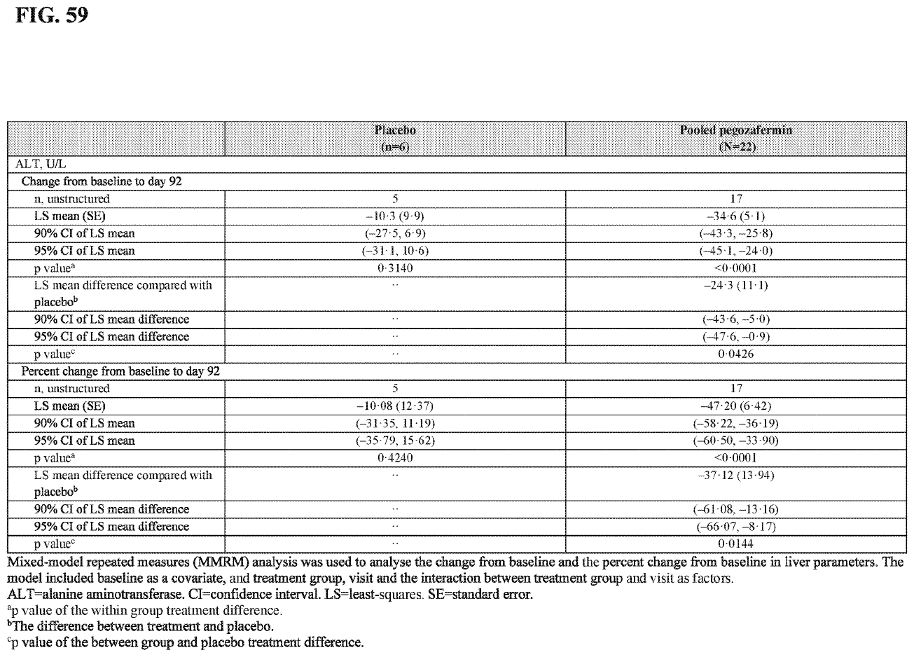

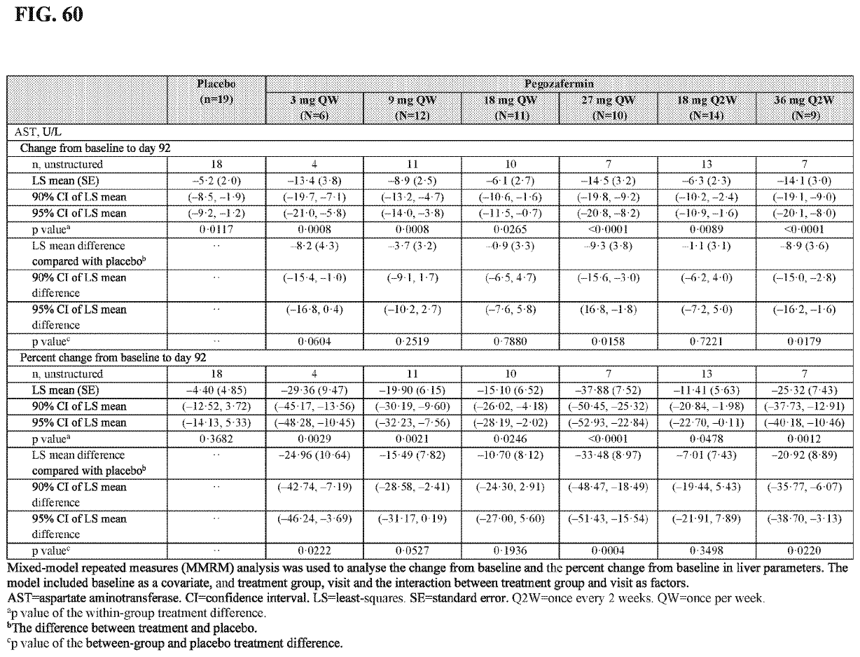

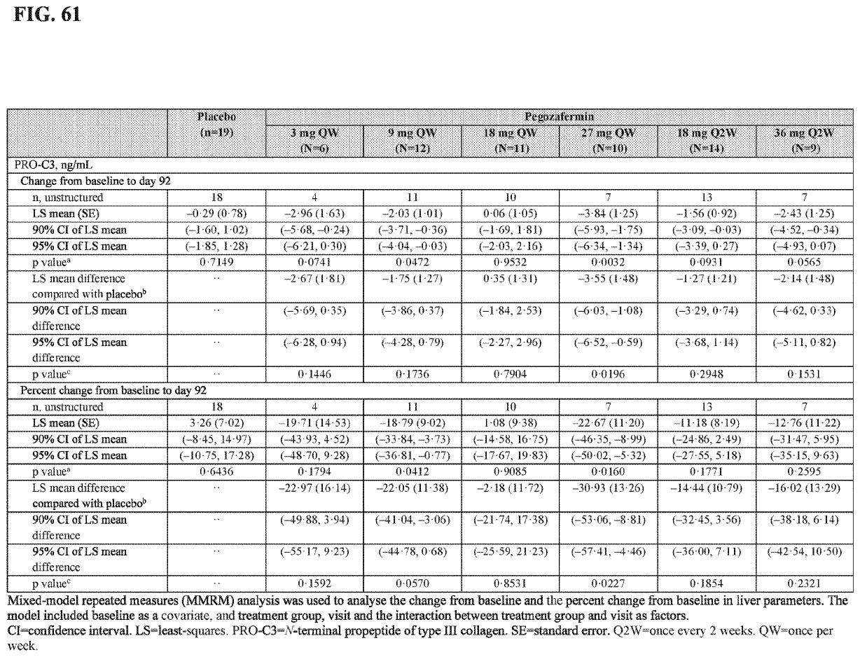

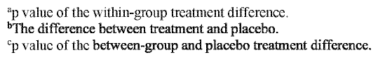

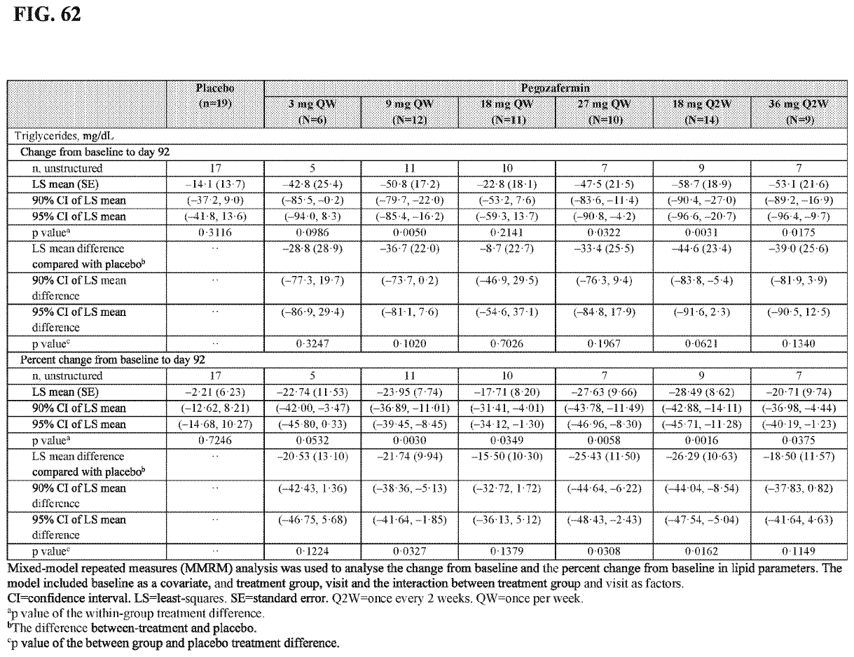

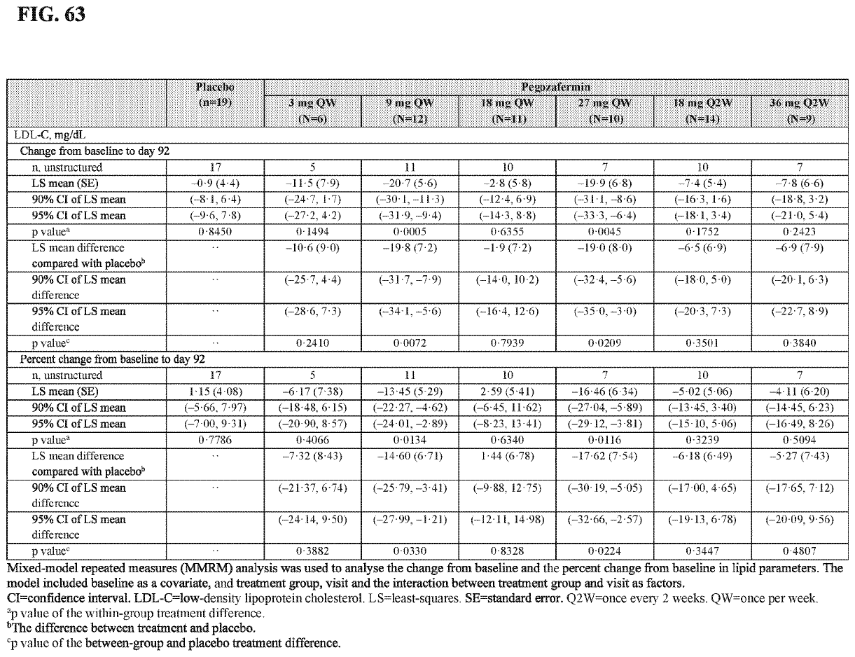

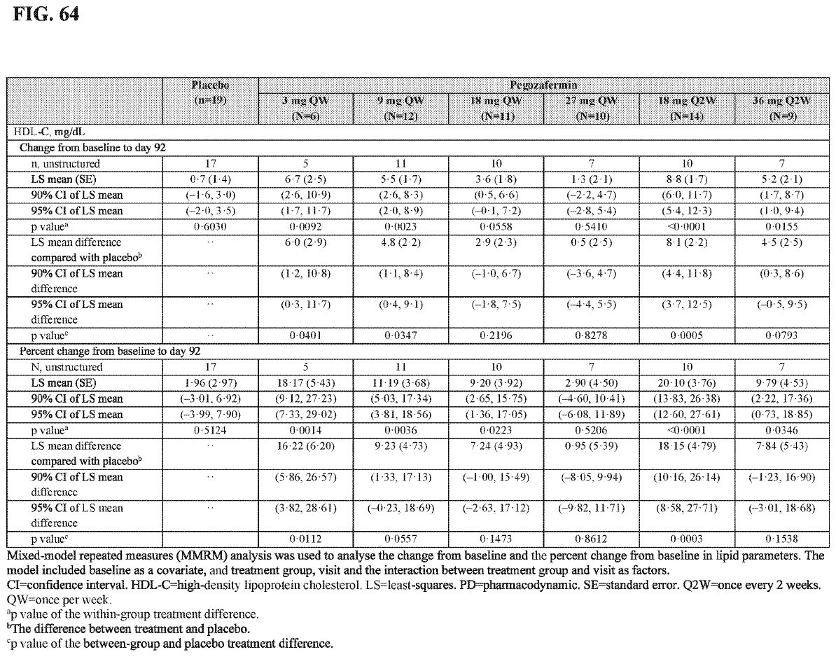

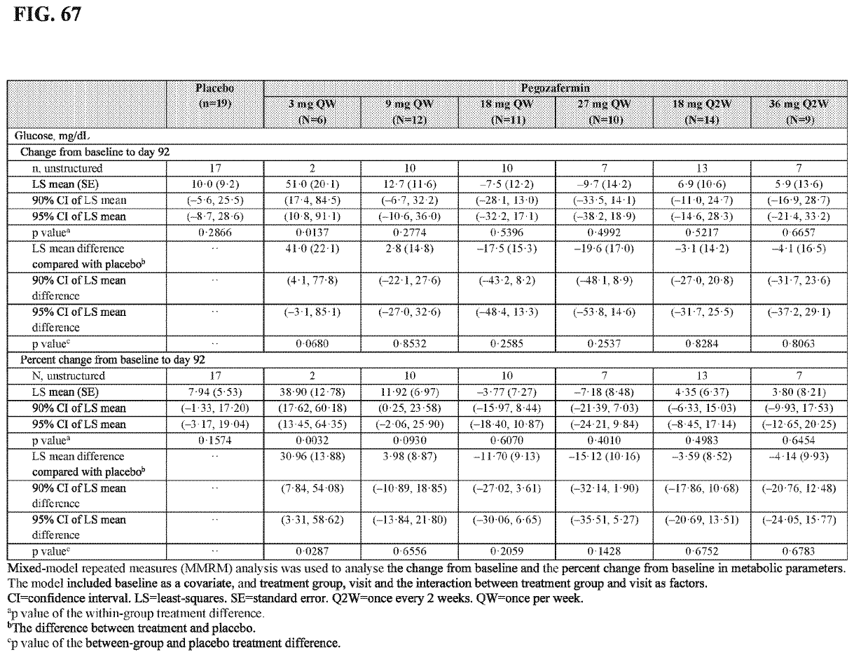

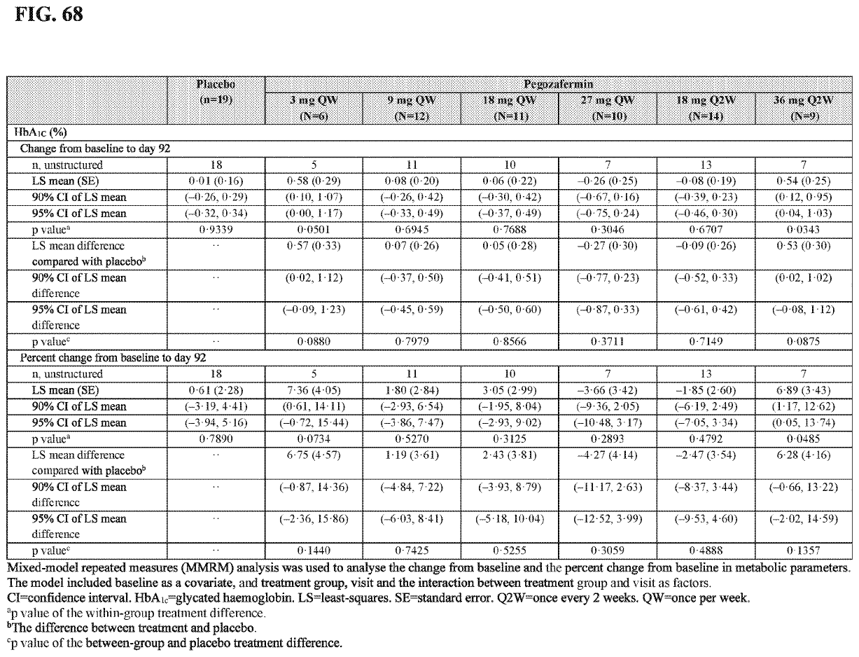

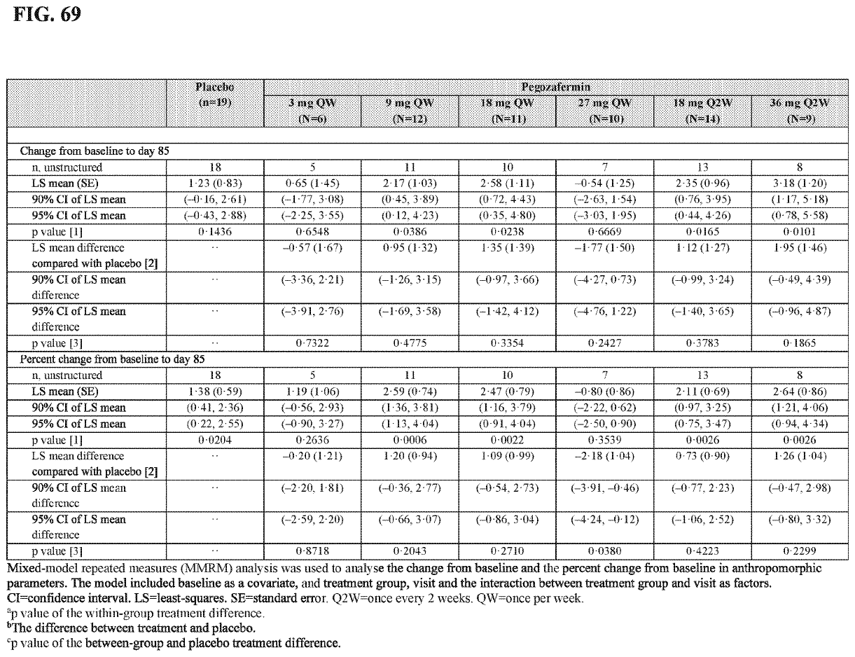

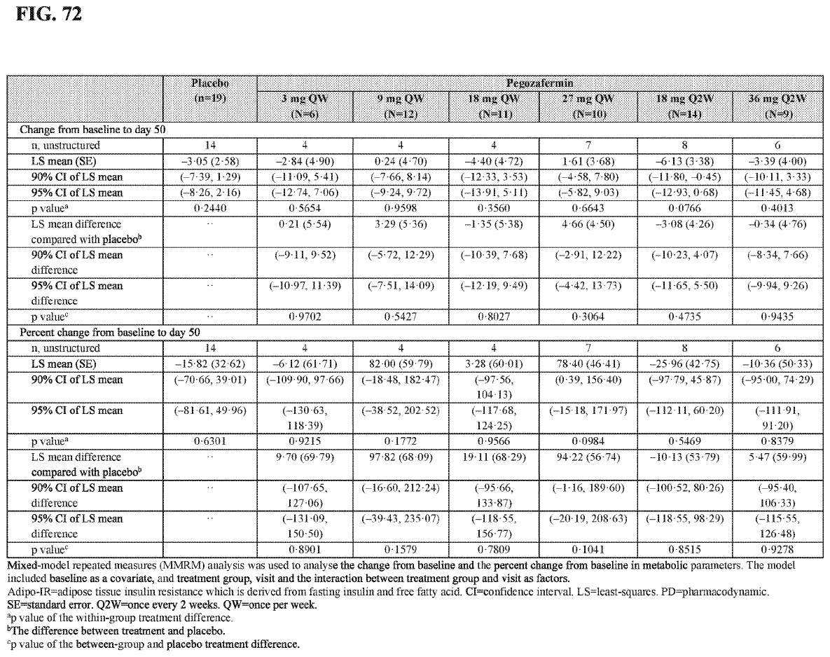

Statistical analysis was performed using SAS version 9.4 or higher (SAS Institute Inc., Cary, NC, USA). Six population analysis sets were defined (FIG. 55). Placebo groups from each cohort were pooled for analysis. Summary descriptive statistics were used to present demographics and baseline characteristics, safety endpoints, and PK and PD parameters. A mixed-model repeated measures (MMRM) analysis was used to analyse changes from baseline and/or percentage changes from baseline in PD endpoints. The MMRM included baseline as a covariate, with treatment group, visit, and the interaction between treatment group and visit as factors. The analyses were implemented using SAS PROC MIXED and the primary analysis was testing the interaction term. The covariance was unstructured. If the model failed to converge, other structures, such as compound symmetry, were considered. Least-squares (LS) means and LS mean differences were presented by visit with corresponding standard error, p values, and two-sided Wald interval 90% and 95% CIs (data reported here relate to 95% CIs, with all 90% and 95% CIs provided in FIGS. 56-72). When strong evidence existed that normality assumptions were violated, non-parametric methods were considered, such as the Wilcoxon Rank Sum test. Response rates were calculated with Miettinen-Nurminen 95% CIs. Differences in response rates between placebo and pegozafermin were analysed using Fisher's exact test. No adjustments for multiplicity or data imputations for missing values were performed in relation to the study outcomes.

Incidences and numbers of AEs were summarised by system organ class and preferred term, and by treatment group and pooled pegozafermin or placebo groups, using descriptive statistics.

Data handling procedures, including data management activities such as case report form and data collection, data review, data reconciliation and database lock were administered by IBM® Clinical Development using an electronic data capture system.

### Results

Of 275 individuals screened for the study, 81 met all eligibility criteria and were randomised to study treatment (pegozafermin, n=62; placebo, n=19) (FIG. 47). Of those randomised to pegozafermin, six were assigned to 3 mg QW, twelve to 9 mg QW, eleven to 18 mg QW, ten to 27 mg QW, fourteen to 18 mg Q2W, and nine to 36 mg Q2W. One participant assigned to placebo in the randomised analysis set inadvertently received a single dose of 3 mg QW pegozafermin. The safety analysis set thus included 63 subjects in the pegozafermin group and 18 in the pooled placebo group. Ten participants (12.3%) prematurely discontinued; reasons included AEs (n=2), non-adherence to study protocols (n=1), withdrawal of consent (n=7). Study interruption due to COVID-19 infection occurred in 11 participants (two receiving placebo [one after end of treatment] and 9 receiving pegozafermin [four after end of treatment]; duration, 6-21 days).

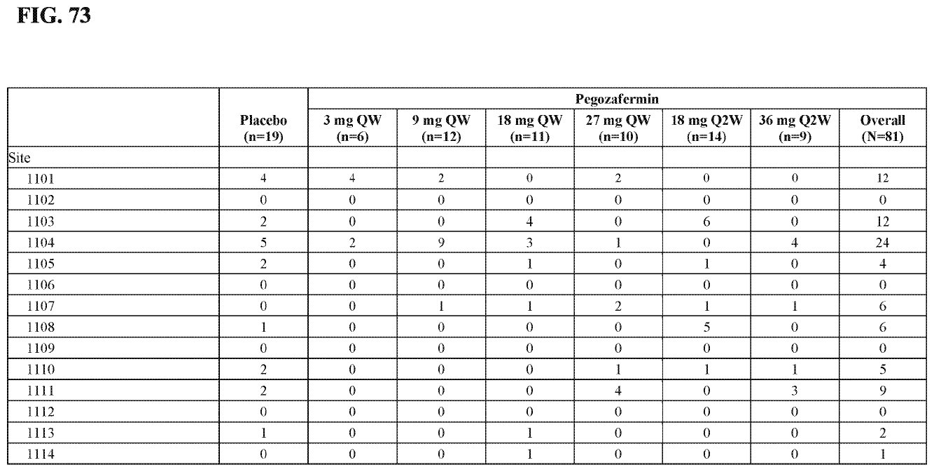

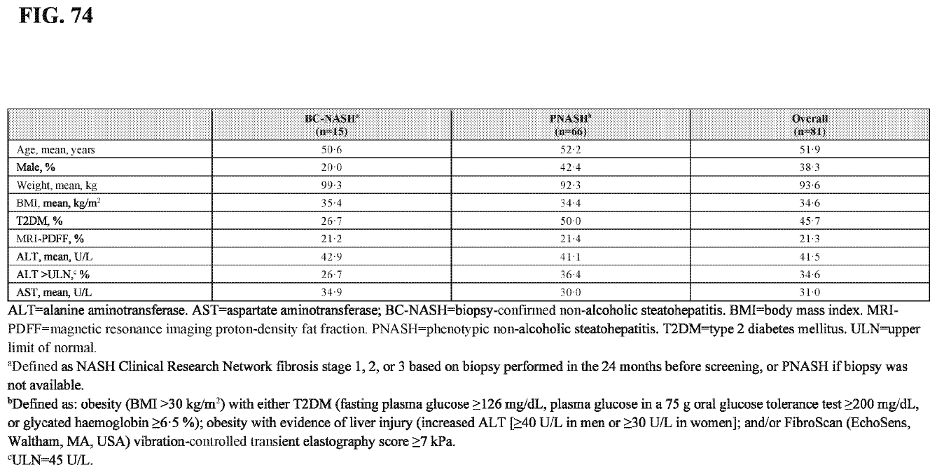

Overall, baseline characteristics were similar among the pooled placebo and pegozafermin cohorts (FIG. 73). Nearly all participants (95.1% [77/81]) met at least one of five criteria associated with a high risk of having NASH; 64.2% (52/81) met at least two of these criteria (table 1). Baseline characteristics were also similar between participants with BC-NASH (n=15) and those with PNASH (n=66), except for T2DM which was less prevalent in the BC-PNASH subpopulation (26.7% [4/15] vs 50.0% [33/66]), and the proportion of male participants which was lower in the BC-PNASH subpopulation (20.0% [3/15] vs 42.4% [28/66]). Within the BC-NASH subgroup, 11 participants were treated with pegozafermin (four in the 18 mg QW cohort and seven in the 18 mg Q2W cohort) and four participants were treated with placebo. See FIGS. 73-75.

Overall, treatment-emergent adverse events (TEAEs) occurred in 40 (63.5%) of 63 participants who received pegozafermin, and 8 (44.4%) of 18 participants who received placebo (table 2), with most (27/40 [68%]) being mild in severity. Two serious, non-drug-related TEAEs due to COVID-19 infection occurred in the pegozafermin group; neither led to treatment discontinuation or study withdrawal. Treatment discontinuations due to AEs occurred in two participants: one in the 27 mg QW cohort who had a grade 2 skin rash deemed as possibly related to treatment; and one (with T2DM) in the 18 mg Q2W cohort who had grade 3 acute hyperglycaemia, grade 1 chest pain, and grade 1 blurred vision, all assessed as unrelated to treatment. The most common AEs (pooled pegozafermin vs pooled placebo) were increased appetite (15.9% [10/63] vs 0%), diarrhoea (12.7% [8/63] vs 22.2% [4/18]), headache (11.1% [7/63] vs 5.6% [1/18]) and nausea (7.9% [5/63] vs 16.7% [3/18]); of these, increased appetite and headache occurred more frequently with pegozafermin than with placebo. Increased appetite was not associated with weight gain. Treatment-related TEAEs were reported in 24 (38.1%) of 63 participants in the pooled pegozafermin group and 5 (27.8%) of 18 participants in the pooled placebo group. No deaths were reported.

Gastrointestinal AEs, including diarrhoea, nausea, and abdominal pain/discomfort, occurred at similar frequencies between the pooled pegozafermin group (19/63; 30.2%) and the pooled placebo group (6/18; 33.3%), with no notable differences observed among dose groups. Mild transient and self-limiting injection-site events (erythema, pain, pruritus, or reaction) were reported in 4 (6.3%) of 63 participants in the pooled pegozafermin group and none in the pooled placebo group.

Overall, ADAs were detected in 41 (65.1%) of 63 participants treated with pegozafermin (range across cohorts: 14.3-78.6%) at any visit. The treatment-induced ADA incidence in the pegozafermin group was 63.9% (39/61); two participants in the pooled pegozafermin group were ADA-positive at baseline. Specificity was mostly to the FGF21 domain (63.5% [40/63]). Specificity to PEG was present in 4.8% (3/63) of pegozafermin-treated participants. ADA emergence appeared to be dose-related, with doses above 3 mg QW eliciting higher titres, and number of ADA positive subjects and ADA titre increasing with duration of treatment. No differences in ADA responses were observed between the QW and Q2W regimens. There was no evidence that pegozafermin PK, PD, or safety profiles were altered in participants with ADAs. No neutralising antibodies were observed.

No clinically significant findings were identified based on laboratory parameters, vital signs, ECG, or physical examination; specifically, no hypersensitivity reactions or tremor were reported, and no clinically relevant changes in blood pressure or heart rate were observed. No clinically significant findings on bone biomarker (C-terminal telopeptide, procollagen type 1 N-terminal propeptide, osteocalcin, and bone-specific alkaline phosphatase) or 24-h urine cortisol assessment were observed.

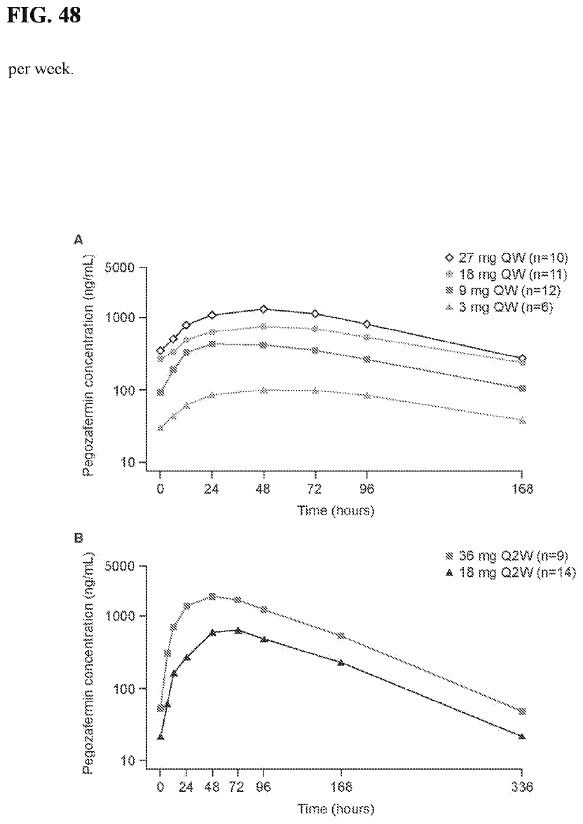

At steady state on day 29, the terminal phases of the concentration-time profiles were generally parallel on semi-logarithmic plots (FIGS. 48A and 48B), suggesting dose-proportional PK, with median ty/2 of approximately 46-68 hours across cohorts. Exposure (AUCₗₐₛₜ)) was also generally dose-proportional across cohorts, with dose-normalized AUCₗₐₛₜ fluctuating over the examined dose range with no discernible dose-related pattern (table 2). Median Cₘₐₓ values ranged from 103 ng/mL to 1674 ng/ml and tₘₐₓ was reached 48-72 hours after dosing across cohorts. Median accumulation ratios ranged from 1.0 to 1.4 for QW regimens and from 1.0 to 1.1 for Q2W regimens.

At week 13, hepatic fat fraction was statistically significantly reduced from baseline for all evaluated pegozafermin doses compared with pooled placebo, with the greatest effect observed in the 27 mg QW cohort (LS mean absolute change in MRI-PDFF of −13.5% vs +1.4%; difference: −14.9% [95% CI: −20.1, −9.7]; p<0.0001). An overall trend towards greater absolute reductions in hepatic fat fraction with increasing pegozafermin doses was also observed. The LS mean placebo-adjusted relative change in hepatic fat fraction from baseline to week 13 was −70.2% (95% CI: −92.5, −47.9; p<0.0001 vs pooled placebo) in the 27 mg QW cohort. Hepatic volume was also statistically significantly reduced from baseline at week 13 in most pegozafermin cohorts compared with pooled placebo, with a LS mean placebo-adjusted relative decrease of −16.4% (95% CI: −24.8, −7.9: p=0.0003 vs pooled placebo), and a LS mean absolute decrease of −0.33L (95% CI: −0.52, −0.15; p=0.0007 vs pooled placebo) observed in the 27 mg QW cohort.

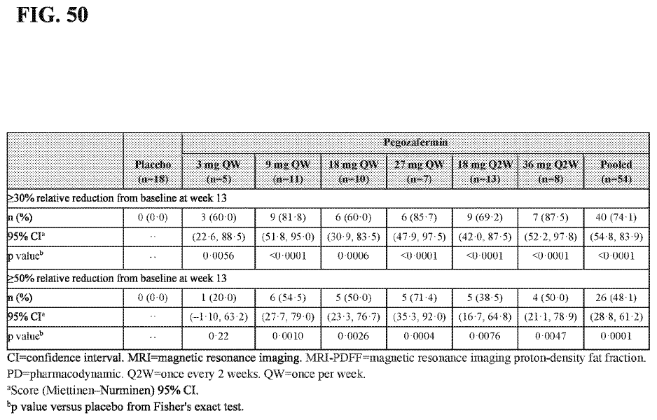

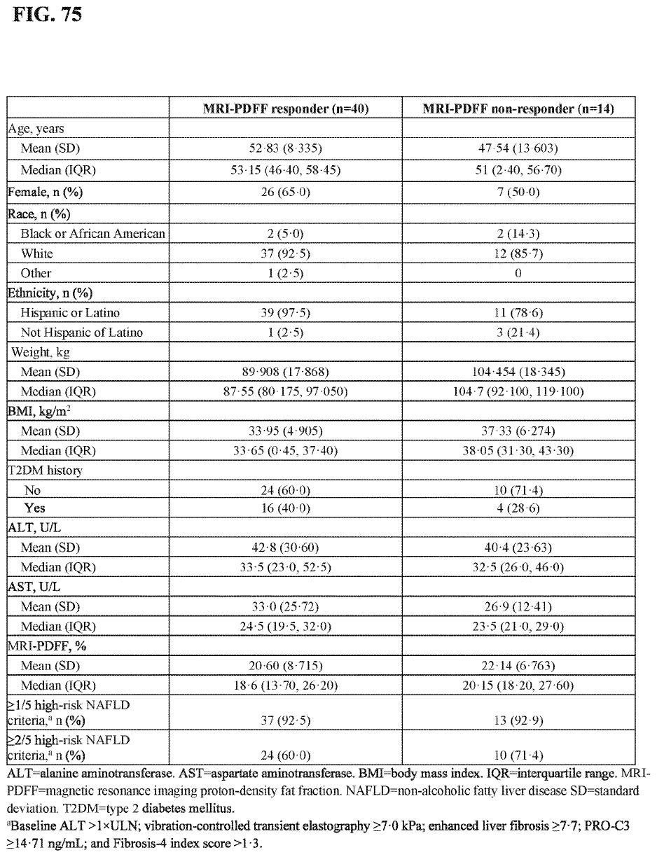

In the 27 mg QW cohort, 85.7% (6/7) of participants had a relative reduction of at least 30% in hepatic fat fraction at week 13 versus none in the pooled placebo group (p<0.0001), and 71.4% (5/7) had a relative reduction of at least 50% versus none in the pooled placebo group (p=0.0004) (ranges across pegozafermin cohorts: 60.0-87.5% for ≥30% relative reduction and 20.0-71.4% for ≥50% relative reduction, See FIG. 50). Baseline characteristics are presented in FIG. 75 for participants who had a ≥30% relative reduction in hepatic fat fraction versus those who did not. In the 27 mg QW cohort, hepatic fat fraction was normalised (<5%) in 42.9% (3/7) of participants versus none in the pooled placebo group (p=0.0152). Changes in all MRI-assessed liver parameters were similar among BC-NASH and PNASH participants treated with pegozafermin or placebo.

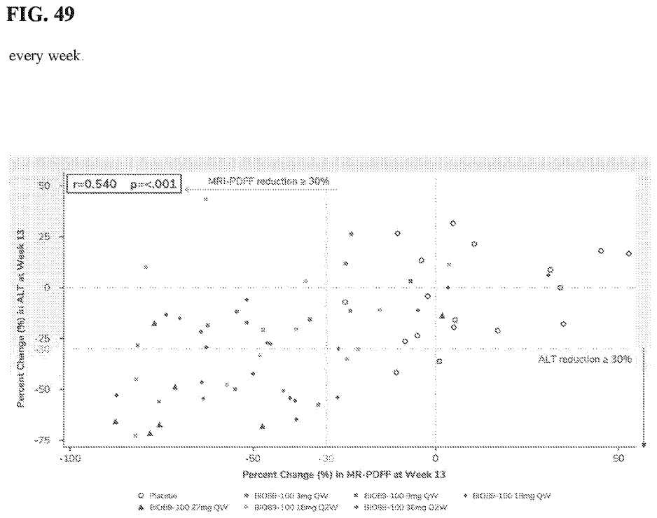

Treatment with pegozafermin statistically significantly reduced ALT levels from baseline at week 13 in most of the dose cohorts, with the largest LS mean relative change of −43.7% (−30.0 U/L absolute change) observed in the 27 mg cohort compared with −4.2% (−3.4 U/L absolute change) with pooled placebo (differences vs pooled placebo of −39.5% [95% CI: −59.9, −19.2]; p=0.0002 and −26.6 U/L [95% CI: −39.2, −13.9]; p<0.0001, respectively). An overall trend towards greater ALT reductions with increasing pegozafermin doses was also observed. Reductions in ALT from baseline at week 13 were particularly pronounced in participants with elevated (defined by the central laboratory as >45 U/L) ALT levels at baseline (LS mean absolute change, −34.6 U/L [n=17] for pooled pegozafermin vs −10.3 U/L for pooled placebo [n=5]; difference of −24.3 U/L [95% CI: −47.6, −0.9]; p=0.0426). A reduction in ALT levels of at least 17 U/L was observed in 71.4% (5/7) of participants in the 27 mg QW group and 57.1% (4/7) of participants in the 36 mg Q2W group, compared with 16.7% (3/18) in the pooled placebo group (p=0.0169 and p=0.0664, respectively, vs pooled placebo). In pegozafermin-treated participants, a relative reduction of at least 30% in hepatic fat fraction was statistically significantly correlated with a relative reduction of at least 30% in ALT levels (r=0.540; p<0.001). (See FIG. 49)

The LS mean relative change from baseline in AST at week 13 was −37.9% (−14.5 U/L absolute change) in the 27 mg QW cohort versus −4.4% (−5.2 U/L absolute change) with pooled placebo (differences of −33.5% [95% CI: −51.4, −15.5]; p=0.0004 and −9.3 U/L [95% CI: −16.8, −1.8]; p=0.0158, respectively).

A week-13 LS mean relative reduction from baseline in PRO-C3 levels of −27.7% was observed in the 27 mg QW group compared with +3.3% for pooled placebo (difference of −30.9% [95% CI: −57.4, −4.5]; p=0.0227), with numerical reductions in PRO-C3 levels also observed in the other pegozafermin cohorts, except for the 18 mg QW cohort.

Reductions in ALT, AST, and PRO-C3 levels with pegozafermin treatment were similar in the BC-NASH and PNASH subpopulations.

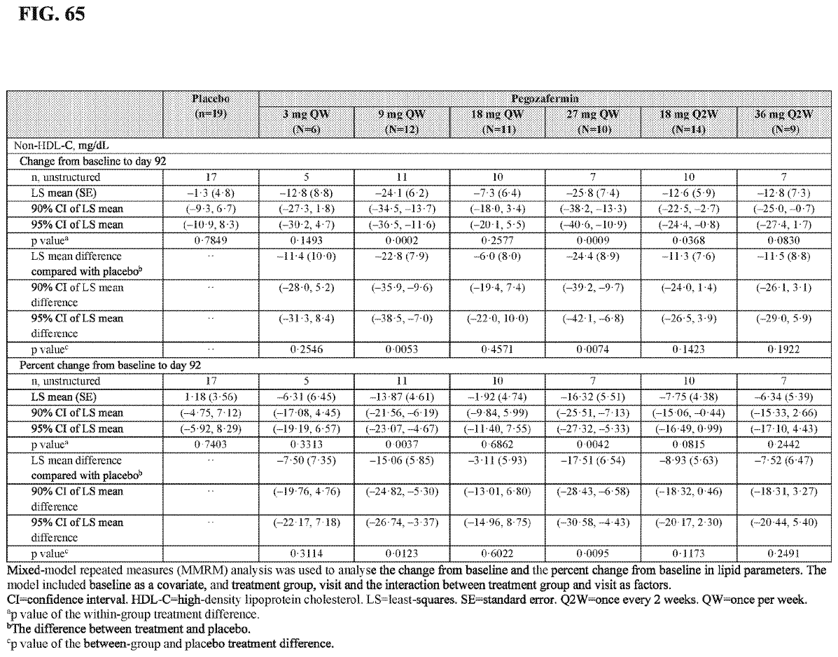

At week 13, a LS mean reduction in triglyceride levels relative to baseline of −27.6% was observed in the 27 mg QW cohort (range across pegozafermin cohorts: 17.7-28.5%) compared with −2.2% for pooled placebo (difference of −25.4% [95% CI: −48.4, −2.4]; p=0.0308). In participants with high baseline triglyceride levels (≥200 mg/dL; n=15 for pooled pegozafermin), triglycerides decreased by 33.1-48.9% at week 13, and 53.3% had triglyceride normalisation (<150 mg/dL). Statistically significant relative reductions from baseline in LS mean LDL-C (−16.5% for the pegozafermin 27 mg QW group vs+1.2% for pooled placebo; difference of −17.6% [95% CI: −32.7, −2.6]; p=0.0224) and increases in LS mean HDL-C levels (highest increase of 20.1% in the 18 mg Q2W group vs+2.0% for pooled placebo; difference of −18.2% [95% CI: −8.6, −27.7]; p=0.0003), were also observed. An LS mean reduction in non-HDL-C levels relative to baseline of −16.3% was observed in the 27 mg QW cohort compared with +1.1% for pooled placebo (difference of −17.5% [95% CI: −30.6, −4.4]; p=0.0095) (FIG. 65).

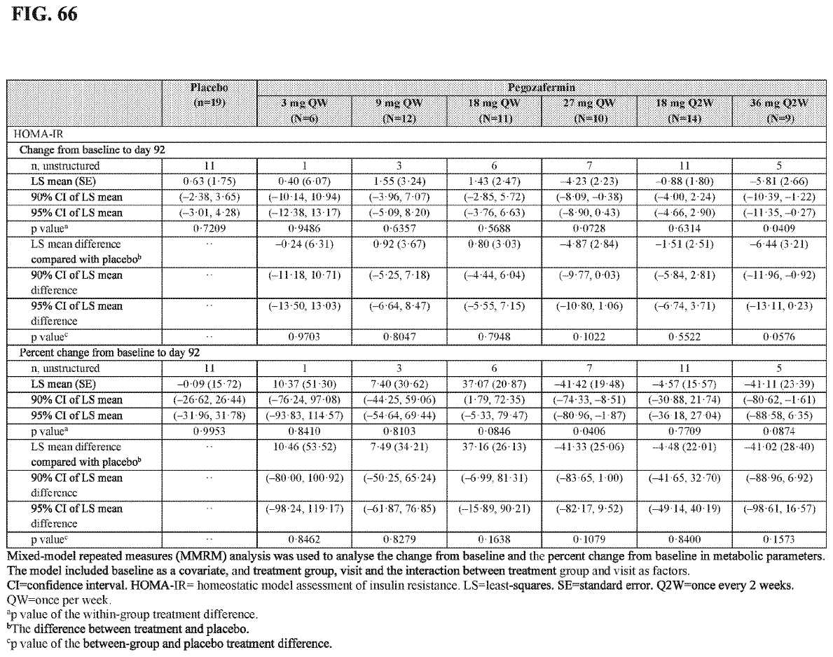

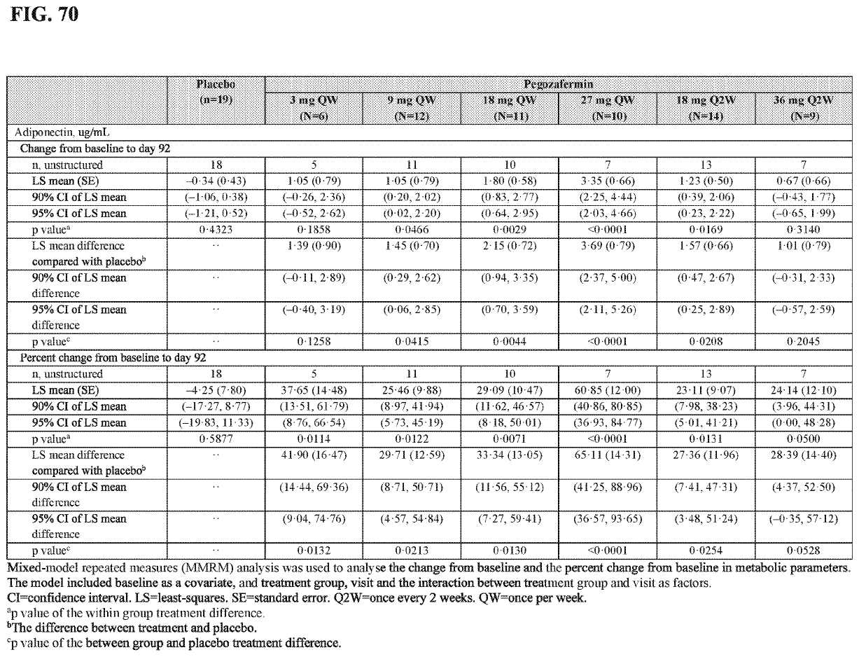

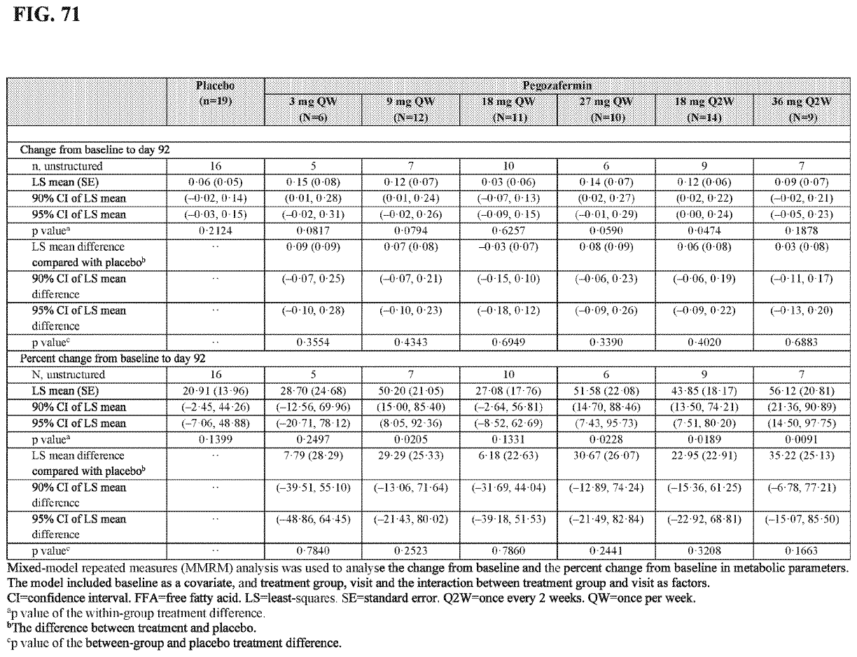

At week 13, greater improvements in insulin sensitivity, fasting plasma glucose, HbA1c, and body weight were observed in cohorts that received higher doses of pegozafermin than in the pooled placebo cohort (FIGS. 66-69). These changes were not statistically significant, except for body weight, which decreased from baseline by an LS mean of 2.2% relative to pooled placebo at week 12 in the 27 mg QW cohort (95% CI: −4.2, −0.12; p=0.0380). In addition, a 65.1% LS mean increase in adiponectin relative to pooled placebo was observed in the 27 mg QW cohort (95% CI: +36.6, +93.7; p<0.0001); in the pooled pegozafermin group a 36.0% increase in adiponectin relative to pooled placebo was observed (95% CI: +17.7, +54.3; p=0.0002). No notable improvements from baseline were observed with pegozafermin versus placebo at week 13 with respect to changes in free fatty acid levels or Adipo-IR (FIGS. 70-71).

### Discussion

In this proof-of-concept, phase 1b/2a multiple ascending dose study, treatment with pegozafermin led to marked improvements in multiple liver-related and metabolic parameters in patients with BC-NASH or PNASH, and was generally well tolerated. These benefits were observed across all tested doses, in QW and Q2W dosing cohorts, with the most prominent effects observed at the highest tested doses: 27 mg QW and 36 mg Q2W.

In the liver, 12 weeks of treatment with pegozafermin led to marked reductions in hepatic fat fraction as assessed by MRI-PDFF. High proportions of participants had relative reductions in hepatic fat fraction above 30% and 50%, which have been shown to correlate with clinically relevant histological outcomes (eg, ≥2-point reduction in NAFLD Activity Score [NAS] and NASH resolution).¹⁰⁻¹² Hepatic volume was also statistically significantly reduced in participants treated with pegozafermin. It was previously shown that in participants with NASH, hepatic volume is highly correlated with MRI-derived measures of hepatic fat burden (including MRI-PDFF and total liver fat index) and with histologic steatosis grade,²⁴ and the observed change in hepatic volume in pegozafermin-treated individuals is aligned with the marked reduction in hepatic fat fraction. It remains to be determined if reduction in hepatic volume will translate into additional, clinically meaningful outcomes, such as reduction in right upper quadrant discomfort. Treatment with pegozafermin also led to statistically significant reductions in ALT and PRO-C3 levels. Elevated ALT levels are correlated with the incidence of steatohepatitis and fibrosis in patients with NASH,²⁵ and ALT reductions of at least 17 U/L have been shown to correlate with histological improvements.²⁶ This ALT threshold was reached by 71% (5/7) of participants in the pegozafermin 27 mg QW group, in whom statistically significant reductions of PRO-C3, a neo-epitope marker of type III collagen formation and an emerging non-invasive biomarker of fibrogenesis and fibrosis,²⁷⁻²⁹ were also observed. These data suggest that pegozafermin has important benefits across multiple liver-related parameters that may predict a beneficial effect on clinically significant histological and other endpoints in NASH. The effect of pegozafermin on NASH histological endpoints (NAS ≥4, NASH CRN fibrosis stage 2 or 3) is currently being evaluated in a Phase 2 study (ENLIVEN, NCT04929483).

NASH is commonly regarded as a hepatic manifestation of the metabolic syndrome, and the nomenclature metabolic-associated fatty liver disease (MAFLD) has been suggested as a potentially more accurate term for this condition.³⁰ An ideal treatment for NASH would thus simultaneously address liver-related parameters (eg, hepatocyte stress, immune cell infiltration, and fibrosis) and the underlying metabolic overload that drives hepatic pathology. Indeed, patients with NASH often have multiple cardiovascular risk factors, and are at high risk for cardiovascular events and cardiovascular mortality, and NAFLD/NASH itself may confer additional cardiovascular risk, particularly in patients with advanced fibrosis.³¹ Importantly, cardiovascular mortality is a leading cause of death in patients with NASH.³¹ In view of these considerations, it is encouraging that 12 weeks of pegozafermin treatment resulted in clinically meaningful metabolic improvements, including reductions in triglycerides, LDL-C and non-HDL-C and increased HDL-C, in addition to significant liver-related benefits. The concurrent reduction in hepatic fat fraction and triglycerides is noteworthy, as it has been suggested that fibrates, which are approved to treat hypertriglyceridemia, may increase hepatic fat content and volume.³² Notably, these benefits occurred in the absence of clinically significant safety concerns, and corroborate previous findings in healthy volunteers.²³

Trends towards improvements in additional metabolic parameters (HOMA-IR, fasting plasma glucose, and HbA1c) were observed at higher pegozafermin doses relative to placebo over the 12-week treatment period, but these benefits did not reach statistical significance. A small, statistically significant, 2.2% weight loss was observed in the 27 mg QW group, which was not secondary to gastrointestinal AEs. This weight loss is unlikely to have significantly contributed to the beneficial effects on serum lipids, which were also observed in other dose groups, in which weight loss did not occur. The mechanism of weight loss has not been investigated in this study; notably, pegozafermin has been shown to increase energy expenditure in CD-1 mice, {Rosenstock, 2020 #19} and to decrease sweetness preference in cynomolgus monkeys. {Rosenstock, 2019 #59} Treatment with pegozafermin resulted in a robust increase (up to 61%) in adiponectin, an insulin-sensitising, anti-inflammatory, anti-fibrotic, anti-atherosclerotic, and hepatoprotective factor predominantly produced by adipocytes.³³ FGF21 potently induces adiponectin gene expression in adipocytes through a peroxisome proliferator-activated receptor (PPAR) γ-dependent mechanism in mice, and a variety of findings suggest that adiponectin is a critical downstream mediator of FGF21, facilitating its pleiotropic effects in major peripheral organs, including the liver, via abundantly expressed adiponectin receptors.³⁴

Two other FGF21 analogues, pegbelfermin (a PEGylated FGF21 analogue) and efruxifermin (an Fc-FGF21 analogue) are in clinical development for the treatment of NASH. FGF21 and its cofactor β-klotho signal via their cognate receptors FGFR1c, FGFR2c, and FGFR3, which are expressed across multiple organs, including the liver, adipose tissue, muscle, pancreas, and brain, contributing to the systemic effects observed in response to FGF21 agonism. Unlike FGF19 analogues, FGF21 does not signal via the FGFR4/β-klotho complex, the activation of which induces suppression of bile acids, leading to increased LDL-C levels.^{35, 36} Gastrointestinal AEs were the most frequently reported events for pegbelfermin¹⁷ and efruxifermin: 37 in contrast, the frequency of gastrointestinal AEs with pegozafermin in the current study was similar to placebo. Tremor, which has been reported with efruxifermin, was not observed.

Overall, the magnitude of the anti-steatotic effect observed with FGF21 analogues is greater than other therapies under clinical evaluation (up to ~70% relative reduction vs 18-58% for other agents), such as thyroid hormone receptor β, PPAR agonists, farnesoid X receptor (FXR) agonists, FGF19, and glucagon-like peptide 1, underscoring the potential of this drug class to target intrahepatocyte fat deposition, the main driver of NASH.^{16, 17, 37}-41 In addition, unlike FGF21 analogues, some other therapies in development for NASH may increase cardiovascular risk by increasing LDL-C (FXR agonists^{38, 42, 43} or FGF19 analogues⁴⁴), increasing triglyceride levels (acetyl-CoA carboxylase inhibitors),⁴⁵ or promoting weight gain and/or fluid retention (PPAR agonists).⁴⁶

The main limitations of this study are the small sample size and relative short treatment duration. Additionally, only a subset of participants had BC-NASH at baseline. The mixing of histological and non-invasive inclusion criteria may have increased the heterogeneity of our study population, although it should be noted that baseline characteristics, including hepatic fat fraction as assessed by MRI-PDFF, were similar in the BC-NASH and PNASH subpopulations, as were the observed treatment effects. Finally, where biopsies were available for the determination of BC-NASH, these were classified by local pathologists. An ongoing phase 2b study (ENLIVEN, NCT04929483), with centrally-read biopsies performed at baseline and after 24 weeks of treatment, followed by a blinded extension phase for a total of 48 weeks of treatment, will further evaluate the efficacy, safety, and tolerability of pegozafermin (15 mg QW, 30 mg QW and 44 mg Q2W dosing) in participants with BC-NASH (NAS ≥4, NASH CRN fibrosis stage 2 or 3).

In summary, in a population with BC-NASH and PNASH, treatment with pegozafermin for 12 weeks resulted in clinically meaningful improvements in liver parameters as assessed by MRI-PDFF, transaminases, and PRO-C3, and metabolic improvements as exhibited by reductions in triglycerides and LDL-C and increases in HDL-C and adiponectin. These beneficial effects were observed in participants who were treated with pegozafermin with QW and Q2W dosing. Overall, treatment was associated with a favourable safety and tolerability profile. Combined, these data indicate that pegozafermin has significant potential as a therapeutic agent for the treatment of NASH and other metabolic diseases, with the potential added advantage of a Q2W dosing option.

### Example 8: Variability in Liver Biopsy Assessment: Data from the Pegozafermin Phase 1b/2a Study in Subjects with Non-Alcoholic Steatohepatitis (NASH)

### Abstract

### Background

Variability in liver biopsy reads is increasingly recognized as a major challenge to drug development in NASH. Biopsy variability was assessed in an open label cohort of a Phase 1b/2a study in NASH, in which pegozafermin (PGZ), a long-acting glycoPEGylated recombinant human FGF21 analog, led to improved histology and significant liver-related (MRI-PDFF, ALT, multiple fibrosis-related non-invasive tests) and cardiometabolic benefits, with favorable safety and tolerability.

### Methods

Twenty (20) subjects with biopsy-proven NASH (NAS ≥4, fibrosis stage F2/F3) received SC PGZ 27 mg QW for 20 weeks; Baseline (BL) and Week 20 (W20) biopsies were available for 19/20 subjects. The primary analysis was conducted by a single central reader (CR), an expert liver pathologist with significant experience reading NASH studies. A post-hoc assessment by a panel of 3 additional board-certified, expert NASH pathologists [pathologists A (PA), B (PB) and C (PC)] was compared to the original read of the same slides by the CR. Biopsy reading by PA, PB and PC was independent; biopsies were scored for lobular inflammation, ballooning, steatosis and fibrosis. The proportion of subjects achieving NAS ≥2, NASH resolution without worsening of fibrosis and fibrosis improvement ≥1 stage without worsening of NASH were compared.

### Results

At BL, mean NAS was 5.4, 5.0 and 4.9 pts., respectively, as assessed by PA, PB and PC, vs. 5.4 pts. by the CR. At Week 20, ≥2 pt NAS reduction occurred in 79%, 79% and 68% of subjects (PA, PB and PC, respectively), vs. 74% (CR). Mean absolute decrease in NAS score was 2.3, 2.4 and 2.2 pts, respectively, vs. 2.4 pts (CR). NASH resolution without worsening of fibrosis occurred in 26%, 42% and 47% of subjects, respectively, vs. 32% (CR). Fibrosis improvement without worsening of NASH occurred in 42%, 32% and 12% of subjects, respectively, vs. 26% (CR); and either NASH resolution or fibrosis improvement occurred in 58%, 63% and 53% of subjects, respectively, vs. 47% (CR).

### Conclusions

PGZ 27 mg QW for 20 weeks led to meaningful changes in key histology endpoints in a cohort of NASH subjects with advanced fibrosis. Analysis limitations included post-hoc-reading and a small sample size; nevertheless, it was found that the % of subjects meeting Guidance-recommended endpoints for NASH pivotal trials varied markedly based on assessments of the 4 pathologists, ranging between 26-47% for NASH resolution without worsening of fibrosis and 12-42% for fibrosis improvement without NASH worsening. PGZ is currently being evaluated in NASH in the ongoing Phase 2b ENLIVEN study, that includes a 3 expert panel read with a consensus charter. Variability in endpoint assessment may increase placebo response and attenuate efficacy signals, and there is a pressing need for alternatives to histological endpoints in NASH clinical trials.

### Introduction

Fibroblast growth factor 21 (FGF21) is an endogenous hormone that regulates lipid and glucose metabolism and energy expenditure.

Pegozafermin (PGZ) is a glycoPEGylated FGF21 analog with a prolonged half-life compared to native FGF21 that is currently being developed for treatment of non-alcoholic steatohepatitis (NASH) and severe hypertriglyceridemia (SHTG).

In a randomized, placebo-controlled Phase 1b/2a POC study in subjects with NASH, PGZ had a significant liver-related and metabolic benefits.

In an open-label cohort of this POC study that included subjects with biopsy-confirmed NASH (NAS ≥4, fibrosis stage F2 or F3; N=20), PGZ 27 mg for 20 weeks led to clinically meaningful histological improvement, as well as significant liver-related (MRI-PDFF, ALT, multiple fibrosis-related non-invasive tests) and cardiometabolic benefits, with favorable safety and tolerability.

Intra-reader and inter-reader variability in liver biopsy reads is increasingly recognized as a major challenge to drug development in NASH.

Assessment of NASH trial biopsy slides by different pathologists (or by the same pathologist at different timepoints) may affect subject eligibility and evaluation of the proportion of subjects who have met histological endpoints.

### Objective

The objective of the study was to assess the impact on histological endpoints in the Phase 1b/2a POC study of assessment by 4 individual expert NASH pathologists.

### Methods

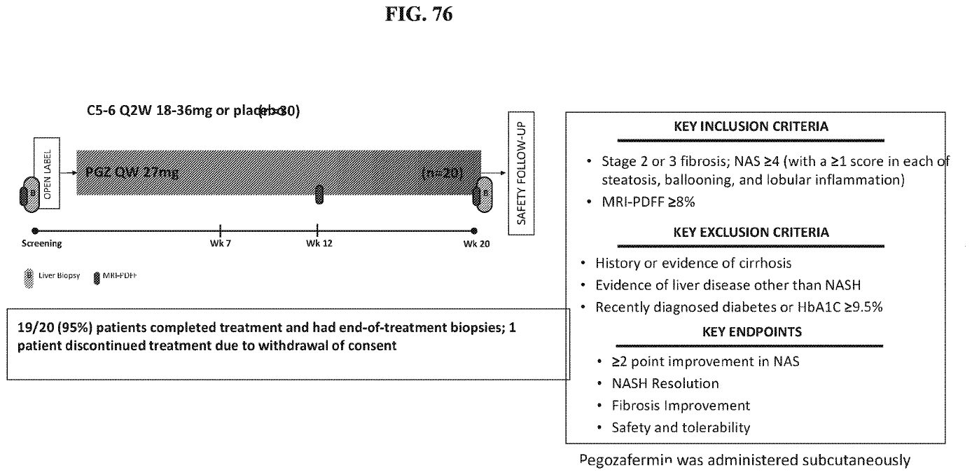

Study design is shown at FIG. 76.

Biopsy Reading: For the primary analysis, biopsies were read centrally at baseline (BL) and end of treatment (EOT) by a single expert liver pathologist with significant experience reading NASH studies.

In a post-hoc exploratory analysis, a panel of 3 additional expert NASH pathologists—Pathologist A, Pathologist B and Pathologist C—assessed the same BL and EOT slides that had been evaluated by the central pathologist. Slides from BL and EOT were mixed, and the pathologists were blinded to the timepoint.

Biopsies were scored for lobular inflammation, ballooning, steatosis, and fibrosis.

The proportion of subjects achieving ≥2-point reduction in NAS, NASH resolution without worsening of fibrosis, and fibrosis improvement ≥1 stage without worsening of NASH were compared.

### Results

Baseline characteristics of all subjects is shown in the table below.

| Parameter | PGZ 27 mg QW |
|---|---|
| Mean or % | (n = 20) |
| Age (years) | 58.4 |
| Female | 75.0% |
| Weight (kg) | 104.6 |
| BMI (kg/m²) | 37.0 |
| Type 2 Diabetes | 85.0% |
| MRI-PDFF (%) | 21.1 |
| ALT (U/L) | 47.1 |
| AST (U/L) | 36.1 |
| Fibroscan VCTE (kPa) | 14.3 |
| Triglycerides (mg/dL) | 170.0 |

Baseline Biopsy reading: The proportion of subjects in NAS grade/stage categories across the study population were generally consistent.
All subjects were deemed eligible by the central reader (primary analysis).
The 3 Panel pathologists assessed fibrosis stage as more advanced than the stage determined by the central reader

| Parameter Mean | | | | |
|---|---|---|---|---|
| or % | Control reader | Pathologist A | Pathologist B | Pathologist C |
| Fibrosis stage | 0/35/65/0 | 0/16/47/37 | 5/11/58/26 | 0/17/50/33 |
| F1/F2/F3/F4 (%) | | | | |

6/19 (32%) subjects were assessed with F4 fibrosis by 2 or more 3 Panel pathologists (putative F4). 4/19 (21%) subjects were assessed with F4 fibrosis by all 3 Panel pathologists.

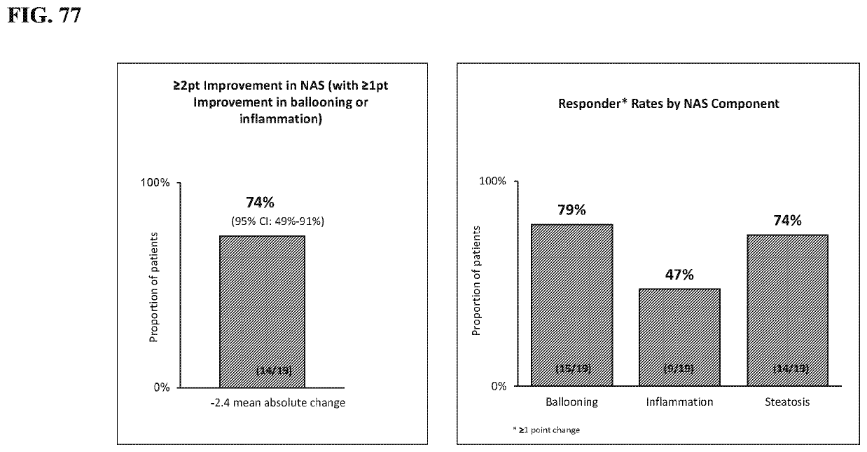

FIG. 77 shows the Primary Analysis (Central Reader): PGZ Robustly Improved NAFLD Activity Score (NAS) and All Components of NAS.

63% of patients had ≥2-point improvement in NAS and no worsening of fibrosis (primary endpoint) (with ≥1-point improvement in ballooning or inflammation.

100% of patients had improvement or no change in ballooning and inflammation.

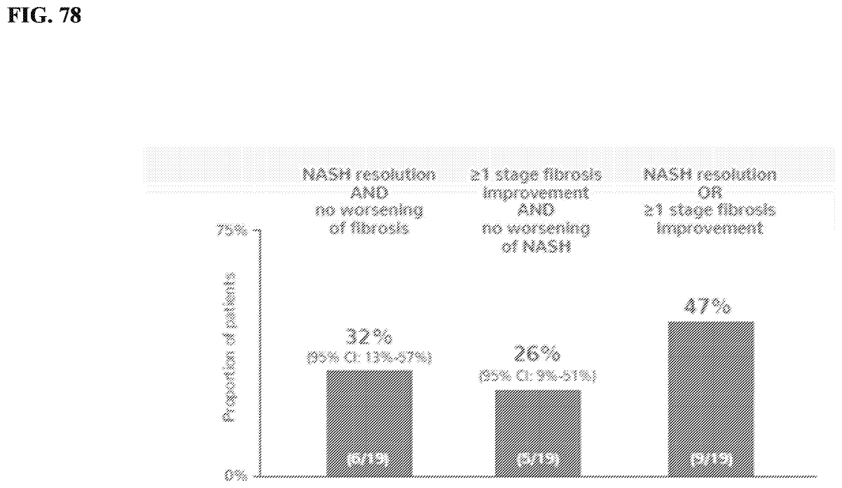

FIG. 78 shows the Primary Analysis (Central Reader): PGZ Demonstrated Clinically Meaningful Changes on Key Histological Efficacy Endpoints.

### Week 20 Biopsy Reading:

Mean EOT scores and mean change from baseline were similar across most parameters per assessment of the 4 pathologists.
There was more variability at EOT compared to baseline in the proportion of subjects assigned to specific histological grade/stage categories by the 4 pathologists across the study population.

The table below shows Week 20 Biopsy Endpoints By Individual Pathologist

| Parameter | Central Reader | Pathologist A | Pathologist B | Pathologist C |
|---|---|---|---|---|
| ≥2-point reduction | 74 | 79 | 79 | 68 |
| in NAS (%) | | | | |
| NASH resolution without | 32 | 26 | 42 | 47 |
| worsening of fibrosis (%) | | | | |
| Fibrosis improvement ≥1 | 26 | 42 | 32 | 12 |
| stage without worsening | | | | |
| of NASH (%) | | | | |

The table below shows the week 20 Central Reader endpoints excluding putative F4 at baseline (**Sensitivity analysis excluding subjects assessed with F4 fibrosis by 2+ Panel pathologists (n=6); cirrhosis was an exclusion criterion in this study.)

| Parameter | All subjects (n = 19) | Excluding putative F4 fibrosis (n = 13)* |
|---|---|---|
| ≥2-point reduction | 74 | 77 |
| in NAS (%) | | |
| NASH resolution | 32 | 46 |
| without | | |
| worsening of fibrosis | | |
| (%) | | |
| Fibrosis | 26 | 38 |
| improvement ≥1 | | |
| stage without worsening | | |
| of NASH (%) | | |

*Sensitivity analysis excluding subjects assessed with F4 fibrosis by 2+ Panel pathologists (n = 6); cirrhosis was an exclusion criterion in this study.

The table below shows Baseline Characteristics—Putative F4 Fibrosis* (*Subjects assessed with F4 fibrosis by 2+ Panel pathologists.)

| | Subjects with putative F4 |
|---|---|
| Parameter | fibrosis |
| Mean or % | (n = 6) |
| Age (years) | 60.9 |
| Female | 100% |
| Weight (kg) | 92.0 |
| BMI (kg/m²) | 33.9 |
| Type 2 diabetes | 83 |
| MRI-PDFF (%) | 18.25 |
| ALT (U/L) | 40.8 |
| AST (U/L) | 34.5 |
| Fibroscan VCTE (kPa) | 18.42 |
| HbA1c (%) | 6.6 |
| Triglycerides (mg/dL) | 161.1 |
| Albumin (g/dL) | 4.33 |
| Platelets (×10³/μL) | 188 |

*Subjects assessed with F4 fibrosis by 2+ Panel pathologists.

The table shows Week 20 Liver Non-Invasive Tests (NITs)—Putative F4 Fibrosis at Baseline*

(*Subjects assessed with F4 fibrosis by 2+ Panel pathologists.

**N=5; one outlier with poor quality measurement was excluded.

***VCTE >20% reduction; FAST score ≤0.35.)

| Parameter | Putative F4 fibrosis |
|---|---|
| Mean or % | (n = 6) |
| Relative liver fat reduction by MRI-PDFF (%) | −71.3 |
| MRI-PDFF 30% responders/MRI-PDFF 50% responders | 100%/100% |
| Change in ALT (U/L) | −23 |
| Percent change in ALT (%) | −50.7 |
| Percent change in AST (%) | −48.7 |
| Change in Fibroscan VCTE score (kPa) | −3.8** |
| Change in FAST score | −0.51** |
| Percent change in FAST score (%) | −78.5** |
| Fibroscan VCTE/FAST response*** (%) | 60**/100** |
| Percent change in adiponectin (%) | 98.7 |

*Subjects assessed with F4 fibrosis by 2+ Panel pathologists.
**N = 5; one outlier with poor quality measurement was excluded.
***VCTE > 20% reduction; FAST score ≤ 0.35.

In putative F4 subjects (n=6), fibrosis improvement ≥1 stage without worsening of NASH ranged from 17% to 57%; NASH resolution without worsening of fibrosis ranged from 20% to 50%.

### Discussion

### Impact on Eligibility:

32% (6/19) of the subjects would have screen failed as F4 at baseline on post-hoc analysis by the 3-panel pathologists rather than F3 as determined by the study central reader.
A consensus approach with 2 or more readers may reduce variability.

### Impact on Study Endpoints:

The proportion of subjects meeting the guideline-recommended endpoints for NASH clinical trials varied between the 4 pathologists, ranging between 26-47% for NASH resolution without worsening of fibrosis and 12-42% for fibrosis improvement ≥1 stage without NASH worsening.
Excluding subjects who were assessed as having putative F4 fibrosis at baseline (by 2+ Panel pathologists), in this post-hoc analysis, study histological endpoints would have been met by a higher proportion of subjects in the primary analysis:
NAS ≥2 points 75% to 77%;
NASH resolution without worsening of fibrosis 32% to 46%;
Fibrosis improvement ≥1 stage without worsening of NASH 26% to 38%. Subjects With Putative F 4 Fibrosis*at Baseline (*Subjects assessed with F4 fibrosis by 2+ Panel pathologists):
All subjects with putative F4 fibrosis had well-compensated cirrhosis (Child Pugh A).

There were no clinical or laboratory findings suggestive of clinically significant portal hypertension or other complications of cirrhosis.
At week 20, there was marked improvement in most liver-related and metabolic NITs in subjects with putative F4 fibrosis.
Safety and tolerability were favorable.
These findings are reassuring in regard to the safety and potential benefit of PGZ in an F4 population.

### Conclusion:

This analysis demonstrates the inter-reader variability in biopsy scoring.
In this post-hoc analysis, a higher proportion of subjects would have met histological endpoints in the primary analysis had the subjects determined to have putative F4 fibrosis at baseline (by 2+ panel pathologists) been excluded.
A marked beneficial effect on liver NITs and metabolic markers with good safety and tolerability was observed in subjects with putative F4 fibrosis at baseline.

Putative F4 subjects were well compensated; no safety issues suggestive of cirrhosis-related complications were reported.
Study limitations include post-hoc analysis and a small sample size.

### Example 9 Pegozafermin Inhibits NASH-Induced Hepatocellular Carcinoma in the STAM™ Mouse Model

Background and aims: Pegozafermin (PGZ), a long-acting glycoPEGylated recombinant human FGF21 analog, led to marked histological and other liver-related benefits, as well as cardiometabolic benefits, with favorable safety and tolerability, in a Phase 1b/2a study in NASH. PGZ does not activate FGFR4, and is not mitogenic. NASH-associated HCC, previously viewed as a complication of cirrhotic NASH, is increasingly diagnosed in pre-cirrhotic NASH. In the STAM™ model, which recapitulates the human NASH-HCC sequence; HCC appears at ¬16 weeks of age and develops universally at ¬20 weeks of age. In HCC prevention studies in this model, treatment typically begins between 6-12 weeks of age. PGZ led to significant improvements in features of NASH, including liver histology, liver transaminases and various metabolic parameters in STAM™ mice. The objective of this study was to evaluate its effect on development of HCC in this model.

Methods: STAM™ mice (12 or 13 weeks old, male, N=20 per group) were treated by vehicle, pegozafermin (previous name BIO89-100), (0.3 mg/kg, 1.0 mg/kg or 3.0 mg/kg) 3 times a week or a positive control, sorafenib (30 mg/kg once daily) for 9 weeks, starting at Week 12 (N=16 per group) or Week 13 (N=4 per group). Sorafenib (a protein kinase inhibitor) slows tumor progression and decreases tumor burden in HCC mouse models. All surviving animals were sacrificed at 20 or 21 weeks of age. At time of sacrifice, number of surviving animals, liver weight, liver weight/body weight ratio and the number of visible tumor nodules on the surface of the liver in surviving mice were assessed.

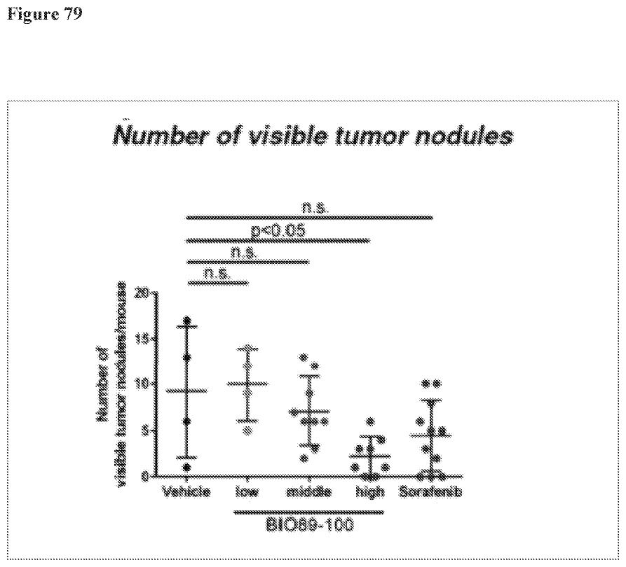

Results: PGZ led to a dose-dependent decrease in the number of macroscopic tumor nodules; the mean (±SD) number of visible tumor nodules per mouse was 9±7, 10±4, 7±4, 2±2 and 5±4 in the vehicle, PGZ 0.3 mg/kg, PGZ 1.0 mg/kg and sorafenib groups, respectively (FIG. 79, p<0.05 for PGZ 3 mg/kg). A decrease in liver weight and liver/body weight ratio was observed in PGZ-treated animals; mean liver weight and liver/body weight ratios were 2391±473 mg and 9.6±2.2; 2498±1120 mg and 11.0±4.6; 1802±391 mg and 7.8±1.7; 1252±210 mg and 6.2±1.1; and 2013±916 mg and 8.4±3.8 for the vehicle, PGZ 0.3 mg/kg, PGZ 1.0 mg/kg and sorafenib groups, respectively (p<0.05 for liver weight and liver/body weight ratio for PGZ 3 mg/kg). Survival was 4/20, 4/20, 9/20, 8/20 and 12/20 in the vehicle, PGZ 0.3 mg/kg, PGZ 1.0 mg/kg and sorafenib groups, respectively; effect on survival was not statistically significant with either PGZ or sorafenib treatment. Immunohistochemistry staining for cleaved caspase 3 (AC3) and proliferating cell nuclear antigen (PCNA) did not differ significantly in vehicle vs. PGZ or sorafenib-treated animals

Conclusions: In STAM™ mice, PGZ treatment led to a decrease in the development of HCC tumor nodules and tumor burden, that was comparable to that achieved with sorafenib. These data suggest that in addition to its beneficial effects on NASH and fibrosis and significant metabolic benefits, which render PGZ a promising treatment for NASH, should these pre-clinical data translate to human use, reducing the risk of HCC may be an additional benefit of PGZ treatment. The mechanism of this anti-tumor effect remains to be elucidated. PGZ is currently being studied in the ENLIVEN Phase 2b clinical trial in NASH.

### Example 10: Phase 2b ENLIVEN Trial of Pegozafermin in Nonalcoholic Steatohepatitis (NASH)

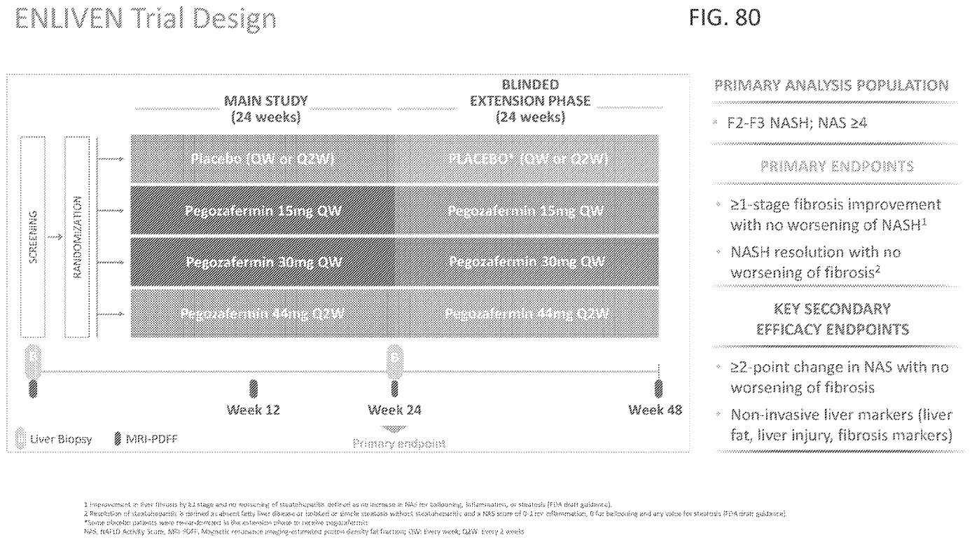

FIG. 80 shows Phase 2b trial design.

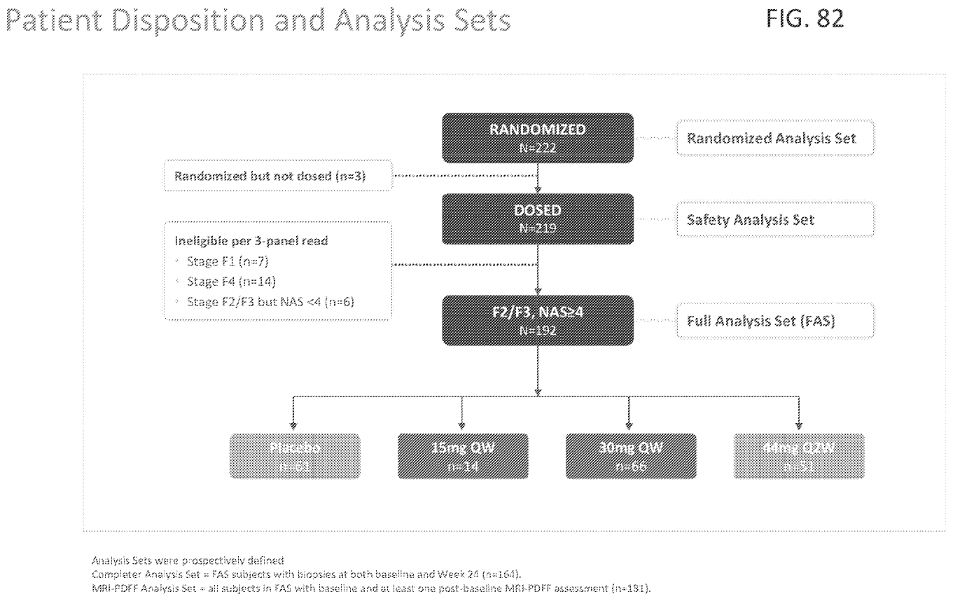

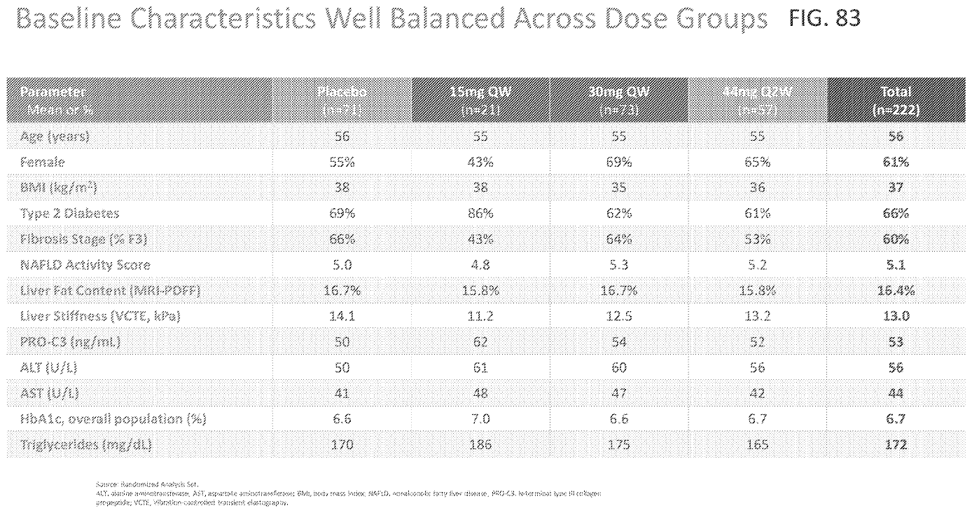

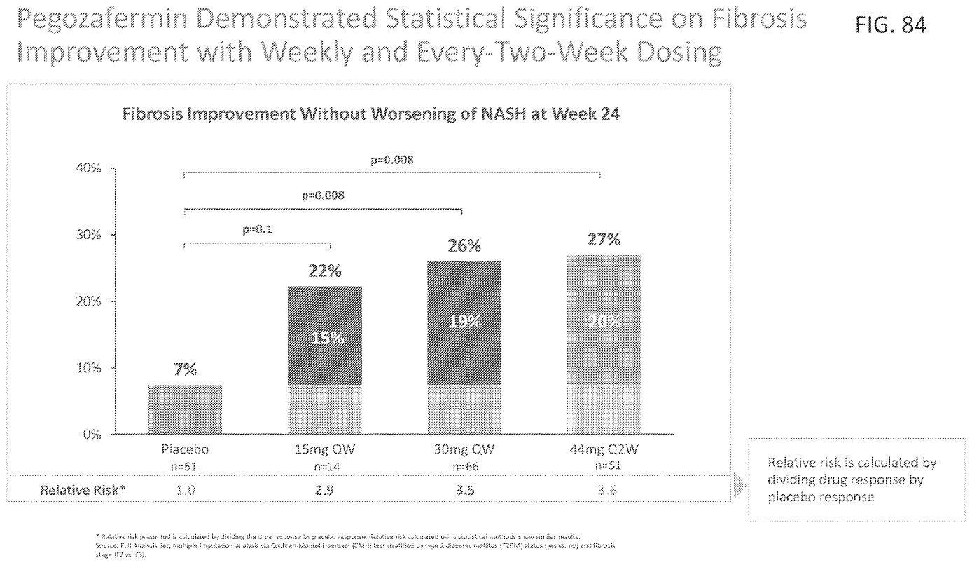

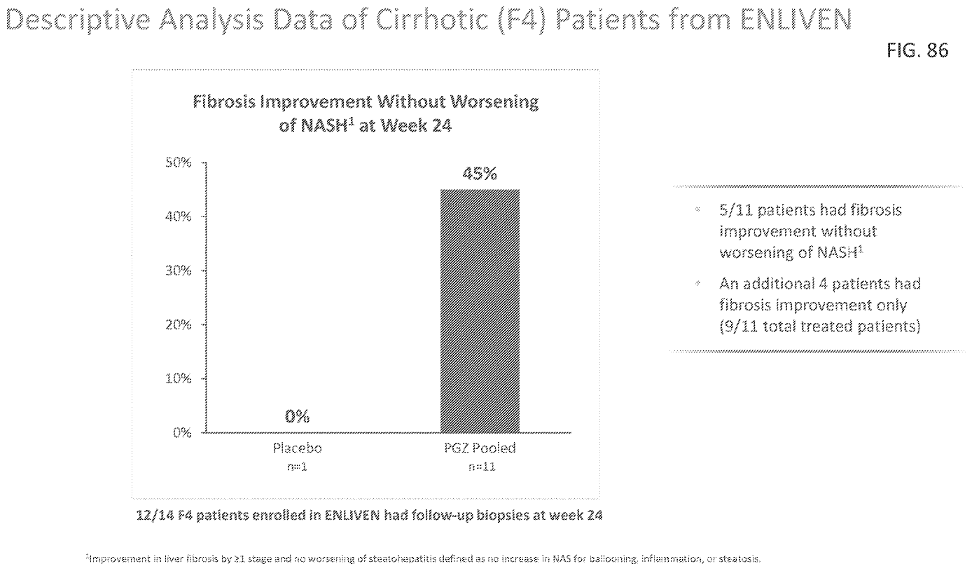

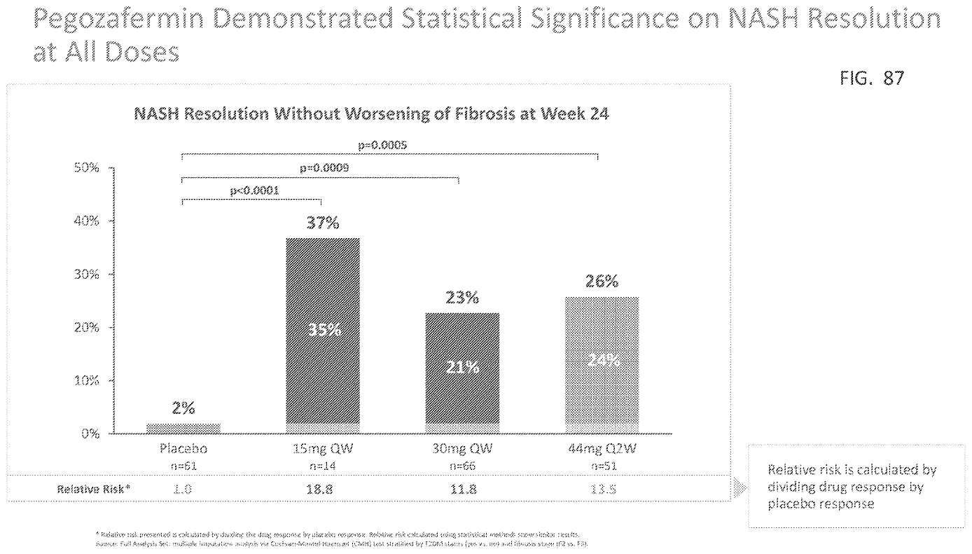

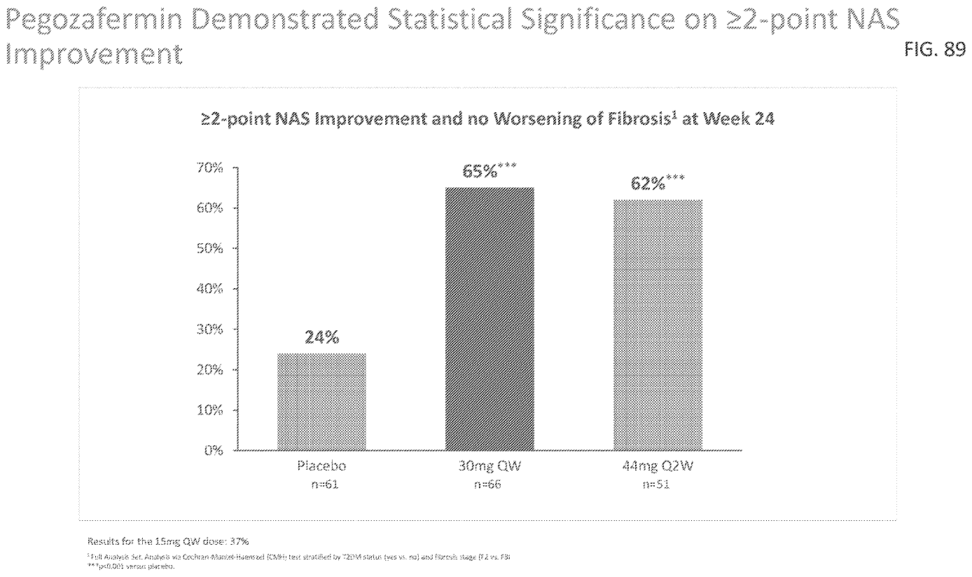

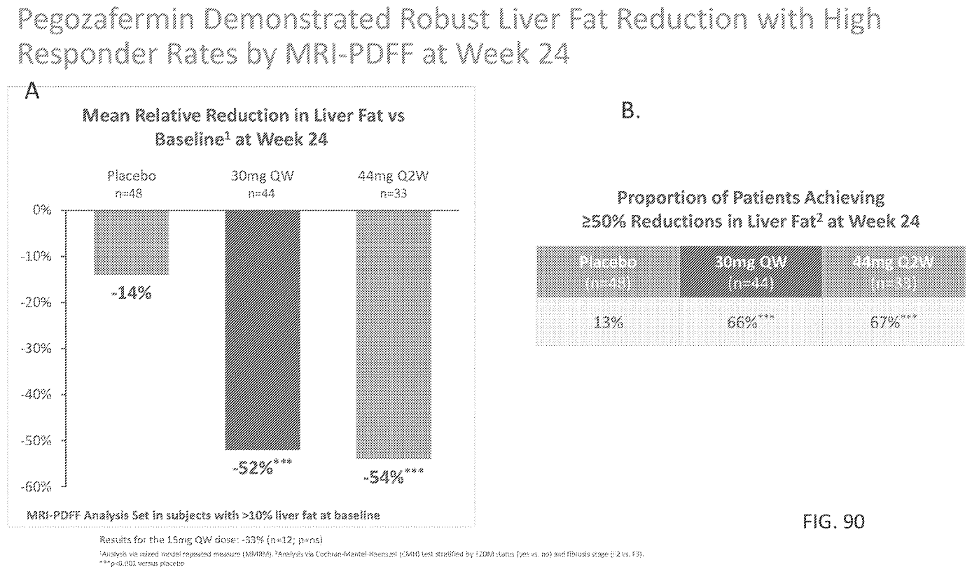

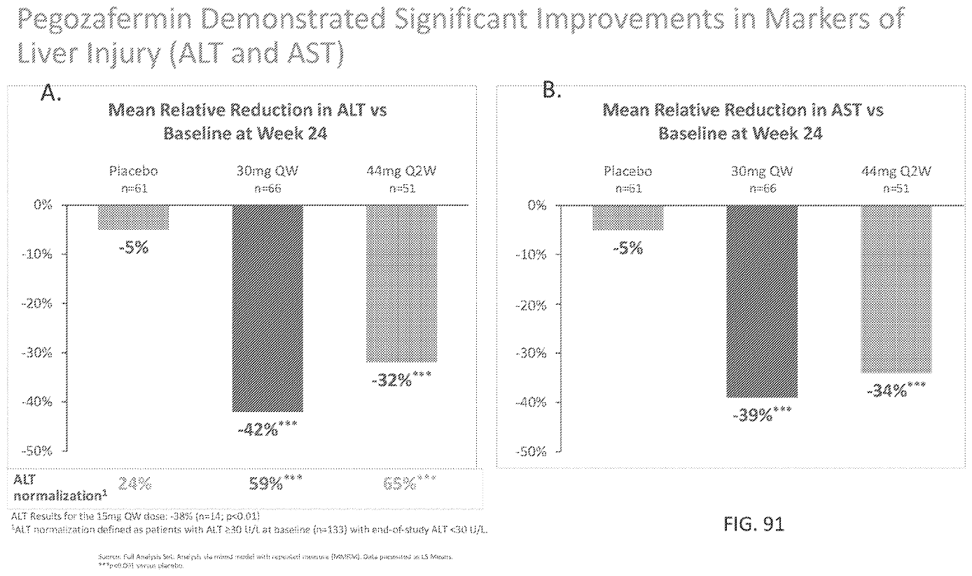

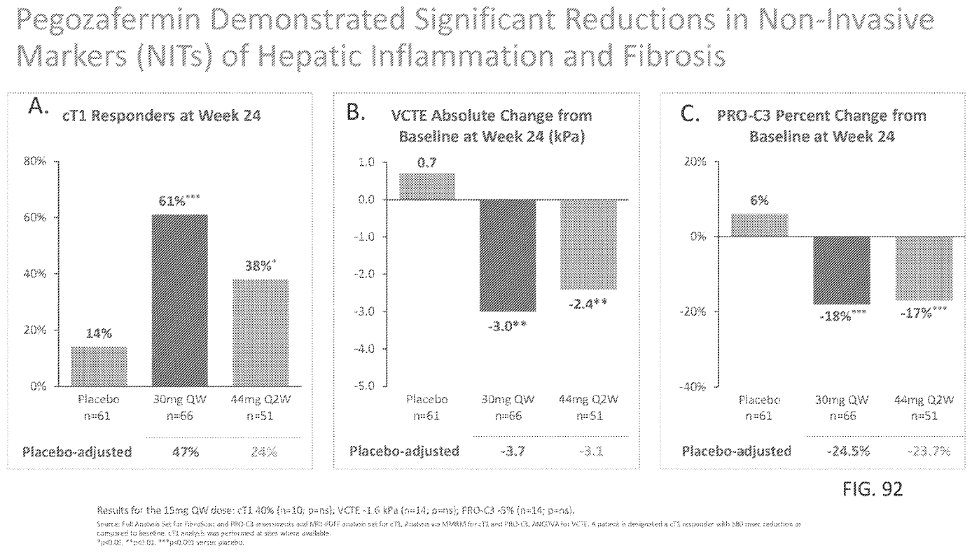

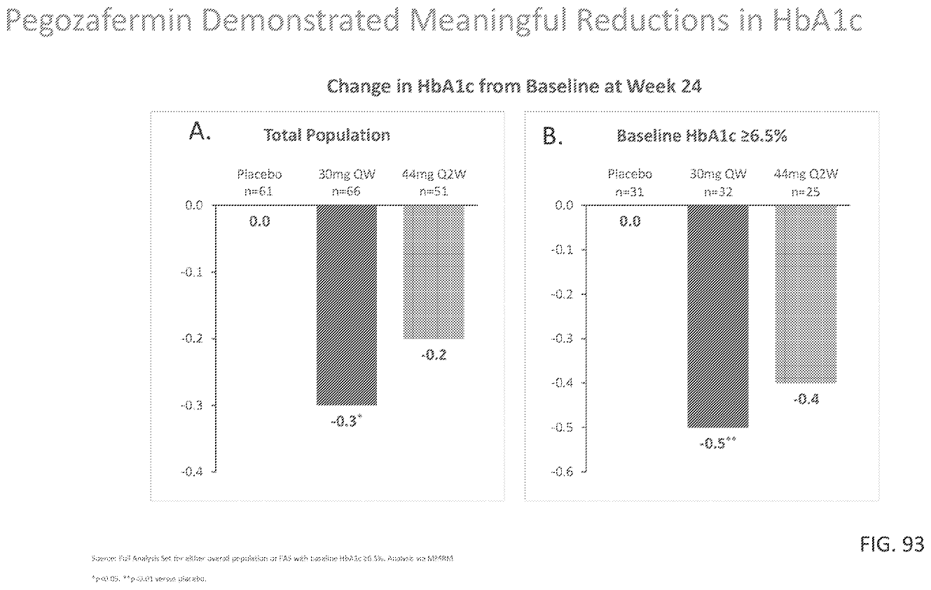

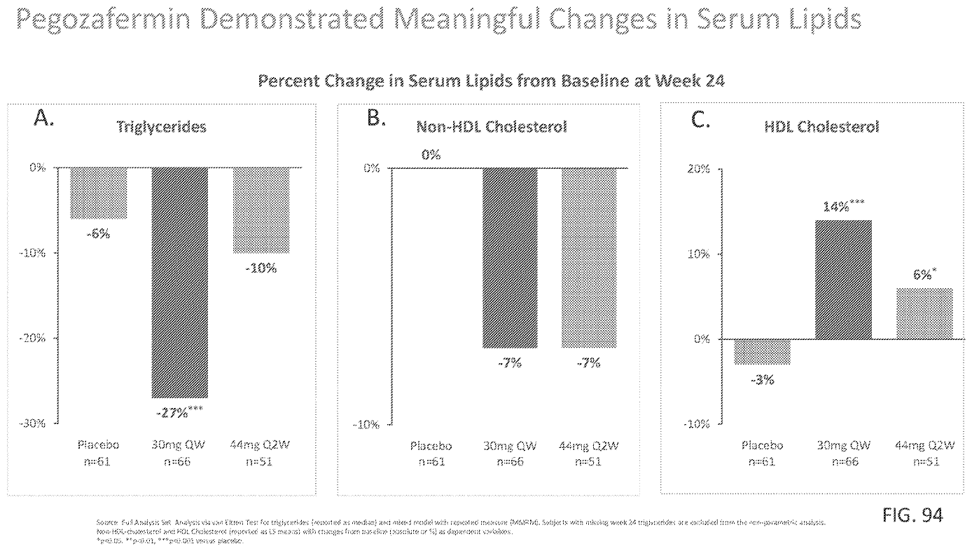

In the study, both the 44 mg every-two-week (Q2W) and 30 mg weekly (QW) doses of mutant Fibroblast Growth Factor-21 (FGF-21) peptide conjugate met, with high statistical significance, both the primary histology endpoints per the U.S. Food and Drug Administration (FDA) guidance on endpoints and statistical analysis. (see FIG. 82-94C)

The 44 mg Q2W and the 30 mg QW dose groups both demonstrated at least one-stage fibrosis improvement without worsening of NASH (27% and 26%, respectively) at 3.5 times the placebo rate (7%) and NASH resolution without worsening of fibrosis (26% and 23%, respectively), between 12 to 14 times the placebo rate (2%).

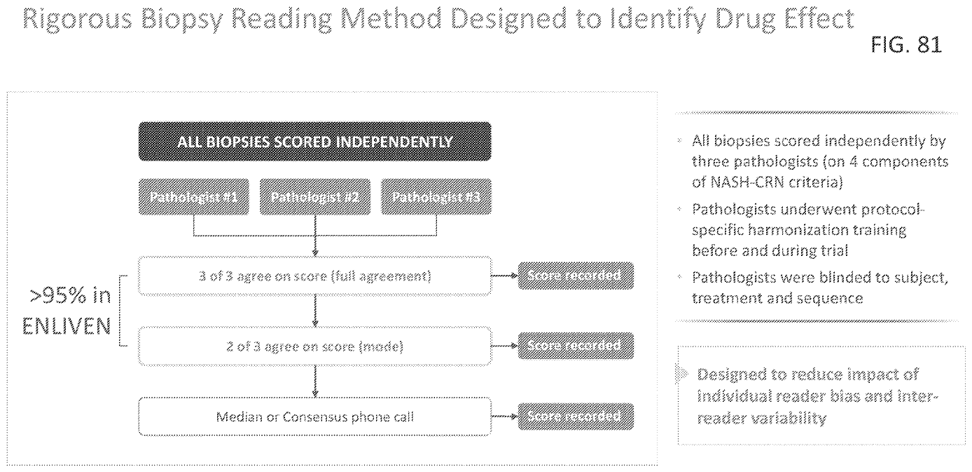

The ENLIVEN study biopsies were scored independently by three expert blinded pathologists to minimize individual reader bias and inter-reader variability. See FIG. 81.

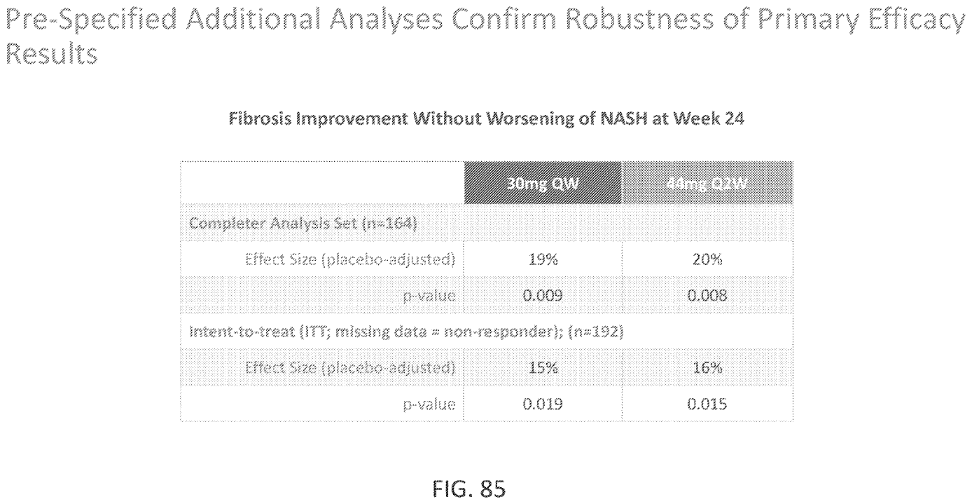

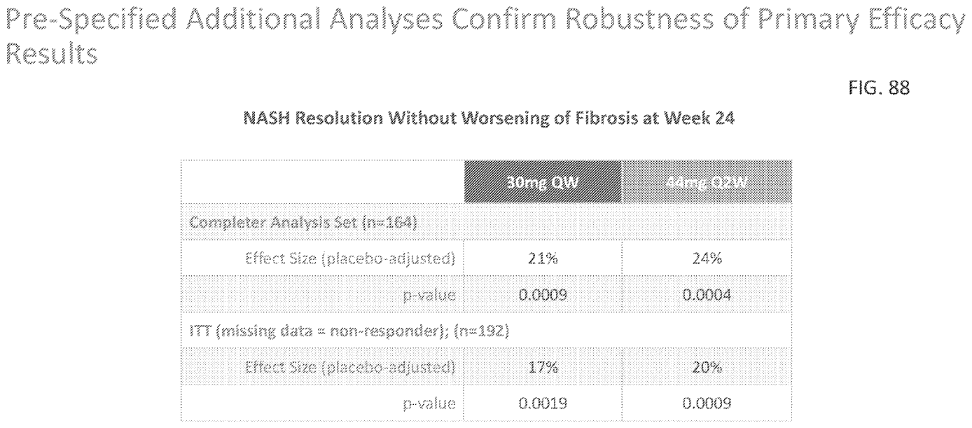

Results were consistent and achieved statistical significance for the 44 mg Q2W and 30 mg QW dose groups using multiple imputation analysis, completers analysis (patients who had baseline and end of treatment biopsies at week 24), and intention-to-treat (ITT) analysis (Phase 3 analysis plan). Using the completers analysis methodology on the fibrosis endpoint, the placebo-adjusted effect size for the 44 mg Q2W and 30 mg QW dose groups was 20% and 19%, respectively (p=0.008 and p=0.009, respectively; FIG. 85), and on the NASH resolution endpoint, the placebo-adjusted effect size for the 44 mg Q2W and 30 mg QW dose groups was 24% and 21%, respectively (p=0.0004 and p=0.0009, respectively, FIG. 88). Results were also statistically significant for both doses on both primary histology endpoints using an ITT analysis that imputes patients with missing biopsies as non-responders.

Meaningful changes were observed compared to baseline in liver fat and other key non-invasive tests (“NITs”) of liver inflammation and fibrosis. Improvements were also observed in HbA1c and across important lipid markers that are important factors for an effective treatment for NASH.

The study also included 14 biopsy-confirmed NASH patients with compensated cirrhosis (F4 patients) who were not part of the primary analysis but continued in the study, 12 of which underwent a follow-up biopsy at week 24. In descriptive analysis of these data, five out of 11 pegozafermin-treated patients experienced at least one-stage improvement in liver fibrosis with no worsening of NASH by week 24 compared with zero out of one patient on placebo. An additional four pegozafermin-treated patients experienced at least one-stage improvement in liver fibrosis.

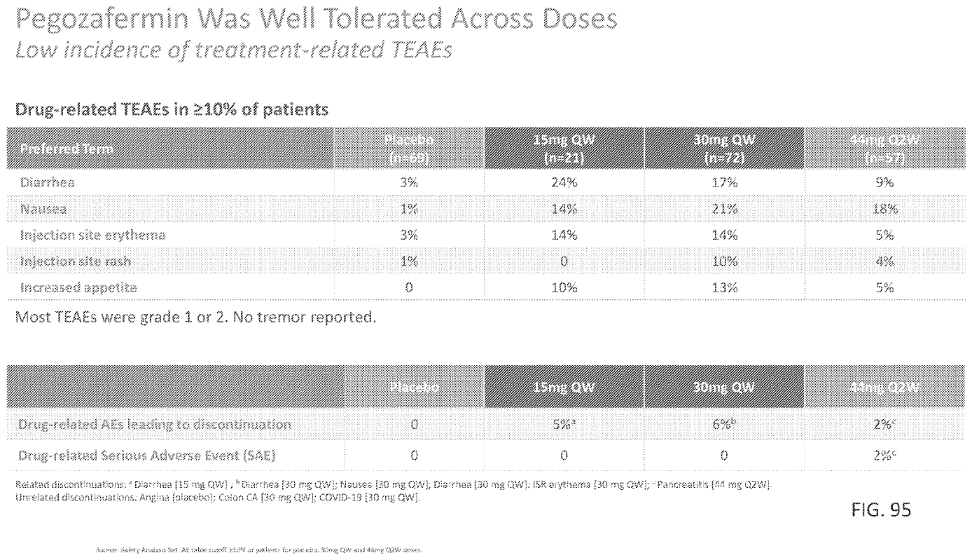

Pegozafermin demonstrated a favorable safety and tolerability profile consistent with prior studies. Across dose groups, the most frequently reported treatment-related adverse events (AEs) were Grade 1 or 2 gastrointestinal events (diarrhea, nausea and increased appetite) most of which were mild to moderate in nature. Rates of treatment-related AEs observed were less frequent with the Q2W dosing regimen. See FIG. 95.

### Example 11: A Randomized, Controlled Trial of the FGF21 Analog Pegozafermin in NASH

### Abstract

### Background

Pegozafermin is a long-acting glycoPEGylated FGF21 analog in development for nonalcoholic steatohepatitis (NASH) and severe hypertriglyceridemia. We aimed to evaluate the efficacy and safety of pegozafermin in patients with noncirrhotic NASH.

### Methods:

ENLIVEN was a phase 2b, 24-week, multicentre, double-blind, randomized, placebo-controlled trial of subcutaneous pegozafermin 15 mg or 30 mg weekly, or 44 mg once every 2 weeks, versus placebo in patients with biopsy-confirmed NASH and F2/F3 fibrosis. The two primary endpoints were proportions of patients achieving: 1. improvement of fibrosis (≥1 stage) with no worsening of NASH at 24 weeks, and 2. NASH resolution without worsening of fibrosis at 24 weeks.

### Results:

Of 222 randomized patients, 219 received treatment. The proportion of patients who achieved the fibrosis improvement endpoint was 7% in the placebo group, 22% with pegozafermin 15 mg weekly (difference versus placebo 14.5 [95% CI-8.7 to 37.6]), 26% with 30 mg weekly (difference 18.9 [5.2 to 32.5]; P=0.009) and 27% with 44 mg every 2 weeks (difference 20.3 [5.3 to 35.4]; P=0.008). The proportion of patients who achieved the NASH resolution endpoint versus placebo (2%) with pegozafermin 15 mg weekly, 30 mg weekly and 44 mg every 2 weeks was 37% (difference 34.7 [10.0 to 59.3]), 23% (difference 20.9 [9.1 to 32.7]) and 26% (difference 23.6 [10.0 to 37.2]), respectively. The most common adverse events associated with pegozafermin were nausea and diarrhea.

### Conclusions

In this phase 2 trial, treatment with pegozafermin led to improvements in fibrosis. These results support advancing pegozafermin into phase 3 development.

Nonalcoholic steatohepatitis (NASH) is characterized by excess fat accumulation, hepatic inflammation and cellular injury, with or without fibrosis. (Rinella et al., Hepatology 2023; 77:1797-835; Loomba et al., Cell 2021; 184:2537-64) It is associated with the metabolic syndrome, and an increased risk of cardiovascular disease. (Pouwels et al., BMC Endocr Disord 2022; 22:63; Shroff et al., Curr Hepatol Rep 2020; 19:315-26; Younossi et al., Hepatology 2016; 64:73-84). The development of significant fibrosis in NASH is associated with increased liver-related outcomes (e.g. progression to cirrhosis and its complications, hepatocellular carcinoma), cardiovascular events, and mortality. (Loomba et al., Cell 2021; 184:2537-64; Dulai et al., Hepatology 2017; 65:1557-65). The prevalence of NASH in adults has been reported as 5.3% globally and 14% in middle-aged Americans (Younossi et al., Hepatology 2023; 77:1335-47; Harrison et al., J Hepatol 2021; 75:284-91), and is rising (Riazi et al., Lancet Gastroenterol Hepatol 2022; 7:851-61; Estes et al., Hepatology 2018; 67:123-33) but there is no approved pharmacological treatment. (Loomba et al., Gastroenterology 2022; 162:680-8).

Fibroblast growth factor 21 (FGF21) regulates lipid and glucose metabolism and energy expenditure. (Lee et al., Clin Endocrinol (Oxf) 2014; 80:57-64.) Pegozafermin, a long-acting glycoPEGylated recombinant FGF21 analog, is being developed for the treatment of NASH and severe hypertriglyceridemia. (Rosenstock et al., J Hepatol 2019; 70: e155; Margalit et al., J Clin Lipidol 2020; 14:585-6; Loomba et al. Lancet Gastroenterol Hepatol 2023; 8:120-32.) A phase 1b/2a study in patients with NASH did not reveal safety concerns and suggested that pegozafermin may improve hepatic steatosis, markers of inflammation and fibrosis, circulating lipids, and glycemic control. (Loomba et al., Lancet Gastroenterol Hepatol 2023; 8:120-32). Benefits on liver histology were observed in an open-label cohort of patients with biopsy-confirmed NASH. (Loomba et al. J Hepatol 2022; 77: S730).

The objective of this study was to evaluate the efficacy and safety of pegozafermin in patients with noncirrhotic NASH.

### Methods

### Trial Design and Oversight

This randomized, placebo-controlled, double-blind, phase 2b trial was conducted at 61 sites in the USA to evaluate the efficacy, safety, and tolerability of pegozafermin over 24 weeks. It included a 12-week screening period and a 24-week treatment period. A placebo-controlled, single-blind, 24-week extension study is ongoing under the same protocol. The trial was conducted in accordance with the Declaration of Helsinki and Council for International Organizations of Medical Sciences International Ethical Guidelines, International Council for Harmonisation Good Clinical Practice Guidelines and applicable laws and regulations. Study protocol and amendments were approved by the institutional review board or independent ethics committee for each site. All participants provided written informed consent. The sponsor (89bio) designed the trial with the academic steering committee, and performed site monitoring, data collection, and data analysis. All authors had access to the data, participated in data interpretation, and vouch for the data and data analyses and for the fidelity of the trial to the protocol, available at NEJM.org. The steering committee and lead author decided to publish the paper. Dr Loomba wrote the first draft, which was further developed with the assistance of a medical writer (funded by the sponsor) under the guidance of the authors.

### Patients

Eligible patients were 21-75 years old, with NASH (Clinical Research Network [CRN] fibrosis stage F2 or F3 and Nonalcoholic fatty liver disease Activity Score [NAS] ≥4, with ≥1 point for steatosis, ballooning and lobular inflammation) confirmed in a biopsy performed at or ≤6 months before screening. There was no criterion for a minimum liver fat content. Key exclusion criteria were liver disease other than NASH, cirrhosis, uncontrolled or newly diagnosed type 2 diabetes, or any illness that, in the opinion of the investigator, might affect the results of the study or pose additional risk to the participant. Clinically relevant abnormalities in laboratory measurements, electrocardiogram, or vital signs were also exclusionary.

### Procedures

Patients were randomly assigned (centrally, using interactive response technology) to placebo once weekly (QW) or once every 2 weeks (Q2W), or pegozafermin 15 mg QW, 30 mg QW or 44 mg Q2W, initially at a 2:1:3:3:3 ratio. Doses were selected based on a maximum effect model using magnetic resonance imaging proton density fat fraction (MRI-PDFF) data from the phase 1b/2a study. After protocol amendment 2, the randomization ratio was updated to 16:8:6:24:15, limiting randomization to the 15 mg QW arm, due to concern about potential sub-optimal histological efficacy with this dose. Randomization was stratified by type 2 diabetes status and fibrosis stage (F2 vs. F3). Patients, investigators, and site personnel were blinded to treatment assignment, but not to dose frequency. Details of study drug administration and lifestyle counseling are provided (Glass et al., J Hepatol 2020; 73:680-93; Pais et al., J Hepatol 2023; S0168-8278 (23) 00189-7). Follow-up biopsy was performed at week 24. Initially, biopsies were assessed by one central pathologist. In response to advances in consensus reading methodology in NASH clinical trials, (Sanyal et al., Hepatology 2021; 74: 968A; Sanyal et al., AASLD The Liver Meeting 2022:5008 (Abstract)) a central three-panel-consensus scoring method replaced the original biopsy reading approach. Biopsies were assessed by three expert liver pathologists who were blinded to patient, treatment, and sequence, and scored using the NAS and the CRN fibrosis staging system. (Kleiner et al., Hepatology 2005; 41:1313-21) A consensus score was derived from the individual reader scores using an algorithm designed to minimize interaction between the readers. Baseline biopsies that were initially assessed by one pathologist were re-read by the panel. Protocol-specified reasons for study discontinuation, withdrawal, or interruption are provided.

### Endpoints

The two primary endpoints, evaluated at week 24 versus baseline, were the proportion of patients with improvement of fibrosis ≥1 stage without worsening of NASH (increase in any of ballooning, inflammation or steatosis) and the proportion of patients with NASH resolution (total absence of ballooning and absent or mild inflammation) without worsening of fibrosis (≥1-stage increase). Key secondary endpoints included the proportion of patients with ≥2-point improvement in NAS and no worsening of fibrosis. Other secondary endpoints included changes from baseline to week 24 in liver parameters (MRI-PDFF, liver chemistry tests and N-terminal type III collagen propeptide [Pro-C3]), and metabolic parameters (adiponectin, serum triglycerides, high-density lipoprotein cholesterol [HDL-C], non-HDL-C, low-density lipoprotein cholesterol [LDL-C] and glycated hemoglobin [HbA1c]). Safety endpoints included the frequency and severity of adverse events (AEs), classified according to Medical Dictionary for Regulatory Activities, version 23.0. Additional safety assessments included safety laboratory parameters, vital signs, electrocardiograms and dual X-ray absorptiometry (DXA) scans.

### Statistical Analysis

A sample size of approximately 184 patients was planned to provide 83-94% power for detection of treatment differences of 30% in the two primary endpoints, based on assumptions of placebo responses and a dropout rate of 15%. To match the intended target population as defined by regulatory authorities, the pre-specified primary efficacy analyses included all patients with F2/F3 fibrosis and NAS ≥4 at baseline who received ≥1 dose of study treatment (full analysis set [FAS]). For primary and key secondary endpoints, results were also analyzed for the FAS plus three randomized but not treated patients with F2/F3 fibrosis and NAS ≥4, and all randomized patients. Safety analyses included all patients who received ≥1 dose of study treatment. Placebo groups were pooled for all analyses.

A multiple imputation strategy and a stratified Cochran-Mantel-Haenszel method were used for analysis of the primary and key secondary endpoints. Sensitivity analyses were conducted to assess the robustness of the primary analysis results. Continuous efficacy endpoints were analyzed using a mixed model repeated measures analysis. There was no pre-specified plan to adjust for multiple comparisons. Comparisons with placebo of the 30 mg and 44 mg dose groups for the first primary outcome (fibrosis improvement) are reported with P-values at a two-sided 0.05 significance level. All other results are reported with only 95% confidence intervals (CIs). The widths of 95% CIs have not been adjusted for multiplicity and should not be used to infer definitive treatment effects. Statistical analyses were conducted using SAS software, version 9.4.

### Results

### Patients

222 patients were randomized, of whom 219 received study treatment and comprised the safety analysis set. Demographics and disease characteristics are presented in Table 7.

TABLE 7
Baseline Demographics and Characteristics of Randomized Patients.*

| | | | | | | | Pegozafermin | | | |
|---|---|---|---|---|---|---|---|---|---|---|
| | | | Pegozafermin | | Pegozafermin | | 44 mg once | | | |
| | Placebo | | 15 mg once | | 30 mg once | | every 2 | | | |
| | (pooled) | | weekly | | weekly | | weeks | | Total | |
| Characteristic | (N = 71) | | (N = 21) | | (N = 73) | | (N = 57) | | (N = 222) | |
| Age - yr | 56.3 ± 9.0 | | 55.0 ± 10.5 | | 55.3 ± 11.2 | | 55.2 ± 11.2 | | 55.6 ± 10.4 | |
| Male sex - no. (%) | 32 | (45) | 12 | (57) | 23 | (32) | 20 | (35) | 87 | (39) |
| White race† - no. | 67 | (94) | 18 | (86) | 69 | (95) | 54 | (95) | 208 | (94) |
| (%) | | | | | | | | | | |
| Hispanic or Latino† | 24 | (34) | 9 | (43) | 32 | (44) | 20 | (35) | 85 | (38) |
| - no. (%) | | | | | | | | | | |
| Body weight - kg | 108.7 ± 20.1 | | 108.0 ± 21.0 | | 95.7 ± 20.2 | | 100.2 ± 20.4 | | 102.2 ± 20.9 | |
| Body mass index - | 38.1 ± 5.6 | | 37.8 ± 4.8 | | 35.1 ± 6.4 | | 36.1 ± 5.5 | | 36.6 ± 5.9 | |
| kg/m² | | | | | | | | | | |
| Type 2 diabetes - no. | 49 | (69) | 18 | (86) | 45 | (62) | 35 | (61) | 147 | (66) |
| (%) | | | | | | | | | | |
| Glycated | 6.6 ± 1.0 | | 7.0 ± 1.2 | | 6.6 ± 1.2 | | 6.7 ± 1.3 | | 6.7 ± 1.2 | |
| hemoglobin - % | | | | | | | | | | |
| Alanine | 49.6 ± 25.7 | | 61.1 ± 34.8 | | 60.0 ± 32.1 | | 56.3 ± 32.0 | | 55.8 ± 30.6 | |
| aminotransferase - | | | | | | | | | | |
| U/L | | | | | | | | | | |
| Aspartate | 40.6 ± 20.2 | | 47.7 ± 27.9 | | 46.7 ± 25.3 | | 41.7 ± 23.3 | | 43.6 ± 23.5 | |
| aminotransferase - | | | | | | | | | | |
| U/L | | | | | | | | | | |
| NASH CRN fibrosis | | | | | | | | | | |
| stage - no. (%) | | | | | | | | | | |
| F1 | 2 | (3) | 3 | (14) | 2 | (3) | 0 | | 7 | (3) |
| F2 | 20 | (28) | 6 | (29) | 21 | (29) | 21 | (37) | 68 | (31) |
| F3 | 47 | (66) | 9 | (43) | 47 | (64) | 30 | (53) | 133 | (60) |
| F4 | 2 | (3) | 3 | (14) | 3 | (4) | 6 | (11) | 14 | (6) |

| | | | | | |
|---|---|---|---|---|---|
| NAS total score | 5.0 ± 1.2 | 4.8 ± 1.2 | 5.3 ± 1.1 | 5.2 ± 1.0 | 5.1 ± 1.1 |
| Liver fat content | 16.7 ± 7.1 | 15.8 ± 6.4 | 16.7 ± 7.0 | 15.8 ± 7.8 | 16.4 ± 7.2 |
| (MRI-PDFF)‡ - % | | | | | |
| Liver stiffness | 14.1 ± 7.7 | 11.2 ± 2.9 | 12.5 ± 4.2 | 13.2 ± 10.3 | 13.0 ± 7.3 |
| (FibroScan vibration- | | | | | |
| controlled transient | | | | | |
| elastography) - kPa | | | | | |
| Pro-C3 - ng/mL | 49.8 ± 17.5 | 61.6 ± 30.7 | 53.6 ± 22.3 | 52.3 ± 18.8 | 52.8 ± 21.1 |
| Triglycerides - | 170.3 ± 84.6 | 186.2 ± 118.7 | 175.0 ± 83.1 | 164.7 ± 77.7 | 171.9 ± 85.8 |
| mg/mL | | | | | |

*Plus-minus signs are means ± SD. CRN, Clinical Research Network; MRI-PDFF, magnetic resonance imaging proton density fat fraction; NAS, Nonalcoholic fatty liver disease Activity Score; NASH, nonalcoholic steatohepatitis; Pro-C3, N-terminal propeptide of type III collagen.
†Patients could be recorded as both White and Hispanic/Latino.
‡Baseline MRI-PDFF data were missing for two patients (3%) in the pooled placebo group and one patient (1%) in the pegozafermin 30 mg once weekly group.
§ Baseline Pro-C3 data were missing for two patients (3%) in the pooled placebo group, one patient (5%) in the pegozafermin 15 mg once weekly group and two patients (3%) in the pegozafermin 30 mg once weekly group.

Most patients were white; African-American patients were under-represented. Mean baseline body mass index and FibroScan vibration-controlled transient elastography (VCTE) were somewhat higher in the placebo group than in the pegozafermin groups. Of the 222 randomized patients, 27 were initially assessed as having F2/F3 fibrosis and NAS ≥4 by a single reader, but later scored as not meeting the study histological inclusion criteria by the three-panel read. These patients were excluded from the FAS, as were three patients who were randomized but not treated. Therefore, 192 patients were included in the FAS. Full agreement or mode determined 91-99% of final biopsy scores.

### Efficacy

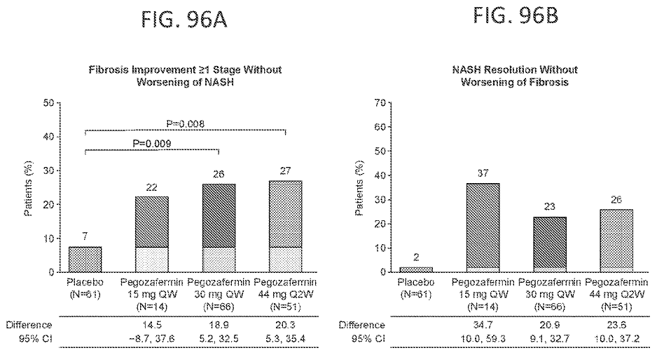

Baseline and 24-week biopsy results were available for 164 patients; outcomes were imputed for the remaining 28 patients in the FAS. After 24 weeks, the proportion of patients who achieved improvement of fibrosis ≥1 stage without worsening of NASH was significantly higher with pegozafermin 30 mg QW (26%; difference [95% CI] 18.9 [5.2-32.5]; P=0.009) and 44 mg Q2W (27%; difference 20.3 [5.3-35.4]; P=0.008) than with placebo (7%) (FIG. 96A). For pegozafermin 15 mg QW, the proportion was 22% (difference 14.5 [−8.7-37.6]). The proportion of patients with NASH resolution without worsening of fibrosis also favored pegozafermin 30 mg QW (23%; difference 20.9 [9.1-32.7]) and 44 mg Q2W (26%; difference 23.6 [10.0-37.2]) versus placebo (2%) (FIG. 96B). For pegozafermin 15 mg QW, the proportion was 37% (difference 34.7 [10.0-59.3]). Results were consistent in pre-specified sensitivity analyses (completer analysis and imputation of missing biopsies as non-response), and in analyses of the FAS plus the three patients with F2/F3 fibrosis and NAS ≥4 who were not treated and all randomized patients. Post-hoc analysis showed 89% of patients treated with pegozafermin who achieved the fibrosis primary endpoint had ≥2-point NAS improvement. In a post-hoc analysis, positive results on fibrosis regression in patients with F4 fibrosis were observed.

Analyses of key secondary endpoints were generally supportive of the primary outcome findings. The proportion of patients with ≥2-point NAS improvement and no worsening of fibrosis was 37% with pegozafermin 15 mg QW, 65% with pegozafermin 30 mg QW, 62% with pegozafermin 44 mg Q2W, and 24% with placebo.

At week 24, the least squares mean percentage change from baseline in hepatic fat fraction (MRI-PDFF) was −27%, −48% and −42% with pegozafermin 15 mg QW, 30 mg QW and 44 mg Q2W, respectively, compared with −5% with placebo (Table 2 and S7). A ≥50% reduction in liver fat versus baseline occurred in 63% and 58% of patients with pegozafermin 30 mg QW and 44 mg Q2W, respectively, compared with 12% of patients on placebo.

Pegozafermin treatment for 24 weeks was associated with reductions in liver chemistry tests (Table 8).

TABLE 8
Changes From Baseline to Week 24 in Selected Liver
and Metabolic End Points (Full Analysis Set).*

| | | Pegozafermin | Pegozafermin | Pegozafermin |
|---|---|---|---|---|
| | Placebo | 15 mg once | 30 mg once | 44 mg once |
| | (pooled) | weekly | weekly | every 2 weeks |
| End point | (N = 61) | (N = 14) | (N = 66) | (N = 51) |
| Alanine aminotransferase | | | | |
| - U/L | | | | |
| Absolute change | −8.8 ± 2.5 | −24.3 ± 5.1 | −26.3 ± 2.4 | −23.5 ± 2.7 |
| Percentage change | −4.6 ± 5.0 | −37.7 ± 10.1 | −41.6 ± 4.8 | −31.8 ± 5.4 |
| Liver fat content (MRI- | | | | |
| PDFF)† - % | | | | |
| Absolute change | −1.5 ± 0.7 | −4.6 ± 1.4 | −8.1 ± 0.7 | −8.2 ± 0.8 |
| Percentage change | −5.0 ± 5.2 | −27.1 ± 10.3 | −48.2 ± 5.1 | −41.9 ± 5.6 |
| Enhanced Liver Fibrosis | | | | |
| test score‡ | | | | |
| Absolute change | 0.2 ± 0.1 | −0.3 ± 0.1 | −0.3 ± 0.1 | −0.3 ± 0.1 |
| Liver stiffness (FibroScan | | | | |
| vibration-controlled | | | | |
| transient elastography)§ | | | | |
| - kPa | | | | |
| Absolute change | 0.8 ± 0.8 | −1.4 ± 1.5 | −3.1 ± 0.8 | −2.4 ± 0.9 |
| Pro-C3 - ng/mL | | | | |
| Absolute change | −1.2 ± 1.8 | −9.9 ± 3.7 | −13.8 ± 1.8 | −11.4 ± 2.0 |
| Percentage change | 6.4 ± 4.1 | −5.4 ± 8.3 | −18.1 ± 4.0 | −17.3 ± 4.4 |
| Iron-corrected T1† - ms | | | | |
| Absolute change | −6.1 ± 11.7 | −46.7 ± 21.3 | −92.4 ± 10.7 | −69.8 ± 12.3 |
| Liver volume - L | | | | |
| Absolute change | −0.1 ± 0.0 | −0.1 ± 0.1 | −0.3 ± 0.0 | −0.2 ± 0.0 |
| Percentage change | −2.5 ± 1.5 | −5.9 ± 3.1 | −12.5 ± 1.5 | −9.5 ± 1.7 |
| Spleen volume - L | | | | |
| Absolute change | 0.0 ± 0.0 | −0.0 ± 0.0 | −0.0 ± 0.0 | −0.0 ± 0.0 |
| Percentage change | 1.3 ± 1.7 | −6.1 ± 3.5 | −9.8 ± 1.7 | −5.2 ± 1.9 |
| Adiponectin - μg/mL | | | | |
| Absolute change | −0.6 ± 0.4 | 1.1 ± 0.7 | 1.1 ± 0.3 | 1.2 ± 0.4 |
| Percentage change | −7.2 ± 5.9 | 20.6 ± 11.9 | 30.0 ± 5.6 | 27.7 ± 6.1 |
| Triglycerides¶ - mg/dL | | | | |
| Absolute change | −7.5 | −8.3 | −40.5 | −14.0 |
| | (−29.0, 19.0) | (−26.8, 10.3) | (−73.0, -7.0) | (−45.5, 7.0) |
| Percentage change | −6.4 | −5.9 | −26.6 | −10.1 |
| | (−17.8, 13.6) | (−22.9, 6.4) | (−39.7, -5.8) | (−28.6, 3.5) |
| Glycated | | | | |
| hemoglobin - % | | | | |
| Absolute change | −0.0 ± 0.1 | −0.1 ± 0.2 | −0.3 ± 0.1 | −0.2 ± 0.1 |

*Plus-minus signs are least squares means ± SE. End points for inclusion in this table were selected based on interest for this type of study in patients with noncirrhotic NASH. Data analyzed using a mixed model with treatment group, week and treatment-by-week interactions as main effects and baseline measurements and stratifications (type 2 diabetes status and fibrosis stage) as covariates. Observed data were used. Enhanced Liver Fibrosis test score, liver stiffness, iron-corrected T1, liver volume and spleen volume were exploratory end points. MRI-PDFF, magnetic resonance imaging proton density fat fraction; NASH, nonalcoholic steatohepatitis; Pro-C3, N-terminal propeptide of type III collagen
†MRI-PDFF analysis set: pooled placebo (N = 57); pegozafermin 15 mg once weekly (N = 14); pegozafermin 30 mg once weekly (N = 61); pegozafermin 44 mg every 2 weeks (N = 49).
‡The Enhanced Liver Fibrosis test score is derived from an algorithm that combines for hyaluronic acid, type III procollagen peptide, and tissue inhibitor of matrix metalloproteinase 1. A score of <7.7 indicates none to mild fibrosis, and a score of ≥11.3 indicates cirrhosis.
§Data analyzed using analysis of covariance with treatment group, baseline measurements and stratifications (type 2 diabetes status and fibrosis stage) as covariates.
¶Data shown as median (Q1, Q3) for non-normal distribution. Analyzed using van Elteren method; patients with missing week 24 values were excluded from the non-parametric analysis.

Alanine aminotransferase (ALT) was normalized (end-of-study ALT <30 U/L in patients with baseline ALT ≥30 U/L) in 59% and 65% of patients in the pegozafermin 30 mg QW and 44 mg Q2W groups, respectively, compared with 24% on placebo (post hoc). The results suggested improvement in corrected T1, which assesses fibro-inflammation, and in the fibrosis markers Enhanced Liver Fibrosis (ELF) test score, FibroScan VCTE, FibroScan-aspartate transaminase (FAST) score, Pro-C3, and Fibrosis-4 (FIB-4) index score, as well as a decrease in liver and spleen volume (Table 8).

The results also suggested that pegozafermin treatment was associated with decreased serum triglycerides and increased HDL-C with pegozafermin 30 mg QW versus placebo (Table 8), and with increased adiponectin levels in all pegozafermin dose groups versus placebo. Results for HbA1c, LDL-C, HDL-C are shown in Table 8. No apparent effect on body weight was observed.

### Safety

Treatment-emergent AEs were reported in 95%, 85%, and 67% of patients on pegozafermin 15 mg QW, 30 mg QW and 44 mg Q2W, respectively, compared with 68% on placebo (Table 9).

TABLE 9
Summary of Treatment-Emergent Adverse Events (Safety Analysis Set).

| | | | Pegozafermin | | Pegozafermin | | Pegozafermin | |
|---|---|---|---|---|---|---|---|---|
| | Placebo | | 15 mg once | | 30 mg once | | 44 mg once | |
| Adverse events - no. | (pooled) | | weekly | | weekly | | every 2 weeks | |
| (%) | (N = 69) | | (N = 21) | | (N = 72) | | (N = 57) | |
| Any adverse event | 47 | (68) | 20 | (95) | 61 | (85) | 38 | (67) |
| Serious adverse events | 3 | (4) | 1 | (5) | 3 | (4) | 6 | (11) |
| Adverse events related to | 20 | (29) | 10 | (48) | 39 | (54) | 24 | (42) |
| study treatment | | | | | | | | |
| Adverse events leading to | 1 | (1) | 1 | (5) | 6 | (8) | 1 | (2) |
| discontinuation of study | | | | | | | | |
| treatment | | | | | | | | |
| Treatment-related adverse | 0 | | 1 | (5) | 4 | (6) | 1 | (2) |
| events leading to | | | | | | | | |
| discontinuation of study | | | | | | | | |
| treatment | | | | | | | | |
| Adverse events from any | | | | | | | | |
| system organ class, | | | | | | | | |
| according to preferred | | | | | | | | |
| term* | | | | | | | | |
| Nausea | 6 | (9) | 4 | (19) | 23 | (32) | 11 | (19) |
| Diarrhea | 4 | (6) | 5 | (24) | 14 | (19) | 8 | (14) |
| Injection site erythema | 3 | (4) | 3 | (14) | 11 | (15) | 3 | (5) |
| Increased appetite | 1 | (1) | 2 | (10) | 10 | (14) | 4 | (7) |
| Vomiting† | 2 | (3) | 1 | (5) | 10 | (14) | 2 | (4) |
| COVID-19† | 6 | (9) | 1 | (5) | 9 | (13) | 5 | (9) |
| Urinary tract infection | 5 | (7) | 0 | | 8 | (11) | 2 | (4) |
| Injection site rash | 1 | (1) | 0 | | 7 | (10) | 2 | (4) |
| Muscle spasms | 1 | (1) | 1 | (5) | 7 | (10) | 0 | |
| Headache | 5 | (7) | 2 | (10) | 6 | (8) | 6 | (11) |

| | | | | | | | |
|---|---|---|---|---|---|---|---|
| Sinusitis | 3 | (4) | 0 | 2 | (3) | 7 | (12) |

| | | | | | | |
|---|---|---|---|---|---|---|
| Lower abdominal pain | 0 | 3 | (14) | 1 | (1) | 0 |

| | | | | | | | |
|---|---|---|---|---|---|---|---|
| Procedural pain | 1 | (1) | 3 | (14) | 0 | 1 | (2) |

*Adverse events with an incidence of at least 10% and experienced by at least three patients in any treatment group are shown.
†Data cut for the main study was Feb. 14, 2023. As of Apr. 24, 2023, one additional case of COVID-19 was reported in the placebo group and one additional case of vomiting in the pegozafermin 30 mg once weekly group.

The most frequent AEs were nausea, diarrhea and injection site erythema. Grade 3 severity AEs were reported in 10%, 4%, and 9% of patients on pegozafermin 15 mg QW, 30 mg QW and 44 mg Q2W, respectively, compared with 9% on placebo. No AEs with severity grade >3 or deaths were reported. Serious AEs were reported in 5%, 4%, and 11% of patients on pegozafermin 15 mg QW, 30 mg QW and 44 mg Q2W, respectively, compared with 4% on placebo. Only one serious AE was considered related to study treatment by the investigator: acute pancreatitis in a patient who received a single dose of pegozafermin 44 mg and had gallbladder sludge on imaging. The clinical course was typical for uncomplicated acute pancreatitis.

Drug-related AEs leading to treatment discontinuation were reported in 5% (diarrhea, N=1), 6% (diarrhea, N=2; nausea, N=1; injection site erythema, N=1), and 2% (pancreatitis, N=1) of patients on pegozafermin 15 mg QW, 30 mg QW and 44 mg Q2W, respectively, compared with 0% on placebo.

No consistent patterns were observed in safety-related laboratory parameters and there were no clinically relevant findings in vital signs or electrocardiograms. No clinically relevant changes were observed in insulin-like growth factor 1, thyroid stimulating hormone or bone biomarkers. No adverse changes were observed in DXA scans after 24 weeks, and fractures, all traumatic, were reported in 3/69 patients in placebo groups and 1/150 patients in pegozafermin groups. No occurrences of potential drug-induced liver injury or tremor were reported.

### Discussion

In this study, treatment with the FGF21 analog pegozafermin at 30 mg QW and 44 mg Q2W for 24 weeks led to significant improvement versus placebo in fibrosis without worsening of NASH. The results also supported benefit on NASH resolution without worsening of fibrosis. Fibrosis progression is a key predictor of clinical outcomes in NASH, including liver-related and all-cause mortality. (Dulai et al, Hepatology 2017; 65:1557-65) Improvement in both steatohepatitis and fibrosis indicates that pegozafermin may impact key aspects of the pathophysiology of NASH.

Marked variability in biopsy reading may contribute to the highly variable placebo response rates in NASH clinical trials. (Ng et al., Hepatology 2022; 75:1647-61) In this study, we used an objective consensus biopsy reading methodology, in which a consensus score, based on the individual scores submitted by three expert pathologists, was determined by a pre-specified algorithm rather than consensus discussion or use of an adjudicator. It is possible that the low placebo rates for the primary endpoints in our study reflected greater accuracy of biopsy reading method, with better estimation of the actual rate of spontaneous regression in NASH (i.e. placebo response). Use of this methodology may have reduced response rates in pegozafermin-treated groups as well.

Corroboration of biopsy findings by noninvasive tests increases confidence in the histological findings. No formal hypothesis testing was conducted for the secondary and exploratory outcomes; however, the results suggest that pegozafermin was associated with improvements in liver fat and in noninvasive markers of liver injury, inflammation and fibrosis, including corrected T1, which assesses fibro-inflammation, and the fibrosis markers ELF, FibroScan VCTE, FAST, Pro-C3, and FIB-4.

NASH is highly associated with the metabolic syndrome. As metabolic derangements contribute to progression of NASH and increase cardiovascular risk, a significant cause of morbidity and mortality in this population,^{3, 4} NASH drugs would ideally improve metabolic comorbidities, but this has not been the case for some classes of therapeutics in development for NASH. (Harrison et al. Gastroenterology 2021; 160:219-31.e1; Neuschwander-Tetri et al., Lancet 2015; 385:956-65; Pockros et al., Liver Int 2019; 39:2082-93; Younossi et al., Lancet 2019; 394:2184-96; Kim et al., Cell Metab 2017; 26:394-406.e6; Francque et al., N Engl J Med 2021; 385:1547-58). In line with findings from previous studies, the results of this trial suggest that pegozafermin may have positive effects on adiponectin, serum triglycerides and HDL-C.

Nausea and diarrhea were the most common AEs with pegozafermin. A single acute pancreatitis serious AE was considered related by the investigator. Effects on bone turnover have been reported in nonclinical studies with FGF21 analogs and in early clinical studies with two FGF21 analogs. (Rader et al., J Clin Endocrinol Metab 2022; 107: e57-e70; Talukdar et al., Cell Metab 2016; 23:427-40; Kim et al., Diabetes Obes Metab 2017; 19:1762-72). There was no signal for reduced bone mass density or fractures in this study, but longer studies are needed to fully assess this potential risk. No hepatotoxicity was observed.

One limitation of this study was its short duration; the single-blind extension study for 24 additional weeks will provide data on longer-term safety and noninvasive biomarker assessments. Another limitation is lack of diversity, as most of the patients were white, potentially limiting the generalizability of the data.

In conclusion, pegozafermin treatment for 24 weeks led to improvements in fibrosis with both weekly and once every 2 weeks dosing in patients with biopsy-confirmed NASH. A potential for dosing once every 2 weeks may increase patient convenience and compliance. Results of the current trial will be informative for guiding dose selection for larger and longer phase 3 studies in NASH.

### Example 12

In this study, subjects were being treated with GLP-1 standard of care therapy (such as semaglutide, dulaglutide, and liraglutide) before initiating the treatment with 44 mg every two-week (Q2W) or 30 mg weekly (QW) doses of mutant Fibroblast Growth Factor-21 (FGF-21) peptide conjugate.

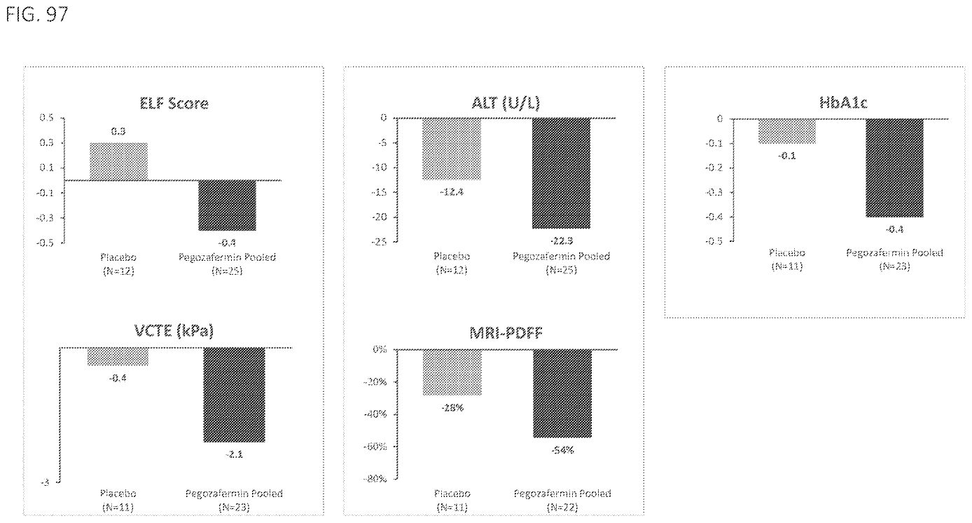

FIG. 97 shows that in patients on background GLP-1 therapy, greater reductions were observed on key markers of liver fibrosis (ELF score, VCTE), liver damage (ALT), glycemic index (HbA1c), and liver fat content (MRI-PDFF), when pegozafermin was added versus placebo. Pooled results include 30 mg and 44 mg groups as results were consistent across both groups.

Tolerability was acceptable with nausea as most common AE. There were no treatment-related discontinuations.

Specific examples of methods and kits have been described herein for purposes of illustration. These are only examples. The technology provided herein can be applied to systems other than the example systems described above. Many alterations, modifications, additions, omissions, and permutations are possible within the practice of this disclosure. This disclosure includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

The embodiments of the disclosure described above are intended to be exemplary only. Those skilled in this art will understand that various modifications of detail may be made to these embodiments, all of which come within the scope of the disclosure.

All publications mentioned herein are hereby incorporated by reference in their entireties. While the foregoing disclosure has been described in some detail for purposes of clarity and understanding, it will be appreciated by one skilled in the art from a reading of the disclosure that various changes in form and detail can be made without departing from the true scope of the disclosure in the appended claims.

### REFERENCES

1. Chalasani N, Younossi Z, Lavine J E, et al. The diagnosis and management of nonalcoholic fatty liver disease: practice guidance from the American Association for the Study of Liver Diseases. Hepatology 2018; 67:328-57.
2. Younossi Z M. The epidemiology of nonalcoholic steatohepatitis. Clin Liver Dis (Hoboken) 2018; 11:92-4.
3. Estes C, Razavi H, Loomba R, Younossi Z, Sanyal A J. Modeling the epidemic of nonalcoholic fatty liver disease demonstrates an exponential increase in burden of disease. Hepatology 2018; 67:123-33.
4. Noureddin M, Vipani A, Bresee C, et al. NASH leading cause of liver transplant in women: updated analysis of indications for liver transplant and ethnic and gender variances. Am J Gastroenterol 2018; 113:1649-59.
5. Khan F Z, Perumpail R B, Wong R J, Ahmed A. Advances in hepatocellular carcinoma: nonalcoholic steatohepatitis-related hepatocellular carcinoma. World J Hepatol 2015; 7:2155-61.
6. Rinella M E. Nonalcoholic fatty liver disease: a systematic review. JAMA 2015; 313:2263-73.
7. Vernon G, Baranova A, Younossi Z M. Systematic review: the epidemiology and natural history of non-alcoholic fatty liver disease and non-alcoholic steatohepatitis in adults. Aliment Pharmacol Ther 2011; 34:274-85.
8. Loomba R, Ratziu V, Harrison S A, NASH Clinical Trial Design International Working Group. Expert panel review to compare FDA and EMA guidance on drug development and endpoints in nonalcoholic steatohepatitis. Gastroenterology 2022; 162:680-8.
9. Taylor R S, Taylor R J, Bayliss S, et al. Association between fibrosis stage and outcomes of patients with nonalcoholic fatty liver disease: a systematic review and meta-analysis. Gastroenterology 2020; 158:1611-25.
10. Loomba R. MRI-proton density fat fraction treatment response criteria in nonalcoholic steatohepatitis. Hepatology 2021; 73:881-3.
11. Tamaki N, Ajmera V, Loomba R. Non-invasive methods for imaging hepatic steatosis and their clinical importance in NAFLD. Nat Rev Endocrinol 2022; 18:55-66.
12. Tamaki N, Munaganuru N, Jung J, et al. Clinical utility of 30% relative decline in MRI-PDFF in predicting fibrosis regression in non-alcoholic fatty liver disease. Gut 2022; 71:983-90.
13. Arner P, Pettersson A, Mitchell P J, et al. FGF21 attenuates lipolysis in human adipocytes—a possible link to improved insulin sensitivity. FEBS Lett 2008; 582:1725-30.
14. Lee D V, Li D, Yan Q, et al. Fibroblast growth factor 21 improves insulin sensitivity and synergizes with insulin in human adipose stem cell-derived (hASC) adipocytes. PLOS One 2014; 9: e111767.
15. Park J G, Xu X, Cho S, et al. CREBH-FGF21 axis improves hepatic steatosis by suppressing adipose tissue lipolysis. Sci Rep 2016; 6:27938.
16. Tillman E J, Rolph T. FGF21: an emerging therapeutic target for non-alcoholic steatohepatitis and related metabolic diseases. Front Endocrinol (Lausanne) 2020; 11. 601290.
17. Sanyal A, Charles E D, Neuschwander-Tetri B A et al. Pegbelfermin (BMS-986036), a PEGylated fibroblast growth factor 21 analogue, in patients with non-alcoholic steatohepatitis: a randomised, double-blind, placebo-controlled, phase 2a trial. Lancet 2019; 392:2705-17.
18. Charles E D, Neuschwander-Tetri B A, Pablo Frias J, et al. Pegbelfermin (BMS-986036), PEGylated FGF21, in patients with obesity and type 2 diabetes: results from a randomized phase 2 study. Obesity 2019; 27:41-9.
19. Geng L, Lam KSL, Xu A. The therapeutic potential of FGF21 in metabolic diseases: from bench to clinic. Nat Rev Endocrinol 2020; 16:654-67.
20. Mu J, Pinkstaff J, Li Z, et al. FGF21 analogs of sustained action enabled by orthogonal biosynthesis demonstrate enhanced antidiabetic pharmacology in rodents. Diabetes 2012; 61:505-12.
21. Rosenstock M, Ayalon M, Mansbach H, Liu Y, Margalit M. LBP-29-BIO89-100, a novel PEG-FGF21 analogue, is efficacious following weekly and every 2-week subcutaneous dosing in spontaneous diabetic cynomolgus monkeys. J Hepatol 2019; 70: e155.
22. Rosenstock M, Rouquet T, Bariohay B, Mansbach H, Margalit M. BIO89-100, a novel glycopegylated FGF21 analog, reduces body weight and fat mass in naive CD-1 mice through an increase in energy expenditure despite an increase in food consumption. J Hepatol 2020; 73: S452.
23. Margalit M, Chen C-Y, Tseng L, Rosenstock M, Mansbach H. BIO89-100, a GlycoPEGylated FGF21 analog, improved serum lipids and extended half-life in a controlled single ascending dose trial in healthy subjects. J Clin Lipidol 2020; 14:585-6.
24. Lin S C, Heba E, Bettencourt R, et al. Assessment of treatment response in non-alcoholic steatohepatitis using advanced magnetic resonance imaging. Aliment Pharmacol Ther 2017; 45:844-54.
25. Hadizadeh F, Faghihimani E, Adibi P. Nonalcoholic fatty liver disease: diagnostic biomarkers. World J Gastrointest Pathophysiolog
26. Loomba R, Sanyal A J, Kowdley K V, et al. Factors associated with histologic response in adult patients with nonalcoholic steatohepatitis. Gastroenterology 2019; 156:88-95.
27. Boyle M, Tiniakos D, Schattenberg J M, et al. Performance of the PRO-C3 collagen neo-epitope biomarker in non-alcoholic fatty liver disease. JHEP Rep 2019; 1:188-98.
28. Daniels S J, Leeming D J, Eslam M, et al. ADAPT: An algorithm incorporating PRO-C3 accurately identifies patients with NAFLD and advanced fibrosis. Hepatology 2019; 69:1075-86.
29. Luo Y, Oseini A, Gagnon R, et al. An evaluation of the collagen fragments related to fibrogenesis and fibrolysis in nonalcoholic steatohepatitis. Sci Rep 2018; 8:12414.
30. Eslam M, Newsome P N, Sarin S K, et al. A new definition for metabolic dysfunction-associated fatty liver disease: an international expert consensus statement. J Hepatol 2020; 73:202-9.
31. Shroff H, VanWagner L B. Cardiovascular disease in nonalcoholic steatohepatitis: screening and management. Curr Hepatol Rep 2020; 19:315-26.
32. Oscarsson J, Onnerhag K, Riserus U, et al. Effects of free omega-3 carboxylic acids and fenofibrate on liver fat content in patients with hypertriglyceridemia and non-alcoholic fatty liver disease: a double-blind, randomized, placebo-controlled study. J Clin Lipidol 2018; 12:1390-403.
33. Yamauchi T, Kamon J, Waki H, et al. The fat-derived hormone adiponectin reverses insulin resistance associated with both lipoatrophy and obesity. Nat Med 2001; 7:941-6.
34. Lin Z, Tian H, Lam S L, Karen, et al. Adiponectin mediates the metabolic effects of FGF21 on glucose homeostasis and insulin sensitivity in mice. Cell Metab 2013; 17:779-89.
35. Sonoda J, Chen M Z, Baruch A. FGF21-receptor agonists: an emerging therapeutic class for obesity-related diseases. Horm Mol Biol Clin Investig 2017; 30: /j/hmbci.2017.30.issue-2/hmbci-2017-0002/hmbci-2017-0002.xml.
36. Harrison S A, Rossi S J, Paredes A H, et al. NGM282 Improves liver fibrosis and histology in 12 weeks in patients with nonalcoholic steatohepatitis. Hepatology 2020; 71:1198-212.
37. Harrison S A, Ruane P J, Freilich B L, et al. Efruxifermin in non-alcoholic steatohepatitis: a randomized, double-blind, placebo-controlled, phase 2a trial. Nat Med 2021; 27:1262-71.
38. Younossi Z M, Ratziu V, Loomba R, et al. Obeticholic acid for the treatment of non-alcoholic steatohepatitis: interim analysis from a multicentre, randomised, placebo-controlled phase 3 trial. Lancet 2019; 394:2184-96.
39. Harrison S A, Rinella M E, Abdelmalek M F, et al. NGM282 for treatment of non-alcoholic steatohepatitis: a multicentre, randomised, double-blind, placebo-controlled, phase 2 trial. Lancet 2018; 391:1174-85.
40. Harrison S A, Bashir M R, Guy C D, et al. Resmetirom (MGL-3196) for the treatment of non-alcoholic steatohepatitis: a multicentre, randomised, double-blind, placebo-controlled, phase 2 trial. Lancet 2019; 394:2012-24.
41. Flint A, Andersen G, Hockings P, et al. Randomised clinical trial: semaglutide versus placebo reduced liver steatosis but not liver stiffness in subjects with non-alcoholic fatty liver disease assessed by magnetic resonance imaging. Aliment Pharmacol Ther 2021; 54:1150-61.
42. Neuschwander-Tetri B A, Loomba R, Sanyal A J et al. Farnesoid X nuclear receptor ligand obeticholic acid for non-cirrhotic, non-alcoholic steatohepatitis (FLINT): a multicentre, randomised, placebo-controlled trial. Lancet 2015; 385:956-65.
43. Pockros P J, Fuchs M, Freilich B, et al. CONTROL: a randomized phase 2 study of obeticholic acid and atorvastatin on lipoproteins in nonalcoholic steatohepatitis patients. Liver Int 2019; 39:2082-93.
44. Harrison S A, Neff G, Guy C D, et al. Efficacy and safety of aldafermin, an engineered FGF19 analog, in a randomized, double-blind, placebo-controlled trial of patients with nonalcoholic steatohepatitis. Gastroenterology 2021; 160:219-31.
45. Kim C W, Addy C, Kusunoki J, et al. Acetyl COA carboxylase inhibition reduces hepatic steatosis but elevates plasma triglycerides in mice and humans: a bedside to bench investigation. Cell Metab 2017; 26:394-406.
46. Francque S M, Bedossa P, Ratziu V, et al. A randomized, controlled trial of the pan-PPAR agonist lanifibranor in NASH. N Engl J Med 2021; 385:1547-58.

1. A method of treating nonalcoholic steatohepatitis (NASH) in a subject in need thereof, comprising:
administering once a week to the subject in need thereof a pharmaceutical composition comprising from 15 mg to 30 mg of a mutant Fibroblast Growth Factor-21 (FGF-21) peptide conjugate and a pharmaceutically acceptable carrier,
wherein the mutant FGF-21 peptide conjugate comprises:
i) a mutant FGF-21 peptide comprising the amino acid sequence of SEQ ID NO: 2,
ii) a glycosyl moiety, and
iii) a 20 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG,
wherein administration of the pharmaceutical composition results in at least one of:
a reduction of liver fat,
an improvement of liver fibrosis score,
resolution of NASH,
≥2 point improvement of NAFLD Activity score,
improvement of VCTE score,
improvement of FAST score,
improvement of FIB-4 score,
a reduction of liver size as assessed by Magnetic resonance imaging-Proton density fat fraction,
a reduction of levels of one or more biomarkers comprising Pro-C3, alanine transaminase (ALT), Enhanced LiverFibrosis (ELF) panel, CK-18, inflammation marker high-sensitivity C-reactive protein (hs-CRP), Hemoglobin Alc (HbA1c), non-HDL-c, LDL-c, and Triglycerides, and
an increase of the levels of HDL-c and/or adiponectin.

2. (canceled)

3. The method of claim 1, wherein the pharmaceutical composition is administered sub-subcutaneously.

4. The method of claim 1, wherein the glycosyl moiety comprises at least one of an N-acetylgalactosamine (GalNAc) residue, a galactose (Gal) residue, a sialic acid (Sia) residue, a 5-amine analogue of a Sia residue, a mannose (Man) residue, mannosamine, a glucose (Glc) residue, an N-acetylglucosamine (GlcNAc) residue, a fucose residue, a xylose residue, or a combination thereof.

5. The method of claim 1, wherein the glycosyl moiety comprises at least one N-acetylgalactosamine (GalNAc) residue, at least one galactose (Gal) residue, at least one sialic acid (Sia) residue, or a combination thereof.

6. The method of claim 5, wherein the at least one Sia residue is a nine-carbon carboxylated sugar.

7. The method of claim 6, wherein the at least one Sia residue is N-acetyl-neuraminic acid (2-keto-5-acetamido-3,5-dideoxy-D-glycero-D-galactononulopyranos-1-onic acid (Neu5Ac), N-glycolylneuraminic acid (Neu5Gc), 2-keto-3-deoxy-nonulosonic acid (KDN), or a 9-substituted sialic acid.

8. The method of claim 7, wherein the 9-substituted sialic acid is 9-O-lactyl-Neu5Ac, 9-O-acetyl-Neu5Ac, 9-deoxy-9-fluoro-Neu5Ac, or 9-azido-9-deoxy-Neu5Ac.

9. The method of claim 1, wherein the glycosyl moiety comprises the structure -GalNAc-Sia-.

10. The method of claim 1, wherein the 20 kDa PEG moiety is attached to the glycosyl moiety by a covalent bond to a linker, wherein the linker comprises at least one amino acid residue.

11. The method of claim 10, wherein the at least one amino acid residue is a glycine (Gly).

12. The method of claim 1, wherein the mutant FGF-21 peptide conjugate comprises the structure-GalNAc-Sia-Gly-PEG (20 kDa).

13. The method of claim 1, wherein the mutant FGF-21 peptide conjugate comprises the structure: 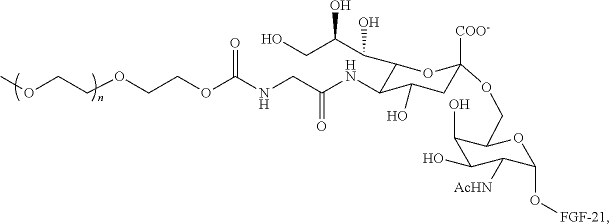
wherein n is an integer selected from 450 to 460.

14. The method of claim 1, wherein the 20 kDa PEG is a linear or branched PEG.

15. The method of claim 1, wherein the 20 kDa PEG is a 20 kDa methoxy-PEG.

16. (canceled)

17. The method of claim 1, comprising administering once a week to the subject in need thereof the pharmaceutical composition comprising from about 25 mg to about 30 mg of the mutant FGF-21 peptide conjugate.

18. The method of claim 1, comprising administering once a week to the subject in need thereof the pharmaceutical composition comprising 30 mg of the mutant FGF-21 peptide conjugate.

19. A method of treating nonalcoholic steatohepatitis (NASH) in a subject in need thereof, comprising:
administering once every two weeks to the subject in need thereof a pharmaceutical composition comprising from 18 mg to 44 mg of a mutant Fibroblast Growth Factor-21 (FGF-21) peptide conjugate and a pharmaceutically acceptable carrier,
wherein the mutant FGF-21 peptide conjugate comprises:
i) a mutant FGF-21 peptide comprising the amino acid sequence of SEQ ID NO: 2,
ii) a glycosyl moiety, and
iii) a 20 kDa polyethylene glycol (PEG), wherein the mutant FGF-21 peptide is attached to the glycosyl moiety by a covalent bond between a threonine at amino acid position 173 of SEQ ID NO: 2 and a first site of the glycosyl moiety and wherein the glycosyl moiety is attached to the 20 kDa PEG by a covalent bond between a second site of the glycosyl moiety and the 20 kDa PEG,
wherein administration of the pharmaceutical composition results in at least one of:
a reduction of liver fat,
an improvement in liver fibrosis score,
NASH resolution,
≥2 point improvement of NAFLD Activity score,
improvement of VCTE score,
improvement of FAST score,
improvement of FIB-4 score,
a reduction of liver size as assessed by Magnetic resonance imaging-Proton density fat fraction,
a reduction of levels of one or more biomarkers comprising Pro-C3, alanine transaminase (ALT), Enhanced Liver Fibrosis (ELF) panel, CK-18, inflammation marker high-sensitivity C-reactive protein (hs-CRP), Hemoglobin Alc (HbA1c), Triglycerides, LDL-c, non-HDL-c, and
an increase of the levels of HDL-c and/or adiponectin.

20. (canceled)

21. The method of claim 19, wherein the pharmaceutical composition is administered sub-subcutaneously.

22. The method of claim 19, wherein the glycosyl moiety comprises at least one of an N-acetylgalactosamine (GalNAc) residue, a galactose (Gal) residue, a sialic acid (Sia) residue, a 5-amine analogue of a Sia residue, a mannose (Man) residue, mannosamine, a glucose (Glc) residue, an N-acetylglucosamine (GlcNAc) residue, a fucose residue, a xylose residue, or a combination thereof.

23. The method of claim 19, wherein the glycosyl moiety comprises at least one N-acetylgalactosamine (GalNAc) residue, at least one galactose (Gal) residue, at least one sialic acid (Sia) residue, or a combination thereof.

24. The method of claim 23, wherein the at least one Sia residue is a nine-carbon carboxylated sugar.

25. The method of claim 24, wherein the at least one Sia residue is N-acetyl-neuraminic acid (2-keto-5-acetamido-3,5-dideoxy-D-glycero-D-galactononulopyranos-1-onic acid (Neu5Ac), N-glycolylneuraminic acid (Neu5Gc), 2-keto-3-deoxy-nonulosonic acid (KDN), or a 9-substituted sialic acid.

26. The method of claim 25, wherein the 9-substituted sialic acid is 9-O-lactyl-Neu5Ac, 9-O-acetyl-Neu5Ac, 9-deoxy-9-fluoro-Neu5Ac, or 9-azido-9-deoxy-Neu5Ac.

27. The method of claim 19, wherein the glycosyl moiety comprises the structure-GalNAc-Sia-.

28. The method of claim 19, wherein the 20 kDa PEG moiety is attached to the glycosyl moiety by a covalent bond to a linker, wherein the linker comprises at least one amino acid residue.

29. The method of claim 28, wherein the at least one amino acid residue is a glycine (Gly).

30. The method of claim 19, wherein the mutant FGF-21 peptide conjugate comprises the structure-GalNAc-Sia-Gly-PEG (20 kDa).

31. The method of claim 19, wherein the mutant FGF-21 peptide conjugate comprises the structure: 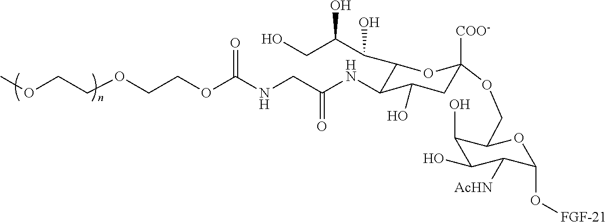
wherein n is an integer selected from 450 to 460.

32. The method of claim 19, wherein the 20 kDa PEG is a linear or branched PEG.

33. The method of claim 19, wherein the 20 kDa PEG is a 20 kDa methoxy-PEG.

34. The method of claim 19, comprising administering once a week to the subject in need thereof the pharmaceutical composition comprising from about 40 mg to about 50 mg of the mutant FGF-21 peptide conjugate.

35. The method of claim 19, comprising administering once every two weeks to the subject in need thereof the pharmaceutical composition comprising 44 mg of the mutant FGF-21 peptide conjugate.
